(19) United States
(12) Patent Application Publication
(10) Pub. No.: US 2026/0365632 A1
(43) Pub. Date: Nov. 19, 2026

(54) PIKfyve Kinase Inhibitors

(71) Applicant: Dunad Therapeutics Ltd., Duxford (GB)

(72) Inventors: Barry John TEOBALD, Duxford (GB); Lou CHUPAK, Duxford (GB); Mirco MENICONI, Duxford (GB); Graham L. SIMPSON, Duxford (GB); Laura D'AGOSTINO, Duxford (GB)

(21) Appl. No.: 19/704,325

(22) Filed: Jun. 10, 2026

Related U.S. Application Data
(63) Continuation of application No. PCT/US2025/059633, filed on Dec. 15, 2025.
(60) Provisional application No. 63/734,396, filed on Dec. 16, 2024.
(60) Provisional application No. 63/857,239, filed on Aug. 4, 2025.

(51) Int. Cl. C07D 401/12; C07D 401/14; C07D 405/14; C07D 409/14; C07D 413/14; C07D 417/14; C07D 471/04; C07D 471/10; C07D 491/08; C07D 491/107; C07D 495/04; C07D 498/04

(52) U.S. Cl. CPC C07D 401/12; C07D 401/14; C07D 405/14; C07D 409/14; C07D 413/14; C07D 417/14; C07D 471/04; C07D 471/10; C07D 491/08; C07D 491/107; C07D 495/04; C07D 498/04

(57) ABSTRACT

The present invention relates to compounds of Formula I useful as inhibitors of phosphatidylinositol-3-phosphate 5-kinase (PIKfyve) as well as their use for treating diseases and disorders associated with PIKfyve. In particular, the PIKfyve inhibitors of the invention comprise a functional group modification that allows for a covalent interaction with PIKfyve. In some embodiments, a compound of the invention forms a covalent bond with an amino acid residue of the PIKfyve protein.

### RELATED APPLICATIONS

This application is a continuation of International Application No. PCT/US2025/059633, which designated the United States and was filed on Dec. 15, 2025, published in English, which claims the benefit of U.S. Provisional Application No. 63/857,239 filed Aug. 4, 2025, and U.S. Provisional Application No. 63/734,396 filed Dec. 16, 2024. The entire contents of the above-referenced applications are incorporated by reference herein.

### BACKGROUND OF THE INVENTION

Phosphatidylinositol-3-phosphate 5-kinase (PIKfyve) is named after its function and domain structure (phosphoinositide kinase for five position containing a FYVE finger) is a lipid kinase that phosphorylates phosphatidylinositol-3-phosphate (PI(3)P), producing PI(3,5)P₂, which is involved in cellular processes including membrane trafficking and cytoskeletal reorganization. Inhibiting PIKfyve can alter both lipid levels and TFEB-driven transcription leading to changes autophagy and resultant increases in autophagic flux and exocytosis and downstream degradation/clearance of protein aggregates (Hung et al., Staats et al., Shi et al., Soares et al., Lucus-Del-Pozo et al.). Inhibiting PIKfyve may be useful in the treatment of various diseases or conditions such as viral infections, autoimmune diseases, inflammatory diseases, cancer, pain, or many neurological diseases. Thus, there is a need for improved inhibitors of PIKfyve.

### SUMMARY OF THE INVENTION

The present invention relates to compounds useful as inhibitors of phosphatidylinositol-3-phosphate 5-kinase (PIKfyve) as well as their use for treating diseases and disorders associated with PIKfyve. In particular, the PIKfyve inhibitors of the invention comprise a functional group modification that allows for a covalent interaction with PIKfyve. In some embodiments, a compound of the invention forms a covalent bond with an amino acid residue of the PIKfyve protein.

One of ordinary skill in the art will recognize that certain reactive functional groups can act as an “warheads.” As used herein, the term “warhead” or “warhead group” refers to a functional group present on a compound of the present invention wherein that functional group is capable of covalently binding to an amino acid residue (such as cysteine, lysine, histidine, or other residues capable of being covalently modified), present in or near the binding pocket of the PIKfyve target protein, thereby inhibiting the PIKfyve protein. In some embodiments, the covalent binding of the compound of the invention to the amino acid of the PIKfyve protein is reversible. In some embodiments, the covalent binding of the compound of the invention to the amino acid of the PIKfyve protein is irreversible.

It will be appreciated that in some embodiments the Linker-Warhead group (Q-W), as defined and described herein, provides such warhead groups for covalently, and reversibly, inhibiting the PIKfyve protein.

It will be appreciated that in some embodiments the Linker-Warhead group (Q-W), as defined and described herein, provides such warhead groups for covalently, and irreversibly, inhibiting the PIKfyve protein.

In some embodiments, a compound of the invention forms a covalent bond with a sulfur atom of a cysteine residue of the PIKfyve protein. In embodiments, the covalent bonding between the compound of the invention and the cysteine residue of PIKfyve is irreversible.

Any suitable method for determining a reversible or an irreversible (e.g., covalently reversible or irreversible) interaction between a compound of the invention and the PIKfyve protein can be used. Methods for identifying if a compound is acting as a reversible or an irreversible inhibitor are known to one of ordinary skill in the art.

In one aspect, the present invention provides a compound of formula (I):

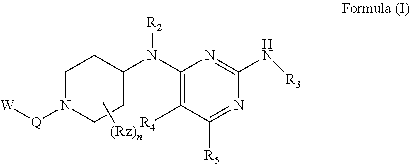

or a pharmaceutically acceptable salt thereof, wherein:
R₂ is independently selected from hydrogen, deuterium (D), halogen, a substituted or unsubstituted alkyl, a substituted or unsubstituted alkoxy, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, a substituted or unsubstituted heteroaryl, and a substituted or unsubstituted heteroalkyl;
R₃ is selected from substituted or unsubstituted aryl, substituted or unsubstituted heteroaryl, substituted or unsubstituted cycloalkyl, or substituted or unsubstituted heterocycle, wherein the aryl, heteroaryl, cycloalkyl, or heterocycle can be optionally fused or bridged;
R₄ and R₅ are each independently selected from hydrogen, D, halogen, substituted or unsubstituted alkyl, substituted or unsubstituted alkoxy, substituted or unsubstituted fluoroalkyl, and —CN;
or R₄ and R₅ can be taken together with the carbons to which they are attached to form a substituted or unsubstituted 4, 5, 6, 7, or 8 membered ring;
or R₂ and R₄ can be taken together to form a substituted or unsubstituted heterocycle; Q is selected from absent, —C(O)—, —CR_{B}R_{C}—, —S(O)₂—, —S(O)—, —NR_{A}—, —C(O)O—, —NR_{A}C(O)—, —C(O)—CR_{B}R_{C}—, and —C(O)NR_{A}—;
R_{B} and R_{C} are independently H, halogen, a substituted or unsubstituted alkyl, substituted or unsubstituted alkenyl, substituted or unsubstituted alkynyl, or substituted or unsubstituted alkoxy, and
R_{A} is hydrogen, substituted or unsubstituted alkyl, substituted or unsubstituted haloalkyl, substituted or unsubstituted heteroalkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl;
or R_{B} and R_{C} can be taken together with the atom to which they are attached to form a substituted or unsubstituted 4, 5, 6, 7, or 8 membered ring;
W is a substituted or unsubstituted alkyl, a substituted or unsubstituted alkenyl, a substituted or unsubstituted aryl or a substituted or unsubstituted heteroaryl;
each R_{Z} is independently selected from D, halogen, —CN, a substituted or unsubstituted alkyl, a substituted or unsubstituted alkoxy, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, a substituted or unsubstituted heteroaryl, and a substituted or unsubstituted heteroalkyl;
alternatively, (R_{Z})ₙ, together with the heterocyclic ring to which R_{Z} is attached, forms a bridged, fused, or spiro ring structure; and n is 0, 1, 2, 3, or 4.

In another aspect, described herein is a pharmaceutical composition comprising a compound as described herein or a salt thereof and one or more of pharmaceutically acceptable carriers.

In another aspect, described herein is a method of inhibiting PIKfyve in a subject (such as a human subject) in need thereof comprising administering an effective amount of a compound of the present invention to the subject.

In yet another aspect, described herein is a method for treating a disease or disorder associated with PIKfyve in a human subject in need thereof comprising administering an effective amount of a compound of the present invention to the subject.

### DETAILED DESCRIPTION OF THE INVENTION

Provided herein are compounds or pharmaceutically acceptable salts thereof, and compositions that inhibit phosphatidylinositol-3-phosphate 5-kinase (PIKfyve). As such, in one aspect, the compounds and compositions provided herein are useful in inhibiting PIKfyve and/or for treating a disease or disorder associated with PIKfyve in a subject (such as a human subject) in need thereof.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting. The terminology used herein is for the purpose of describing particular embodiments only and is not intended to be limiting.

The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of 10%, including ±5%, ±1%, and ±0.1% from the specified value, as such variations are appropriate to perform the disclosed methods.

The term “treat,” “treated,” “treating,” or “treatment” refers to an approach for obtaining beneficial or desired results including but not limited to therapeutic benefit. By therapeutic benefit is meant eradication or amelioration of the underlying state, disease, or disorder being treated. Also, a therapeutic benefit includes the diminishment or alleviation of at least one symptom associated or caused by the state, disorder or disease being treated.

As used herein, the term “prevent” or “prevention” means no disorder or disease development if none had occurred, or no further disorder or disease development if there had already been development of the disorder or disease. Also considered is the ability of one to prevent one, some, or all of the symptoms associated with the disorder or disease.

As used herein, the term “patient,” “individual” or “subject” includes a mammal. The mammal can be e.g., any mammal, e.g., a human, a primate, a mouse, a rat, a dog, a cat, a cow, a horse, a goat, a camel, a sheep, or a pig. Preferably, the mammal is a human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “administration” of a compound or a composition to a subject includes any route of introducing or delivering the agent to a subject to perform its intended function. Administration can be carried out by any suitable oral or non-oral route as described herein. Administration includes self-administration and the administration by another.

The term “co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

The term “alkyl”, unless otherwise specified, refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (isopropyl), n-butyl, n-pentyl, and 1,1-dimethylethyl (t-butyl). The term “C₁₋₆ alkyl” refers to an alkyl group as defined above having 1 to 6 carbon atoms. The term “C₁₋₃ alkyl” refers to an alkyl group as defined above having 1 to 3 carbon atoms. In appropriate circumstances, the term “alkyl” refers to a hydrocarbon chain radical as mentioned above which is bivalent.

The term “alkenyl”, unless otherwise specified, refers to an aliphatic hydrocarbon group containing one or more carbon-carbon double bonds and which may be a straight or branched or branched chain having about 2 to about 10 carbon atoms, e.g., ethenyl, 1-propenyl, 2-propenyl (allyl), iso-propenyl, 2-methyl-1-propenyl, 1-butenyl, and 2-butenyl. The term “C₂₋₆ alkenyl” refers to an alkenyl group as defined above having 2 to 6 carbon atoms. In appropriate circumstances, the term “alkenyl” refers to a hydrocarbon group as mentioned above which is bivalent.

The term “alkynyl”, unless otherwise specified, refers to a straight or branched chain hydrocarbyl radical having at least one carbon-carbon triple bond, and having in the range of 2 to up to 12 carbon atoms (with radicals having in the range of 2 to up to 10 carbon atoms presently being preferred) e.g., ethynyl, propynyl, and butnyl. The term “C₂₋₆ alkynyl” refers to an alkynyl group as defined above having 2 to 6 carbon atoms. In appropriate circumstances, the term “alkynyl” refers to a hydrocarbyl radical as mentioned above which is bivalent.

The term “alkoxy” unless otherwise specified, denotes an alkyl, cycloalkyl, or cycloalkylalkyl group as defined herein attached via an oxygen linkage to the rest of the molecule. The term “substituted alkoxy” refers to an alkoxy group where the alkyl constituent is substituted (i.e., —O-(substituted alkyl)). For example, “alkoxy” refers to the group —O-alkyl, including from 1 to 8 carbon atoms of a straight, branched, cyclic configuration and combinations thereof attached to the parent structure through an oxygen atom. Examples include methoxy, ethoxy, propoxy, isopropoxy, cyclopropyloxy, and cyclohexyloxy. In appropriate circumstances, the term “alkoxy” refers to a group as mentioned above which is bivalent.

The term “cycloalkyl”, unless otherwise specified, denotes a non-aromatic mono or multicyclic ring system of about 3 to 12 carbon atoms such as cyclopropyl, cyclobutyl, cyclopentyl, and cyclohexyl. Examples of multicyclic cycloalkyl groups include perhydronaphthyl, adamantyl and norbornyl groups, bridged cyclic groups, and sprirobicyclic groups, e.g., spiro[4.4]non-2-yl. The term “C₃₋₆ cycloalkyl” refers to a cycloalkyl group as defined above having 3 to 6 carbon atoms.

The term “cycloalkylalkyl”, unless otherwise specified, refers to a cyclic ring-containing radical containing in the range of about 3 up to 8 carbon atoms directly attached to an alkyl group which is then attached to the main structure at any carbon from the alkyl group, such as cyclopropylmethyl, cyclobutylethyl, and cyclopentylethyl.

The term “cycloalkenyl”, unless otherwise specified, refers to cyclic ring-containing radicals containing in the range of about 3 to 8 carbon atoms with at least one carbon-carbon double bond such as cyclopropenyl, cyclobutenyl, and cyclopentenyl.

The term “cycloalkenylalkyl” refers to a cycloalkenyl group directly attached to an alkyl group which is then attached to the main structure at any carbon from the alkyl group.

The term “aryl”, unless otherwise specified, refers to aromatic radicals having in the range of 6 to 20 carbon atoms such as phenyl, naphthyl, tetrahydronaphthyl, indanyl, and biphenyl.

The term “arylalkyl”, unless otherwise specified, refers to an aryl group as defined above directly bonded to an alkyl group as defined above, e.g., —CH₂C₆H₅ and —C₂H₅C₆H₅.

The term “heterocyclic ring”, unless otherwise specified, refers to a non-aromatic 3- to 15-member ring radical which consists of carbon atoms and at least one heteroatom selected from nitrogen, phosphorus, oxygen and sulfur. For purposes of this invention, the heterocyclic ring radical may be a mono-, bi-, tri- or tetracyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized. The heterocyclic ring radical may be attached to the main structure at any heteroatom or carbon atom. Examples of such heterocyclic radicals include, but are not limited to, piperidinyl, piperazinyl, 2-oxopiperazinyl, 2-oxopiperidinyl, 2-oxopyrrolidinyl, 2-oxoazepinyl, azepinyl, 4-piperidonyl, pyrrolidinyl, oxazolinyl, morpholinyl, quinuclidinyl, octahydroindolyl, octahydroisoindolyl, decahydroisoquinolyl, tetrahydrofuryl, tetrahydropyranyl, thiamopholinyl, thiamorpholinyl sulfoxide, thiamopholinyl sulfone, and dioxaphospholanyl.

The term “heterocyclyl”, unless otherwise specified, refers to a heterocylic ring radical as defined above. The heterocylcyl ring radical may be attached to the main structure at any heteroatom or carbon ring atom. In appropriate circumstances, the term “heterocyclyl” refers to a hydrocarbon chain radical as mentioned above which is bivalent. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

The term “heterocyclylalkyl”, unless otherwise specified, refers to a radical of the formula —R^{w}-heterocyclyl where R^{w} is an alkylene chain as defined herein. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined herein for an alkylene chain. The heterocyclyl part of the heterocyclylalkyl radical is optionally substituted as defined herein for a heterocyclyl group.

The term “heteroaryl”, unless otherwise specified, refers to an optionally substituted 5 to 14-member aromatic ring having one or more heteroatoms selected from N, O, and S as ring atoms. The heteroaryl may be a mono-, bi- or tricyclic ring system. Examples of such “heteroaryl” radicals include, but are not limited to, oxazolyl, thiazolyl, imidazolyl, pyrrolyl, furanyl, pyridinyl, pyrimidinyl, pyrazinyl, benzofuranyl, indolyl, benzothiazolyl, benzoxazolyl, carbazolyl, quinolyl, isoquinolyl, azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, tetrazoyl, tetrahydroisoquinolyl, pyridazinyl, oxazolidinyl, triazolyl, indanyl, isoxazolyl, isoxazolidinyl, thiazolinyl, thiazolidinyl, isothiazolyl, isothiazolidinyl, isoindolyl, indolinyl, isoindolinyl, benzimidazolyl, thiadiazolyl, benzopyranyl, thienyl, benzothienyl, oxadiazolyl, chromanyl, and isochromanyl. The term “5 or 6-membered heteroaryl” refers to a heteroaryl having 5- or 6-ring atoms. The term “5-6 or 6-5 membered bicyclic heteroaryl” refers to a bicyclic heteroaryl with a five- membered ring fused to a six-membered ring, where the 5-membered ring is bound to the rest of the molecule (referred to as a “5-6 membered bicyclic heteroaryl”) or the 6-membered ring is bound to the rest of the molecule (referred to as a “6-5 membered bicyclic heteroaryl”). The term “6-6 membered bicyclic heteroaryl” refers to a bicyclic heteroaryl with a six membered ring fused to a another six-membered ring, where one of the 6-membered rings is bound to the rest of the molecule. The heteroaryl ring radical may be attached to the main structure at any heteroatom or carbon atom. The term “substituted heteroaryl” also includes ring systems substituted with one or more oxide (—O—) substituents, such as pyridinyl N-oxides.

The term “heteroarylalkyl”, unless otherwise specified, refers to a radical of the formula —R^{w}-heteroaryl, where R^{w} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined herein for an alkylene chain. The heteroaryl part of the heteroarylalkyl radical is optionally substituted as defined herein for a heteroaryl group.

The term “cyclic ring” refers to a cyclic ring containing 3 to 10 carbon atoms.

The term “substituted” unless otherwise specified, refers to substitution with any one or any combination of the following substituents which may be the same or different and are independently selected from hydrogen, hydroxy, halogen, carboxyl, cyano, nitro, oxo (═O), thio (═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted heteroaryl, substituted or unsubstituted heteroarylalkyl, substituted or unsubstituted heterocyclic ring, substituted heterocyclylalkyl ring, substituted or unsubstituted guanidine, —COOR^{a}, —C(O)R^{a}, —C(S)R^{a}, —C(O)NR^{a}R^{b}, —C(O)NR^{a}(OR^{b}), —NR^{b}R^{c}, —NR^{a}CONR^{b}R^{c}, —N(R^{a})SOR^{b}, —N(R^{a})SO₂R^{b}, ═N—NR^{a}R^{b}, —NR^{a}C(O)OR^{b}, —NR^{a}R^{b}, —NR^{a}C(O)R^{b}, —NR^{a}C(S)R^{b}, —NR^{a}C(S)NR^{b}R^{c}, —SONR^{a}R^{b}, —SO₂NR^{a}R^{b}, —OR^{a}, —OR^{a}C(O)NR^{b}R^{c}, —OR^{a}C(O)OR^{b}, —OC(O)R^{a}, —OC(O)NR^{a}R^{b}, —R^{a}NR^{b}C(O)R^{c}, —ROR^{b}, —R^{a}C(O)OR^{b}, —R^{a}C(O)NR^{b}R^{c}, —R^{a}C(O)R^{a}, —ROC(O)R^{b}, —SR^{a}, —SOR^{a}, —SO₂R^{a}, and —ONO₂, wherein R^{a}, R^{b} and R^{c} in each of the above groups can be hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenyl, substituted or unsubstituted amino, substituted or unsubstituted heteroaryl, substituted or unsubstituted heteroarylalkyl, substituted or unsubstituted heterocyclic ring, or substituted heterocyclylalkyl ring, or any two of R^{a}, R^{b} and R^{c} may be joined to form a substituted or unsubstituted saturated or unsaturated 3-10 membered ring, which may optionally include heteroatoms which may be the same or different and are selected from 0, NR^{a} (e.g., R^{a} can be hydrogen or C₁₋₆ alkyl) or S. Substitution or the combinations of substituents envisioned by this invention are preferably those that result in the formation of a stable or chemically feasible compound. The term stable as used herein refers to the compounds or the structure that are not substantially altered when subjected to conditions to allow for their production, detection and preferably their recovery, purification and incorporation into a pharmaceutical composition. The substituents in the aforementioned “substituted” groups cannot be further substituted. For example, when the substituent on “substituted alkyl” is “substituted aryl”, the substituent on “substituted aryl” cannot be “substituted alkenyl”.

The term “halo”, “halide”, or, alternatively, “halogen” means fluoro, chloro, bromo or iodo.

The terms “haloalkyl”, “haloalkenyl”, “haloalkynyl”, and “haloalkoxy” include alkyl, alkenyl, alkynyl and alkoxy structures that are substituted with one or more halogen groups or with combinations thereof. For example, the terms “fluoroalkyl” and “fluoroalkoxy” include haloalkyl and haloalkoxy groups, respectively, in which the halogen is fluorine.

Certain of the compounds described herein contain one or more asymmetric centers and can thus give rise to enantiomers, diastereomers, and other stereoisomeric forms that can be defined, in terms of absolute stereochemistry, as (R)- or (S)-. The present chemical entities, pharmaceutical compositions and methods are meant to include all such possible isomers, including racemic mixtures, optically pure forms and intermediate mixtures.

Additionally, the instant invention also includes the compounds which differ only in the presence of one or more isotopically enriched atoms for example replacement of hydrogen with deuterium or tritium, the replacement of a carbon by ¹³C- or ¹⁴C-enriched carbon, or the replacement of a nitrogen by ¹⁵N. The compounds of the present invention may also contain unnatural proportions of atomic isotopes at one or more of atoms that constitute such compounds. For example, the compounds may be radiolabeled with radioactive isotopes, such as for example tritium, iodine-125 (¹²⁵I) or carbon-14 (¹⁴C). All isotopic variations of the compounds of the present invention, whether radioactive or not, are encompassed within the scope of the present invention.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

### Compounds

In one aspect, the present invention provides a compound of formula (I):

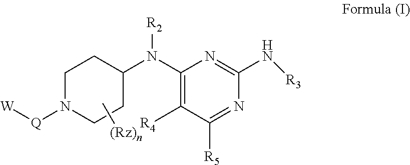

or a pharmaceutically acceptable salt thereof, wherein
R₂ is independently selected from hydrogen, deuterium (D), halogen, a substituted or unsubstituted alkyl, a substituted or unsubstituted alkoxy, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, a substituted or unsubstituted heteroaryl, and a substituted or unsubstituted heteroalkyl;
R₃ is selected from substituted or unsubstituted aryl, substituted or unsubstituted heteroaryl, substituted or unsubstituted cycloalkyl, or substituted or unsubstituted heterocycle, wherein the aryl, heteroaryl, cycloalkyl, or heterocycle can be optionally fused or bridged;
R₄ and R₅ are each independently selected from hydrogen, D, halogen, substituted or unsubstituted alkyl, substituted or unsubstituted alkoxy, substituted or unsubstituted fluoroalkyl, and —CN;
or R₄ and R₅ can be taken together with the carbons to which they are attached to form a substituted or unsubstituted 4, 5, 6, 7, or 8 membered ring;
or R₂ and R₄ can be taken together to form a substituted or unsubstituted heterocycle; Q is selected from absent, —C(O)—, —CR_{B}R_{C}—, —S(O)₂—, —S(O)—, —NR_{A}—, —C(O)O—, —NR_{A}C(O)—, —C(O)—CR_{B}R_{C}—, and —C(O)NR_{A}—;
R_{B} and R_{C} are independently H, halogen, a substituted or unsubstituted alkyl, substituted or unsubstituted alkenyl, substituted or unsubstituted alkynyl, or substituted or unsubstituted alkoxy, and R_{A} is hydrogen, substituted or unsubstituted alkyl, substituted or unsubstituted haloalkyl, substituted or unsubstituted heteroalkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl;
or R_{B} and R_{C} can be taken together with the atom to which they are attached to form a substituted or unsubstituted 4, 5, 6, 7, or 8 membered ring;
W is a substituted or unsubstituted alkyl, a substituted or unsubstituted alkenyl, a substituted or unsubstituted aryl or a substituted or unsubstituted heteroaryl;
each R_{Z} is independently selected from D, halogen, —CN, a substituted or unsubstituted alkyl, a substituted or unsubstituted alkoxy, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, a substituted or unsubstituted heteroaryl, and a substituted or unsubstituted heteroalkyl;
alternatively, (R_{Z})ₙ, together with the heterocyclic ring to which R_{Z} is attached, forms a bridged, fused, or spiro ring structure; and
n is 0, 1, 2, 3, or 4.

In embodiments, R₂ is hydrogen, a substituted or unsubstituted alkyl, a substituted or unsubstituted alkoxyl, a substituted or unsubstituted heterocycloalkyl, or a substituted or unsubstituted heteroalkyl.

In embodiments, R₂ is H, a C₁₋₆ alkyl, a substituted or unsubstituted heterocycloalkyl (e.g., oxetane, azetidine), —C₁₋₆ alkyl-OR_{A}, —C₁₋₆ alkyl-NR_{B}R_{C}, or —C₁₋₆ alkyl-C(O)NR_{B}R_{C}; wherein R_{A}is hydrogen, C₁₋₄ alkyl, C₁₋₄ fluoroalkyl, a C₃₋₈ cycloalkyl, a heterocycloalkyl (e.g., oxetane, azetidine), an aryl; R_{B} and R_{C} are independently selected from H, C₁₋₄ alkyl, C₁₋₄ fluoroalkyl; or R_{B} and R_{C} can be taken together with the nitrogen to which they are attached to form a substituted or unsubstituted 4, 5, 6, 7, or 8 membered ring.

In embodiments, R₂ is —C₁₋₄ alkyl-OR_{A}; wherein R_{A} is H, —CH₃, —CF₃, —C₂H₅, —C₃H₇, —C(CH₃)₃, —CHF₂, or a C₃₋₆ cycloalkyl.

In embodiments, R₂ is —C₁₋₆ alkyl-NR_{B}R_{C}, R_{B} and R_{C} are independently selected from H, C₁₋₄ alkyl, C₁₋₄ fluoroalkyl, or R_{B} and R_{C} forms a substituted or unsubstituted azetidine ring, wherein the substituents are selected from C₁₋₄ alkyl and halogen.

In any of the preceding embodiments, R₃ is a substituted or unsubstituted aryl, or a substituted or unsubstituted heteroaryl. In any of the preceding embodiments, R₃ is a substituted or unsubstituted aryl. In any of the preceding embodiments, R₃ is a substituted or unsubstituted heteroaryl.

In any of the preceding embodiments, R₃ is a substituted or unsubstituted heterocycle.

In any of the preceding embodiments, R₃ is a substituted or unsubstituted cycloalkyl.

In any of the preceding embodiments, R₃ is a phenyl. In any of the preceding embodiments, R₃ is a substituted phenyl, wherein the substituents are selected from halogen (preferably F, Cl), C₁₋₄ alkyl (preferably methyl or ethyl), C₁₋₄ fluoroalkyl, —OR_{A}, —NR_{B}R_{C}, —NR_{A}C(O)R_{B}, —C(O)NR_{B}R_{C}, wherein R_{A}, R_{B}, and R_{C} are independently selected from H and a C₁₋₄ alkyl.

In any of the preceding embodiments, R₃ is a substituted phenyl.

In any of the preceding embodiments, R₃ is selected from one of the following by removal of a hydrogen atom:

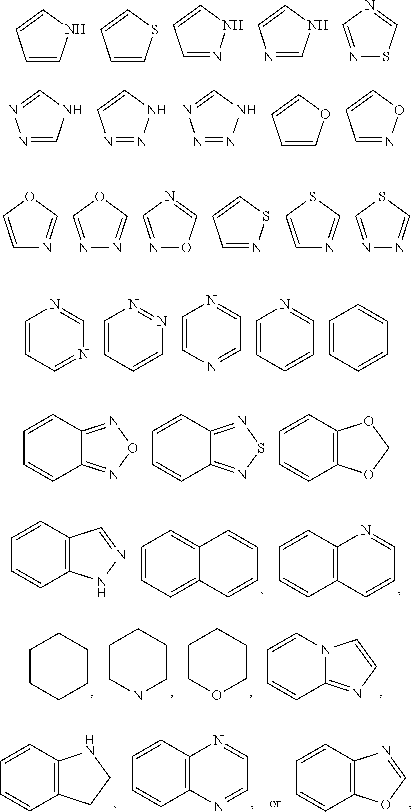

wherein each of these groups is unsubstituted, or substituted with one to three groups selected from halogen (preferably F, Cl), C₁₋₄ alkyl (preferably methyl or ethyl), C₁₋₄ fluoroalkyl, —OR_{A}, —NR_{B}R_{C}, —NR_{A}C(O)R_{B}, —C(O)NR_{B}R_{C}, a C₃₋₈ cycloalkyl, a heterocycloalkyl; wherein R_{A}, R_{B}, and R_{C} are independently selected from H and a C₁₋₄ alkyl.

In any of the preceding embodiments, R₃ is substituted or unsubstituted pyridine, substituted or unsubstituted indole, substituted or unsubstituted pyrrole, substituted or unsubstituted thiophene.

In any of the preceding embodiments, R₃ is substituted or unsubstituted pyridine, substituted or unsubstituted indole, substituted or unsubstituted pyrrole, substituted or unsubstituted thiophene, wherein the substituents are selected from F, Cl, methyl, ethyl, —CF₃, —OH, —NH₂, —OC₁₋₄ alkyl, —N(C₁₋₄ alkyl)₂, —NHC(O)CH₃, —C(O)NHCH₃, —C(O)NH₂, a 5- or 6-membered heterocycloalkyl comprising at least one nitrogen ring atom, such as:

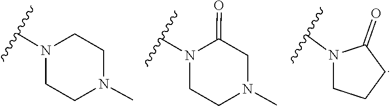

In any of the preceding embodiments, R₄ is selected from hydrogen or halogen. In any of the preceding embodiments, R₅ is selected from hydrogen or halogen.

In embodiments, R₂ and R₄ can be taken together to form a substituted or unsubstituted heterocycle. In embodiments the heterocycle can be a lactam.

In any of the preceding embodiments, Q is absent, —C(O)—, —S(O)₂—, or —S(O)—. In any of the preceding embodiments, Q is absent, —C(O)—, or —S(O)₂—. In any of the preceding embodiments, Q is absent. In any of the preceding embodiments, Q is —C(O)—. In any of the preceding embodiments, Q is —S(O)₂—. In any of the preceding embodiments, Q is —S(O)—.

In any of the preceding embodiments, W is a substituted or unsubstituted aryl or a substituted or unsubstituted heteroaryl.

In any of the preceding embodiments, W is a substituted or unsubstituted phenyl or a substituted or unsubstituted 5- or 6-membered heteroaryl, wherein the heteroaryl comprises at least one nitrogen ring atom.

In any of the preceding embodiments, W is a substituted or unsubstituted phenyl, a substituted or unsubstituted pyridine, a substituted or unsubstituted pyridazine, a substituted or unsubstituted pyrimidine, a substituted or unsubstituted pyrazine, a substituted or unsubstituted 1,2,4-triazine, or a substituted or unsubstituted 1,3,5-triazine.

In any of the preceding embodiments, W is a substituted or unsubstituted phenyl, a substituted or unsubstituted pyridine, or a substituted or unsubstituted pyrimidine, which, when substituted, comprises one, two, three, four, or five substituents selected from halogen (preferably, F, Cl), a haloalkyl (preferably a fluoroalkyl such as —CF₃), a substituted or unsubstituted alkyl, a substituted or unsubstituted alkenyl, a substituted or unsubstituted alkoxy, a substituted or unsubstituted cycloalkyl, a substituted or unsubstituted heterocycloalkyl, —CN, —CONR_{D}R_{E}, —COOR_{F}, —NR_{D}C(O)R_{F}, —C(O)NR_{D}-C₁₋₈ alkyl-NR_{D}C(O)R_{F}, —NR_{D}C(O)—C₁₋₈ alkyl-CONR_{D}R_{E}, —C(O)NR_{D}-C₁₋₈ alkyl-CONR_{D}R_{E}, —NR_{D}C(O)—C₁₋₈ alkyl-NR_{D}C(O)R_{F}, a substituted or unsubstituted (PEG)₁₋₂₀, —C(O)NR_{D}—C₁₋₈ alkyl-NR_{D}C(O)-(PEG)₁₋₂₀-NR_{D}C(O)R_{F}, —NR_{D}C(O)C₁₋₈ alkyl-C(O)NR_{D}-(PEG)₁₋₂₀-CONR_{D}R_{E}; wherein R_{D}, R_{E}, and R_{F} are independently selected from H, D, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl, a substituted or unsubstituted (PEG)₁₋₂₀; or R_{D} and R_{E} can be taken together with the nitrogen to which they are attached to form a substituted or unsubstituted 4, 5, 6, 7, or 8 membered ring; wherein (PEG)₁₋₂₀ represents a polyethylene glycol that comprises 1 to 20 repeat unit (—CH₂CH₂O—).

In any of the preceding embodiments, W is a substituted phenyl, a substituted pyridine, or a substituted pyrimidine, comprising one, two, three, four, or five substituents.

In any of the preceding embodiments, W is a substituted phenyl, a substituted pyridine, or a substituted pyrimidine, comprising one, two, three, four, or five substituents selected from F, Cl, —C₁₋₆ alkyl, —C₃₋₈ cycloalkyl, 3- to 8-membered heterocycloalkyl, —C₁₋₆ alkenyl, —C₁₋₆ alkoxy, —C₁₋₆ fluoroalkyl, —C₃₋₈ fluoro-substituted cycloalkyl, a 3- to 8-membered fluoro-substituted heterocycloalkyl, —CONR_{D}R_{E}, —CN, —C₁₋₆ fluoro-substituted alkoxy, —C(O)NR_{D}—C₁-4 alkyl-NR_{D}C(O)-(PEG)₁₋₈-NR_{D}C(O)R_{F}, R_{D}, R_{E}, and R_{F} are independently H, substituted or unsubstituted alkyl, substituted or unsubstituted cycloalkyl, substituted or unsubstituted heterocycloalkyl.

In any of the preceding embodiments, W is a substituted phenyl, substituted by one to five substituents selected from halogen (preferably F, Cl), C₁₋₄ alkyl (preferably methyl or ethyl), C₁₋₄ fluoroalkyl, —CF₃, —OCF₃, —CN, —CH═CH₂, —CONR_{D}R_{E}, —C₃₋₆ cycloalkyl (such as cyclopropyl), 3- to 6-membered heterocycloalkyl (such as oxetane), a fluoro-substituted C₃₋₆ cycloalkyl (such as fluoro-substituted cyclopropyl), a fluoro-substituted 3- to 6-membered heterocycloalkyl (such as a fluoro-substituted oxetane); wherein R_{D} and R_{E} are independently H, C₁₋₄ alkyl, or C₁₋₄ fluoroalkyl.

In any of the preceding embodiments, W is a substituted pyridine, substituted by one to five substituents.

In any of the preceding embodiments, W is a substituted pyridine, substituted by one to five substituents selected from halogen (preferably F, Cl), C₁₋₄ alkyl (preferably methyl or ethyl), C₁₋₄ fluoroalkyl, —CF₃, —OCF₃, —CN, —CH═CH₂, —CONR_{D}R_{E}, C₃₋₆ cycloalkyl (such as cyclopropyl), 3- to 6-membered heterocycloalkyl (such as oxetane), a fluoro-substituted C₃₋₆ cycloalkyl (such as fluoro-substituted cyclopropyl), a fluoro-substituted 3- to 6-membered heterocycloalkyl (such as a fluoro-substituted oxetane); wherein R_{D} and R_{E} are independently H, C₁₋₄ alkyl, or C₁₋₄ fluoroalkyl.

In any of the preceding embodiments, W is a substituted pyrimidine, substituted by one to five substituents.

In any of the preceding embodiments, W is a substituted pyrimidine, substituted by one to five substituents selected from halogen (preferably F, Cl), C₁₋₄ alkyl (preferably methyl or ethyl), C₁₋₄ fluoroalkyl, —CF₃, —OCF₃, —CN, —CH═CH₂, —CONR_{D}R_{E}, C₃₋₆ cycloalkyl (such as cyclopropyl), 3- to 6-membered heterocycloalkyl (such as oxetane), a fluoro-substituted —C₃₋₆ cycloalkyl (such as fluoro-substituted cyclopropyl), a fluoro-substituted 3- to 6-membered heterocycloalkyl (such as a fluoro-substituted oxetane); wherein R_{D} and R_{E} are independently H, C₁₋₄ alkyl, or C₁₋₄ fluoroalkyl.

In any of the preceding embodiments, at least one of the substituents is —CONH—C₁₋₄ alkyl-NHC(O)R_{F}-(PEG)₁₋₈-NHC(O)R_{F}, wherein R_{F} comprises a fused ring structure.

In any of the preceding embodiments, each R_{Z} is independently selected from halogen, —CN, a substituted or unsubstituted alkyl, a substituted or unsubstituted alkoxy; and n is 1, 2, 3, or 4.

In any of the preceding embodiments, each R_{Z} is independently selected from halogen (preferably F, Cl) and C₁₋₄ alkyl (preferably methyl or ethyl).

In any of the preceding embodiments, n is 0.

In any of the preceding embodiments, the compound of the invention forms a covalent bond with an amino acid residue in the PIKfyve protein.

In any of the preceding embodiments, the compound of the invention forms a covalent bond with a sulfur atom of a cysteine residue in the PIKfyve protein.

In any of the preceding embodiments, the compound of the invention is an irreversible inhibitor of the PIKfyve protein.

### Pharmaceutical Composition

One or more compounds according to the invention can be part of a pharmaceutical composition suitable for use in a subject, such as a human. The pharmaceutical composition may comprise at least one pharmaceutically acceptable excipient or carrier.

The pharmaceutical composition may also include at least one additional active agent or therapy. Such agent or therapy may be useful for treating or preventing the disease or condition and does not diminish the activity of the compound according to the invention. Agent(s) useful for treating or preventing the disease or condition include, but are not limited to, small molecules, peptides vaccines, antigens, antibodies, preferably monoclonal antibodies, cytotoxic agents, kinase inhibitors, allergens, antibiotics, siRNA molecules, antisense oligonucleotides, TLR antagonist (e.g. antagonists of TLR3 and/or TLR7 and/or antagonists of TLR8 and/or antagonists of TLR9), chemotherapeutic agents (both traditional chemotherapy and modem targeted therapies), targeted therapeutic agents, activated cells, peptides, proteins, gene therapy vectors, peptide vaccines, protein vaccines, DNA vaccines, adjuvants, and co-stimulatory molecules (e.g. cytokines, chemokines, protein ligands, trans-activating factors, peptides or peptides comprising modified amino acids), or combinations thereof. In embodiments, the agent can be an alkylating agent, an intercalating agent, a tubulin binding agent, a corticosteroid, or any combination of any of the foregoing. Examples of additional active agents include, but are not limited to, ibrutinib, rituximab, doxorubicin, prednisolone, vincristine, velcade, and everolimus, In one embodiment, the at least one additional active agent is a therapeutic agent selected from cyclophosphamide, hydroxydaunorubicin (also referred to as doxorubicin) vincristine, prednisone, prednisolone, and any combination of any of the foregoing.

A pharmaceutical composition can be provided as a dosage unit form, such as an ampoule, a vial, a suppository, a dragee, a tablet, or a capsule. The pharmaceutical compositions can take any suitable form (e.g., liquids, aerosols, solutions, inhalants, mists, sprays; or solids, powders, ointments, pastes, creams, lotions, gels, patches and the like) for administration by any desired route (e.g., pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration, in the form of a sterile aqueous solution or dispersion suitable for administration by either direct injection or by addition to sterile infusion fluids for intravenous infusion, or in the form of a lotion, cream, foam, patch, suspension, solution, or suppository for transdermal or transmucosal administration.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added. For oral administration in a capsule form, useful diluents include lactose and dried corn starch. When aqueous suspensions and/or emulsions are administered orally, the compound of the present invention may be suspended or dissolved in an oily phase is combined with emulsifying and/or suspending agents. If desired, certain sweetening and/or flavoring and/or coloring agents may be added.

A pharmaceutical composition can be in the form of a tablet. The tablet can comprise a unit dosage of a compound of the present invention together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

### Dosage

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms of the invention are dictated by and directly dependent on (a) the unique characteristics of the disclosed compound and the particular therapeutic effect to be achieved, and (b) the limitations inherent in the art of compounding/formulating such a disclosed compound for the treatment of a patient in need thereof.

### Methods of Treatment

The compounds of the present invention are useful as PIKfyve kinase inhibitors. In accordance with the methods described herein, a “subject in need of” is a subject having a disease, disorder or condition, or a subject having an increased risk of developing a disease, disorder or condition relative to the population at large. The subject in need thereof can be one that is “non-responsive” or “refractory” to a currently available therapy for the disease or disorder, for example cancer. In this context, the terms “non-responsive” and “refractory” refer to the subject's response to therapy as not clinically adequate to relieve one or more symptoms associated with the disease or disorder.

In one embodiment, the invention provides a method of treating a subject having a neurological disease. The method comprises administering to the subject an effective amount of a compound of the present invention (or a pharmaceutical composition of the present invention) to the subject.

A “neurological disease” is any disease that causes electrical, genetic, biochemical, or structural abnormalities in the brain, spine, or neurons. For example, a neurological disease may be a neurodegenerative disease. The neurodegenerative disease may result in motor neuron degeneration, for example. The neurological disease may be amyloid lateral sclerosis, Huntington's disease, Alzheimer's disease, or frontotemporal dementia, for example. Further examples of neurological diseases include, but are not limited to, Parkinson's disease, multiple sclerosis, peripheral myopathy, Rasmussen's encephalitis, attention deficit hyperactivity disorder, autism, central pain syndromes, anxiety, and/or depression, for example. Neurodegenerative diseases result in the progressive destruction of neurons that affects neuronal signaling. For example, a neurodegeneration may be amyotrophic lateral sclerosis, Alzheimer's disease, Huntington's disease, Friedreich's ataxia, Lewy body disease, Parkinson's disease, spinal muscle atrophy, primary lateral sclerosis, progressive muscle atrophy, progressive bulbar palsy, and pseudobulbar palsy.

The neurological disease may be one that has neuronal death generated by intracellular aggregates. In certain embodiments, the method includes treating amyotrophic lateral sclerosis (ALS). In certain embodiments, the method includes treating frontotemporal dementia (FTD). In certain embodiments, the method includes treating a neurological disease that is associated with aberrant endosomal trafficking. In certain embodiments, the method includes treating a neurological disease that is associated with aberrant lysosomal trafficking.

In further embodiments, the method includes treating a subject who has a (GGGGCC)ₙ repeat expansion in the C9ORF72 gene. The C9ORF72 gene (also known as chromosome 9 open reading frame 72, FTDALS1, ALSFTD, and FTDALS) is located on the short (p) arm of chromosome 9 at position 21.2. C9ORF72 is a 481 amino acid protein with a molecular mass of 54328 Da, which may undergo post-translational modifications of ubiquitination and phosphorylation. The expression levels of C9ORF72 may be highest in the central nervous system and the protein localizes in the cytoplasm of neurons as well as in presynaptic terminals. C9ORF72 plays a role in endosomal and lysosomal trafficking regulation and has been shown to interact with RAB proteins that are involved in autophagy and endocytic transport. C9ORF72 activates RAB5, a GTPase that mediates early endosomal trafficking. Mutations in C9ORF72 have been associated with ALS and FTD. The GGGGCC repeat expansion ((GGGGCC)ₙ) in C9ORF72 may be present in subjects suffering from a neurological disease. For example, (GGGGCC)ₙ hexanucleotide expansion is the most common cause of amyotrophic lateral sclerosis (ALS) and frontotemporal dementia (FTD), accounting for about 10% of each worldwide. The (GGGGGCC)ₙ hexanucleotide expansion may be located between exons 1a and 1b of C9ORF72. The (GGGGCC)ₙ hexanucleotide expansion may be present in a neurological disease, wherein n is greater than 25, wherein n is greater than 30, wherein n is greater than 35, wherein n is greater than 40, wherein n is greater than 45, wherein n is greater than 50, wherein n is greater than 55, wherein n is greater than 60, wherein n is greater than 65, or wherein n is greater than 70, for example. n may be between 25 and 100, between 29 and 95, between 30 and 90, between 35 and 85, between 40 and 80, between 45 and 75, between 50 and 70, between 55 and 65, or between 55 and 60, for example.

In further embodiments, the subject is haploinsufficient for C9ORF72. In further embodiments, the method includes treating patients who have a 50% or greater reduction in C9ORF72 protein activity. In further embodiments, the method includes a C9ORF72 gene product that comprises a dipeptide repeat resulting from the (GGGGCC)ₙ expansion. In further embodiments, the method includes a gain-of-function or loss of function mutation resulting from the (GGGGCC)ₙ expansion. In further embodiments, the neurological disease is associated with neuronal hyperexcitability.

In one embodiment, the invention provides a method of treating a subject having amyotrophic lateral sclerosis (ALS) comprising administering to the subject an effective amount of a compound of the present invention (or a pharmaceutical composition of the present invention).

In one embodiment, the invention provides a method of treating a subject having frontotemporal dementia (FTD) comprising administering to the subject an effective amount of a compound of the present invention (or a pharmaceutical composition of the present invention).

In one embodiment, the invention provides a method of treating a subject having Alzheimer's disease comprising administering to the subject an effective amount of a compound of the present invention (or a pharmaceutical composition of the present invention).

In one embodiment, the invention provides a method of treating a subject having Parkinson's disease comprising administering to the subject an effective amount of a compound of the present invention (or a pharmaceutical composition of the present invention).

In one embodiment, the invention provides a method of treating a subject having Huntington's disease comprising administering to the subject an effective amount of a compound of the present invention (or a pharmaceutical composition of the present invention).

In one embodiment, the invention provides a method of treating a subject having Charcot-Marie-Tooth disease (CMT) comprising administering to the subject an effective amount of a compound of the present invention (or a pharmaceutical composition of the present invention).

The invention also provides methods for treating a cell proliferative disease, a cancer, or a viral infection in a subject, preferably a human subject, in need of such treatment, by administering an effective amount of a compound of the present invention or a pharmaceutical composition comprising the same, to the subject.

In one embodiment, the invention provides a method of treating a subject having a viral infection. The method comprises administering to the subject an effective amount of a compound of the present invention (or a pharmaceutical composition of the present invention) to the subject. Viral infections include, but are not limited to, infections such as those caused by a virus selected from measles, Ebola virus (EboV), Marburg virus (MarV), borna disease, human immunodeficiency virus (HIV), severe acute respiratory system virus (SARS), middle east respiratory syndrome virus (MERS), JC polyomavirus (JC), BK polyomavirus (BK), Herpes Simplex Virus (HSV), Venezuelan equine encephalitis virus (VEEV) and Lymphocytic choriomeningitis virus (LCMV). The viral infection can be caused by any type of virus such as RNA and DNA viruses. In one embodiment, the virus is Ebola virus. In another embodiment, the virus is middle east respiratory syndrome virus (MERS). In yet another embodiment, the virus is JC polyomavirus (JC). In yet another embodiment, the virus is BK polyomavirus (BK). In yet another embodiment, the virus is Herpes Simplex Virus (HSV). In yet another embodiment, the virus is Marburg virus (MarV). In yet another embodiment, the virus is Venezuelan equine encephalitis virus (VEEV). In yet another embodiment, the virus is Lymphocytic choriomeningitis virus (LCMV).

In one embodiment, the invention provides a method of treating a subject having pain. The method comprises administering to the subject an effective amount of a compound of the present invention (or a pharmaceutical composition of the present invention) to the subject. Examples of conditions to be treated include, but are not limited to, nociceptive pain (pain transmitted across intact neuronal pathways), neuropathic pain (pain caused by damage to neural structures), pain from nerve injury (neuromas and neuromas in continuity), pain from neuralgia (pain originating from disease and/or inflammation of nerves), pain from myalgias (pain originating from disease and/or inflammation of muscle), pain associated with painful trigger points, pain from tumors in soft tissues, pain associated with neurotransmitter-dysregulation syndromes (disruptions in quantity/quality of neurotransmitter molecules associated with signal transmission in normal nerves) and pain associated with orthopedic disorders such as conditions of the foot, knee, hip, spine, shoulders, elbow, hand, head and neck.

In one embodiment, the invention provides a method of treating a subject having an inflammatory disease. The method comprises administering to the subject an effective amount of a compound of the present invention (or a pharmaceutical composition of the present invention) to the subject.

“Inflammatory disease” in the context of the present teachings encompasses, without limitation, any disease, as defined herein, resulting from the biological response of vascular tissues to harmful stimuli, including but not limited to such stimuli as pathogens, damaged cells, irritants, antigens and, in the case of autoimmune disease, substances and tissues normally present in the body. Examples of inflammatory disease include rheumatoid arthritis (RA), atherosclerosis, asthma, autoimmune diseases, chronic inflammation, chronic prostatitis, glomerulonephritis, hypersensitivities, inflammatory bowel disease, pelvic inflammatory disease, reperfusion injury, transplant rejection, celiac disease, colitis, irritable bowel syndrome, intestinal hyperplasia, metabolic syndrome, obesity, diabetes, liver disease, hepatic steatosis, fatty liver disease, non-alcoholic fatty liver disease (NAFLD), and non-alcoholic steatohepatitis (NASH), and vasculitis.

In one embodiment, the invention provides a method of treating a subject having an autoimmune disease. The method comprises administering to the subject an effective amount of a compound of the present invention (or a pharmaceutical composition of the present invention) to the subject.

“Autoimmune disease” encompasses any disease, as defined herein, resulting from an immune response against substances and tissues normally present in the body. Examples of suspected or known autoimmune diseases include rheumatoid arthritis, juvenile idiopathic arthritis, seronegative spondyloarthropathies, ankylosing spondylitis, psoriatic arthritis, antiphospholipid antibody syndrome, autoimmune hepatitis, Behçet's disease, bullous pemphigoid, coeliac disease, Crohn's disease, dermatomyositis, Goodpasture's syndrome, Graves' disease, Hashimoto's disease, idiopathic thrombocytopenic purpura, IgA nephropathy, Kawasaki disease, systemic lupus erythematosus, mixed connective tissue disease, multiple sclerosis, myasthenia gravis, polymyositis, primary biliary cirrhosis, psoriasis, scleroderma, Sjogren's syndrome, ulcerative colitis, vasculitis, Wegener's granulomatosis, temporal arteritis, Takayasu's arteritis, Henoch-Schonlein purpura, leucocytoclastic vasculitis, polyarteritis nodosa, Churg-Strauss Syndrome, and mixed cryoglobulinemic vasculitis.

In one embodiment, the invention provides a method of treating a subject having a lysosomal storage disorder. The method comprises administering to the subject an effective amount of a compound of the present invention (or a pharmaceutical composition of the present invention) to the subject.

Lysosomal storage diseases/disorders (LSDs) includes over forty metabolic disorders, many of which involve genetic defects in various lysosomal hydrolases. Representative lysosomal storage diseases and the associated defective enzymes (in parenthesis) include, but are not limited to, Aspartylglucosaminuria (Aspartylglucosaminidase), Fabry (alpha.-Galactosidase A), Infantile Batten Disease* (CNL1) (Palmitoyl Protein Thioesterase), Classic Late Infantile Batten Disease* (CNL2) (Tripeptidyl Peptidase), Juvenile Batten Disease* (CNL3) (Lysosomal Transmembrane Protein), Batten, other forms* (CNL4-CNL8) (Multiple gene products), Cystinosis (Cysteine transporter), Farber (Acid ceramidase), Fucosidosis (Acid.alpha.-L-fucosidase), Galactosidosialidosis (Protective protein/cathepsin A), Gaucher types 1, 2*, and 3* (Acid.beta.-glucosidase, G.sub.M1 gangliosidosis* (Acid.beta.-galactosidase), Hunter* (Iduronate-2-sulfatase), Hurler-Scheie* (alpha.-L-Iduronidase), Krabbe* (Galactocerebrosidase), alpha.-Mannosidosis* (Acid.alpha.-mannosidase), beta.-Mannosidosis* (Acid.beta.-mannosidase), Maroteaux-Lamy (Arylsulfatase B), Metachromatic leukodystrophy* (Arylsulfatase A), Morquio A (N-Acetylgalactosamine-6-sulfate), Morquio B (Acid.beta.-galactosidase), Mucolipidosis II/III* (N-Acetylglucosamine-1-phoshpate transferase) Niemann-Pick A*, B (Acid sphingomyelinase), Niemann-Pick C* (NPC-1), Pompe* (Acid.alpha.-glucosidase), Sandhoff* (.beta.-Hexosaminidase B), Sanfilippo A* (Heparan N-sulfatase), Sanfilippo B* (.alpha.-N-Acetylglucosaminidase), Sanfilippo C* (Acetyl-CoA: alpha.-glucosaminide), Sanfilippo D* (N-Acetylglucosamine-6-sulfate), Schindler Disease* (.alpha.-N-Acetylgalactosaminidase), Schindler-Kanzaki (alpha.-N-Acetylgalactosaminidase), Sialidosis (.alpha.-Neuramidase), Sly* (.beta.-Glucuronidase), Tay-Sachs* (.beta.-Hexosaminidase A), and Wolman* (Acid Lipase). (*CNS involvement).

In one embodiment, the invention provides a method of treating a subject having cancer. The method comprises administering to the subject an effective amount of a compound of the present invention (or a pharmaceutical composition of the present invention) to the subject. In one aspect of the methods described here, the subject in need thereof is a subject having cancer whose cancer is refractory to standard therapy or whose cancer has recurred following standard treatment.

In one embodiment, the cancer is brain cancer, glioma, sarcoma, breast cancer, lung cancer, non-small-cell lung cancer, mesothelioma, appendiceal cancer, genitourinary cancers, renal cell carcinoma, prostate cancer, bladder cancer, testicular cancer, penile cancer, cervical cancer, ovarian cancer, von Hippel Lindau disease, head and neck cancer, gastrointestinal cancer, hepatocellular carcinoma, gallbladder cancer, esophageal cancer, gastric cancer, colorectal cancer, pancreatic cancer, neuroendocrine tumors, thyroid tumor, pituitary tumor, adrenal tumor, hematological malignancy, or leukemia. In another embodiment, the cancer is a melanoma.

In one embodiment the cancer is a lymphoma. In one embodiment, the lymphoma is a B cell lymphoma. In one embodiment, the B cell lymphoma is selected from the group consisting of a Hodgkin's B cell lymphoma and a non-Hodgkin's B cell lymphoma. In one embodiment, the B cell lymphoma is a non-Hodgkin's B cell lymphoma selected from the group consisting of DLBCL, follicular lymphoma, marginal zone lymphoma (MZL) or mucosa associated lymphatic tissue lymphoma (MALT), small cell lymphocytic lymphoma (overlaps with chronic lymphocytic leukemia) and mantle cell lymphoma. In one embodiment, the B cell lymphoma is a non-Hodgkin's B cell lymphoma selected from the group consisting of Burkitt's lymphoma, Primary mediastinal (thymic) large B-cell lymphoma, Lymphoplasmacytic lymphoma, which may manifest as Waldenstrom macroglobulinemia, Nodal marginal zone B cell lymphoma (NMZL), Splenic marginal zone lymphoma (SMZL), Intravascular large B-cell lymphoma, Primary effusion lymphoma, Lymphomatoid granulomatosis, T cell/histiocyte-rich large B-cell lymphoma, Primary central nervous system lymphoma, Primary cutaneous diffuse large B-cell lymphoma, leg type (Primary cutaneous DLBCL, leg type), EBV positive diffuse large B-cell lymphoma of the elderly, Diffuse large B-cell lymphoma associated with inflammation, Intravascular large B-cell lymphoma, ALK-positive large B-cell lymphoma, and Plasmablastic lymphoma.

In a preferred embodiment of any of the methods described herein, the subject is a human subject.

In any of the methods described herein, the method may further comprise also administering an effective amount of a potassium channel activator, an inhibitor of a glutamate receptor (such as the receptor NMDA, AMPA, or kainite) (e.g., AP5, CNQX, and NBQX), or any combination of any of the foregoing.

In any of the methods described herein, the method may further comprise administering an effective amount of one or more pharmaceutically active agents such as immunostimulatory agents, anti-viral agents, antibiotics, anti-fungal agents, anti-parasitic agents, anti-tumor agents, cytokines, chemokines, growth factors, anti-angiogenic factors, chemotherapeutic agents, antibodies and gene silencing agents. Preferably, the pharmaceutically active agent is selected from the group consisting of an immunostimulatory agent, an anti-bacterial agent, an anti-viral agent, an anti-inflammatory agent and an anti-tumor agent. The more than one pharmaceutically active agents may be of the same or different category.

In certain embodiments, the one or more pharmaceutically active agents includes but are not limited to, an antigen, an anti-viral vaccine, an anti-bacterial vaccine, and/or an anti-tumor vaccine, wherein the vaccine can be prophylactic and/or therapeutic.

According to some of any of the embodiments described herein, in any of the methods and uses as described herein, the method may further comprise an additional therapy for treating the disease associated with a tumor (e.g., cancer).

According to some of any of the embodiments described herein, in any of the methods and uses as described herein, the additional therapy for treating the disease associated with a tumor is an anti-cancer therapy.

Suitable anti-cancer therapy includes, for example, chemotherapy, radiotherapy, phototherapy and/or photodynamic therapy, surgery, nutritional therapy, ablative therapy, combined radiotherapy and chemotherapy, brachiotherapy, proton beam therapy, immunotherapy, cellular therapy and photon beam radiosurgical therapy, and any combination of the foregoing.

Chemotherapeutic drugs (e.g., anti-cancer drugs) that may optionally be co-administered to the subject prior to, concomitant with and/or subsequent to one or more compounds as described herein in any of the respective embodiments include, but are not limited to acivicin, aclarubicin, acodazole, acronine, adozelesin, aldesleukin, altretamine, ambomycin, ametantrone, aminoglutethimide, amsacrine, anastrozole, anthramycin, asparaginase, asperlin, azacitidine, azetepa, azotomycin, batimastat, benzodepa, bicalutamide, bisantrene, bisnafide, bizelesin, bleomycin, brequinar, bropirimine, busulfan, cactinomycin, calusterone, caracemide, carbetimer, carboplatin, carmustine, carubicin, carzelesin, cedefingol, chlorambucil, cirolemycin, cisplatin, cladribine, crisnatol, cyclophosphamide, cytarabine, dacarbazine, dactinomycin, daunorubicin, decitabine, dexormaplatin, dezaguanine, diaziquone, docetaxel, doxorubicin, droloxifene, dromostanolone, duazomycin, edatrexate, eflornithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, gemcitabine, hydroxyurea, idarubicin, ifosfamide, ilmofosine, interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine, megestrol, melengestrol, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nocodazole, nogalamycin, ormaplatin, oxisuran, paclitaxel, pegaspargase, peliomycin, pentamustine, peplomycin, perfosfamide, pipobroman, piposulfan, piroxantrone, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, verteporfin, vinblastine, vincristine, vindesine, vinepidine, vinglycinate, vinleurosine, vinorelbine, vinrosidine, vinzolidine, vorozole, zeniplatin, zinostatin, zorubicin, and any pharmaceutically acceptable salts thereof.

In some embodiments, the anti-cancer therapy comprises immunotherapy, including, for example, checkpoint inhibitors, CAR-T cell therapy, and/or vaccine adjuvants (e.g., interferon or saponin), and immune-adjuvants, such as aluminum salts, organic adjuvants, and genomic material-based adjuvants, such as CpG. Anti-tumor immunity can be further augmented by inhibition of immune suppressor cells.

According to some embodiments, the anti-cancer therapy comprises administration of an anti-cancer immune modulator agent. As used herein, the term “anti-cancer immune modulator agent” refers to an agent capable of eliciting an immune response (e.g. T cell, NK cell) against a cancerous cell. Exemplary anti-cancer immune modulator agents include, but are not limited to, a cancer antigen, a cancer vaccine, an anti-cancer antibody, a cytokine capable of inducing activation and/or proliferation of a T cell and an immune-check point regulator.

Alternatively or additionally, such modulators may be immune stimulators such as immune-check point regulators which are of specific value in the treatment of cancer. As used herein the term “immune-check point regulator” refers to a molecule that modulates the activity of one or more immune-check point proteins in an agonistic or antagonistic manner resulting in activation of an immune cell. As used herein the term “immune-check point protein” refers to a protein that regulates an immune cell activation or function. Immune check-point proteins can be either co-stimulatory proteins (i.e. transmitting a stimulatory signal resulting in activation of an immune cell) or inhibitory proteins (i.e. transmitting an inhibitory signal resulting in suppressing activity of an immune cell). According to some embodiments, the immune check-point protein regulates activation or function of a T cell. Numerous checkpoint proteins are known in the art and include, but not limited to, PD1, PDL-1, B7H2, B7H4, CTLA-4, CD80, CD86, LAG-3, TIM-3, KIR, IDO, CD19, OX40, 4-1BB (CD137), CD27, CD70, CD40, GITR, CD28 and ICOS (CD278).

According to some embodiments, the anti-cancer therapy comprises a surgical procedure, for example, resection or excision of at least a portion of the tumor.

In any of the methods described herein, the compounds (or composition thereof) can be administered by any suitable route, such as an oral, intravenous, or subcutaneous route.

### Examples

### Preparation of the Compounds

Compounds of the present invention can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. The following descriptions of synthetic methods are designed to illustrate, but not to limit, general procedures for the preparation of compounds of the present invention.

Compounds of the present invention can be conveniently prepared by a variety of methods familiar to those skilled in the art. The compounds of this invention with each of the formulae described herein may be prepared according to the following procedures from commercially available starting materials or starting materials which can be prepared using literature procedures. These procedures show the preparation of representative compounds of this invention.

The synthetic processes of the invention can tolerate a wide variety of functional groups; therefore various substituted starting materials can be used. The processes generally provide the desired final compound at or near the end of the overall process, although it may be desirable in certain instances to further convert the compound to a pharmaceutically acceptable salt, polymorph, hydrate, solvate or co-crystal thereof.

### LCMS Methods

Method 1 Instrument: Waters Acquity UPLC I class Series with SQD 2 Mass detector, Column: Zorbax Eclipse Plus C18; 2.1×50 mm, 1.8μ. Gradient [time (min)/solvent B (%)]: 0.0/5, 0.2/5,2.5/98, 3.5/98, 3.6/5, 4.0/5. (Solvent A=0.1% Formic acid in H2O; Solvent B=0.05% Formic acid in ACN; Injection volume 1 μL (may vary); UV detection 210 to 400 nm; Column temperature 40 °C; 0.8 mL/min.

Method 2 Instrument: Agilent Technologies 1290 Infinity II Series with MSD 6135B mass detector, Column: Zorbax Eclipse Plus C18; 2.1×50 mm 1.8μ. Gradient [time (min)/solvent B (%)]: 0.0/5, 0.2/5,2.5/98, 3.5/98, 3.6/5, 4.0/5. (Solvent A=0.1% Formic acid in H2O; Solvent B=0.05% Formic acid in ACN; Injection volume 1 μL (may vary); UV detection 210 to 400 nm; Column temperature 40 °C; 0.8 mL/min.

Method 3 Instrument: Shimadzu N series UPLC with LCMS 2020 Mass Detector, Column: X bridge C8; 3.5μ, 4.6×50 mm. Gradient [time (min)/solvent B (%)]: 0.0/10, 0.2/10,2.5/98, 3.5/98, 3.6/10, 4.0/10. (Solvent A=5 mM Ammonium Bicarbonate in H2O; Solvent B=ACN; Injection volume 1 μL (may vary); UV detection 210 to 400 nm; Column temperature 40 °C; 1.0 mL/min.

Method 4 Instrument: Waters Acquity UPLC I class Series with SQD 2 Mass detector, Column: Acquity UPLC BEH C18; 1.7μ, 2.1×50 mm. Gradient [time (min)/solvent B (%)]: 0.0/5, 0.2/5,2.5/98, 3.5/98, 3.6/5, 4.0/5. (Solvent A=0.1% Formic acid in H₂O; Solvent B=0.05% Formic acid in ACN; Injection volume 1 μL (may vary); UV detection 210 to 400 nm; Column temperature 35 °C; 0.8 mL/min. Alternatively, for UV inactive compounds, an ELSD detector was employed with the above instrument to visualize compounds.

Method 5 Instrument: Shimadzu N series UPLC with LCMS 2020 Mass Detector, Column: Acquity UPLC BEHC18(2.1×50) mm, 1.7μ. Gradient [time (min)/solvent B (%)]: 0.0/5, 0.2/5, 2.5/98, 3.5/98, 3.6/5, 4.0/5. (Solvent A=5 mM Ammonium Acetate in H2O; Solvent B=100% ACN; Injection volume 1 μL (may vary); UV detection 210 to 400 nm; Column temperature 35 °C; 0.6 mL/min.

### UPLC-MS Methods

Method 6 (4 minutes) Instrument: Waters Acquity UPLC I class Series with SQD 2 Mass detector, Column: Zorbax Eclipse Plus C18; 2.1×50 mm, 1.8μ, 2.1×50 mm. Gradient [time (min)/solvent B (%)]: 0.0/5, 0.2/5,2.5/98, 3.5/98, 3.6/5, 4.0/5. (Solvent A=0.1% Formic acid in H2O; Solvent B=0.05% Formic acid in ACN; Injection volume 1 μL; UV detection 210 to 400 nm; Column temperature 40 °C; 0.8 mL/min.

Method 7 (3 minutes) Instrument: Waters Acquity UPLC I class Series with SQD 2 Mass detector, Column: Acquity UPLC BEH C18; 1.7μ, 2.1×30 mm. Gradient [time (min)/solvent B (%)]: 0.0/5, 0.4/5,1.8/100, 2.4/100, 2.6/5, 3.0/5. (Solvent A=0.1% Formic acid in H2O; Solvent B=0.05% Formic acid in ACN; Injection volume 1 μL; UV detection 210 to 400 nm; Column temperature 30 °C; 1.0 mL/min.

Method 8 Instrument: Shimadzu N series UPLC with LCMS 2020 Mass Detector, Column: Phenomenex kinetex EVO C18 2.6μ, 3.0×50 mm. Gradient [time (min)/solvent B (%)]: 0.0/5, 0.2/5,2.50/95, 3.55/95, 3.6/5, 4.0/5. (Solvent A=5 mM Ammonium bicarbonate in H2O; Solvent B=ACN; Injection volume 1 μL (may vary); UV detection 210 to 400 nm; Column temperature 40 °C; 0.8 mL/min.

Method 9 Instrument: Agilent Technologies 1290 Infinity II Series with MSD 6135B mass detector, Column: Zorbax Eclipse Plus C18; 2.1×50 mm 1.8μ. Gradient [time (min)/solvent B (%)]: 0.0/5, 0.2/5,2.5/95, 3.5/95, 3.6/5, 4.0/5. (Solvent A=0.1% Formic acid in H2O; Solvent B=0.05% Formic acid in ACN; Injection volume 1 μL (may vary); UV detection 210 to 400 nm; Column temperature 40 °C; 0.8 mL/min.

### HPLC Methods

Method A: Instrument: Shimadzu LC; Prominence-I Series LC-2050C, Column: X-Select CSHC18, 4.6×150 mm; 3.5μ. Gradient [time (min)/solvent B (%)]:0.0/10, 2.0/10, 12/100,17.0/100,18.0/10, 20.0/10 (Solvent A=1 mL of FA in 1000 mL of Milli-Q Water; Solvent B=ACN); Injection volume 1 μL (may vary); UV detection 210 to 400 nm; Column temperature 35 °C; 1.2 mL/min.

Method B: Instrument: Shimadzu LC; Prominence-I Series LC-2050C, Column: Atlantis T3 C18, 4.6×150 mm; 3.0μ. Gradient [time (min)/solvent B (%)]:0.0/10, 2.0/10, 12/100,17.0/100,18.0/10, 20.0/10 (Solvent A=1 mL of FA in 1000 mL of Milli-Q Water; Solvent B=ACN); Injection volume 1 μL (may vary); UV detection 210 to 400 nm; Column temperature 35 °C; 1.2 mL/min.

Method C: Instrument: Shimadzu LC; Prominence-I Series LC-2050C, Column: X-bridgeC18, 4.6×150 mm; 5.0μ. Gradient [time (min)/solvent B (%)]:0.0/5, 2.0/5, 12/100,17.0/100,18.0/5, 20.0/5. (Solvent A=5 mM Ammonium Bicarbonate in Milli-Q Water; Solvent B=ACN); Injection volume 1 μL (may vary); UV detection 210 to 400 nm; Column temperature 35 °C; 1.2 mL/min.

Method D: Instrument: Shimadzu LC; Prominence-I Series LC-2050C, Column: X-bridgeC18, 4.6×150 mm; 3.5μ. Gradient [time (min)/solvent B (%)]:0.0/5, 2.0/5, 12/100,17.0/100,18.0/5, 20.0/5. (Solvent A=5 mM Ammonium Bicarbonate in Milli-Q Water; Solvent B=ACN); Injection volume 1 μL (may vary); UV detection 210 to 400 nm; Column temperature 35 °C; 1.2 mL/min.

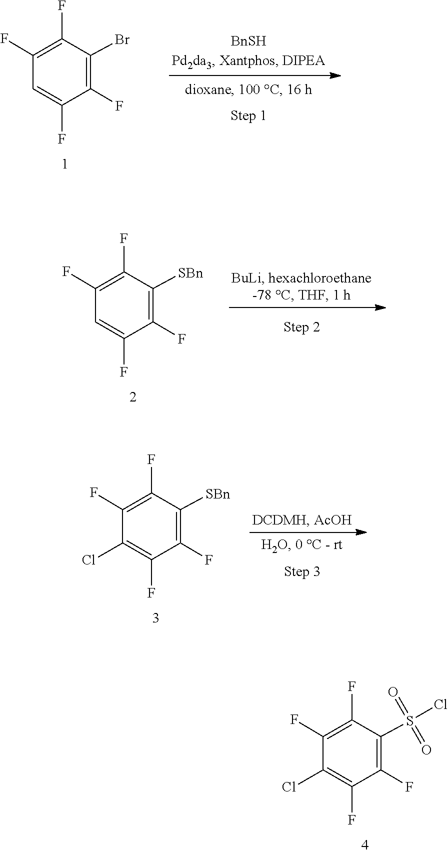

### Benzyl(2,3,5,6-tetrafluorophenyl)sulfane (2)

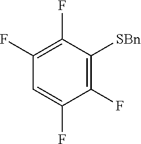

In a 20 mL vial, 3-bromo-1,2,4,5-tetrafluorobenzene (0.3 g, 1.310 mmol) in DIPEA (0.685 ml, 3.93 mmol) and dioxane (3.0 ml) were added under a nitrogen atmosphere. Reaction mixture was degassed and benzyl mercaptan (0.146 g, 1.179 mmol) followed by XantPhos (0.152 g, 0.262 mmol) and Pd₂(dba)₃ (0.120 g, 0.131 mmol) were introduced at rt. The resulting reaction mixture was heated at 110 °C for 15 h. The reaction was monitored by TLC. After completion of the reaction, volatiles were removed under reduced pressure to get the crude. The obtained crude compound was purified by isolera column chromatography using 100-200 mesh silicagel and eluted with 100% Hexane to get compound benzyl(2,3,5,6-tetrafluorophenyl)sulfane (220 mg, 0.808 mmol, 61.7%). 1H-NMR (400 MHz, DMSO-d6): δ 7.30-7.24 (m, 5H), 7.00 (s, 1H), 4.15 (s, 2H).

### Benzyl(4-chloro-2 3,5,6-tetrafluorophenyl)sulfane (3)

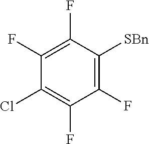

To a stirred solution of benzyl(2,3,5,6-tetrafluorophenyl)sulfane (0.39 g, 1.432 mmol) in THF (4 ml) was added BuLi (1.6 M in Hexane) (0.985 ml, 1.576 mmol) at −78 °C under nitrogen, followed by stirring for 1 h at −78 °C. A solution of hexachloroethane (1.695 g, 7.16 mmol) in 2 mL of THF was introduced at −78 °C then the reaction mixture was allowed to stir for 1 h at 0 °C. The reaction was monitored by TLC, quenched with ice water (3 mL) and extracted with ethyl acetate (2×50 mL). The combined organic layers were dried over anhydrous sodium sulphate, filtered and concentrated under reduced pressure affording crude product. The crude product was purified column chromatography using 100-200 mesh silica gel in 100% Hexane as eluent to get benzyl(4-chloro-2,3,5,6-tetrafluorophenyl)sulfane (215 mg, 0.701 mmol, 48.9%).

LCMS: No ionization. ¹H-NMR (400 MHz, DMSO-d6): δ 7.28-7.23 (m, 5H), 4.12 (s, 2H).

### 4-chloro-2,3,5,6-tetrafluorobenzenesulfonyl chloride (4)

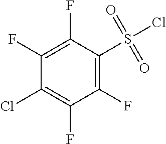 [SMILES: O=S(=O)(Cl)c1c(F)c(F)c(Cl)c(F)c1F]

To a stirred solution of benzyl(4-chloro-2,3,5,6-tetrafluorophenyl)sulfane (215 mg, 0.701 mmol) in mixture of acetic acid (4.5 mL) and water (0.5 mL), 1,3-dichloro-5,5-dimethylimidazolidine-2,4-dione (276 mg, 1.402 mmol) was added at 0 °C. The resulting reaction mixture was stirred for 1 h at rt. The reaction was monitored by TLC: quenched with ice water (3 mL) and extracted with hexane (3×50 mL). The combined organic layers were dried over anhydrous sodium sulphate, filtered and concentrated under reduced pressure affording 4-chloro-2,3,5,6-tetrafluorobenzenesulfonyl chloride (175 mg, 0.618 mmol, 88%).

LCMS:47%, RT: 1.14, m/z: 263 (M−H+) [corresponding sulfonic acid mass].

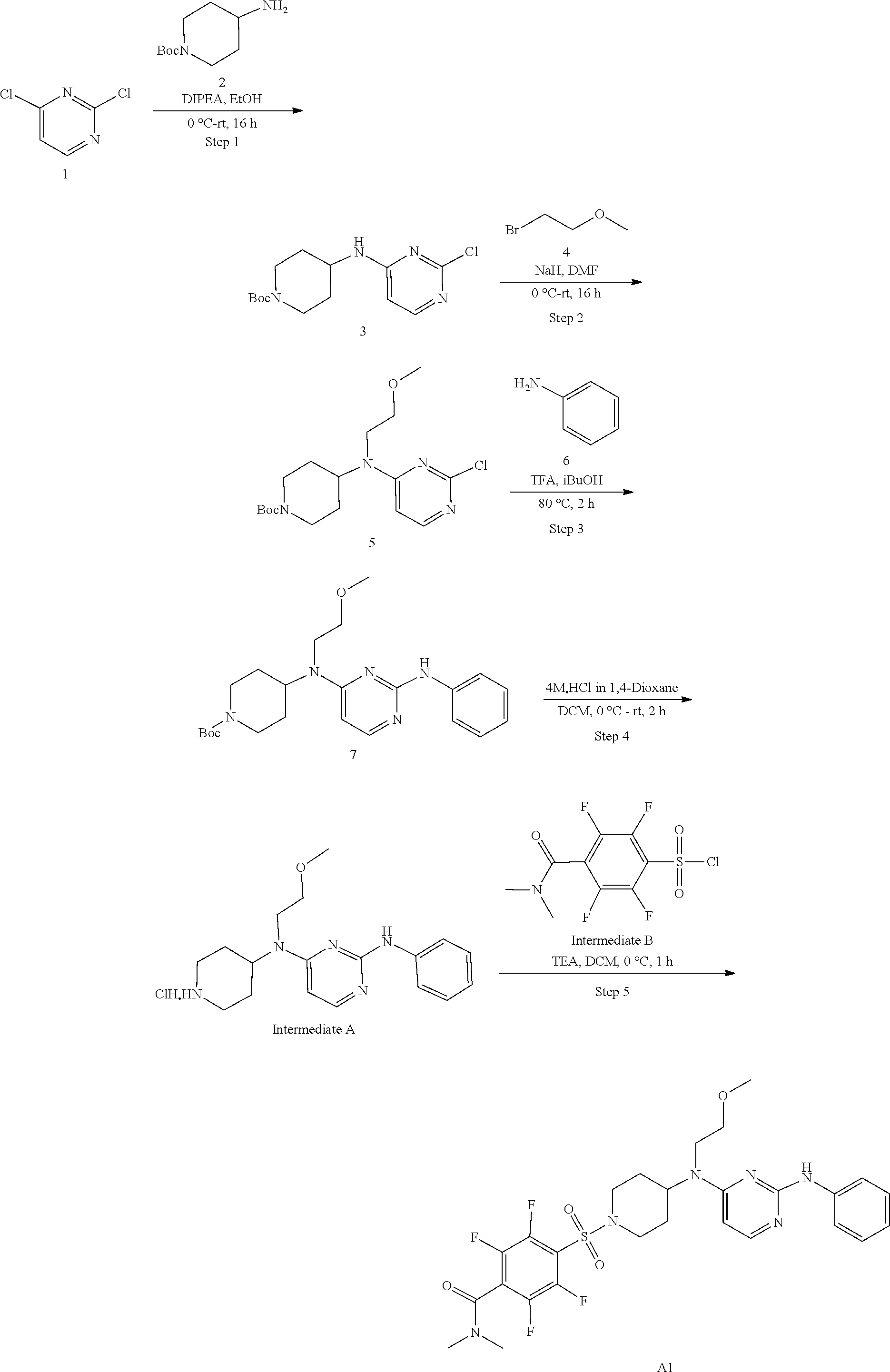

### Synthesis of tert-butyl 4-((2-chloropyrimidin-4-yl)amino)piperidine-1-carboxylate (3)

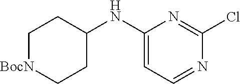 [SMILES: CC(C)(C)OC(=O)N1CCC(Nc2ccnc(Cl)n2)CC1]

In an oven-dried 500 mL two-neck round-bottom flask equipped with N₂ balloon and a rubber septum, 2,4-dichloropyrimidine (15 g, 101 mmol) and EtOH (200 ml) were added. The reaction mixture was cooled to 0 °C, tert-butyl 4-aminopiperidine-1-carboxylate (20.17 g, 101 mmol) and DIPEA (13.01 g, 101 mmol) were introduced at the same temperature then the resulting reaction mixture was stirred for 16 h at 25 °C. The reaction was monitored by TLC. After completion of the reaction volatiles were removed under reduced pressure. The crude mass was purified by flash column chromatography using 40% Ethyl acetate in hexane to get tert-butyl 4-((2-chloropyrimidin-4-yl)amino)piperidine-1-carboxylate (20 g, 62.0 mmol, 61.6% yield) as an off white solid.

LCMS: 95.5%, RT: 1.67 min, m/z=313.1 (M+H)⁺; ¹H-NMR (400 MHz, DMSO-d₆): δ 7.89-7.85 (m, 2H), 6.41 (d, J=4.4 Hz, 1H), 4.04-3.84 (m, 3H), 2.91 (brs, 2H), 1.83 (d, J=11.2 Hz, 2H), 1.40 (s, 9H), 1.35-1.24 (m, 2H).

### Synthesis of tert-butyl 4-((2-chloropyrimidin-4-yl)(2-methoxyethyl)amino)piperidine-1-carboxylate (5)

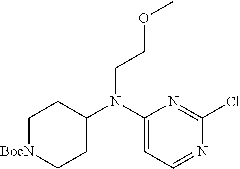 [SMILES: COCCN(c1ccnc(Cl)n1)C1CCN(C(=O)OC(C)(C)C)CC1]

To an oven dried 250 mL two-neck round-bottom flask equipped with N₂ balloon and a rubber septum, tert-butyl 4-((2-chloropyrimidin-4-yl)amino)piperidine-1-carboxylate (6 g, 19.18 mmol) and DMF (100 mL) were added. The reaction mixture was cooled to 0 °C, sodium hydride (2.30 g, 57.5 mmol) was added portion wise at the same temperature, stirred for 20 min, then DMF solution of 1-bromo-2-methoxyethane (9.01 mL, 96 mmol) in (10 mL) was introduced. The resulting reaction mixture was stirred for 16 h at 25 °C. The reaction was quenched with ice water (100 mL) and extracted with Ethyl acetate (2×100 mL). The combined organic layers were dried over anhydrous sodium sulphate, filtered and concentrated under reduced pressure to afford crude product. The crude mass was purified by flash column chromatography using 50-60% Ethyl acetate in hexanes to afford tert-butyl 4-((2-chloropyrimidin-4-yl)(2-methoxyethyl)amino)piperidine-1-carboxylate (6.0 g, 16.21 mmol, 84.6% yield) as an off-white solid.

LCMS: 92.4%, RT: 1.912, m/z=371.1 (M+H); ¹H-NMR (400 MHz, DMSO-d₆): δ 8.07 (d, J=6.0 Hz, 1H), 6.79 (brs, 1H), 4.07-4.01 (m, 2H), 3.55-3.48 (m, 3H), 3.45-3.40 (m, 2H), 3.26 (s, 3H), 2.81 (brs, 2H), 1.64 (s, 4H), 1.41 (s, 9H).

### Synthesis of tert-butyl 4-((2-methoxyethyl)(2-(phenylamino)pyrimidin-4-yl)amino)piperidine-1-carboxylate (7)

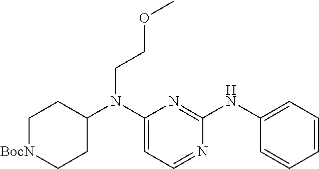 [SMILES: COCCN(c1ccnc(Nc2ccccc2)n1)C1CCN(C(=O)OC(C)(C)C)CC1]

To an oven dried 250 mL two-neck round-bottom flask equipped with N₂ balloon, tert-butyl 4-((2-chloropyrimidin-4-yl)(2-methoxyethyl)amino)piperidine-1-carboxylate (10.5 g, 28.3 mmol), isobutanol (100 mL), aniline (2.58 mL, 28.3 mmol) and TFA (2.4 mL, 31.1 mmol) were added at room temperature. The reaction mixture was stirred for 2 h at 80 °C. The reaction was monitored by TLC. After completion of the reaction, the reaction mixture was concentrated under reduced pressure. The obtained crude was purified by flash column chromatography using 75% Ethyl acetate in hexane to afford tert-butyl 4-((2-methoxyethyl)(2-(phenylamino)pyrimidin-4-yl)amino)piperidine-1-carboxylate (6.3 g, 14.29 mmol, 50.5% yield).

LCMS: 97.05%, RT: 1.405, m/z=428.2 (M+H)⁺; ¹H-NMR (400 MHz, DMSO-d₆): δ 10.03 (s, 1H), 7.98 (d, 7.2 Hz, 1H), 7.55 (d, J=8.0 Hz, 2H), 7.39 (t, J=7.2 Hz, 2H), 7.15 (t, J=7.6 Hz, 1H), 6.50 (brs, 1H), 4.09-4.00 (m, 2H), 3.66-3.63 (m, 2H), 3.51-3.41 (m, 3H), 3.24 (s, 3H), 2.82 (brs, 2H), 1.70-1.63 (m, 4H), 1.41 (s, 9H).

### Synthesis of N⁴-(2-methoxyethyl)-N²-phenyl-N⁴-(piperidin-4-yl)pyrimidine-2,4-diamine hydrochloride (Intermediate A)

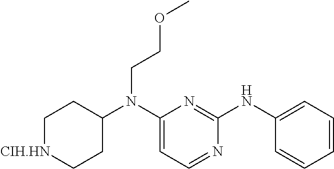

To an oven dried 50 mL round-bottom flask equipped with N₂ balloon, tert-butyl 4-((2-methoxyethyl)(2-(phenylamino)pyrimidin-4-yl)amino)piperidine-1-carboxylate (1 g, 2.339 mmol) and DCM (10 mL) were added. 4M HCl in dioxane (5 mL, 165 mmol) at 25 °C was added and the reaction mixture was stirred for 2 h. The reaction was monitored by TLC. After completion of the reaction volatiles were removed under reduced pressure to get crude. Thus obtained crude was triturated with MTBE to afford N4-(2-methoxyethyl)-N2-phenyl-N4-(piperidin-4-yl)pyrimidine-2,4-diamine hydrochloride (810 mg, 2.226 mmol, 95% yield) as an off white solid.

LCMS: 98.16%, RT: 1.728, m/z=328.1 [(M−HCl)+H]⁺.

### Example A1 Synthesis of 2,3,5,6-tetrafluoro-4-((4-((2-methoxyethyl)(2-(phenylamino)pyrimidin-4-yl)amino)piperidin-1-yl)sulfonyl)-N,N-dimethylbenzamide

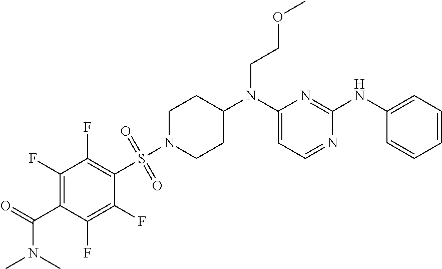 [SMILES: COCCN(c1ccnc(Nc2ccccc2)n1)C1CCN(S(=O)(=O)c2c(F)c(F)c(C(=O)N(C)C)c(F)c2F)CC1]

To an oven dried 50 mL two-neck round-bottom flask equipped with N₂ balloon, N⁴-(2-methoxyethyl)-N²-phenyl-N⁴-(piperidin-4-yl)pyrimidine-2,4-diamine hydrochloride (350 mg, 0.827 mmol) in DCM (15 mL) were added TEA (251 mg, 2.480 mmol) and stirred for 10 minutes. The resulted solution was cooled to −78° c followed by 4-(dimethylcarbamoyl)-2,3,5,6-tetrafluorobenzenesulfonyl chloride (317 mg, 0.992 mmol) solution in DCM (5 mL) was added at the same temperature and further stirred for 1 h at −78 °C. The reaction was monitored by TLC; quenched with aqueous saturated NaHCO₃ solution (15 mL) and extracted with DCM (2×15 mL). The combined organic layers were dried over anhydrous sodium sulphate, filtered and concentrated under reduced pressure affording crude product. The crude mass was purified by prep HPLC (Method C) to afford 2,3,5,6-tetrafluoro-4-((4-((2-methoxyethyl)(2-(phenylamino)pyrimidin-4-yl)amino)piperidin-1-yl)sulfonyl)-N,N-dimethyl benzamide (275 mg, 0.446 mmol, 53.9% yield) as an off white solid.

The compounds in the following table 1 were prepared in the same manner as A1 from the appropriate starting materials.

| | | LCMS | LCMS | |
|---|---|---|---|---|
| | | Retention | Mass | LCMS |
| Cmp | Structure | Time | Obs | method |
| A2 | 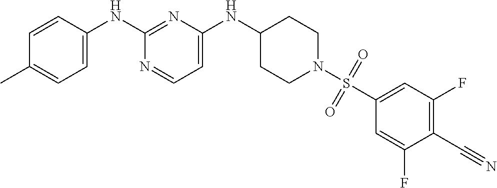 [SMILES: Cc1ccc(Nc2nccc(NC3CCN(S(=O)(=O)c4cc(F)c(C#N)c(F)c4)CC3)n2)cc1] 19F NMR (376 MHz, DMSO-d6): δ −101.62 to −101.64 (2F) 1H-NMR (400 MHz, DMSO-d6): δ 8.98 (bs, 1H), 7.93 (d, J = 7.20 Hz, 2H), 7.77 (d, J = 5.60 Hz, 1H), 7.54 (d, J = 8.00 Hz, 2H), 7.39 (s, 1H), 7.00 (d, J = 8.40 Hz, 2H), 5.91 (d, J = 6.00 Hz, 1H), 3.78 (s, 1H), 3.68 (d, J = 12.00 Hz, 2H), 2.72-2.67 (m, 2H), 2.23 (s, 3H), 2.00 (d, J = 10.80 Hz, 2H), 1.48 (q, J = 10.80 Hz, 2H). | 1.785 | 485.1 (M + H)+ | Method- 9 |
| A3 | 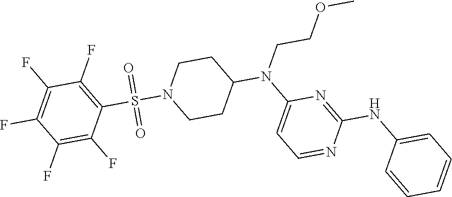 [SMILES: COCCN(c1ccnc(Nc2ccccc2)n1)C1CCN(S(=O)(=O)c2c(F)c(F)c(F)c(F)c2F)CC1] 19F NMR (376 MHz, DMSO-d6): δ −135.59 (2F), −146.79 (1F), −159.36 (2F) 1H-VT-NMR (400 MHz, DMSO-d6): δ 8.69 (s, 1H), 7.95 (d, J = 6.00 Hz, 1H), 7.67 (d, J = 7.60 Hz, 2H), 7.19 (t, J = 7.60 Hz, 2H), 6.87 (t, J = 7.6 Hz, 1H), 6.15 (d, J = 6.0 Hz, 1H), 4.27-4.23 (m, 1H), 3.89 (d, J = 12.00 Hz, 2H), 3.56-3.47 (m, 4H), 3.30 (s, 3H), 2.91-2.87 (m, 2H), 1.93- 1.86 (m, 4H) | 1.632 | 558.1 [M + H]+ | Method- 3 |
| A4 | 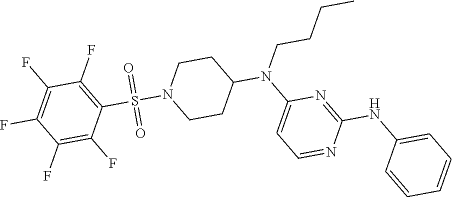 [SMILES: CCCCN(c1ccnc(Nc2ccccc2)n1)C1CCN(S(=O)(=O)c2c(F)c(F)c(F)c(F)c2F)CC1] 19F NMR (376 MHz, DMSO-d6): δ −135.55 (2F), −146.69 to −146.81 (1F), −159.26 to −159.43 (2F). 1H-NMR (400 MHz, DMSO-d6): δ 8.99 (s, 1H), 7.93 (d, J = 6.00 Hz, 1H), 7.68 (d, J = 7.60 Hz, 2H), 7.16 (t, J = 8.00 Hz, 2H), 6.85 (t, J = 7.20 Hz, 1H), 6.08 (d, J = 5.60 Hz, 1H), 3.85 (d, J = 11.60 Hz, 2H), 3.29-3.26 (m, 2H), 2.83 (s, 2H), 2.34-2.33 (m, 1H), 1.86-1.85 (m, 4H), 1.51-1.49 (m, 2H), 1.39-1.33 (m, 2H), 0.93 (t, J = 7.60 Hz, 3H). | 1.824 | 556.2 (M + H)+ | Method- 2 |
| A5 | 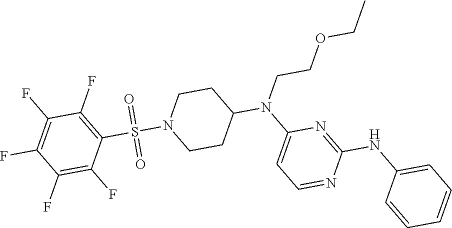 [SMILES: CCOCCN(c1ccnc(Nc2ccccc2)n1)C1CCN(S(=O)(=O)c2c(F)c(F)c(F)c(F)c2F)CC1] 19F NMR (376 MHz, DMSO-d6): δ −135.62 to −135.56 (2F), −146.86 to −146.75 (1F), −159.46 to −159.29 (2F) 1H-NMR (400 MHz, DMSO-d6): δ 9.02 (s, 1H), 7.95 (d, J = 6.40 Hz, 1H), 7.68 (d, J = 8.00 Hz, 2H), 7.17 (t, J = 7.60 Hz, 2H), 6.85 (t, J = 7.20 Hz, 1H), 6.16 (d, J = 6.40 Hz, 1H), 4.22 (m, 1H), 3.84 (d, J = 11.60 Hz, 2H), 3.53-3.42 (m, 6H), 2.84 (s, 2H), 1.88 (s, 4H), 1.13 (t, J = 6.80 Hz, 3H) ppm. | 1.711 | 572.2 (M + H)+ | Method- 2 |
| A6 | 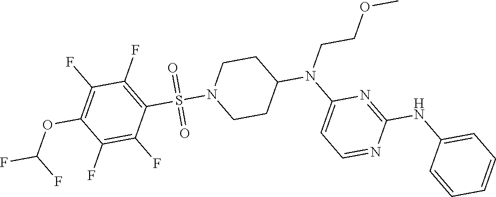 [SMILES: COCCN(c1ccnc(Nc2ccccc2)n1)C1CCN(S(=O)(=O)c2c(F)c(F)c(OC(F)F)c(F)c2F)CC1] 19F NMR(376 MHz, DMSO-d6): δ −81.80 to −82.02 (2F), −135.84 to −135.89 (2F), −151.26 to −151.30 (2F). 1H-VT-NMR (400 MHz, DMSOd6): δ 8.70 (s, 1H), 7.94 (d, J = 6.00 Hz, 1H), 7.66 (d, J = 8.00 Hz, 2H), 7.58-7.17 (m, 3H), 6.87 (t, J = 7.60 Hz, 1H), 6.16 (d, J = 6.00 Hz, 1H), 4.27 (brs, 1H), 3.90 (d, J = 12.00 Hz, 2H), 3.56-3.47 (m, 4H), 3.30 (s, 3H), 2.95-2.88 (m, 2H), 1.92-1.87 (m, 4H). | 2.357 | 606.2 [M + H]+ | Method- 5 |
| A7 | 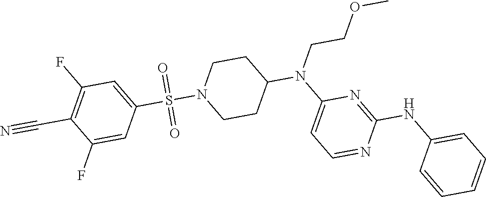 [SMILES: COCCN(c1ccnc(Nc2ccccc2)n1)C1CCN(S(=O)(=O)c2cc(F)c(C#N)c(F)c2)CC1] 19F NMR (376 MHz, DMSO-d6): δ −101.47 (2F). 1H-VT-NMR(400 MHz, DMSOd6): δ 8.68 (s, 1H), 7.93 (d, J = 6.4 Hz, 1H), 7.85 (d, J = 6.8 Hz, 2H), 7.64 (d, J = 7.6 Hz, 2H), 7.15 (t, J = 7.6 Hz, 2H), 6.86 (t, J = 7.6 Hz, 1H), 6.13 (d, J = 6.0 Hz, 1H), 4.22 (brs, 1H), 3.87 (d, J = 12.0 Hz, 2H), 3.53-3.43 (m, 4H), 3.29 (s, 3H), 2.68-2.60 (m, 2H), 1.88-1.76 (m, 4H). | 1.54 | 529.2 (M + H)+ | Method- 2 |
| A8 | 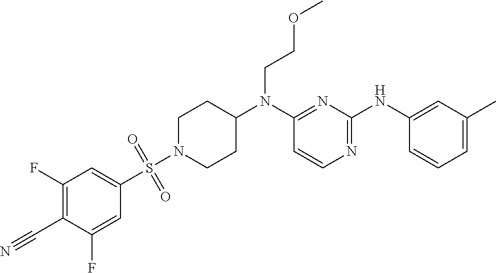 [SMILES: COCCN(c1ccnc(Nc2cccc(C)c2)n1)C1CCN(S(=O)(=O)c2cc(F)c(C#N)c(F)c2)CC1] 19F NMR (376 MHz, DMSO-d6): δ −101.44 (2F) 1H-NMR (400 MHz, DMSO-d6): δ 8.60 (s, 1H), 7.93 (d, J = 6.00 Hz, 1H), 7.85 (d, J = 6.80 Hz, 2H), 7.46-7.43 (m, 2H), 7.03 (t, J = 8.00 Hz, 1H), 6.69 (d, J = 7.60 Hz, 1H), 6.12 (d, J = 6.40 Hz, 1H), 4.24 (bs, 1H), 3.88 (d, J = 12.00 Hz, 2H), 3.52-3.45 (m, 4H), 3.29 (s, 3H), 2.68-2.61 (m, 2H), 2.23 (s, 3H), 1.88-1.78 (m, 4H) ppm. | 1.66 | 543.3 [M + H]+ | Method- 4 |
| A9 | 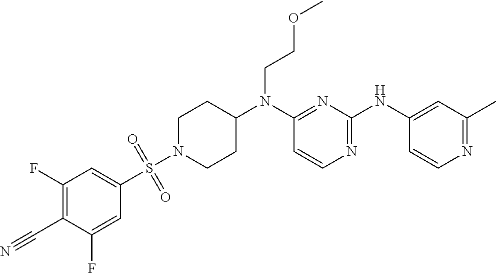 [SMILES: COCCN(c1ccnc(Nc2ccnc(C)c2)n1)C1CCN(S(=O)(=O)c2cc(F)c(C#N)c(F)c2)CC1] 19F NMR (376 MHz, DMSO-d6): δ −101.43 (2F). 1H-VT-NMR (400 MHz, DMSO-d6): δ 9.14 (s, 1H), 8.11 (d, J = 6.00 Hz, 1H), 8.00 (d, J = 6.40 Hz, 1H), 7.85 (d, J = 7.20 Hz, 2H), 7.56 (d, J = 2.00 Hz, 1H), 7.45-7.43 (m, 1H), 6.26 (d, J = 6.40 Hz, 1H), 4.22 (bs, 1H), 3.90 (d, J = 12.40 Hz, 2H), 3.57-3.47 (m, 4H), 3.30 (s, 3H), 2.73- 2.66 (m, 2H), 2.33 (s, 3H), 1.89-1.79 (m, 4H) ppm. | 1.425 | 544.2 [M + H]+ | Method- 2 |
| A10 | 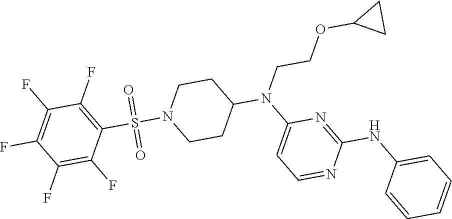 [SMILES: O=S(=O)(c1c(F)c(F)c(F)c(F)c1F)N1CCC(N(CCOC2CC2)c2ccnc(Nc3ccccc3)n2)CC1] 19F NMR (376 MHz, DMSO-d6): δ −135.64 to −135.75 (2F), −147.02 to −147.14 (1F), −159.46 to −159.63 (2F). 1H-VT-NMR (400 MHz, DMSO-d6): δ 8.81 (s, 1H), 7.95 (d, J = 6.00 Hz, 1H), 7.65 (d, J = 7.60 Hz, 2H), 7.21 (t, J = 8.00 Hz, 2H), 6.91 (t, J = 7.20 Hz, 1H), 6.19 (d, J = 6.00 Hz, 1H), 4.26 (bs, 1H), 3.88 (d, J = 12.00 Hz, 2H), 3.59-3.53 (m, 4H), 3.33-3.30 (m, 1H), 2.92-2.85 (m, 2H), 1.89-1.85 (m, 4H), 0.47-0.42 (m, 4H) ppm. | 1.735 | 584.2 (M + H)+ | Method- 2 |
| A11 | 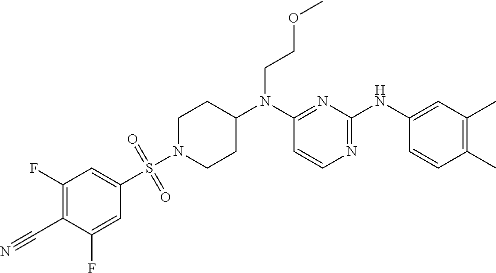 [SMILES: COCCN(c1ccnc(Nc2ccc(C)c(C)c2)n1)C1CCN(S(=O)(=O)c2cc(F)c(C#N)c(F)c2)CC1] 19F NMR (376 MHz, DMSO-d6): δ −101.22 (2F). 1H-VT-NMR (400 MHz, DMSO-d6): δ 8.48 (s, 1H), 7.92-7.85 (m, 3H), 7.40-7.34 (m, 2H), 6.91 (d, J = 8.00 Hz, 1H), 6.09 (d, J = 6.00 Hz, 1H), 4.25 (bs, 1H), 3.89 (d, J = 12.00 Hz, 2H), 3.50-3.44 (m, 4H), 3.29 (s, 3H), 2.68-2.61 (m, 2H), 2.15-2.06 (m, 6H), 1.88-1.83 (m, 4H) ppm. | 1.673 | 557.2 (M + H)+ | Method- 2 |
| A12 | 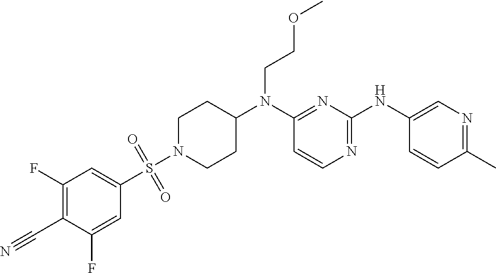 [SMILES: COCCN(c1ccnc(Nc2ccc(C)nc2)n1)C1CCN(S(=O)(=O)c2cc(F)c(C#N)c(F)c2)CC1] 19F-NMR (376 MHz, DMSO-d6): δ −101.25 to δ −101.23 (2F). 1H-VT-NMR (400 MHz, DMSO-d6): δ 8.77 (s, 1H), 8.65 (d, J = 2.40 Hz, 1H), 7.97-7.93 (m, 2H), 7.85 (d, J = 6.80 Hz, 2H), 7.03 (d, J = 8.40 Hz, 1H), 6.15 (d, J = 6.40 Hz, 1H), 4.25-4.20 (m, 1H), 3.89 (d, J = 12.00 Hz, 2H), 3.52-3.44 (m, 4H), 3.29 (s, 3H), 2.70-2.64 (m, 2H), 2.38 (s, 3H), 1.88-1.79 (m, 4H) ppm. | 1.328 | 544.2 [M + H]+ | Method- 2 |
| A13 | 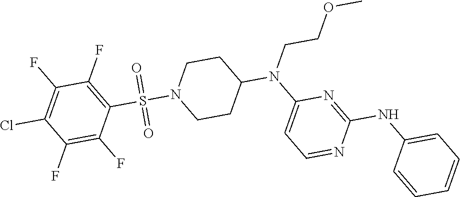 [SMILES: COCCN(c1ccnc(Nc2ccccc2)n1)C1CCN(S(=O)(=O)c2c(F)c(F)c(Cl)c(F)c2F)CC1] 19F NMR (376 MHz, DMSO-d6): δ −135.32 to −135.25 (2F), −138.84 to −138.77 (2F). 1H-VT-NMR (400 MHz, DMSO-d6): 8.67 (s, 1H), 7.94 (d, J = 6.0 Hz, 1H), 7.65 (dd, J = 8.8, 1.2 Hz, 2H), 7.65 (dd, J = 7.6, 0.8 Hz, 2H), 6.87 (t, J = 7.2 Hz , 1H), 6.15 (d, J = 6.4 Hz, 1H), 4.26-4.24 (m, 1H), 3.91-3.88 (m, 2H), 3.56- 3.53 (m, 2H), 3.50-3.46 (m, 2H), 3.30 (s, 3H), 2.95-2.88 (m, 2H), 1.89-1.85 (m, 4H) ppm. | 1.725 | 574.1 [M + H]+ | Method- 2 |
| A14 | 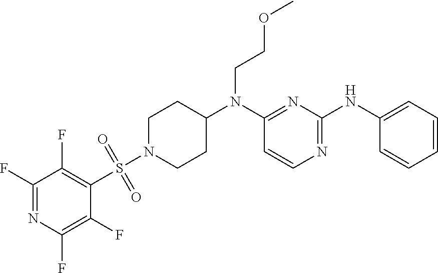 [SMILES: COCCN(c1ccnc(Nc2ccccc2)n1)C1CCN(S(=O)(=O)c2c(F)c(F)nc(F)c2F)CC1] 19F NMR (376 MHz, DMSO-d6): δ −89.18 to δ −89.07 (2F), δ −137.22 to δ −137.11 (2F). 1H-VT-NMR (400 MHz, DMSO-d6): δ 8.69 (s, 1H), 7.95 (d, J = 6.00 Hz, 1H), 7.67 (d, J = 7.60 Hz, 2H), 7.20 (t, J = 8.00 Hz, 2H), 6.88 (t, J = 7.20 Hz, 1H), 6.15 (d, J = 6.00 Hz, 1H), 4.26 (bs, 1H), 3.92 (d, J = 12.40 Hz, 2H), 3.57-3.47 (m, 4H), 3.30 (s, 3H), 3.03-2.99 (m, 2H), 1.92- 1.88 (m, 4H). | 1.668 | 541.1 [M + H]+ | Method- 2 |
| A15 | 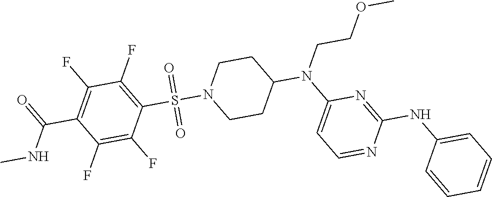 [SMILES: CNC(=O)c1c(F)c(F)c(S(=O)(=O)N2CCC(N(CCOC)c3ccnc(Nc4ccccc4)n3)CC2)c(F)c1F] 19F NMR (376 MHz, DMSO-d6): δ −135.14 to −135.06 (2F), −139.792 to −139.70 (2F) 1H-VT-NMR (400 MHz, DMSO-d6): δ 8.68 (bs, 2H), 7.94 (d, J = 6.40 Hz, 1H), 7.66 (d, J = 8.00 Hz, 2H), 7.18 (t, J = 7.60 Hz, 2H), 6.89 (t, J = 7.20 Hz, 1H), 6.16 (d, J = 6.00 Hz, 1H), 4.28-4.23 (m, 1H), 3.91 (d, J = 12.00 Hz, 2H), 3.57-3.47 (m, 4H), 3.30 (s, 3H), 2.95-2.86 (m, 5H), 1.90-1.86 (m, 4H) ppm. | 1.53 | 597.31 (M + H)+ | Method- 1 |
| A16 | 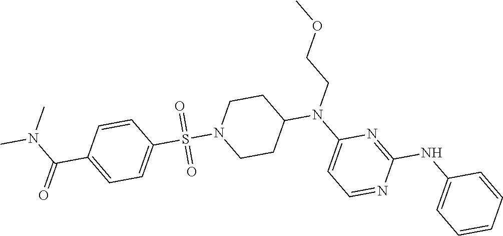 [SMILES: COCCN(c1ccnc(Nc2ccccc2)n1)C1CCN(S(=O)(=O)c2ccc(C(=O)N(C)C)cc2)CC1] 1H-VT-NMR (400 MHz, DMSO-d6): δ 8.68 (s, 1H), 7.92-7.85 (m, 3H), 7.69 (d, J = 7.6 Hz, 2H), 7.61 (d, J = 8.0 Hz, 2H), 7.07 (t, J = 7.6 Hz, 2H), 6.86 (t, J = 7.2 Hz, 1H), 6.13 (d, J = 6.0 Hz, 1H), 4.28-4.26 (m, 1H), 3.85 (d, J = 11.6 Hz, 2H), 3.55-3.44 (m, 4H), 3.29 (s, 3H), 2.99 (s, 6H), 2.50-2.40 (m, 2H), 1.89-1.77 (m, 4H). | 1.363 | 539.2 [M + H]+ | Method- 2 |
| A17 | 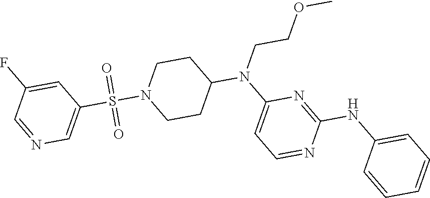 [SMILES: COCCN(c1ccnc(Nc2ccccc2)n1)C1CCN(S(=O)(=O)c2cncc(F)c2)CC1] 19F NMR (376 MHz, DMSO): δ −123.769 to −123.787 (1F). 1H-VT-NMR (400 MHz, DMSO-d6): δ 8.97 (d, J = 2.8 Hz, 1H), 8.85 (s, 1H), 8.67 (s, 1H), 8.18- 8.13 (m, 1H), 7.92 (d, J = 6.0 Hz, 1H), 7.62 (d, J = 7.2 Hz, 2H), 7.11 (t, J = 7.2 Hz, 2H), 6.85 (t, J = 7.2 Hz, 1H), 6.13 (d, J = 6.0 Hz, 1H), 4.26- 4.22 (m, 1H), 3.90 (d, J = 12.00 Hz, 2H), 3.54- 3.43 (m, 4H), 3.29 (s, 3H), 2.61-2.51 (m, 2H), 1.87-1.76 (m, 4H). | 1.406 | 487.1 [M + H]+ | Method- 2 |
| A18 | 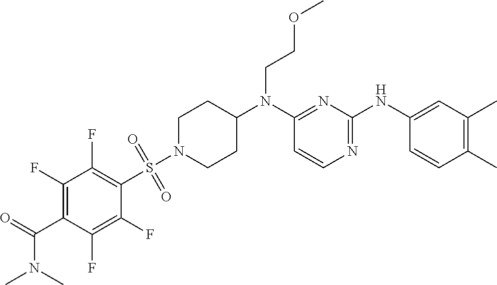 [SMILES: COCCN(c1ccnc(Nc2ccc(C)c(C)c2)n1)C1CCN(S(=O)(=O)c2c(F)c(F)c(C(=O)N(C)C)c(F)c2F)CC1] 19F NMR (376 MHz, DMSO-d6): δ −134.50 to −134.47 (2F), −140.30 to −140.21 (2F). 1H-NMR (400 MHz, DMSO-d6): δ 8.82 (s, 1H), 7.92 (d, J = 6.00 Hz, 1H), 7.44 (s, 1H), 7.37 (d, J = 1.60 Hz, 1H), 6.92 (d, J = 8.40 Hz, 1H), 6.12 (d, J = 6.00 Hz, 1H), 4.38 (m, 1H), 3.87- 3.84 (m, 2H), 3.54-3.44 (m, 4H), 3.27 (s, 3H), 3.08 (s, 3H), 2.98 (s, 3H), 2.90 (m, 2H), 2.12 (s, 6H), 1.86 (m, 4H) | 1.56 | 639.3 [M + H]+ | Method- 4 |
| A19 | 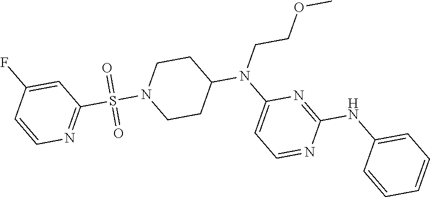 [SMILES: COCCN(c1ccnc(Nc2ccccc2)n1)C1CCN(S(=O)(=O)c2cc(F)ccn2)CC1] 19F NMR (376 MHz, DMSO): δ −98.12 (1F). 1H-VT-NMR (400 MHz, DMSO-d6): δ 8.89- 8.84 (m, 1H), 8.69 (s, 1H), 7.93 (d, J = 6.00 Hz, 1H), 7.87-7.83 (m, 1H), 7.71-7.64 (m, 3H), 7.15 (t, J = 7.2 Hz, 2H), 6.88 (t, J = 7.2 Hz, 1H), 6.14 (d, J = 6.0 Hz, 1H), 4.35-4.31 (m, 1H), 3.93 (d, J = 12.00 Hz, 2H), 3.51-3.42 (m, 4H), 3.29 (s, 3H), 2.92-2.83 (m, 2H), 1.82-1.74 (m, 4H). | 1.381 | 487.1 [M + H]+ | Method- 2 |
| A20 | 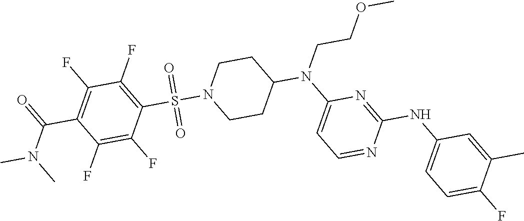 19F NMR (376 MHz, DMSO-d6): δ −127.37 (1F), −134.51 (2F), −140.24 to −140.33 (2F). 1H-VT-NMR (400 MHz, DMSO-d6): δ 8.62 (s, 1H), 7.93 (d, J = 6.4 Hz, 1H), 7.56-7.52 (m, 1H), 7.48-7.42 (m, 1H), 6.92 (t, J = 11.2 Hz, 1H), 6.15 (d, J = 6.0 Hz, 1H), 4.25 (brs, 1H), 3.90 (d, J = 12.0 Hz, 2H), 3.57-3.52 (m, 2H), 3.50-3.45 (m, 2H), 3.28 (s, 3H), 3.09-3.06 (m, 2H), 2.98 (bs, 4H), 2.97-2.90 (m, 2H), 2.18 (d, J = 1.2 Hz, 3H), 1.88-1.84 (m, 4H). | 2.528 | 643.2 [M + H]+ | Method- 3 |
| A21 | 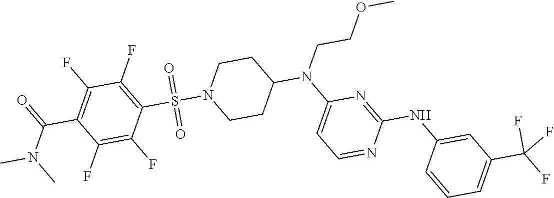 [SMILES: COCCN(c1ccnc(Nc2cccc(C(F)(F)F)c2)n1)C1CCN(S(=O)(=O)c2c(F)c(F)c(C(=O)N(C)C)c(F)c2F)CC1] 19F NMR (376 MHz, DMSO-d6): δ −61.02 (3F), −134.45 to −134.48 (2F), −140.22 to −140.32 (2F). 1H-NMR (400 MHz, DMSO-d6): δ 9.40 (s, 1H), 8.14 (s, 1H), 8.00 (d, J = 6.40 Hz, 1H), 7.91 (d, J = 7.60 Hz, 1H), 7.40 (t, J = 8.00 Hz, 1H), 7.18 (d, J = 7.60 Hz, 1H), 6.25 (d, J = 5.60 Hz, 1H), 4.20 (brs, 1H), 3.86 (d, J = 11.20 Hz, 2H), 3.58 (bs, 2H), 3.46 (t, J = 6.40 Hz, 2H), 3.27 (s, 3H), 3.08 (s, 3H), 2.98 (s, 3H), 2.95-2.81 (m, 2H), 1.90-1.81 (m, 4H) ppm. | 2.61 | 679.1 [M + H]+ | Method- 3 |
| A22 | 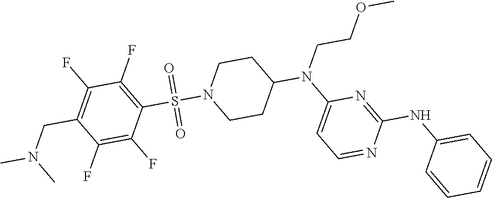 [SMILES: COCCN(c1ccnc(Nc2ccccc2)n1)C1CCN(S(=O)(=O)c2c(F)c(F)c(CN(C)C)c(F)c2F)CC1] 19F NMR (376 MHz, DMSO-d6): δ −136.77 to −136.71 (2F), δ −139.78 to −139.72 (2F). 1H-NMR (400 MHz, DMSO-d6): δ 8.69 (s, 1H), 7.94 (d, J = 6.00 Hz, 1H), 7.66 (d, J = 7.60 Hz, 2H), 7.17 (t, J = 8.00 Hz, 2H), 6.86 (t, J = 7.20 Hz, 1H), 6.16 (d, J = 6.00 Hz, 1H), 4.32- 4.28 (m, 1H), 3.92 (d, J = 12.00 Hz, 2H), 3.70 (s, 2H), 3.53-3.46 (m, 4H), 3.29 (s, 3H), 2.92- 2.86 (m, 2H), 2.25 (s, 6H), 1.88-1.84 (m, 4H) ppm. | 2.56 | 597.2 (M + H)+ | Method- 3 |
| A23 | 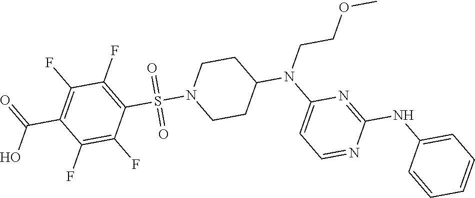 [SMILES: COCCN(c1ccnc(Nc2ccccc2)n1)C1CCN(S(=O)(=O)c2c(F)c(F)c(C(=O)O)c(F)c2F)CC1] 19F NMR (376 MHz, DMSO-d6): δ −136.71 to −136.76 (2F), −141.69 to −141.75 (2F). 1H-NMR (400 MHz, DMSO-d6): δ 9.00 (s, 1H), 7.93 (d, J = 6.4 Hz, 1H), 7.66 (d, J = 8.00 Hz, 2H), 7.15-7.13 (m, 2H), 6.88-6.84 (m, 1H), 6.18-6.16 (m, 1H), 5.80 (t, J = 6.0 Hz, 1H), 4.66 (d, J = 5.6 Hz, 2H), 3.85 (d, J = 12.4 Hz, 2H), 3.54-5.52 (m, 1H), 3.47-3.40 (m, 2H), 3.28 (s, 3H), 2.81-2.79 (m, 4H), 1.85- 1.83 (m, 4H). | 1.466 | 570.2 [M++H] | Method- 2 |
| A24 | 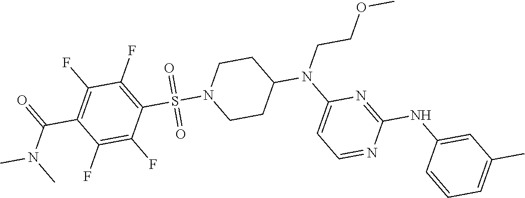 19F NMR (376 MHz, DMSO-d6): δ −134.33 to −134.42 (2F), −140.21 to −140.25 (2F) 1H-NMR (400 MHz, DMSO-d6): δ 8.95 (s, 1H), 7.94 (d, J = 6.00 Hz, 1H), 7.51-7.43 (m, 2H), 7.03 (t, J = 7.60 Hz, 1H), 6.68 (d, J = 7.20 Hz, 1H), 6.15 (d, J = 6.00 Hz, 1H), 3.85 (d, J = 10.80 Hz, 2H), 3.56-3.46 (m, 5H), 3.27 (s, 3H), 3.08 (s, 3H), 2.99 (s, 3H), 2.88 (bs, 2H), 2.21 (s, 3H), 1.88-1.75 (m, 4H). | 2.5 | 625.2 [M + H]+ | Method- 3 |
| A25 | 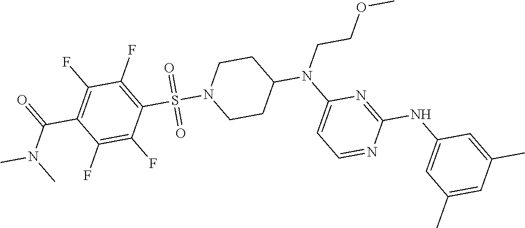 19F NMR (376 MHz, DMSO-d6): δ −134.35 to −134.45 (2F), −140.21 (2F). 1H-NMR (400 MHz, DMSO-d6): δ 8.86 (s, 1H), 7.93 (d, J = 6.00 Hz, 1H), 7.29 (s, 2H), 6.49 (s, 1H), 6.14 (d, J = 5.60 Hz, 1H), 4.56 (bs, 1H), 3.86 (d, J = 10.80 Hz, 2H), 3.56-3.44 (m, 4H), 3.27 (s, 3H), 3.08 (s, 3H), 2.99 (s, 3H), 2.90- 2.78 (m, 2H), 2.16 (bs, 6H), 1.89-1.78 (m, 4H) ppm. | 1.69 | 639.2 [M + H]+ | Method- 2 |
| A26 | 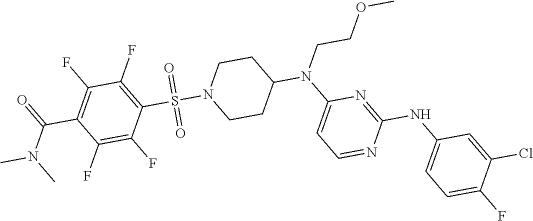 [SMILES: COCCN(c1ccnc(Nc2ccc(F)c(Cl)c2)n1)C1CCN(S(=O)(=O)c2c(F)c(F)c(C(=O)N(C)C)c(F)c2F)CC1] 19F NMR (376 MHz, DMSO-d6): δ −126.87 (1F), −134.44 to −134.47 (2F), −140.20 (2F). 1H-NMR (400 MHz, DMSO-d6): δ 9.27 (s, 1H), 8.04 (q, J = 2.40 Hz, 1H), 7.97 (d, J = 6.40 Hz, 1H), 7.52 (d, J = 5.60 Hz, 1H), 7.23 (t, J = 9.20 Hz, 1H), 6.22 (d, J = 5.60 Hz, 1H), 4.51 (s, 1H), 3.87 (d, J = 10.40 Hz, 2H), 3.56 (s, 4H), 3.27 (s, 3H), 3.08 (s, 3H), 2.91 (d, J = 10.00 Hz, 5H), 1.88 (d, J = 12.40 Hz, 4H). | 1.62 | 663.2 [M + H]+ | Method- 4 |
| A27 | 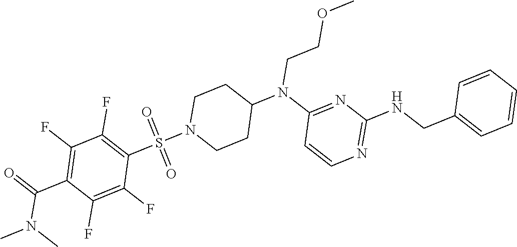 [SMILES: COCCN(c1ccnc(NCc2ccccc2)n1)C1CCN(S(=O)(=O)c2c(F)c(F)c(C(=O)N(C)C)c(F)c2F)CC1] 19F NMR (376 MHz, DMSO-d6): δ −134.51 to −134.42 (2F), −140.36 to −140.27 (2F). 1H-NMR (400 MHz, DMSO-d6): δ 7.80 (d, J = 6.40 Hz, 1H), 7.20-7.28 (m, 5H), 6.07 (d, J = 6.40 Hz, 1H), 4.49 (d, J = 6.40 Hz, 2H), 4.20- 4.14 (m, 1H), 3.82 (d, J = 12.00 Hz, 2H), 3.49- 3.39 (m, 2H), 3.39-3.34 (m, 2H), 3.24 (s, 3H), 3.19 (s, 3H), 3.08 (bs, 4H), 2.84 (t, J = 12.00 Hz, 2H), 1.89-1.74 (m, 4H) | 2.443 | 625.7 [M + H]+ | Method- 3 |
| A88 | 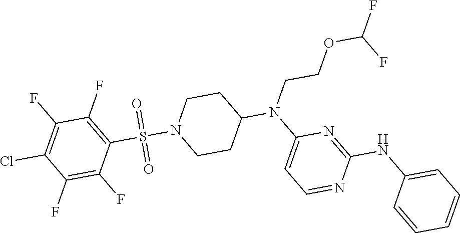 [SMILES: O=S(=O)(c1c(F)c(F)c(Cl)c(F)c1F)N1CCC(N(CCOC(F)F)c2ccnc(Nc3ccccc3)n2)CC1] 19FNMR (376 MHz, DMSO-d6): δ −135.91 (2F), −138.02 (1F), −138.79 (2F), −142.15 (1F). 1H-VT-NMR(400 MHz, DMSO-d6): δ 8.84 (s, 1H), 7.97 (d, J = 6.4 Hz, 1H), 7.64 (d, J = 7.6 Hz, 2H), 7.21 (t, J = 7.2 Hz, 2H), 6.91 (t, J = 7.2 Hz, 1H), 6.63 (t, J = 76 Hz, 1H), 6.23 (d, J = 6.0 Hz, 1H), 4.25-4.15 (m, 1H), 3.97 (t, J = 6.4 Hz, 2H), 3.88 (d, J = 12.0 Hz, 2H), 3.67 (t, J = 6.4 Hz, 2H), 2.95-2.88 (m, 2H), 1.90-1.86 (m, 4H). | 1.79 | 610.1 [M + H]+ | Method- 2 |
| A89 | 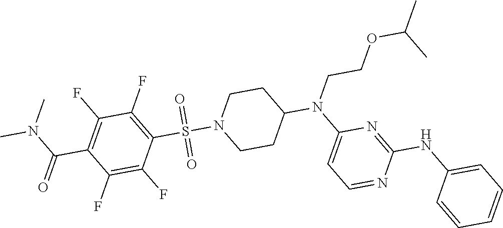 19F NMR(376 MHz, DMSO-d6): δ −82.45 (2F), 134.34 (2F), −140.25 (2F) 1H-VT-NMR(400 MHz, DMSO-d6): δ 8.73 (s, 1H), 7.97 (d, J = 6.0 Hz, 1H), 7.65 (d, J = 7.6 Hz, 2H), 7.19 (t, J = 7.6 Hz, 2H), 6.89 (t, J = 7.2 Hz, 1H), 6.64 (t, J = 76.4 Hz, 1H), 6.20 (d, J = 6.0 Hz, 1H), 4.27-4.22 (m, 1H), 4.00-3.88 (m, 4H), 3.66 (t, J = 6.4 Hz, 2H), 3.09 (s, 3H), 3.03-2.90 (m, 5H), 1.91-1.86 (m, 4H). | 2.56 | 647.1 [M + H]+ | Method- 3 |
| A90 | 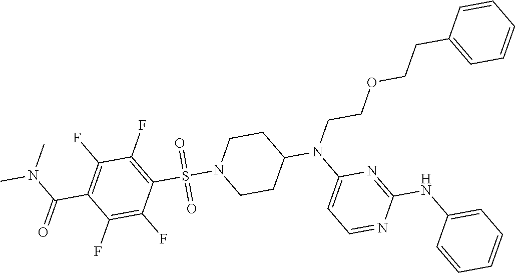 [SMILES: CN(C)C(=O)c1c(F)c(F)c(S(=O)(=O)N2CCC(N(CCOCCc3ccccc3)c3ccnc(Nc4ccccc4)n3)CC2)c(F)c1F] 19F NMR(376 MHz, DMSO-d6): δ −134.33 (2F), −140.29 (2F) 1H-VT-NMR(400 MHz, DMSO-d6): δ 8.66 (s, 1H), 7.92 (d, J = 6.0 Hz, 1H), 7.65 (d, J = 7.6 Hz, 2H), 7.30-7.13 (m, 7H), 6.87 (t, J = 7.2 Hz, 1H), 6.13 (d, J = 6.0 Hz, 1H), 4.30-4.20 (m, 1H), 3.88 (d, J = 12.0 Hz, 2H), 3.66 (t, J = 6.8 Hz, 2H), 3.57-3.50 (m, 4H), 3.10 (s, 3H), 2.98 (s, 3H), 2.92-2.80 (m, 4H), 1.90-1.80 (m, 4H). | 1.718 | 701.2 [M + H]+ | Method- 2 |
| A99 | 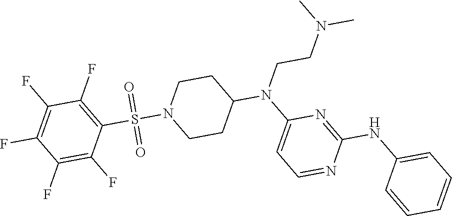 [SMILES: CN(C)CCN(c1ccnc(Nc2ccccc2)n1)C1CCN(S(=O)(=O)c2c(F)c(F)c(F)c(F)c2F)CC1] 19F-NMR (376 MHz, DMSO-d6): δ −135.61 (2F), −146.75 (1F), −159.33 (2F) 1H-VT-NMR (400 MHz, DMSO-d6): δ 8.68 (s, 1H), 7.95 (d, J = 6.0 Hz, 1H), 7.68 (d, J = 7.6 Hz, 2H), 7.19 (t, J = 7.6 Hz, 2H), 6.87 (t, J = 7.20 Hz, 1H), 6.11 (d, J = 6.4 Hz, 1H), 4.30 (bs, 1H), 3.91 (d, J = 12.00 Hz, 2H), 3.44 (t, J = 7.60 Hz, 2H), 2.93-2.87 (m, 2H), 2.40 (t, J = 7.60 Hz, 2H), 2.22 (s, 6H), 1.89-1.83 (m, 4H). | 1.952 | 571.4 (M + H)+ | Method- 5 |
| A100 | 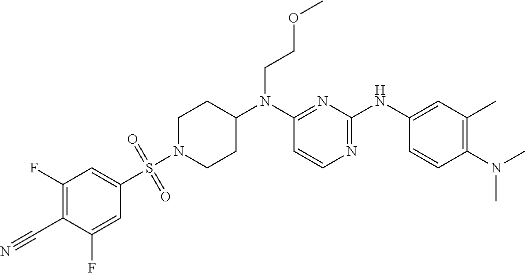 [SMILES: COCCN(c1ccnc(Nc2ccc(N(C)C)c(C)c2)n1)C1CCN(S(=O)(=O)c2cc(F)c(C#N)c(F)c2)CC1] 19F NMR (376 MHz, DMSO-d6): δ −101.33 (2F) 1H-NMR (400 MHz, DMSO-d6): δ 8.47 (s, 1H), 7.90-7.84 (m, 3H), 7.42-7.35 (m, 2H), 6.90 (d, J = 8.00 Hz, 1H), 6.09 (d, J = 6.40 Hz, 1H), 4.26 (bs, 1H), 3.89 (d, J = 8.00 Hz, 2H), 3.50-3.44 (m, 4H), 3.29 (s, 3H), 2.67-2.61(m, 2H), 2.59 (s, 6H), 2.15 (s, 3H), 1.88-1.82 (m, 4H) ppm. | 1.354 | 586.2 (M + H)+ | Method- 2 |
| A101 | 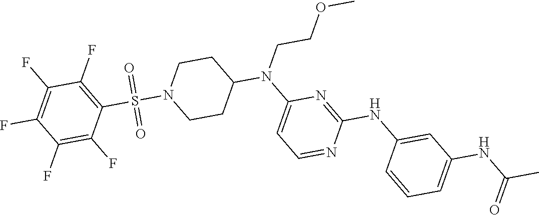 [SMILES: COCCN(c1ccnc(Nc2cccc(NC(C)=O)c2)n1)C1CCN(S(=O)(=O)c2c(F)c(F)c(F)c(F)c2F)CC1] 19F NMR (376 MHz, DMSO-d6): δ −135.69 (2F), −146.66 (1F), 159.31 (2F). 1H-VT-NMR (400 MHz, DMSO-d6): δ 9.54 (s, 1H), 8.65 (s, 1H), 7.94 (d, J = 6.0 Hz, 1H), 7.82 (s, 1H), 7.38-7.35 (m, 1H), 7.09-7.06 (m, 2H), 6.14 (d, J = 6.40 Hz, 1H), 4.30 (bs, 1H), 3.87 (d, J = 12.00 Hz, 2H), 3.55-3.51 (m, 2H), 3.49- 3.46 (m, 2H), 3.29 (s, 3H), 2.93-2.87 (m, 2H), 2.01 (s, 3H), 1.86-1.81 (m, 4H) ppm. | 1.503 | 615.2 [M + H]+ | Method- 2 |
| A102 | 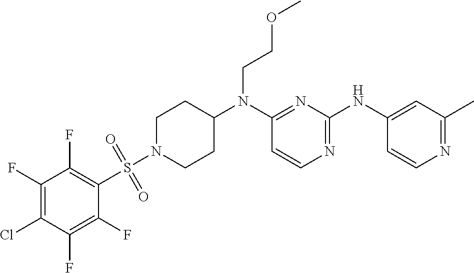 [SMILES: COCCN(c1ccnc(Nc2ccnc(C)c2)n1)C1CCN(S(=O)(=O)c2c(F)c(F)c(Cl)c(F)c2F)CC1] 19F NMR (376 MHz, DMSO-d6): δ −135.26 (2F), −138.74 to −138.78 (2F). 1H-VT-NMR (400 MHz, DMSO-d6): δ 9.28 (s, 1H), 8.14 (t, J = 4.00 Hz, 1H), 8.02 (d, J = 6.00 Hz, 1H), 7.61 (bs, 1H), 7.50 (d, J = 5.60 Hz, 1H), 6.30 (d, J = 6.00 Hz, 1H), 4.26-4.33 (m, 1H), 3.91 (d, J = 11.60 Hz, 2H), 3.62-3.49 (m, 4H), 3.30 (s, 3H), 2.99-2.95 (m, 2H), 2.37 (s, 3H), 1.92-1.87 (m, 4H). | 1.65 | 589.22 (M + H)+ | Method- 1 |
| A103 | 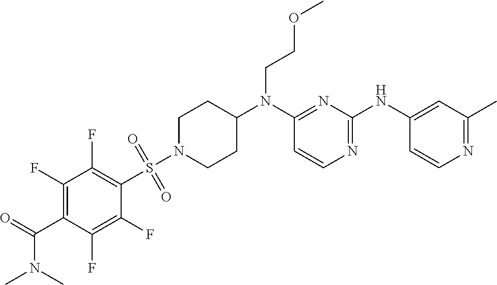 [SMILES: COCCN(c1ccnc(Nc2ccnc(C)c2)n1)C1CCN(S(=O)(=O)c2c(F)c(F)c(C(=O)N(C)C)c(F)c2F)CC1] 19F NMR (376 MHz, DMSO-d6): δ −134.45 (2F), −140.31 to −140.21 (2F). 1H-VT-NMR (400 MHz, DMSO-d6): δ 10.50 (s, 1H), 8.33 (d, J = 6.80 Hz, 1H), 8.14 (d, J = 6.40 Hz, 1H), 8.06 (s, 1H), 7.91 (dd, J = 2.00, 6.80 Hz, 1H), 6.54 (d, J = 6.00 Hz, 1H), 4.25- 4.23 (m, 1H), 3.93 (d, J = 12.00 Hz, 2H), 3.67- 3.64 (m, 2H), 3.54-3.48 (m, 2H), 3.30 (s, 3H), 3.09 (s, 3H), 3.03-2.92 (m, 5H), 2.58 (s, 3H), 1.99-1.88 (m, 4H) ppm. | 1.431 | 626.2 [M + H]+ | Method- 2 |
| A104 | 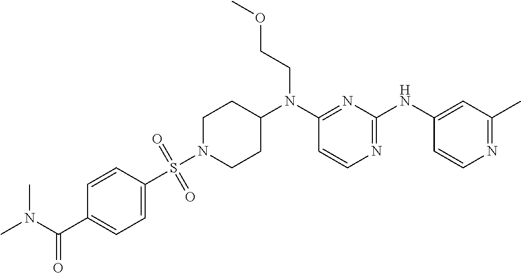 1H-NMR (400 MHz, DMSO-d6): δ 9.56 (s, 1H), 8.08 (s, 1H), 7.99 (d, J = 5.60 Hz, 1H), 7.85 (d, J = 7.60 Hz, 2H), 7.70-7.63 (m, 3H), 7.44 (bs, 1H), 6.31 (bs, 1H), 6.31 (d, J = 6.00 Hz, 1H), 4.55-4.41 (m, 1H), 3.83 (bs, 2H), 3.54- 3.45 (m, 5H), 3.33-3.28 (m, 3H), 3.04 (s, 3H), 2.92 (s, 3H), 2.27 (m, 3H), 1.86-1.78 (m, 4H). | 1.18 | 554.2 [M + H]+ | Method- 2 |
| A105 | 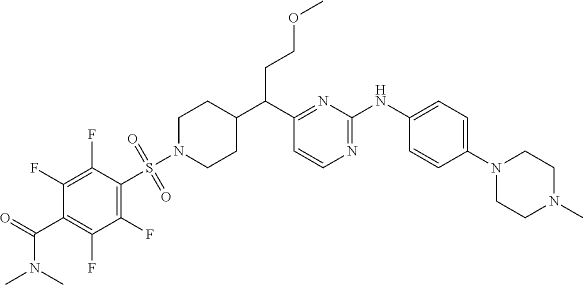 19F NMR (376 MHz, DMSO-d6): δ −134.78 (2F), −140.41 (2F). 1H-VT-NMR (400 MHz, DMSO-d6): δ 8.43 (s, 1H), 7.90 (d, J = 2.0 Hz, 1H), 7.52 (d, J = 8.8 Hz, 2H), 6.84 (d, J = 9.2 Hz, 2H), 6.10 (d, J = 6.0 Hz, 1H), 4.23 (brs, 1H), 3.91 (d, J = 12.4 Hz, 2H), 3.54-3.45 (m, 4H), 3.28 (s, 3H), 3.18- 3.05 (m, 5H), 3.00-2.97 (m, 4H), 2.69-2.66 (m, 4H), 2.40-2.36 (m, 3H), 1.95-1.84 (m, 4H) | 1.17 | 709.3 [M + H]+ | Method- 4 |
| A106 | 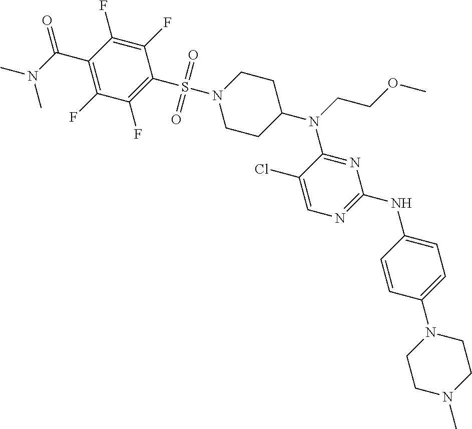 [SMILES: COCCN(c1nc(Nc2ccc(N3CCN(C)CC3)cc2)ncc1Cl)C1CCN(S(=O)(=O)c2c(F)c(F)c(C(=O)N(C)C)c(F)c2F)CC1] 19F NMR (376 MHz, DMSO-d6): δ −134.82 to −134.73 (2F), −140.56 to −140.47 (2F). 1H-NMR (400 MHz, DMSO-d6): δ 9.10 (s, 1H), 8.01 (s, 1H), 7.44 (d, J = 9.20 Hz, 2H), 6.84 (d, J = 9.20 Hz, 2H), 4.19-4.13 (m, 1H), 3.83 (d, J = 11.60 Hz, 2H), 3.63 (t, J = 6.00 Hz, 2H), 3.41-3.38 (m, 2H), 3.22 (s, 3H), 3.06-3.03 (m, 7H), 2.97-2.91 (m, 5H), 2.50-2.44 (m, 4H), 2.22 (s, 3H), 1.96-1.88 (m, 4H). | 1.47 | 743.3 [M + H]+ | Method- 2 |
| A107 | 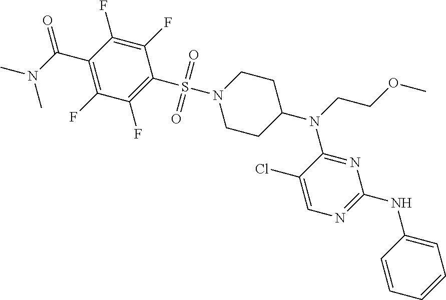 [SMILES: COCCN(c1nc(Nc2ccccc2)ncc1Cl)C1CCN(S(=O)(=O)c2c(F)c(F)c(C(=O)N(C)C)c(F)c2F)CC1] 19F NMR (376 MHz, DMSO-d6): δ −134.53 to −134.44 (2F), −140.43 to −140.34 (2F). 1H-NMR (400 MHz, DMSO-d6): δ 9.35 (s, 1H), 8.07 (s, 1H), 7.60 (d, J = 8.00 Hz, 2H), 7.20 (t, J = 8.00 Hz, 2H), 6.92 (t, J = Hz, 1H), 4.26- 4.20 (m, 1H), 3.83 (d, J = 11.20 Hz, 2H), 3.67 (t, J = 6.00 Hz, 2H), 3.42 (t, J = 6.40 Hz, 2H), 3.23 (s, 3H), 3.07 (s, 3H), 2.97 (s, 3H), 2.88- 2.84 (m, 2H), 1.91-1.88 (m, 4H). | 2.11 | 645.2 [M + H]+ | Method- 2 |
| A108 | 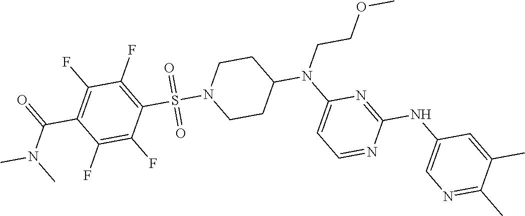 19F NMR (376 MHz, DMSO-d6): δ −134.46 to −134.37 (2F), −140.22 to −140.19 (2F). 1H-VT-NMR (400 MHz, DMSO-d6): δ 9.09 (s, 1H), 8.01 (d, J = 6.00 Hz, 1H), 7.37 (s, 2H), 6.28 (d, J = 6.00 Hz, 1H), 4.32 (bs, 1H), 3.93 (d, J = 11.60 Hz, 2H), 3.61 (t, J = 6.00 Hz, 2H), 3.51 (t, J = 6.40 Hz, 2H), 3.30 (s, 3H), 3.09 (bs, 3H), 2.98 (bs, 5H), 2.32 (s, 6H), 1.92-1.87 (m, 4H). | 1.43 | 640.2 [M + H]+ | Method- 2 |
| A109 | 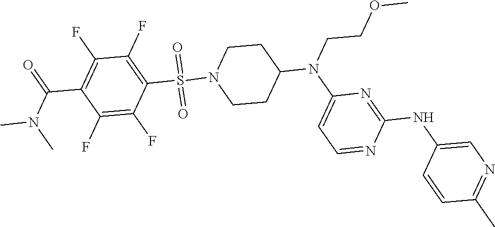 19F NMR (376 MHz, DMSO): δ −134.53 (2F), −140.22 to −140.31 (2F). 1H-VT-NMR (400 MHz, DMSO-d6): δ 8.76 (s, 1H), 8.65 (d, J = 2.40 Hz, 1H), 7.99 (dd, J = 8.4, 2.4 Hz, 1H), 7.94 (d, J = 6.00 Hz, 1H), 7.05 (d, J = 8.40 Hz, 1H), 6.18 (d, J = 6.00 Hz, 1H), 4.31-4.20 (m, 1H), 3.91 (d, J = 12.00 Hz, 2H), 3.56-3.46 (m, 4H), 3.29 (s, 3H), 3.08 (s, 3H), 2.98-2.93 (m, 5H), 2.38 (s, 3H), 1.90-1.85 (m, 4H) ppm. | 1.305 | 626.2 [M + H]+ | Method- 2 |
| A110 | 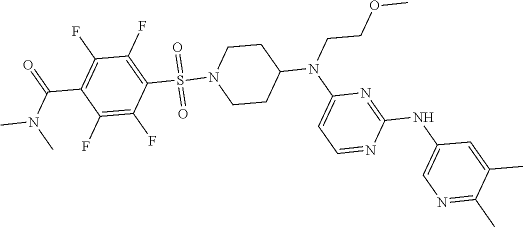 19F-NMR (376 MHz, DMSO-d6): δ −134.48 to −134.39 (2F), −140.24 to −140.20 (2F). 1H-NMR (400 MHz, DMSO-d6): δ 9.01 (s, 1H), 8.42 (d, J = 2.40 Hz, 1H), 7.95-7.90 (m, 2H), 6.18 (d, J = 5.60 Hz, 1H), 3.86 (d, J = 10.80 Hz, 2H), 3.55-3.43 (m, 5H), 3.27 (s, 3H), 3.08 (s, 3H), 2.98 (s, 3H), 2.95-2.85 (m, 2H), 2.31 (s, 3H), 2.15 (bs, 3H), 1.88-1.84 (m, 4H) ppm. | 1.27 | 640.2 [M + H]+ | Method- 2 |
| A111 | 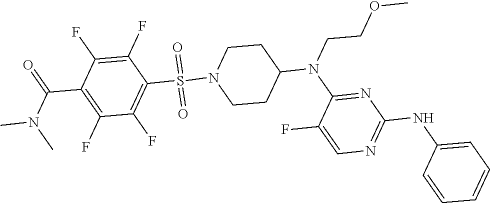 [SMILES: COCCN(c1nc(Nc2ccccc2)ncc1F)C1CCN(S(=O)(=O)c2c(F)c(F)c(C(=O)N(C)C)c(F)c2F)CC1] 19F NMR (376 MHz, DMSO-d6): δ −134.39 to −134.47(2F), −140.27 to −140.36 (2F), −159.40 (1F) 1H-VT-NMR (400 MHz, DMSO-d6): δ 8.80 (s, 1H), 7.96 (d, J = 7.20 Hz, 1H), 7.17 (d, J = 8.00 Hz, 2H), 7.18 (t, J = 8.00 Hz, 2H), 6.89 (t, J = 7.20 Hz, 1H), 4.39-4.33 (m, 1H), 3.92 (d, J = 12.00 Hz, 2H), 3.64-3.61 (m, 2H), 3.51-3.48 (m, 2H), 3.28 (s, 3H), 3.09 (s, 3H), 2.98 (s, 3H), 2.93-2.87 (m, 2H), 1.95-1.89 (m, 4H). | 2.53 | 629.3 [M + H]+ | Method- 3 |
| A112 | 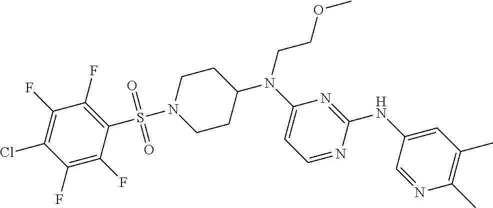 [SMILES: COCCN(c1ccnc(Nc2cnc(C)c(C)c2)n1)C1CCN(S(=O)(=O)c2c(F)c(F)c(Cl)c(F)c2F)CC1] 19F NMR (376 MHz, DMSO-d6): δ −135.13 to −135.18 (2F), −138.66 to −138.71 (2F). 1H-VT-NMR (400 MHz, DMSO-d6): δ 8.68 (s, 1H), 8.47 (s, 1H), 7.94 (d, J = 6.00 Hz, 1H), 7.83 (s, 1H), 6.16 (d, J = 6.00 Hz, 1H), 4.26-4.24 (m, 1H), 3.89 (d, J = 11.60 Hz, 2H), 3.53-3.46 (m, 4H), 3.28 (s, 3H), 2.94-2.87(m, 2H), 2.33 (s, 3H), 2.18 (s, 3H), 1.8-1.84 (s, 4H) ppm. | 1.41 | 603.2 [M + H]+ | Method- 2 |

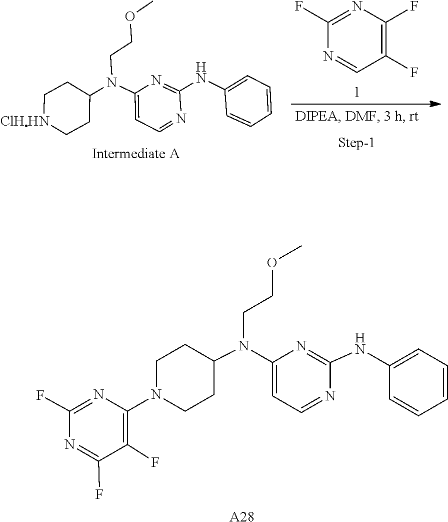

### Example A28 Synthesis of N4-(1-(2,5-difluoropyrimidin-4-yl)piperidin-4-yl)-N4-(2-methoxyethyl)-N2-phenylpyrimidine-2,4-diamine

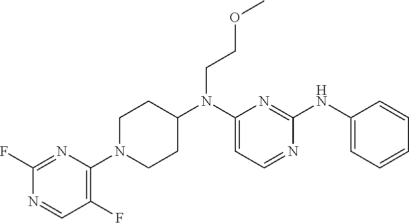 [SMILES: COCCN(c1ccnc(Nc2ccccc2)n1)C1CCN(c2nc(F)ncc2F)CC1]

In an oven-dried 8 mL pressure vial, N⁴-(2-methoxyethyl)-N²-phenyl-N⁴-(piperidin-4-yl)pyrimidine-2,4-diamine hydrochloride (200 mg, 0.611 mmol), diisopropylamine (0.532 ml, 3.05 mmol), 2,4,5-trifluoropyrimidine (73.7 mg, 0.550 mmol) and DMF (10 mL) were added at room temperature and further stirred for 3 h at the same temperature. The reaction was monitored by TLC and LCMS, quenched with cold water (15 ml) and extracted with ethyl acetate (2×15 ml). The combined organic layers were washed with water (2×20 mL) and dried over anhydrous sodium sulphate, filtered and concentrated under reduced pressure affording crude product. The crude mass was purified by prep HPLC (Method-A) and product containing fraction was lyophilized to get N⁴-(1-(2,5-difluoropyrimidin-4-yl)piperidin-4-yl)-N⁴-(2-methoxyethyl)-N²-phenylpyrimidine-2,4-diamine (27 mg, 0.060 mmol, 9.81% yield)) as an off white solid.

EXAMPLE A29 was prepared in the same manner as A28 from the appropriate starting materials.

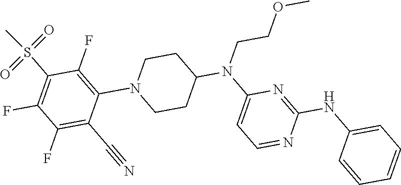 [SMILES: COCCN(c1ccnc(Nc2ccccc2)n1)C1CCN(Cc2c(F)c(S(C)(=O)=O)c(F)c(F)c2C#N)CC1]

¹⁹F NMR (376 MHz, DMSO-d6): δ −120.83 (1F), −133.00 (1F), −141.51 (1F). ¹HNMR (400 MHz, DMSO-d6): δ 9.05 (s, 1H), 7.97 (d, J=6.0 Hz, 1H), 7.74 (d, J=7.6 Hz, 2H), 7.24 (t, J=7.6 Hz, 2H), 6.89 (t, J=7.2 Hz, 1H), 6.23 (brs, 1H), 3.59-3.48 (m, 9H), 3.39-3.34 (m, 3H), 3.29 (s, 3H), 1.91-1.80 (m, 4H). LCMS (Method 2): RT: 1.55, m/z=561.2 (M+H)+

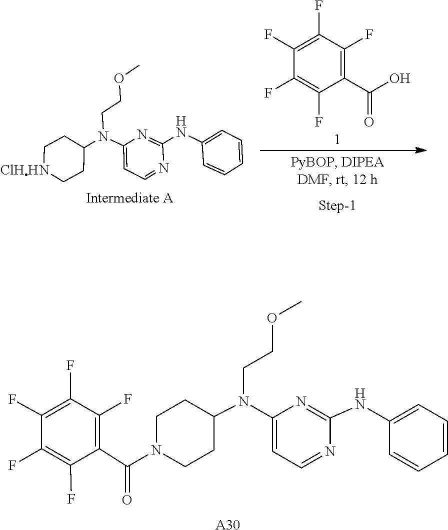

### Example A30 (4-((2-methoxyethyl)(2-(phenylamino)pyrimidin-4-yl)amino)piperidin-1 yl)(perfluorophenyl)methanone

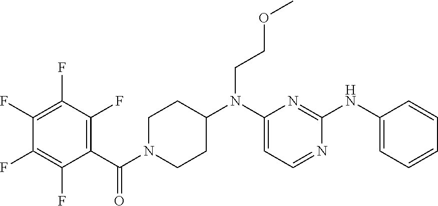 [SMILES: COCCN(c1ccnc(Nc2ccccc2)n1)C1CCN(C(=O)c2c(F)c(F)c(F)c(F)c2F)CC1]

To an oven dried 50 mL two-neck round-bottom flask equipped with N₂ balloon, 2,3,4,5,6-pentafluorobenzoic acid (117 mg, 0.550 mmol), DMF (10 mL), PyBOP (358 mg, 0.687 mmol), N4-(2-methoxyethyl)-N2-phenyl-N4-(piperidin-4-yl)pyrimidine-2,4-diamine hydrochloride (150 mg, 0.458 mmol) and DIPEA (0.240 ml, 1.374 mmol) were added at 0 °C. The resulting reaction mixture was stirred for 12 h at room temperature. The reaction was monitored by TLC; quenched with ice cold water (30 mL) and extracted with ethyl acetate (3×50 mL). The combined organic layers were dried over anhydrous sodium sulphate, filtered and concentrated under reduced pressure to afford crude product. The crude mass was purified by prep HPLC (Method C) to give (4-((2-methoxyethyl)(2-(phenylamino)pyrimidin-4-yl)amino)piperidin-1-yl)(perfluorophenyl)methanone (40.01 mg, 0.076 mmol, 16.63% yield) as an off-white solid.

The compounds in the following table 2 were prepared in the same manner as A30 from the appropriate starting materials.

| | | | | LCMS | | |
|---|---|---|---|---|---|---|
| | | | | Retention | LCMS Mass | LCMS |
| Cmp | Structure | 19F NMR | 1H NMR | Time | Obs | method |
| A2 | 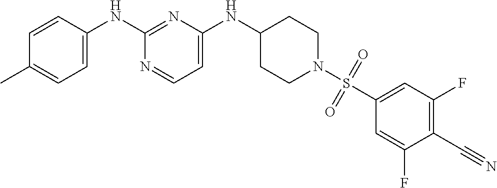 [SMILES: Cc1ccc(Nc2nccc(NC3CCN(S(=O)(=O)c4cc(F)c(C#N)c(F)c4)CC3)n2)cc1] | 19F NMR (376 MHz, DMSO-d6): δ −101.62 to −101.64 (2F) | 1H-NMR (400 MHz, DMSO-d6): δ 8.98 (bs, 1H), 7.93 (d, J = 7.20 Hz, 2H), 7.77 (d, J = 5.60 Hz, 1H). 7.54 (d, J = 8.00 Hz, 2H), 7.39 (s, 1H) 7.00 (d, J = 8.40 Hz, 2H), 5.91 (d, J = 6.00 Hz, 1H), 3.78 (s, 1H), 3.68 (d, J = 12.00 Hz, 2H), 2.72-2.67 (m, 2H), 2.23 (s, 3H), 2.00 (d, J = 10.80 Hz, 2H). 1.48 (g, J = 10.80 Hz, 2H) | 1.785 | 485.1 (M + H)+ | Method-9 |
| A3 | 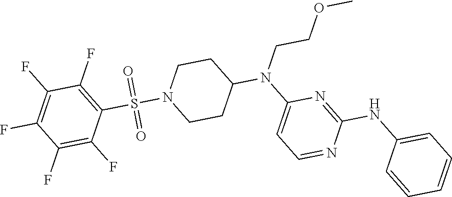 | 19F NMR (376 MHz, DMSO-d6): δ −135.59 (2F), −146.79 (1F), −159.36 (2F) | 1H-VT-NMR (400 MHz, DMSO-d6): δ 8.69 (s, 1H), 7.95 (d, J = 6.00 Hz, 1H), 7.67 (d, J = 7.60 Hz, 2H), 7.19 (t J = 7.60 Hz, 2H). 6.87 (t J = 7.6 Hz, 1H), 6.15 (d, J = 6.0 Hz 1H), 4.27-4.23 (m, 1H), 3.89 (d, J = 12.00 Hz, 2H), 3.56-3.47 (m, 4H), 3.30 (s, 3H), 2.91-2.87 (m, 2H), 1.93-1.86 (m, 4H) | 1.832 | 658.1 [M + H]+ | Method-3 |
| A4 | 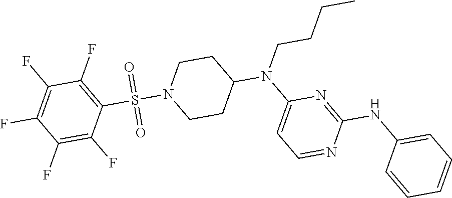 [SMILES: CCCCN(c1ccnc(Nc2ccccc2)n1)C1CCN(S(=O)(=O)c2c(F)c(F)c(F)c(F)c2F)CC1] | 19F NMR (376 MHz, DMSO-d6): δ −135.55 (2F), −146.69 to −146.81 (1F), −159.28 to −159.43 (2F). | 1H-NMR (400 MHz, DMSO-d6): δ 8.99 (s, 1H), 7.93 (d, J = 6.00 Hz, 1H), 7.68 (d, J = 7.60 Hz, 2H), 7.16 (t, J = 8.00 Hz, 2H), 6.85 (t, J = 7.20 Hz, 1H), 6.08 (d, J = 5.60 Hz, 1H), 3.85 (d, J = 11.60 Hz, 2H), 3.29-3.26 (m, 2H), 2.83 (s, 2H), 2.34-2.33 (m, 1H), 1.86-1.85 (m, 4H), 1.51- 1.49 (m, 2H), 1.39-1.33 (m, 2H), 0.93 (t, J = 7.60 Hz, 3H). | 1.824 | 556.2 (M + H)+ | Method-2 |
| A5 | 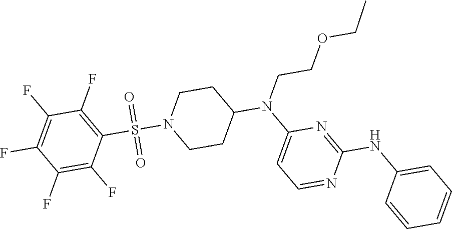 [SMILES: CCOCCN(c1ccnc(Nc2ccccc2)n1)C1CCN(S(=O)(=O)c2c(F)c(F)c(F)c(F)c2F)CC1] | 19F NMR (376 MHz, DMSO-d6): δ −135.62 to 135.56 (2F), −146.86 to −146.75 (1F), −159.46 to −159.29 (2F) | 1H-NMR (400 MHz, DMSO 08): 6 9.02 (8, 1H), 7.95 (d, J = 6.40 Hz, 1H). 7.68 (d, J = 8.00 Hz, 2H), 7.17 (t J = 7.60 Hz, 2H), 6.85 (t, J = 7.20 Hz, 1H), 8.16 (d. J = 6,40 Hz, 1H). 4.22 (m, 1H), 3.84 (0, J = 11.60 Hz, 2H), 3.53 - 3.42 (m. 6H). 2.84 (s, 2H), 1.88 ($, 4H), 1.13 (t, J = 6.80 Hz, 3H) ppm. | 1.711 | 572.2 (M + H)+ | Method-2 |
| A6 | 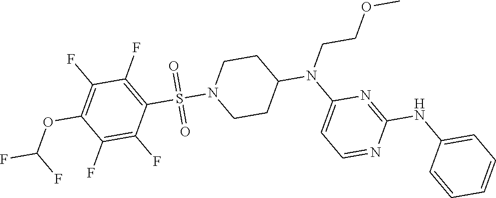 [SMILES: COCCN(c1ccnc(Nc2ccccc2)n1)C1CCN(S(=O)(=O)c2c(F)c(F)c(OC(F)F)c(F)c2F)CC1] | 19F NMR (376 MHz, DMSO-d6): δ −81.80 to −82.02 (2F), −135.84 to − | 1H-VT-NMR (400 MHz) DMSOd6): δ 8.70 (s, 1H), 7.94 (d, J = 6.00 Hz, 1H), 7.66 (d, J = 8.00 Hz, 2H), 7.58-7.17 (m, 3H), 6.87 (t, J = 7.60 Hz, 1H), 6.16 (d, J = 6.00 Hz, 1H), 4.27 (brs, | 2.357 | 606.2 [M + H]+ | Method-5 |

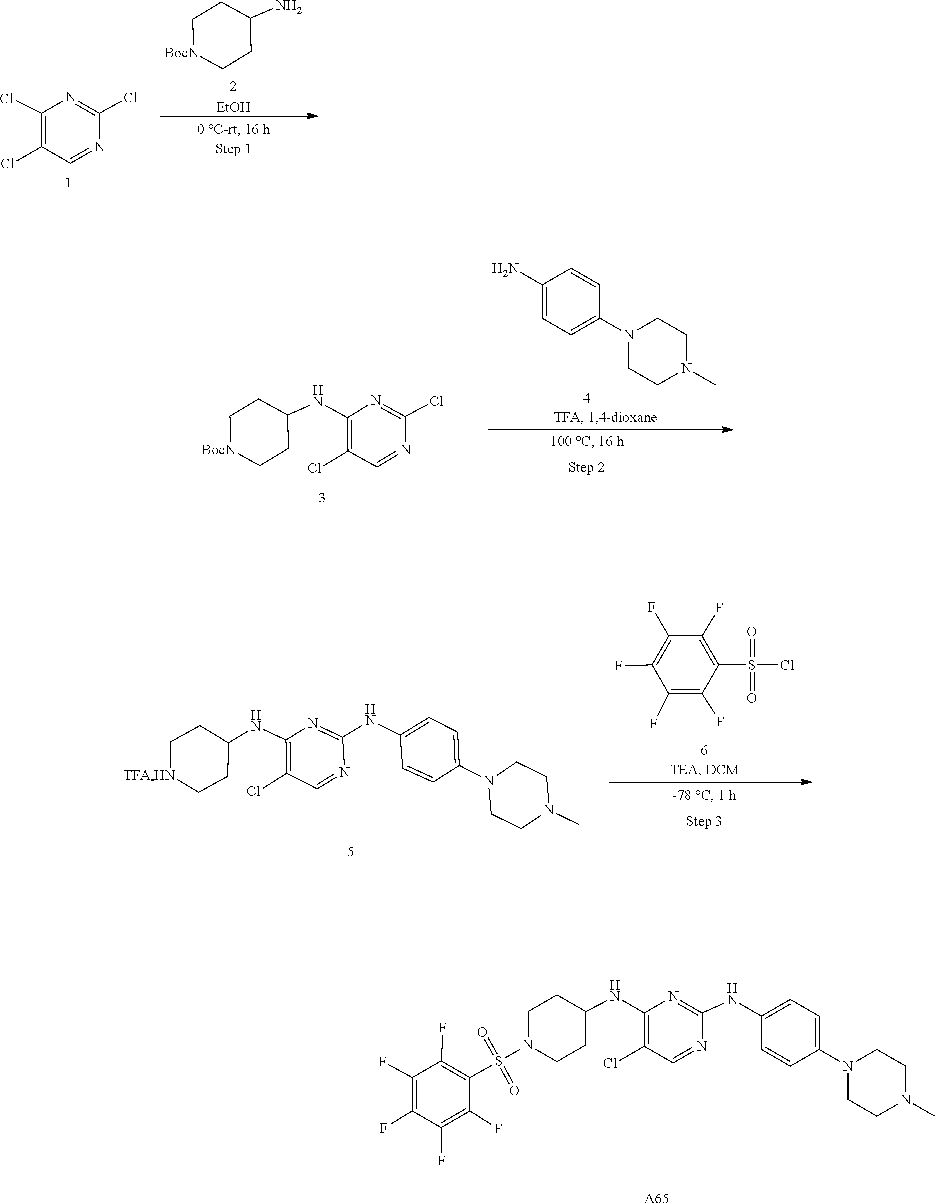

### tert-butyl 4-((2,5-dichloropyrimidin-4-yl)amino)piperidine-1-carboxylate (3)

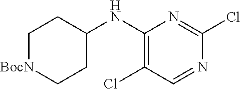 [SMILES: CC(C)(C)OC(=O)N1CCC(Nc2nc(Cl)ncc2Cl)CC1]

In an oven-dried, 500 mL, two-neck round-bottom flask equipped with a N₂ balloon and a rubber septum, 2,4,5-trichloropyrimidine (10.0 g, 54.5 mmol) and ethanol (100 ml) were added. The solution was cooled to 0 °C and tert-butyl 4-aminopiperidine-1-carboxylate (16.38 g, 82 mmol) was added. The reaction mixture was stirred under nitrogen atmosphere for 16 h at 50 °C. The reaction was monitored by LCMS. After completion of the reaction, ice water (100 ml) was added and extracted with ethyl acetate (2×80 ml). The combined organic portions were washed with water (2×50 ml) followed by brine solution (50 ml) and then dried over anhydrous Na₂SO₄, filtered and concentrated under reduced pressure. The obtained crude material was purified by flash column chromatography using 30-40% ethyl acetate in hexanes to provide tert-butyl 4-((2,5-dichloropyrimidin-4-yl)amino)piperidine-1-carboxylate (8.1 g, 22.63 mmol, 41.5%) as off white solid. LC-MS (ES+): 97.02%, RT: 2.55 min, m z 347.2 [M+H]⁺.

### 1-(4-((5-chloro-2-((4-(4-methylpiperazin-1-yl)phenyl)amino)pyrimidin-4-yl)amino)-11⁴-piperidin-1-yl)-2,2,2-trifluoroethan-1-one (5)

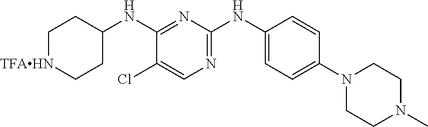

In an oven dried 25 mL two-neck round-bottom flask equipped with N₂ balloon, a solution of tert-butyl 4-((2,5-dichloropyrimidin-4-yl)amino)piperidine-1-carboxylate (0.2 g, 0.576 mmol) and 4-(4-methylpiperazin-1-yl)aniline (0.132 g, 0.691 mmol) was dissolved in 1,4-Dioxane (2 mL). TFA (2.399 ml, 31.1 mmol) was added. The resulting reaction mixture was stirred at 100 °C for 16 h. The reaction was monitored by TLC. After completion of the reaction volatiles were removed under reduced pressure to afford 1-(4-((5-chloro-2-((4-(4-methylpiperazin-1-yl)phenyl)amino)pyrimidin-4-yl)amino)-11⁴-piperidin-1-yl)-2,2,2-trifluoroethan-1-one (0.260 g, 0.647 mmol, 90%). LC-MS (ES+): 76%, RT: 0.28 min, m z 402.2 [M+H]⁺.

### Example A65 5-chloro-N²-(4-(4-methylpiperazin-1-yl)phenyl)-N⁴-(1-((perfluorophenyl)sulfonyl)piperidin-4-yl)pyrimidine-2,4-diamine

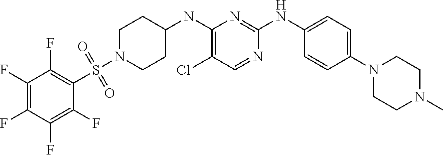 [SMILES: CN1CCN(c2ccc(Nc3ncc(Cl)c(NC4CCN(S(=O)(=O)c5c(F)c(F)c(F)c(F)c5F)CC4)n3)cc2)CC1]

To an oven dried 50 mL two-neck round-bottom flask equipped with N₂ balloon, 1-(4-((5-chloro-2-((4-(4-methylpiperazin-1-yl)phenyl)amino)pyrimidin-4-yl)amino)-11⁴-piperidin-1-yl)-2,2,2-trifluoroethan-1-one (0.250 g, 0.622 mmol), triethylamine (0.874 mL, 6.22 mmol) and DCM (3.0 mL) were added and stirred for 10 minutes at 0 °C. The resulting reaction mixture was cooled to −78. A solution of 2,3,4,5,6-pentafluorobenzenesulfonyl chloride (0.166 g, 0.622 mmol) in DCM (3 mL) was introduced and stirring continued for 1 h at −78 °C. The reaction was monitored by TLC and LCMS. Apon completion, the reaction was quenched with ice water (3 mL) and extracted with DCM (2×10 mL). The combined organic layers were dried over anhydrous sodium sulphate, filtered and concentrated under reduced pressure affording crude product. Thus obtained crude product was purified by prep HPLC to give 5-chloro-N²-(4-(4-methylpiperazin-1-yl)phenyl)-N⁴-(1-((perfluorophenyl)sulfonyl)piperidin-4-yl)pyrimidine-2,4-diamine (58.9 mg, 0.091 mmol, 14.68%) as off white solid.

The compounds in the following table 3 were prepared in the same manner as A65 from the appropriate starting materials.

| | | LCMS | | |
|---|---|---|---|---|
| | | Retention | LCMS | LCMS |
| Cmp | Structure | Time | Mass Obs | method |
| A66 | 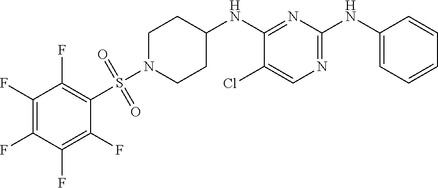 [SMILES: O=S(=O)(c1c(F)c(F)c(F)c(F)c1F)N1CCC(Nc2nc(Nc3ccccc3)ncc2Cl)CC1] 19F NMR (376 MHz, DMSO-d6): δ −135.82 to −135.89 (2F), −146.96 (1F), −159.50 to -159.61 (2F). 1H-NMR (400 MHz, DMSO-d6): δ 9.18 (s, 1H), 7.96 (s, 1H), 7.66 (d, J = 8.0 Hz, 2H), 7.20 (t, J = 8.4 Hz, 2H), 6.98 (d, J = 7.6 Hz, 1H), 6.89 (d, J = 7.2 Hz, 1H), 4.00-3.97 (m, 1H), 3.81 (d, J = 12.0 Hz, 2H), 2.86 (d, J = 11.6 Hz, 2H), 2.05-2.02 (m, 2H), 1.76-1.68 (m, 2H). | 4.769 | 534.0 (M + H)+ | Method- 9 |
| A67 | 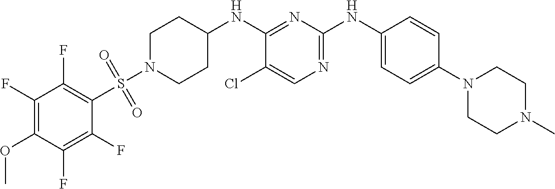 [SMILES: COc1c(F)c(F)c(S(=O)(=O)N2CCC(Nc3nc(Nc4ccc(N5CCN(C)CC5)cc4)ncc3Cl)CC2)c(F)c1F] 19F NMR (376 MHz, DMSO-d6): δ −137.94 to −137.99 (2F), −168.76 to −168.82 (2F). 1H-NMR (400 MHz, DMSO-d6): δ 8.96 (s, 1H), 7.90 (s, 1H), 7.51 (d, J = 8.80 Hz, 2H), 6.88 (d, J = 7.60 Hz, 1H), 6.81 (d, J = 9.20 Hz, 2H), 4.21 (s, 3H), 3.95-3.91 (m, 1H), 3.80-3.77 (m, 2H), 3.04- 3.01 (m, 4H), 2.86-2.80 (m, 2H), 2.48-2.45 (m, 4H), 2.28 (s, 3H), 2.07-2.04 (m, 2H), 1.75-1.71 (m, 2H). | 1.469 | 644.0 (M + H)+ | Method- 9 |
| A68 | 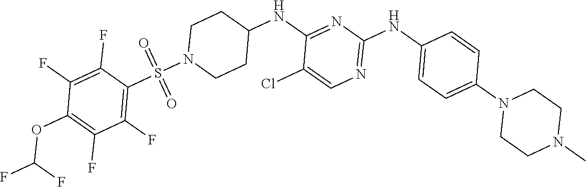 [SMILES: CN1CCN(c2ccc(Nc3ncc(Cl)c(NC4CCN(S(=O)(=O)c5c(F)c(F)c(OC(F)F)c(F)c5F)CC4)n3)cc2)CC1] 19F-NMR (376 MHz, DMSO-d6): δ −151.76 to −151.70 (2F), −136.31 to −136.24 (2F), −81.89 to −81.67 (2F) 1H-NMR (400 MHz, DMSO-d6): δ 8.96 (s, 1H), 7.90 (s, 1H), 7.51 (d, J = 9.2 Hz, 2H), 7.44 (s, 1H), 6.89-6.80 (m, 3H), 3.94 (d, J = 7.6 Hz, 1H), 3.81 (d, J = 11.6 Hz, 2H), 3.02 (s, 4H), 2.91 (t, J = 11.6 Hz, 2H), 2.51 (s, 4H), 2.24 (s, 3H), 2.07 (d, J = 10.8 Hz, 2H), 1.72 (g, J = 11.6 Hz, 2H) | 1.559 | 680.0 (M + H)+ | Method- 9 |
| A69 | 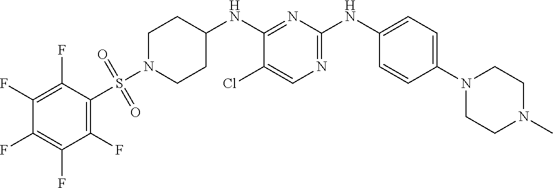 19F NMR (376 MHz, DMSO-d6): δ −146.09 to −145.42 (1F), −145.39 to −145.27 (1F), −133.42 to −133.22 (1F), −130.83 (1F), −50.50 to 50.89 (3F). 1H-NMR (400 MHz, DMSO-d6): δ 8.98 (s, 1H), 8.15 (s, 1H), 7.91 (s, 1H), 7.51 (d, J = 9.2 Hz, 2H), 6.94 (d, J = 7.6 Hz, 1H), 6.85 (d, J = 9.2 Hz, 2H), 4.10-4.07 (m, 1H), 3.86 (d, J = 12.8 Hz, 2H), 3.04 (t, J = 14.4 Hz, 6H), 2.51 (s, 3H), 2.25 (s, 3H), 2.01 (d, J = 10.8 Hz, 2H), 1.70 (q, J = 3.2 Hz, 2H). | 1.551 | 682.0 (M + H+)+ | Method- 9 |
| A70 | 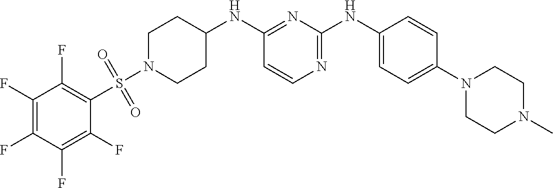 [SMILES: CN1CCN(c2ccc(Nc3nccc(NC4CCN(S(=O)(=O)c5c(F)c(F)c(F)c(F)c5F)CC4)n3)cc2)CC1] 19F NMR (376 MHz, DMSO-d6): −135.80 to −135.864 (2F) −147.07 to −147.13 (1F), −159.65 to −159.76 (2F). 1H-NMR (400 MHz, DMSO-d6): δ 8.66 (s, 1H), 7.75 (d, J = 5.6 Hz, 1H), 7.55 (d, J = 8.8 Hz, 2H), 7.11 (s, 1H), 6.80 (d, J = 9.2 Hz, 2H), 5.84 (d, J = 6.0 Hz, 1H), 3.81-3.70 (m, 3H), 3.03-3.00 (m, 4H), 2.96-2.91 (m, 2H), 2.47-2.45 (m, 4H), 2.23 (s, 3H), 2.11 (d, J = 12.0 Hz, 2H), 1.52-1.47 (m, 2H). | 1.309 | 598.2 (M + H)+ | Method- 9 |
| A71 | 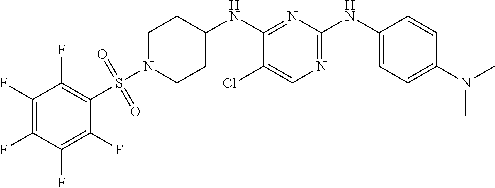 [SMILES: CN(C)c1ccc(Nc2ncc(Cl)c(NC3CCN(S(=O)(=O)c4c(F)c(F)c(F)c(F)c4F)CC3)n2)cc1] 19F NMR (376 MHz, DMSO-d6): δ −135.79 to −135.85 (2F), −147.22 (1F), −159.56 to −159.73 (2F). 1H-NMR (400 MHz, DMSO-d6): δ 8.86 (s, 1H), 7.88 (d, J = 6.4 Hz, 1H), 7.45 (d, J = 8.8 Hz, 2H), 6.86 (d, J = 7.2 Hz, 1H), 6.64 (d, J = 8.8 Hz, 2H), 3.93-3.90 (m, 1H), 3.80 (d, J = 12.0 Hz, 2H), 2.88-2.81 (m, 8H), 2.07-2.05 (m, 2H), 1.67- 1.75 (m, 2H). | 3.91 | 576.9 (M + H)⁺ | Method- 9 |
| A72 | 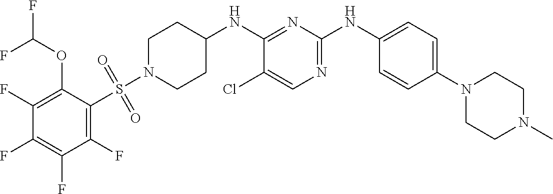 [SMILES: CN1CCN(c2ccc(Nc3ncc(Cl)c(NC4CCN(S(=O)(=O)c5c(F)c(F)c(F)c(F)c5OC(F)F)CC4)n3)cc2)CC1] 19F-NMR (376 MHz, DMSO-d6): δ −81.99 (2F), −133.94 (1F), −147.71 (1F), −149.34 (1F), −155.42 (1F). 1H-NMR (400 MHz, DMSO-d6): δ 8.97 (s, 1H), 7.90 (s, 1H), 7.51 (d, J = 9.2 Hz, 2H), 7.12 (t, J = 72.8 Hz, 1H), 6.88 (d, J = 7.6 Hz, 1H), 6.81 (d, J = 8.8 Hz, 2H), 3.94-3.90 (m, 1H), 3.79 (d, J = 12.0 Hz, 2H), 3.03 (t, J = 4.8 Hz, 4H), 2.84 (t, J = 11.6 Hz, 2H), 2.45 (t, J = 4.8 Hz, 4H), 2.23 (s, 3H), 2.06 (d, J = 10.8 Hz, 2H), 1.69 (dd, J = 3.6, 12.2 Hz, 2H). | 1.532 | 680.0 (M + H)+ | Method- 9 |
| A73 | 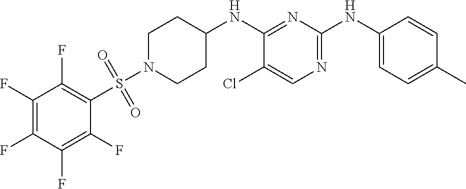 [SMILES: Cc1ccc(Nc2ncc(Cl)c(NC3CCN(S(=O)(=O)c4c(F)c(F)c(F)c(F)c4F)CC3)n2)cc1] 19F-NMR (376 MHz, DMSO-d6): δ −135.80 (2F), −146.91 to −147.06 (1F), −159.50 to −159.67 (2F) 1H-NMR (400 MHz, DMSO-d6): δ 9.10 (s, 1H), 7.94 (s, 1H), 7.53 (d, J = 7.6 Hz, 2H), 7.00-6.95 (m, 3H), 3.97-3.93 (m, 1H), 3.82-3.79 (m, 2H), 2.88-2.80 (m, 2H), 2.22 (s, 3H), 2.06-2.00 (m, 2H), 1.76-1.70 (m, 2H). | 2.269 | 548.0 (M + H)+ | Method- 9 |
| A74 | 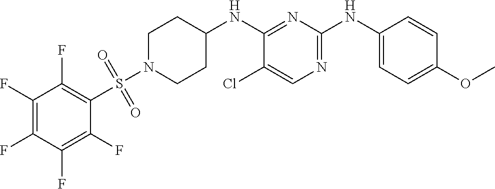 [SMILES: COc1ccc(Nc2ncc(Cl)c(NC3CCN(S(=O)(=O)c4c(F)c(F)c(F)c(F)c4F)CC3)n2)cc1] 19F-NMR (376 MHz, DMSO-d6): δ −135.84 (2F), −147.01 to −147.13 (1F), −159.50 to −159.67 (2F) 1H-NMR (400 MHz, DMSO-d6): δ 9.00 (s, 1H), 7.91 (s, 1H), 7.53 (d, J = 8.80 Hz, 2H), 6.91 (d, J = 7.20 Hz, 1H), 6.79 (d, J = 9.20 Hz, 2H), 3.95- 3.92 (m, 1H), 3.80 (d, J = 12.00 Hz, 2H), 3.70 (s, 3H), 2.85 (t, J = 12.00 Hz, 2H), 2.05-1.99 (m, 2H), 1.76-1.67 (m, 2H). | 2.066 | 564.0 (M + H)+ | Method- 9 |
| A75 | 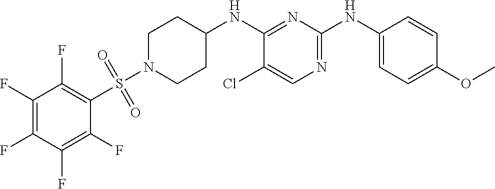 19F NMR (376 MHz, DMSO-d6): δ −119.92 (1F), −135.73 (2F), −147.11 (1F), −159.65 (2F) 1H-NMR (400 MHz, DMSO-d6): δ 8.10 (dd, J = 6.00, 8.8 Hz, 1H), 7.97 (s, 1H), 7.65 (s, 1H), 7.10 (d, J = 7.20 Hz, 1H), 7.02 (dd, J = 2.80, 10.40 Hz, 1H), 6.84-6.79 (m, 1H), 3.94-3.90 (m, 1H), 3.79 (d, J = 11.60 Hz, 2H), 2.89-2.80 (m, 6H), 2.51 (s, 4H), 2.22 (s, 3H), 2.05-1.99 (m, 2H), 1.75-1.67 (m, 2H). | 3.617 | 650.0 (M + H)+ | Method- 9 |
| A76 | 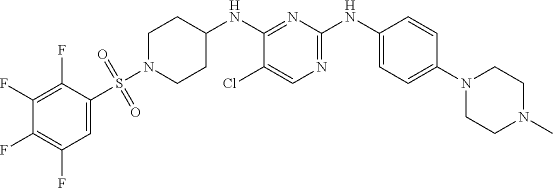 [SMILES: CN1CCN(c2ccc(Nc3ncc(Cl)c(NC4CCN(S(=O)(=O)c5cc(F)c(F)c(F)c5F)CC4)n3)cc2)CC1] 19F NMR (376 MHz, DMSO-d6): δ −133.95 (1F), −137.04 (1F), −147.85 (1F), −151.95 (1F) 1H-NMR (400 MHz, DMSO-d6): δ 8.97 (s, 1H), 7.92-7.84 (m, 2H), 7.55-7.49 (m, 2H), 6.87-6.74 (m, 3H), 3.94-3.90 (m, 1H), 3.78 (d, J = 12.00 Hz, 2H), 3.04-3.01 (m, 4H), 2.76 (t, J = 12.00 Hz, 2H), 2.46 (t, J = 4.80 Hz, 4H), 2.08 (s, 3H), 2.03-2.00 (m, 2H), 1.72-1.64 (m, 2H). | 1.46 | 614.0 (M + H)+ | Method- 9 |
| A77 | 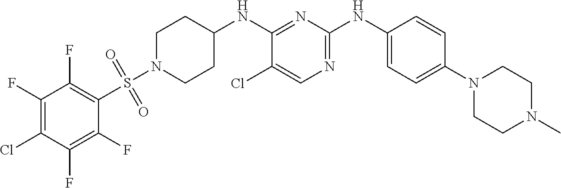 [SMILES: CN1CCN(c2ccc(Nc3ncc(Cl)c(NC4CCN(S(=O)(=O)c5c(F)c(F)c(Cl)c(F)c5F)CC4)n3)cc2)CC1] 19F NMR (376 MHz, DMSO-d6): δ −135.59 to δ −135.52 (2F), −139.27 to −139.21 (2F) 1H-NMR (400 MHz, DMSO-d6): δ 8.98 (s, 1H), 7.90 (s, 1H), 7.51 (d, J = 9.20 Hz, 2H), 6.85 (m, 3H), 3.90 (m, 1H), 3.79 (d, J = 11.60 Hz, 2H), 3.02 (t, J = 4.80 Hz, 4H), 2.91 (t, J = 12.00 Hz, 2H), 2.48-2.45 (m, 4H), 2.24 (s, 3H), 2.07 (m, 2H), 1.71 (m, 2H) | 1.611 | 648.3 (M + H)+ | Method- 9 |
| A78 | 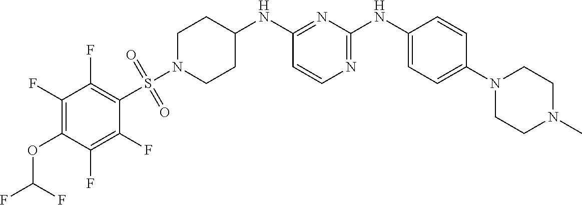 [SMILES: CN1CCN(c2ccc(Nc3nccc(NC4CCN(S(=O)(=O)c5c(F)c(F)c(OC(F)F)c(F)c5F)CC4)n3)cc2)CC1] 19F NMR (376 MHz, DMSO-d6): δ −81.67 to −81.89 (2F), −136.19 (2F), −151.68 (2F) VT-NMR (400 MHz, DMSO-d6): δ 8.34 (bs, 1H), 7.76 (d, J = 6.00 Hz, 1H), 7.55-7.20 (m, 3H), 6.89- 6.81 (m, 3H), 5.89 (d, J = 6.00 Hz, 1H), 3.87 (bs, 1H), 3.75 (d, J = 12.40 Hz, 2H), 3.08 (s, 6H), 2.68- 2.61 (m, 4H), 2.35-2.34 (m, 3H), 2.11-2.06 (m, 2H), 1.62-1.43 (m, 2H). | 1.195 | 646.1 (M + H)+ | Method- 3 |
| A98 | 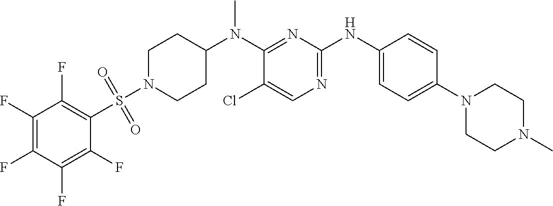 [SMILES: CN1CCN(c2ccc(Nc3ncc(Cl)c(N(C)C4CCN(S(=O)(=O)c5c(F)c(F)c(F)c(F)c5F)CC4)n3)cc2)CC1] 19F NMR (376 MHz, DMSO-d6): δ −135.95 (2F), δ −147.14 (1F), δ −159.69 (2F). 1H-NMR (400 MHz, DMSO-d6): δ 9.08 (s, 1H), 7.99 (s, 1H), 7.47 (d, J = 9.2 Hz, 2H), 6.83 (d, J = 9.2 Hz, 2H), 4.16-4.12 (m, 1H), 3.84 (d, J = 12.4 Hz, 2H), 3.03 (t, J = 5.2 Hz, 4H), 2.97 (s, 3H), 2.86 (m, 2H), 2.46-2.44 (m, 4H), 2.23 (s, 3H), 1.92 (m, 4H). | 1.657 | 646.0 (M + H)+ | Method- 9 |
| A113 | 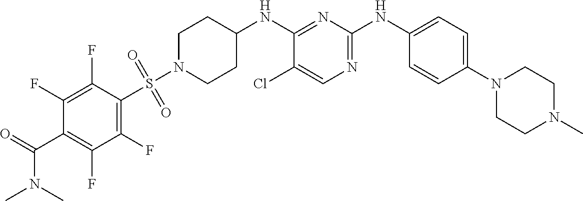 19F NMR (376 MHz, DMSO-d6): δ −134.92 to −134.83 (2F), −140.70 to −140.60 (2F). 1H-NMR (400 MHz, DMSO-d6): δ 8.96 (s, 1H), 7.90 (s, 1H), 7.52 (d, J = 8.80 Hz, 2H), 6.89-6.81 (m, 3H), 3.98-3.95 (m, 1H), 3.80 (d, J = 11.60 Hz, 2H), 3.01-2.95 (m, 12H), 2.57 (bs, 4H), 2.27 (bs, 3H), 2.08 (d, J = 10.80 Hz, 2H), 1.74-1.69 (m, 2H). | 1.261 | 685.2 [M + H]+ | Method- 2 |

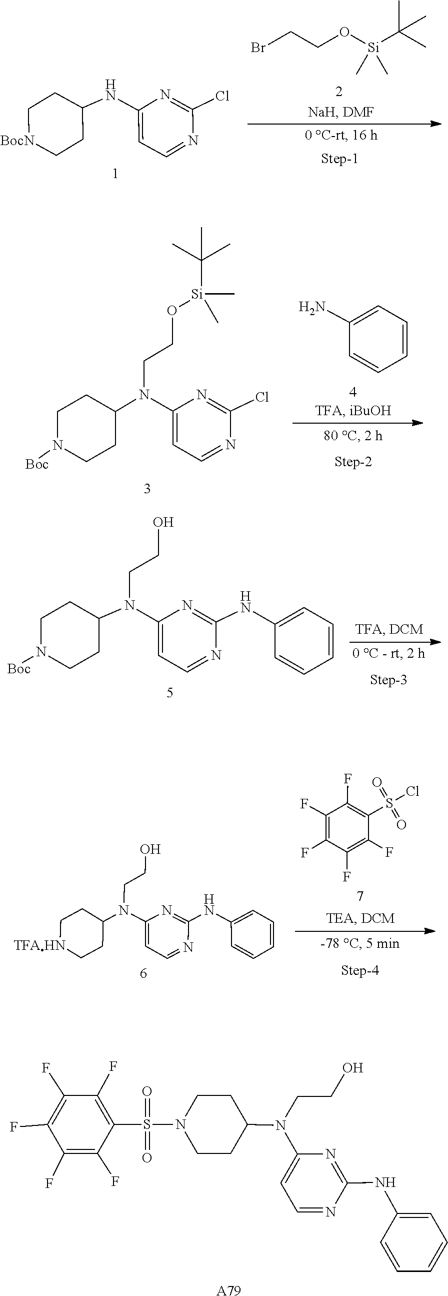

### Synthesis of tert-butyl 4-((2-((tert-butyldimethylsilyl)oxy)ethyl)(2-chloropyrimidin-4-yl)amino)piperidine-1-carboxylate (3)

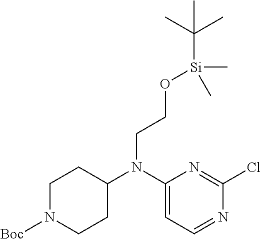 [SMILES: CC(C)(C)OC(=O)N1CCC(N(CCO[Si](C)(C)C(C)(C)C)c2ccnc(Cl)n2)CC1]

To an oven dried 50 mL two-neck round-bottom flask equipped with N₂ balloon and a rubber septum, tert-butyl 4-((2-chloropyrimidin-4-yl)amino)piperidine-1-carboxylate (500 mg, 1.598 mmol) and DMF (3 mL) were added. The reaction mixture was cooled to 0 °C, sodium hydride (320 mg, 7.99 mmol) was added by portion-wise at the same temperature and stirred for 20 minutes. A DMF (2 mL) solution of (2-bromoethoxy)(tert-butyl)dimethylsilane (1.372 mL, 6.39 mmol) was added and the resulting reaction mixture was stirred for 16 h at 25 °C. The reaction was monitored by TLC; quenched with ice water (100 mL) and extracted with ethyl acetate (2×100 mL). The combined organic layers were dried over anhydrous sodium sulphate, filtered and concentrated under reduced pressure affording crude product. The crude mass was purified by flash column chromatography using 30-40% ethyl acetate in hexane to afford tert-butyl 4-((2-((tert-butyldimethylsilyl)oxy)ethyl)(2-chloropyrimidin-4-yl)amino)piperidine-1-carboxylate (300 mg, 0.630 mmol, 39.4%) as a colorless gum.

LCMS: 98.23%, RT: 2.67, m/z=471.2 (M+H)⁺.

### Synthesis of tert-butyl 4-((2-hydroxyethyl)(2-(phenylamino)pyrimidin-4-yl)amino)piperidine-1-carboxylate (5)

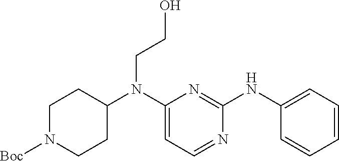 [SMILES: CC(C)(C)OC(=O)N1CCC(N(CCO)c2ccnc(Nc3ccccc3)n2)CC1]

To an oven dried 25 mL two-neck round-bottom flask equipped with N₂ balloon, tert-butyl 4-((2-((tert-butyldimethylsilyl)oxy)ethyl)(2-chloropyrimidin-4-yl)amino)piperidine-1-carboxylate (280 mg, 0.594 mmol), isobutanol (4 mL), aniline (83 mg, 0.892 mmol) and TFA (0.364 mL, 4.75 mmol) were added at room temperature. Then the reaction mixture was stirred for 16 h at 80 °C. The reaction was monitored by TLC. After completion, the reaction mixture was concentrated under reduced pressure to give crude tert-butyl 4-((2-hydroxyethyl)(2-(phenylamino)pyrimidin-4-yl)amino)piperidine-1-carboxylate (380 mg, 0.242 mmol, 40.8%) as a dark liquid.

LCMS: 77.68%, RT: 1.42, m/z=414.2 (M+H)⁺.

### Synthesis of 2,2,2-trifluoro-1-(4-((2-hydroxyethyl)(2-(phenylamino)pyrimidin-4-yl)amino)-114-piperidin-1-yl)ethan-1-one (6)

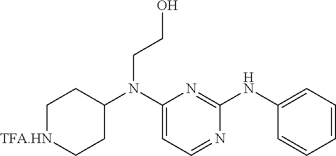

To an oven dried 25 mL round-bottom flask equipped with N₂ balloon, tert-butyl 4-((2-hydroxyethyl)(2-(phenylamino)pyrimidin-4-yl)amino)piperidine-1-carboxylate (380 mg, 0.242 mmol), 1,2-Dichloroethane (4 mL) and TFA (314 mg, 2.76 mmol) were added at room temperature. The resulting reaction mixture was stirred for 3 h at 80 °C. The reaction was monitored by TLC. After completion of the reaction volatiles were removed under reduced pressure to afford 2,2,2-trifluoro-1-(4-((2-hydroxyethyl)(2-(phenylamino)pyrimidin-4-yl)amino)-114-piperidin-1-yl)ethan-1-one (350 mg, 0.558 mmol, 60.8%) as a black gum.

LCMS: 50%, RT: 0.36 min, m/z=314.1 [(M-TFA)+H]⁺.

### Example A79 Synthesis of 2-((1-((perfluorophenyl)sulfonyl)piperidin-4-yl)(2-(phenylamino)pyrimidin-4-yl)amino)ethan-1-ol

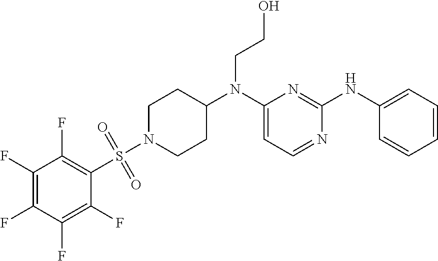 [SMILES: O=S(=O)(c1c(F)c(F)c(F)c(F)c1F)N1CCC(N(CCO)c2ccnc(Nc3ccccc3)n2)CC1]

To an oven dried 25 mL two-neck round-bottom flask equipped with N₂ balloon, 2,2,2-trifluoro-1-(4-((2-hydroxyethyl)(2-(phenylamino)pyrimidin-4-yl)amino)-114-piperidin-1-yl)ethan-1-one (200 mg, 0.638 mmol) in DCM (5 mL) were added TEA (0.356 mL, 2.55 mmol) and stirred for 10 minutes. The resulting solution was cooled to −78° c followed by addition of 3,4,5,6-pentafluorobenzenesulfonyl chloride (187 mg, 0.702 mmol) solution in DCM (2 mL) at the same temperature and further stirring for 1 h at −78 °C. The reaction was monitored by TLC; quenched with aqueous saturated NaHCO₃ solution (15 mL) and extracted with DCM (2×15 mL). The combined organic layers were dried over anhydrous sodium sulphate, filtered and concentrated under reduced pressure affording crude product. The crude mass was purified by prep HPLC (Method C) to afford 2-((1-((perfluorophenyl)sulfonyl)piperidin-4-yl)(2-(phenylamino)pyrimidin-4-yl)amino)ethan-1-ol (42.8 mg, 0.076 mmol, 11.90%) as an off white solid.

The compounds in the following table 4 were prepared in the same manner as A79 from the appropriate starting materials.

| | | LCMS | LCMS | |
|---|---|---|---|---|
| | | Retention | Mass | LCMS |
| CMP | Structure | Time | Obs | method |
| A80 | 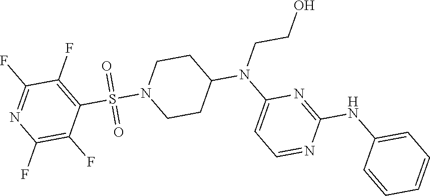 [SMILES: O=S(=O)(c1c(F)c(F)nc(F)c1F)N1CCC(N(CCO)c2ccnc(Nc3ccccc3)n2)CC1] 19F NMR (376 MHz, DMSO-d6): δ −89.06 (2F), −137.8 to −137.17 (2F). 1H-VT-NMR (400 MHz, DMSO-d6): δ 9.62 (s, 1H), 7.92 (d, J = 7.20 Hz, 1H), 7.50 (d, J = 8.00 Hz, 2H), 7.36 (t, J = 8.00 Hz, 2H), 7.14 (d, J = 7.2 Hz, 1H), 6.61 (d, J = 7.2 Hz, 1H), 4.26 (bs, 1H), 3.92-3.89 (m, 4H), 3.75-3.72 (m, 2H), 2.94-2.92 (m, 2H), 2.05-1.88 (m, 4H) ppm. | 4.41 | 527.25 [M + H]+ | Method- 1 |
| A81 | 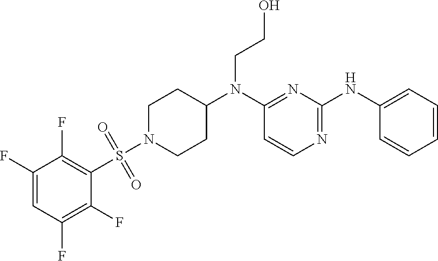 [SMILES: O=S(=O)(c1c(F)c(F)cc(F)c1F)N1CCC(N(CCO)c2ccnc(Nc3ccccc3)n2)CC1] 19F-NMR (376 MHz, DMSO-d6): δ −136.28 to −136.26 (4F) 1H-VT-NMR (400 MHz, DMSO-d6): δ 8.67 (s, 1H), 8.26 (quintet, J = 8.8 Hz, 1H), 7.96 (d, J = 6.40 Hz, 1H), 7.66 (d, J = 7.60 Hz, 2H), 7.16 (t, J = 7.60 Hz, 2H), 6.86 (t, J = 7.20 Hz, 1H), 6.16 (d, J = 6.00 Hz, 1H), 4.53 (bs, 1H), 4.36 (bs, 1H), 3.91 (d, J = 12.00 Hz, 2H), 3.55-3.53 (m, 2H), 3.44-3.41 (m, 2H), 2.89- 2.82 (m, 2H), 1.89-1.85 (m, 4H) ppm. | 1.51 | 526.1 [M + H]+ | Method- 2 |
| A82 | 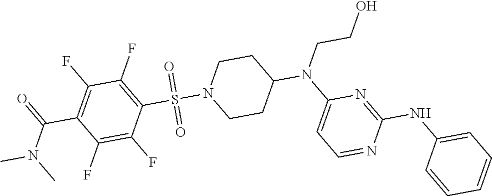 [SMILES: CN(C)C(=O)c1c(F)c(F)c(S(=O)(=O)N2CCC(N(CCO)c3ccnc(Nc4ccccc4)n3)CC2)c(F)c1F] 19F NMR (376 MHz, DMSO-d6): δ −134.42 to −134.33 (2F), −140.31 to −140.22 (2F) 1H-NMR (400 MHz, DMSO-d6): δ 8.99 (s, 1H), 7.94 (d, J = 6.00 Hz, 1H), 7.67 (d, J = 7.60 Hz, 2H), 7.14 (s, 2H), 6.85 (t, J = 7.20 Hz, 1H), 6.17 (d, J = 6.00 Hz, 1H), 4.77-4.47 (m, 2H), 3.86 (d, J = 10.80 Hz, 2H), 3.51-3.47 (m, 4H), 3.08 (s, 3H), 2.99 (s, 3H), 2.88- 2.86 (m, 2H), 1.88-1.84 (m, 4H). | 1.444 | 597.1 [M + H]+ | Method- 2 |
| A83 | 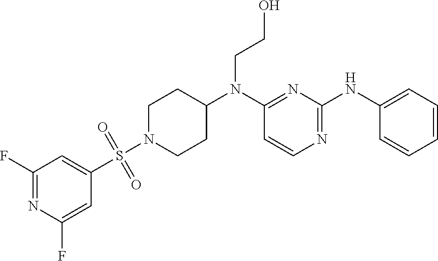 [SMILES: O=S(=O)(c1cc(F)nc(F)c1)N1CCC(N(CCO)c2ccnc(Nc3ccccc3)n2)CC1] 19F NMR (376 MHz, DMSO-d6): δ −65.119 (2F). 1H-VT-NMR (400 MHz, DMSO-d6): δ 8.66 (s, 1H), 7.92 (d, J = 6.00 Hz, 1H), 7.64 (d, J = 8.00 Hz, 2H), 7.56 (s, 2H), 7.14 (t, J = 8.00 Hz, 2H), 6.85 (t, J = 7.20 Hz, 1H), 6.15 (d, J = 6.00 Hz, 1H), 4.52-4.31 (m, 2H), 3.89 (d, J = 12.00 Hz, 2H), 3.55-3.51 (m, 2H), 3.43-3.40 (m, 2H), 2.68-2.62 (m, 2H), 1.89- 1.80 (m, 4H) ppm. | 1.403 | 491.1 [M + H]+ | Method- 2 |
| A84 | 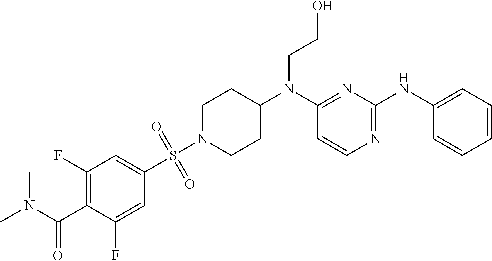 [SMILES: CN(C)C(=O)c1c(F)cc(S(=O)(=O)N2CCC(N(CCO)c3ccnc(Nc4ccccc4)n3)CC2)cc1F] 19F NMR (376 MHz, DMSO-d6): δ −110.38 (2F) 1H-VT-NMR (400 MHz, DMSO-d6): δ 8.67 (s, 1H), 7.92 (d, J = 6.00 Hz, 1H), 7.65-7.63 (m, 4H), 7.12 (t, J = 8.00 Hz, 2H), 6.87 (t, J = 7.60 Hz, 1H), 6.16 (d, J = 6.00 Hz, 1H), 4.52 (bs, 1H), 4.35-4.32 (m, 1H), 3.88 (d, J = 12.00 Hz, 2H), 3.55-3.52 (m, 2H), 3.42- 3.38 (m, 2H), 3.09 (s, 3H), 2.93 (s, 3H), 2.59-2.56 (m, 2H), 1.88-1.78 (m, 4H) ppm. | 1.328 | 561.1 [M + H]+ | Method- 2 |
| A85 | 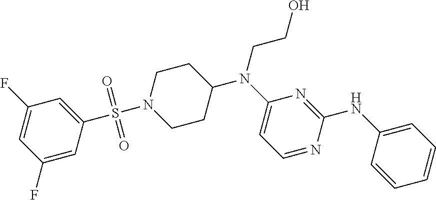 [SMILES: O=S(=O)(c1cc(F)cc(F)c1)N1CCC(N(CCO)c2ccnc(Nc3ccccc3)n2)CC1] 19F-NMR(376 MHz, DMSO-d6): δ −105.99 (2F). 1H-VT-NMR(400 MHz, DMSO-d6): δ 8.67 (s, 1H), 7.91 (d, J = 6.0 Hz, 1H), 7.73-7.68 (m, 1H), 7.62 (d, J = 7.60 Hz, 2H), 7.55-7.50 (m, 2H), 7.1 (t, J = 8.00 Hz, 2H), 6.84 (t, J = 7.2 Hz, 1H), 6.15 (d, J = 6.00 Hz, 1H), 4.54-4.51 (m, 1H), 4.34-2.32 (m, 1H), 3.85 (d, J = 12.00 Hz, 2H), 3.53 - 3.47 (m, 2H), 3.41- 3.37 (m, 2H), 2.34-2.32 (m 2H), 1.83-1.80 (m, 4H). | 1.475 | 490.1 [M + H]+ | Method- 2 |

### Synthesis of tert-butyl 4-((2-chloropyrimidin-4-yl)(2-cyclopropoxyethyl)amino)piperidine-1-carboxylate

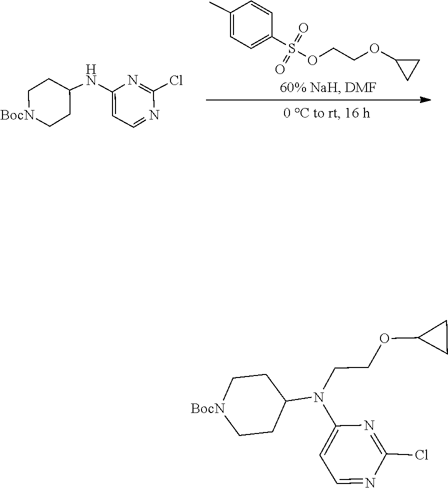 [SMILES: CC(C)(C)OC(=O)N1CCC(Nc2ccnc(Cl)n2)CC1 ; Cc1ccc(S(=O)(=O)OCCOC2CC2)cc1 ; CC(C)(C)OC(=O)N1CCC(N(CCOC2CC2)c2ccnc(Cl)n2)CC1]

To an oven dried 50 mL two-neck round-bottom flask equipped with N₂ balloon and a rubber septum, tert-butyl 4-((2-chloropyrimidin-4-yl)amino)piperidine-1-carboxylate (300 mg, 0.959 mmol) and DMF (15 mL) were added. The reaction mixture was cooled to 0 °C and sodium hydride (115 mg, 2.88 mmol) was added by portion wise at the same temperature. After stirring for 20 min, a DMF (2 mL) solution of 2-cyclopropoxyethyl 4-methylbenzenesulfonate (492 mg, 1.918 mmol) was introduced. The resulting reaction mixture was stirred for 16 h at 25 °C. The reaction was monitored by TLC then it was quenched with ice water (100 mL) and extracted with ethyl acetate (2×30 mL). The combined organic layers were dried over anhydrous sodium sulphate, filtered and concentrated under reduced pressure affording crude product. The crude mass was purified by flash column chromatography using 30-40% ethyl acetate in hexane to afford tert-butyl 4-((2-chloropyrimidin-4-yl)(2-cyclopropoxyethyl)amino)piperidine-1-carboxylate (100 mg, 0.227 mmol, 23.64% yield) as an off white solid.

LCMS: 87.68%, RT: 2.16, m/z=397.28 (M+H)⁺.

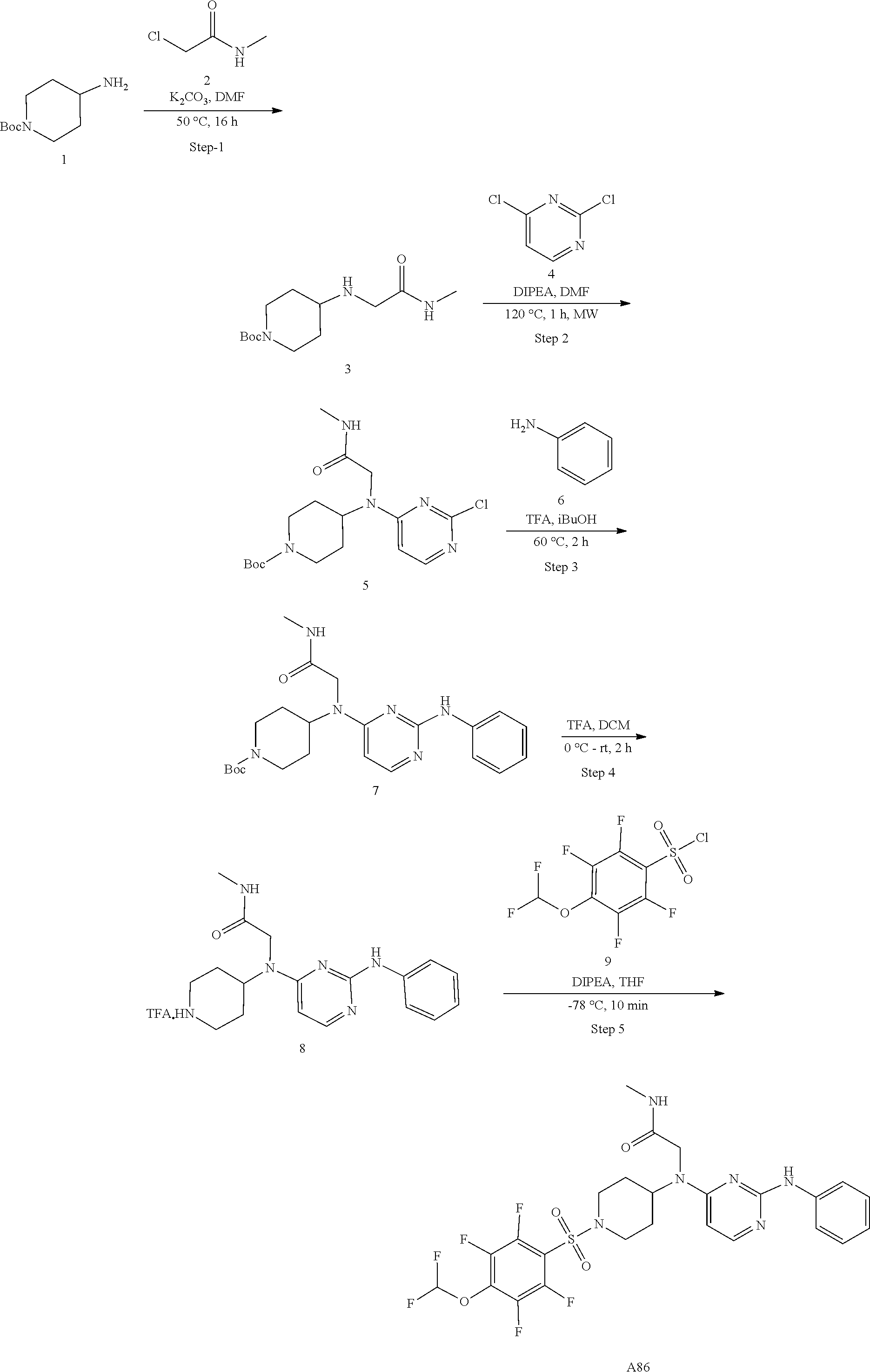

### Synthesis of tert-butyl 4-((2-(methylamino)-2-oxoethyl)amino)piperidine-1-carboxylate (3)

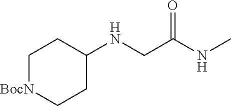 [SMILES: CNC(=O)CNC1CCN(C(=O)OC(C)(C)C)CC1]

To an oven dried 25 mL two-neck round-bottom flask equipped with N₂ balloon, tert-butyl 4-aminopiperidine-1-carboxylate (1 g, 4.99 mmol), DMF (10 mL), K₂CO₃ (1.380 g, 9.99 mmol) and 2-chloro-N-methylacetamide (0.537 g, 4.99 mmol) were added. The resulting reaction mixture was stirred under nitrogen atmosphere for 16 h at 50 °C. The reaction was monitored by TLC and ELSD-LCMS. After complete conversion, the reaction mixture was diluted with ice cold water (30 mL), extracted with ethyl acetate (2×20 mL). The combined organic layers were dried over Na₂SO₄ and evaporated under reduced pressure to get crude tert-butyl 4-((2-(methylamino)-2-oxoethyl)amino)piperidine-1-carboxylate (520 mg, 4.99 mmol, 34.5%) as a brown liquid. LCMS(ELSD): 91.64%, RT: 0.91 min, m/z=272.2 (M+H)⁺.

### Synthesis of tert-butyl 4-((2-chloropyrimidin-4-yl)(2-(methylamino)-2-oxoethyl)amino)piperidine-1-carboxylate (5)

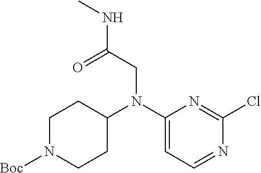 [SMILES: CNC(=O)CN(c1ccnc(Cl)n1)C1CCN(C(=O)OC(C)(C)C)CC1]

In a 20 mL microwave vial tert-butyl 4-((2-(methylamino)-2-oxoethyl)amino)piperidine-1-carboxylate (250 mg, 0.921 mmol), 2,4-dichloropyrimidine (165 mg, 1.106 mmol), DIPEA (0.642 mL, 3.69 mmol) and were combined in DMF (5 mL). The reaction mixture was irradiated at 120 °C for 1 h in a microwave reactor. The reaction was monitored by LCMS. After completion of the reaction, the reaction mixture was quenched with ice cold water (15 mL) and extracted with ethyl acetate (2×15 mL). The combined organics were washed with water (2×15 mL) followed by brine (20 mL), dried over Na₂SO₄ and concentrated under reduced pressure. The obtained crude compound was purified by column chromatography using neutral alumina and eluting with 10% MeOH in/DCM to afford tert-butyl 4-((2-chloropyrimidin-4-yl)(2-(methylamino)-2-oxoethyl)amino)piperidine-1-carboxylate (280 mg, 0.715 mmol, 78%) as an off white solid.

LCMS: 98.43%, RT: 1.519 min, m/z=484.1 (M+H)+.

### Synthesis of tert-butyl 4-((2-(methylamino)-2-oxoethyl)(2-(phenylamino)pyrimidin-4-yl)amino)piperidine-1-carboxylate (7)

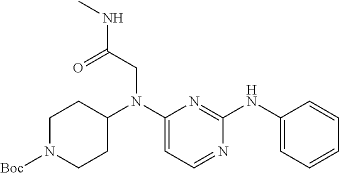 [SMILES: CNC(=O)CN(c1ccnc(Nc2ccccc2)n1)C1CCN(C(=O)OC(C)(C)C)CC1]

To an oven dried 25 mL two-neck round-bottom flask equipped with N₂ balloon, tert-butyl 4-((2-chloropyrimidin-4-yl)(2-(methylamino)-2-oxoethyl)amino)piperidine-1-carboxylate (392 mg, 1.020 mmol), Isobutanol (5 mL), aniline (95 mg, 1.020 mmol) and TFA (0.156 mL, 2.040 mmol) were added at room temperature. Then the reaction mixture was stirred for 2 h at 60 °C. The reaction was monitored by TLC. After completion of the reaction, reaction mixture was concentrated under reduced pressure to get the crude. Thus obtained crude was purified by reverse phase column chromatography by using method [Column: ¹⁸C 50 gm column, Mobile phase A: 0.1% FA in milli-Q water; Mobile phase B: CH₃CN] and fraction was lyophilized to afford tert-butyl 4-((2-(methylamino)-2-oxoethyl)(2-(phenylamino)pyrimidin-4-yl)amino)piperidine-1-carboxylate (375 mg, 0.843 mmol, 83%) as an off white solid. LCMS: 99.16%, RT: 1.353, m/z=44.3 (M+H)+.

### N-methyl-2-((2-(phenylamino)pyrimidin-4-yl)(1-(2,2,2-trifluoroacetyl)-114-piperidin-4-yl)amino)acetamide (8)

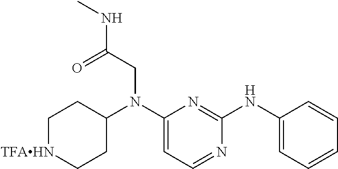

To an oven dried 25 mL round-bottom flask equipped with N₂ balloon, tert-butyl 4-((2-(methylamino)-2-oxoethyl)(2-(phenylamino)pyrimidin-4-yl)amino)piperidine-1-carboxylate (375 mg, 0.843 mmol), DCM (5 mL) and TFA (165 mg, 1.70 mmol) were added at room temperature. The resulting reaction mixture was stirred for 2 h at rt. The reaction was monitored by TLC. After completion of the reaction, volatiles were removed under reduced pressure to afford N-methyl-2-((2-(phenylamino)pyrimidin-4-yl)(1-(2,2,2-trifluoroacetyl)-114-piperidin-4-yl)amino)acetamide (150 mg, 0.342 mmol, 40%) as a black gum.

### Example A86 Synthesis of 2-((1-((4-(difluoromethoxy)-2,3,5,6-tetrafluorophenyl)sulfonyl)piperidin-4-yl)(2-(phenylamino)pyrimidin-4-yl)amino)-N-methylacetamide

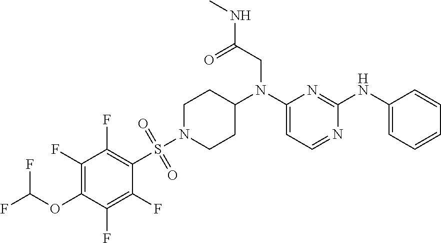 [SMILES: CNC(=O)CN(c1ccnc(Nc2ccccc2)n1)C1CCN(S(=O)(=O)c2c(F)c(F)c(OC(F)F)c(F)c2F)CC1]

To an oven dried 25 mL two-neck round-bottom flask equipped with N₂ balloon, N-methyl-2-((2-(phenylamino)pyrimidin-4-yl)(1-(2,2,2-trifluoroacetyl)-114-piperidin-4-yl)amino)acetamide (75 mg, 0.220 mmol) in THF (5 mL) were added DIPEA (0.269 mL, 1.542 mmol) and the mixture was stirred for 10 minutes. The resultant solution was cooled to −78° c and a 4-(difluoromethoxy)-2,3,5,6-tetrafluorobenzenesulfonyl chloride (104 mg, 0.330 mmol) solution in DCM (2 mL) was added at the same temperature and further stirred for 10 min at −78 °C. The reaction was monitored by TLC, quenched with aqueous saturated NaHCO₃ solution (15 mL) and extracted with DCM (2×15 mL). The combined organic layers were dried over anhydrous sodium sulphate, filtered and concentrated under reduced pressure affording crude product. The crude mass was purified by prep HPLC (Method C) to afford 2-((1-((4-(difluoromethoxy)-2,3,5,6-tetrafluorophenyl)sulfonyl)piperidin-4-yl)(2-(phenylamino)pyrimidin-4-yl)amino)-N-methylacetamide (29.3 mg, 0.047 mmol, 21.38%) as an off white solid.

### Example A87 2-((1-((4-chloro-2,3,5,6-tetrafluorophenyl)sulfonyl)piperidin-4-yl)(2-(phenylamino)pyrimidin-4-yl)amino)-N-methylacetamide A87 was prepared a similar method to that used to prepared A86

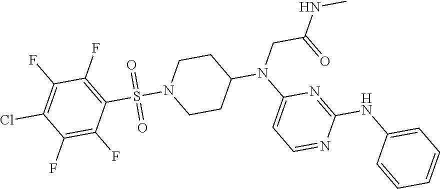 [SMILES: CNC(=O)CN(c1ccnc(Nc2ccccc2)n1)C1CCN(S(=O)(=O)c2c(F)c(F)c(Cl)c(F)c2F)CC1]

¹⁹F NMR (376 MHz, DMSO-d6): −138.83 to −138.787 (2F), −135.26 to −135.22 (2F). ¹H-VT-NMR (400 MHz, DMSO-d6): δ 8.93 (s, 1H), 7.95 (d, J=6.00 Hz, 1H), 7.61 (d, J=7.60 Hz, 3H), 7.21 (t, J=8.40 Hz, 2H), 6.93 (t, J=7.20 Hz, 1H), 6.14 (d, J=6.00 Hz, 1H), 4.34-4.32 (m, 1H), 4.04 (s, 2H), 3.88 (d, J=12.40 Hz, 2H), 2.91 (t, J=12.00 Hz, 2H), 2.62 (d, J=4.80 Hz, 3H), 1.93-1.90 (m, 2H), 1.81-1.72 (m, 2H) ppm. LCMS Method 1, RT: 1.62 min, m/z=587.2 (M+H)⁺.

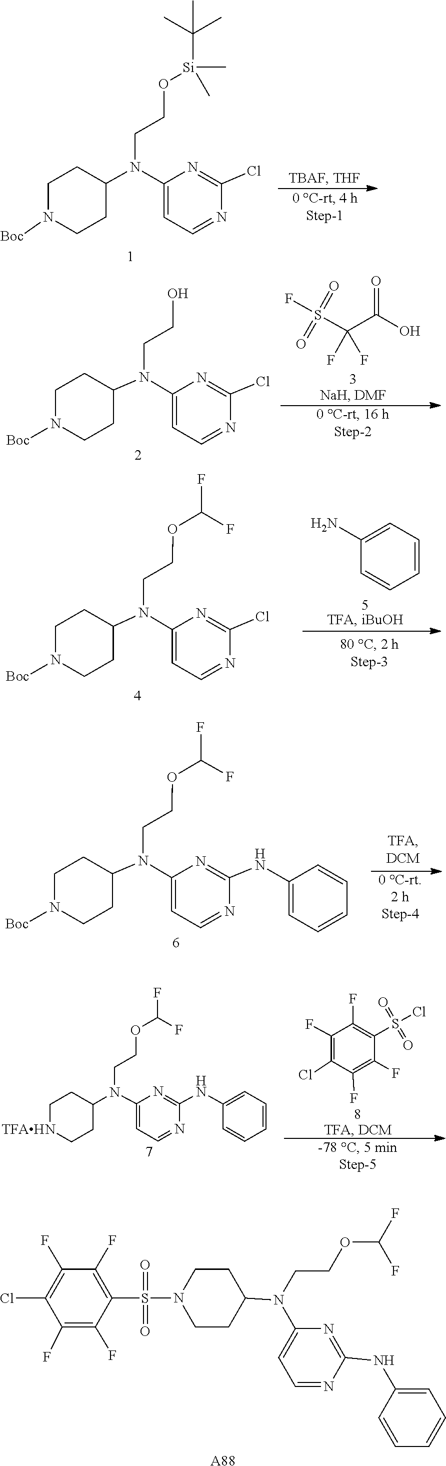

### Synthesis of tert-butyl 4-((2-chloropyrimidin-4-yl)(2-hydroxyethyl)amino)piperidine-1-carboxylate (2)

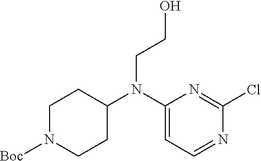 [SMILES: CC(C)(C)OC(=O)N1CCC(N(CCO)c2ccnc(Cl)n2)CC1]

To an oven dried 100 mL two-neck round-bottom flask equipped with N₂ balloon and a rubber septum, tert-butyl 4-((2-((tert-butyldimethylsilyl)oxy)ethyl)(2-chloropyrimidin-4-yl)amino)piperidine-1-carboxylate (1.7 g, 3.61 mmol) and THF (20 ml) were added. The resulting solution was cooled to 0 °C followed by dropwise addition of TBAF (5.41 ml, 5.41 mmol) in THF (1M). The reaction was allowed to stir for 4 h at RT. The reaction was monitored by TLC and LCMS, quenched with ice water (3 mL) and extracted with ethyl acetate (2×20 mL). The combined organic layers were dried over anhydrous sodium sulphate, filtered and concentrated under reduced pressure affording crude product. The crude product was triturated with n-pentane to afford tert-butyl 4-((2-chloropyrimidin-4-yl)(2-hydroxyethyl)amino)piperidine-1-carboxylate (1.2 g, 3.26 mmol, 90%) as an off white solid.

LCMS: 97.02%, RT: 1.64, m/z=357.1 (M+H)⁺.

### Synthesis of tert-butyl 4-((2-chloropyrimidin-4-yl)(2-(difluoromethoxy)ethyl)amino)piperidine-1-carboxylate (4)

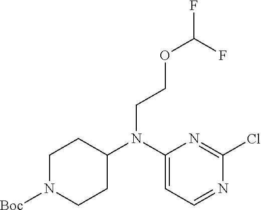 [SMILES: CC(C)(C)OC(=O)N1CCC(N(CCOC(F)F)c2ccnc(Cl)n2)CC1]

To an oven dried 40 mL vail, tert-butyl 4-((2-chloropyrimidin-4-yl)(2-hydroxyethyl)amino)piperidine-1-carboxylate (900 mg, 2.52 mmol), acetonitrile (12 mL), and copper iodide (96 mg, 0.504 mmol) were added. The resulting reaction mixture was stirred for 10 min at 50 °C. To this mixture a solution of 2,2-difluoro-2-(fluorosulfonyl)acetic acid (898 mg, 5.04 mmol) in acetonitrile (2 mL) was added at 50 °C and stirred for 2 h at the same temperature. The reaction was monitored by TLC. After completion the reaction, volatiles were removed under reduced pressure to get the crude which was purified by flash column chromatography using silica gel and eluted with 20% ethyl acetate in hexane giving tert-butyl 4-((2-chloropyrimidin-4-yl)(2-(difluoromethoxy)ethyl)amino)piperidine-1-carboxylate (100 mg, 0.211 mmol, 8.38%) as a brown thick liquid. LCMS: 86.36%, RT: 2.04 min, m/z=407.1 (M+H)⁺.

Intermediate tert-butyl 4-((2-chloropyrimidin-4-yl)(2-(difluoromethoxy)ethyl)amino)piperidine-1-carboxylate was used to prepare compounds A88 and A89 using a similar protocol as used for A1.

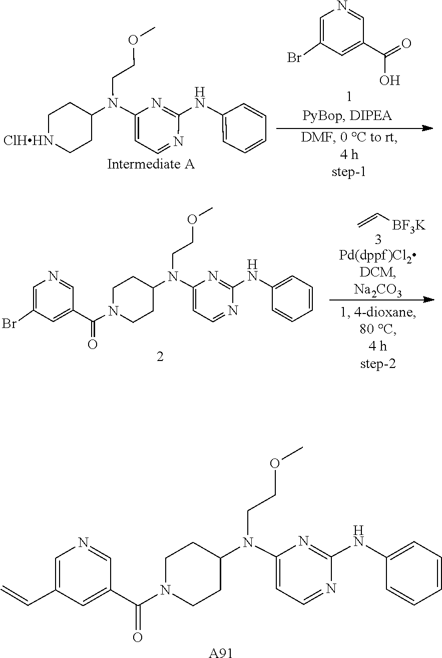

### Synthesis of (5-bromopyridin-3-yl)(4-((2-methoxyethyl)(2-(phenylamino)pyrimidin-4-yl)amino)piperidin-1-yl)methanone (3)

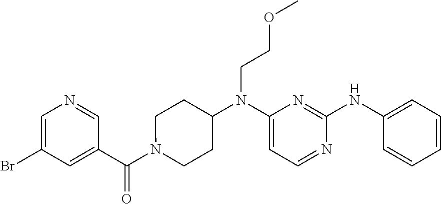 [SMILES: COCCN(c1ccnc(Nc2ccccc2)n1)C1CCN(C(=O)c2cncc(Br)c2)CC1]

To an oven dried 50 mL two-neck round-bottom flask equipped with N₂ balloon, 5-bromonicotinic acid (139 mg, 0.687 mmol), DMF (8 mL), PyBOP (536 mg, 1.031 mmol), N⁴-(2-methoxyethyl)-N²-phenyl-N⁴-(piperidin-4-yl)pyrimidine-2,4-diamine hydrochloride (250 mg, 0.687 mmol) and DIPEA (266 mg, 2.061 mmol) were added at 0 °C. The resulting reaction mixture stirred for 4 h at room temperature. The reaction was monitored by TLC, quenched with ice cold water (30 mL) and extracted with ethyl acetate (3×50 mL). The combined organic layers were dried over anhydrous sodium sulphate, filtered and concentrated under reduced pressure to afford crude product. The crude mass was purified by column chromatography by using silica gel 100-200 and eluted with 100% ethyl acetate to afford (5-bromopyridin-3-yl)(4-((2-methoxyethyl)(2-(phenylamino)pyrimidin-4-yl)amino)piperidin-1-yl)methanone (270 mg, 0.327 mmol, 47.6%) as a light brown gum.

LCMS: 62.45%, RT: 1.24 min, m/z=513.1 (M+2)⁺.

### Example A91 Synthesis of (4-((2-methoxyethyl)(2-(phenylamino)pyrimidin-4-yl)amino)piperidin-1-yl)(5-vinylpyridin-3-yl)methanone A91

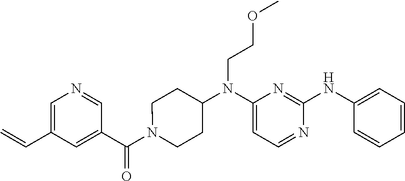 [SMILES: C=Cc1cncc(C(=O)N2CCC(N(CCOC)c3ccnc(Nc4ccccc4)n3)CC2)c1]

In a 40 mL reaction vessel, (5-bromopyridin-3-yl)(4-((2-methoxyethyl)(2-(phenylamino)pyrimidin-4-yl)amino)piperidin-1-yl)methanone (200 mg, 0.391 mmol), trifluoro(vinyl)-14-borane, potassium salt (105 mg, 0.782 mmol), Na₂CO₃ (124 mg, 1.173 mmol) and dioxane (4 mL) were added at 25°. After degassing the reaction mixture with nitrogen for 5 min, PdCl₂(dppf)·CH₂Cl₂ adduct (15.97 mg, 0.020 mmol) was added and further degassed for 5 min. The resulting reaction mixture stirred for 4 h at 80 °C. The reaction was monitored by TLC and LCMS. After completion, the volatiles were removed under reduced pressure. The obtained residue was diluted with water (20 mL), extracted with Ethyl acetate (2×15 mL) and the combined organic layers were washed with water (20 mL) followed by brine (20 mL), dried over Na₂SO₄, concentrated under reduced pressure providing crude product. The obtained crude was purified by prep HPLC to afford (4-((2-methoxyethyl)(2-(phenylamino)pyrimidin-4-yl)amino)piperidin-1-yl)(5-vinylpyridin-3-yl)methanone (32 mg, 0.069 mmol, 17.67%) as an off white solid.

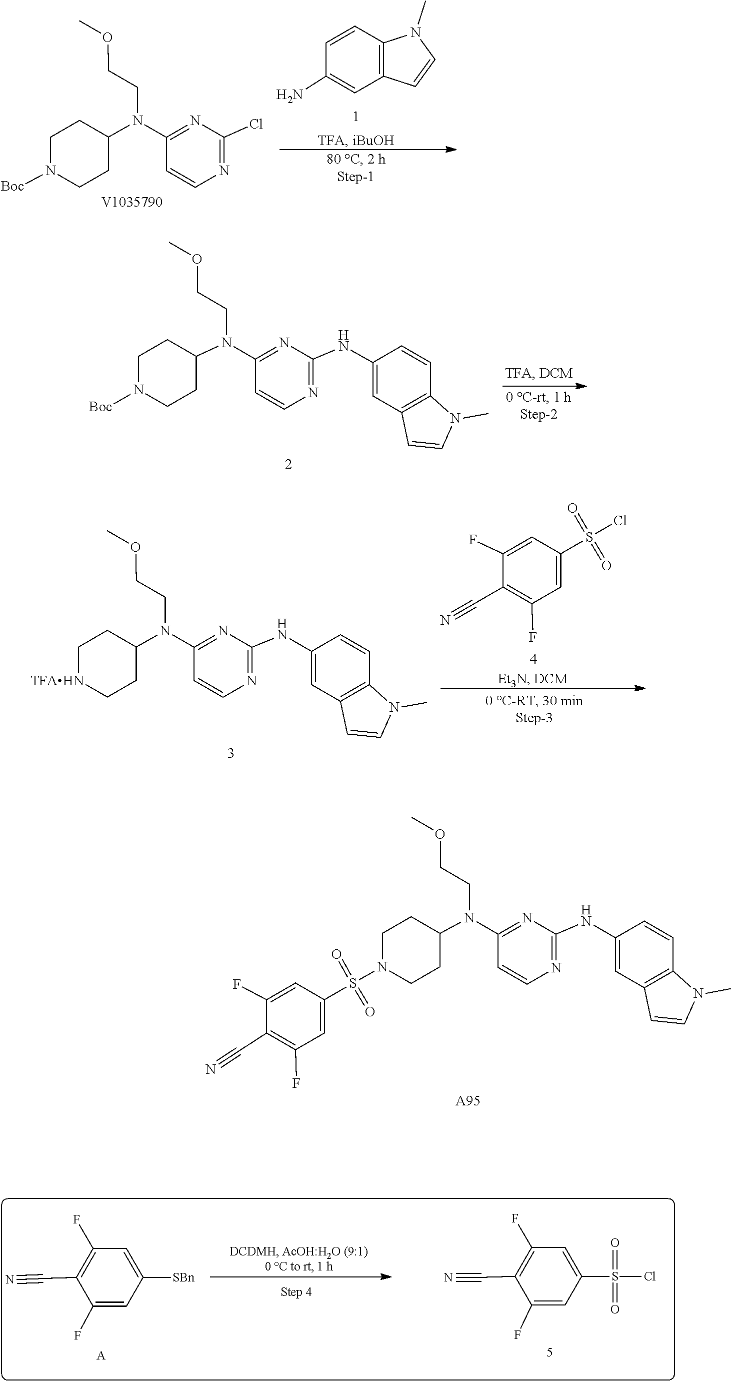

### tert-butyl 4-((2-methoxyethyl)(2-((1-methyl-1H-indol-5-yl)amino)pyrimidin-4-yl)amino)piperidine-1-carboxylate (2)

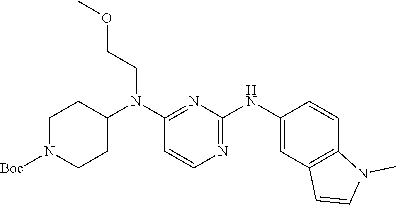 [SMILES: COCCN(c1ccnc(Nc2ccc3c(ccn3C)c2)n1)C1CCN(C(=O)OC(C)(C)C)CC1]

An oven-dried 50 mL two-neck round-bottom flask equipped with N₂ balloon and a rubber septum was charged with a solution of 1-methyl-1H-indol-5-amine (197 mg, 1.348 mmol), tert-butyl 4-((2-chloropyrimidin-4-yl)(2-methoxyethyl)amino)piperidine-1-carboxylate (500 mg, 1.348 mmol) in iBuOH (5 mL) and TFA (154 mg, 1.348 mmol) was added and the reaction stirred for 2 h at 80 °C. The reaction was monitored by LCMS, quenched with ice water (10 mL) and extracted with ethyl acetate (2×20 mL). The combined organic layers were dried over anhydrous sodium sulphate, filtered and concentrated under reduced pressure afforded the crude product. Thus obtained crude product was purified by flash column chromatography using 70% ethyl acetate/hexane as eluent to provide tert-butyl 4-((2-methoxyethyl)(2-((1-methyl-1H-indol-5-yl)amino)pyrimidin-4-yl)amino)piperidine-1-carboxylate (300 mg, 0.610 mmol, 45.2%) as white solid. LCMS (Method): 97.65%, RT: 1.57, m/z=481.3 (M+H)+.

### 2,2,2-trifluoro-1-(4-((2-methoxyethyl)(2-((1-methyl-1H-indol-5-yl)amino)pyrimidin-4-yl)amino)-114-piperidin-1-yl)ethan-1-one (3)

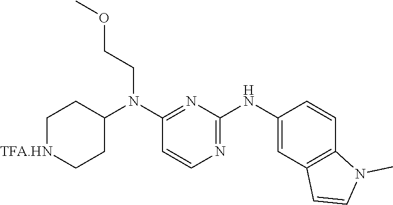

In a 10 mL Round bottom flask a mixture of tert-butyl 4-((2-methoxyethyl)(2-((1-methyl-1H-indol-5-yl)amino)pyrimidin-4-yl)amino)piperidine-1-carboxylate (100 mg, 0.208 mmol) in DCM (5 ml) was combined with TFA (0.016 ml, 0.208 mmol) at 0 °C. The resulting reaction mixture was stirred for 1 h at 25 °C. The reaction was monitored by TLC. After completion of the reaction, volatiles were removed under reduced pressure to afford 2,2,2-trifluoro-1-(4-((2-methoxyethyl)(2-((1-methyl-1H-indol-5-yl)amino)pyrimidin-4-yl)amino)-114-piperidin-1-yl)ethan-1-one (60 mg, 0.069 mmol, 33.3%). LCMS: 44.6%, RT: 0.87, m/z=381.2 (M+H)+.

### Example A95 2,6-difluoro-4-((4-((2-methoxyethyl)(2-((1-methyl-1H-indol-5-yl)amino)pyrimidin-4-yl)amino)piperidin-1-yl)sulfonyl)benzonitrile

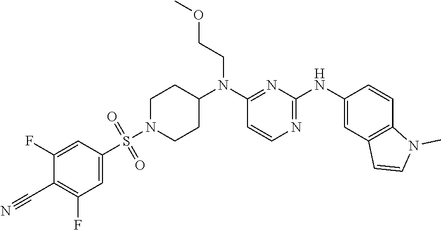 [SMILES: COCCN(c1ccnc(Nc2ccc3c(ccn3C)c2)n1)C1CCN(S(=O)(=O)c2cc(F)c(C#N)c(F)c2)CC1]

In an oven-dried 10 mL two-neck round-bottom flask equipped with N₂ balloon and a rubber septum, 2,2,2-trifluoro-1-(4-((2-methoxyethyl)(2-((1-methyl-1H-indol-5-yl)amino)pyrimidin-4-yl)amino)-114-piperidin-1-yl)ethan-1-one (84 mg, 0.221 mmol) in DCM (5 mL) was combined with TEA (44.7 mg, 0.442 mmol) and stirred for 5 min. The resulting reaction mixture was cooled to −78 °C followed by addition of 4-cyano-3,5-difluorobenzenesulfonyl chloride (52.5 mg, 0.221 mmol). The reaction was stirred for 30 min at the same temperature. The reaction was monitored by LCMS, quenched with ice water (10 mL) and extracted with DCM (2×20 mL). The combined organic layers were dried over anhydrous sodium sulphate, filtered and concentrated under reduced pressure. Thus obtained crude was purified by prep-HPLC to get 2,6-difluoro-4-((4-((2-methoxyethyl)(2-((1-methyl-1H-indol-5-yl)amino)pyrimidin-4-yl)amino)piperidin-1-yl)sulfonyl)benzonitrile (5.5 mg, 0.093 mmol, 42.1%) as white solid. 19F NMR (376 MHz, DMSO-d6): δ −101.37 (2F). 1H-NMR (400 MHz, DMSO-d6): δ 8.63 (s, 1H), 7.94 (d, J=7.2 Hz, 2H), 7.85 (d, J=6.0 Hz, 1H), 7.43 (s, 1H), 7.18 (d, J=7.6 Hz, 1H), 6.36 (d, J=8.0 Hz, 1H), 6.01 (d, J=6.0 Hz, 1H), 4.26 (bs, 1H), 3.86 (d, J=10.8 Hz, 2H), 3.43-3.40 (m, 4H), 3.27 (s, 3H), 3.11 (t, J=8.0 Hz, 2H), 2.70-2.69 (m, 1H), 2.62 (s, 3H), 2.55-2.51 (m, 1H), 1.82-1.75 (m, 4H). LCMS (Method 2): RT: 1.63, m/z=584.2 (M+H)+.

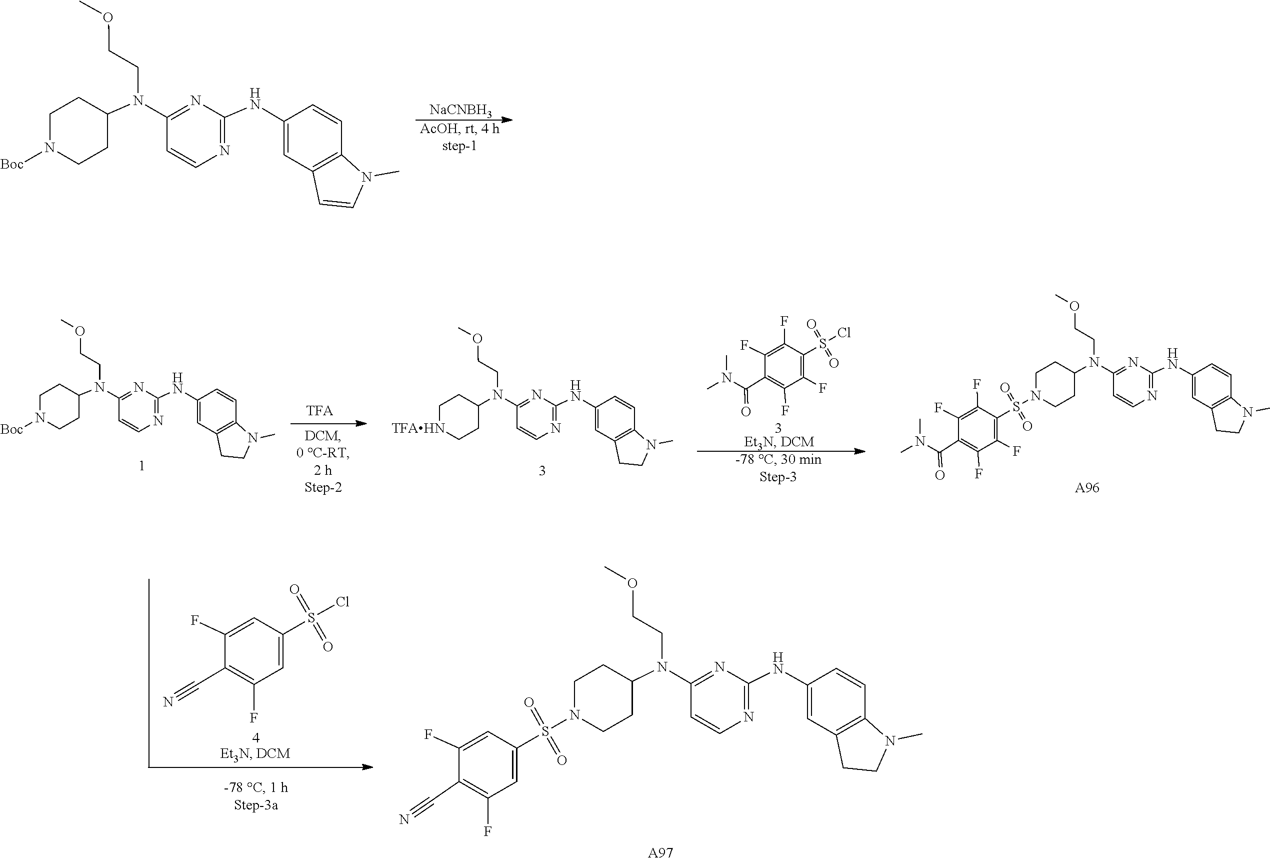

### tert-butyl 4-((2-methoxyethyl)(2-((1-methylindolin-5-yl)amino)pyrimidin-4-yl)amino)piperidine-1-carboxylate (1)

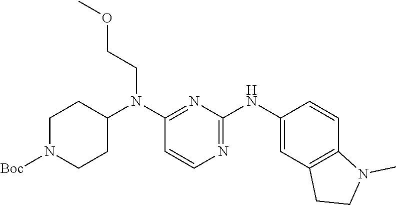 [SMILES: COCCN(c1ccnc(Nc2ccc3c(c2)CCN3C)n1)C1CCN(C(=O)OC(C)(C)C)CC1]

In a 10 mL vial a mixture of tert-butyl 4-((2-methoxyethyl)(2-((1-methyl-1H-indol-5-yl)amino)pyrimidin-4-yl)amino)piperidine-1l-carboxylate (280 mg, 0.583 mmol) were combined in acetic acid (5 ml) and NaCNBH₄ (110 mg, 1.748 mmol) was added at 0 °C. The resulting reaction mixture was stirred for 2 h at 25 °C. The reaction was monitored by TLC & LCMS, quenched with ice water (10 mL) and extracted with ethyl acetate (3×20 mL). The combined organic layers were dried over anhydrous sodium sulphate, filtered and concentrated under reduced pressure affording crude product (130 mg, 0.269 mmol, 46%) as yellow solid. LCMS: 84.59%, RT: 1.58, m/z=483.3 (M+H)+.

### 2,2,2-trifluoro-1-(4-((2-methoxyethyl)(2-((1-methylindolin-5-yl)amino)pyrimidin-4-yl)amino)-114-piperidin-1-yl)ethan-1-one (2)

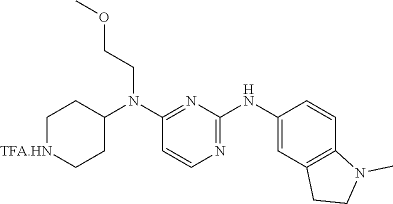

In a 10 mL Round bottom flask tert-butyl 4-((2-methoxyethyl)(2-((1-methylindolin-5-yl)amino)pyrimidin-4-yl)amino)piperidine-1-carboxylate (130 mg, 0.269 mmol) was dissolved in DCM (5 mL). TFA (52 mg, 0.528 mmol) was added at 0 °C. The resulting reaction mixture was stirred for 2 h at 25 °C. The reaction was monitored by TLC & LCMS.

After completion of the reaction volatiles were removed under reduced pressure to afford 2,2,2-trifluoro-1-(4-((2-methoxyethyl)(2-((1-methylindolin-5-yl)amino)pyrimidin-4-yl)amino)-11⁴-piperidin-1-yl)ethan-1-one (90 mg, 0.207 mmol, 70%); LCMS: 87.89%, RT: 0.73, m/z=383.2 (M+H)+.

### Example A96 2,3,5,6-tetrafluoro-4-((4-((2-methoxyethyl)(2-((1-methylindolin-5-yl)amino)pyrimidin-4-yl)amino)piperidin-1-yl)sulfonyl)-N,N-dimethylbenzamide

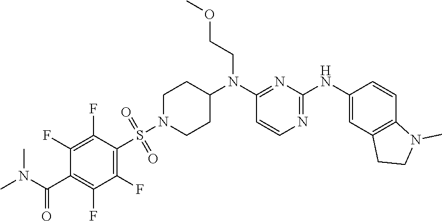

In a 10 mL vial, 2,2,2-trifluoro-1-(4-((2-methoxyethyl)(2-((1-methylindolin-5-yl)amino)pyrimidin-4-yl)amino)-11⁴-piperidin-1-yl)ethan-1-one (90 mg, 0.207 mmol) was dissolved in DCM (5 mL) and TEA was added (53.4 mg, 0.528 mmol) at 0 °C followed by 4-(dimethylcarbamoyl)-2,3,5,6-tetrafluorobenzenesulfonyl chloride (84 mg, 0.264 mmol) at the same temperature. The resulting reaction mixture was stirred for 30 min at rt. The reaction was monitored by TLC & LCMS, quenched with ice water (10 mL) and extracted with DCM (2×10 mL). The combined organic layers were dried over anhydrous sodium sulphate, filtered and concentrated under reduced pressure affording crude product. The crude product was purified by prep HPLC to afford 2,3,5,6-tetrafluoro-4-((4-((2-methoxyethyl)(2-((1-methylindolin-5-yl)amino)pyrimidin-4-yl)amino)piperidin-1-yl)sulfonyl)-N,N-dimethylbenzamide (20 mg, 0.030 mmol, 11.22%) as a white solid. ¹⁹F NMR (376 MHz, DMSO-d6): δ −134.570 (2F), −139.889 to −139.980 (2F). ¹H-NMR (400 MHz, DMSO-d6): δ 8.62 (s, 1H), 7.87 (d, J=6.00 Hz, 1H), 7.44 (s, 1H), 7.21 (d, J=8.00 Hz, 1H), 6.39 (d, J=8.40 Hz, 1H), 6.05 (d, J=6.00 Hz, 1H), 3.86 (d, J=11.20 Hz, 2H), 3.51 (bs, 2H), 3.43 (t, J=6.00 Hz, 2H), 3.27 (s, 3H), 3.13 (t, J=8.00 Hz, 2H), 3.07 (s, 3H), 2.98-2.92 (m, 5H), 2.76 (t, J=7.20 Hz, 2H), 2.62 (s, 3H), 2.53 (s, 1H), 1.86-1.79 (m, 4H). LCMS (Method 4): RT: 1.46, m/z=666.3 (M+H)+.

### Example A97 2,6-difluoro-4-((4-((2-methoxyethyl)(2-((1-methylindolin-5-yl)amino)pyrimidin-4-yl)amino)piperidin-1-yl)sulfonyl)benzonitrile

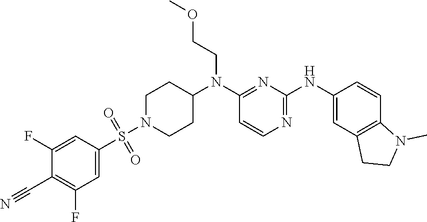 [SMILES: COCCN(c1ccnc(Nc2ccc3c(c2)CCN3C)n1)C1CCN(S(=O)(=O)c2cc(F)c(C#N)c(F)c2)CC1]

The title compound was prepared in a manner similar to A96. 19F NMR (376 MHz, DMSO-d6): δ −101.37 (2F). 1H-NMR (400 MHz, DMSO-d6): δ 8.63 (s, 1H), 7.94 (d, J=7.2 Hz, 2H), 7.85 (d, J=6.0 Hz, 1H), 7.43 (s, 1H), 7.18 (d, J=7.6 Hz, 1H), 6.36 (d, J=8.0 Hz, 1H), 6.01 (d, J=6.0 Hz, 1H), 4.26 (bs, 1H), 3.86 (d, J=10.8 Hz, 2H), 3.43-3.40 (m, 4H), 3.27 (s, 3H), 3.11 (t, J=8.0 Hz, 2H), 2.70-2.69 (m, 1H), 2.62 (s, 3H), 2.55-2.51 (m, 1H), 1.82-1.75 (m, 4H). LCMS (Method 2): RT: 1.63, m/z=584.2 (M+H)+.

The following intermediates were used in the preparation of A54.

### N,N-dimethyl-5-nitropyridin-2-amine (3)

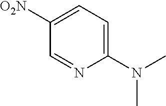

To a 25 mL round bottom flask solution of 2-chloro-5-nitropyridine (1.5 g, 9.46 mmol) in Isobutanol (5 mL), triethyl amine (3.96 mL, 28.4 mmol) followed by dimethylamine hydrochloride (0.849 g, 10.41 mmol) were added. The resulting reaction mixture was heated in a microwave reactor at 100 °C for 2 h. The reaction was monitored by LCMS, quenched with ice water (3 mL) and extracted with ethyl acetate (2×50 mL). The combined organic layers were dried over anhydrous sodium sulphate, filtered and concentrated under reduced pressure affording N,N-dimethyl-5-nitropyridin-2-amine (1.3 g, 7.7 mmol, 81%) as pale yellow gum; LCMS: 99.67%, RT: 1.33, m/z=168 (M+H)⁺.

### N²,N²-dimethylpyridine-2,5-diamine (4)

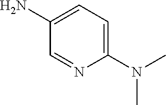

N,N-dimethyl-5-nitropyridin-2-amine (1.4 g, 8.37 mmol) was dissolved in methanol (20 mL) and purged with nitrogen for 2 min. Pd/C (0.446 g, 4.19 mmol) was added and the reaction mixture was stirred under H₂ (1 atm) pressure overnight. The reaction was monitored by LCMS, filtered through celite bed and volatiles were removed under reduced pressure to give N₂,N₂-dimethylpyridine-2,5-diamine (900 mg 5.97 mmol, 71.3%) as white solid.

LCMS: 91.94%, RT: 0.21, m/z=138.06 (M+H)⁺.

### 1-(4-((2-((6-(dimethylamino)pyridin-3-yl)amino)pyrimidin-4-yl)(2-methoxyethyl)amino)-11⁴-piperidin-1-yl)-2,2,2-trifluoroethan-1-one (5)

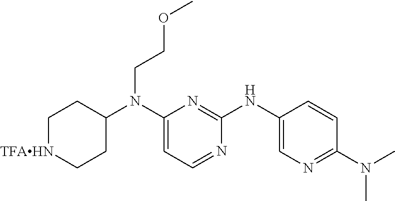

In an oven-dried 100 mL two-neck round-bottom flask equipped with N₂ balloon and a rubber septum, tert-butyl 4-((2-chloropyrimidin-4-yl)(2-methoxyethyl)amino)piperidine-1-carboxylate (616 mg, 1.660 mmol) and N²,N²-dimethylpyridine-2,5-diamine (253 mg, 1.844 mmol) where dissolved in isobutanol (5 mL). TFA (0.565 mL, 7.38 mmol) was added and the reaction was stirred for 4 h at 80 °C. The reaction was monitored by LCMS. After completion of the reaction, volatiles were removed under reduced pressure. The crude was purified by reverse phase by using RediSep R_{f} Gold® silica (100 g column) [Mobile phase A: 10 mM ammonium bicarbonate in milli-Q water; Mobile phase B: CH₃CN] while the desire product was eluted at 10-20% of the mobile phase; and the fractions were lyophilized to afford 1-(4-((2-((6-(dimethylamino)pyridin-3-yl)amino)pyrimidin-4-yl)(2-methoxyethyl)amino)-1□4-piperidin-1-yl)-2,2,2-trifluoroethan-1-one (500 mg, 1.252 mmol, 67.9%) as a brown solid.

LCMS: 93.9%, RT: 1.68, m/z=372.2 (M+H)⁺.

1-(4-((2-((6-(dimethylamino)-5-methylpyridin-3-yl)amino)pyrimidin-4-yl)(2-methoxyethyl)amino)-114-piperidin-1-yl)-2,2,2-trifluoroethan-1-one (5) was prepared using a similar strategy and intermediates and used to make A55:

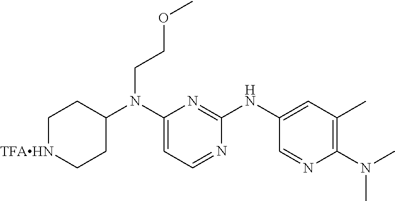

In an oven-dried 100 mL two-neck round-bottom flask equipped with N₂ balloon and a rubber septum, tert-butyl 4-((2-chloropyrimidin-4-yl)(2-methoxyethyl)amino)piperidine-1-carboxylate (845 mg, 2.280 mmol) and N²,N²,3-trimethylpyridine-2,5-diamine ((450 mg, 2.59 mmol) were dissolved in isobutanol (5 mL), TFA (0.814 ml, 10.13 mmol) was added and the reaction stirred for 4 h at 80 °C. The reaction was monitored by LCMS. After completion of the reaction, volatiles were removed under reduced pressure. Thus obtained crude was purified by reverse phase by using RediSep R_{f} Gold® silica gel (100 g column) [Mobile phase A: 10 mM ammonium bicarbonate in milli-Q water; Mobile phase B: CH₃CN] while the desire product was eluted at 10-20% of the mobile phase; and the fractions were lyophilized to afford 1-(4-((2-((6-(dimethylamino)-5-methylpyridin-3-yl)amino)pyrimidin-4-yl)(2-methoxyethyl)amino)-114-piperidin-1-yl)-2,2,2-trifluoroethan-1-one (550 mg, 1.063 mmol, 42.0%) as a brown solid.

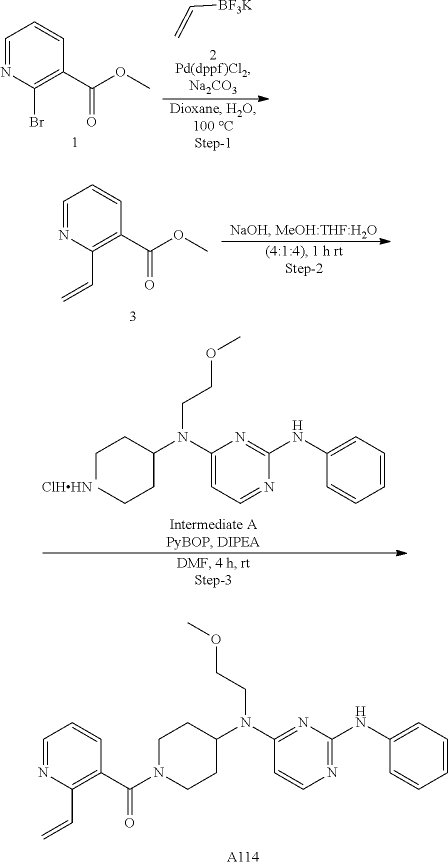

### methyl 2-vinylnicotinate (3)

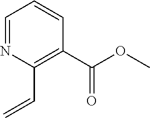

In a 40 mL reaction vail, methyl 2-bromonicotinate (500 mg, 2.31 mmol), trifluoro(vinyl)-14-borane, potassium salt (620 mg, 4.63 mmol), Na₂CO₃ (735 mg, 6.94 mmol) and dioxane (8 mL) were added at 25 °C. After degassing the reaction mixture with nitrogen for 5 min, PdCl₂(dppf)·CH₂Cl₂ adduct (15.97 mg, 0.020 mmol) was added and further degassed for 5 min. The resulting reaction mixture stirred for 4 h at 80 °C. The progress of the reaction was monitored by TLC and LCMS. After completion reaction, volatiles were removed under reduced pressure. The obtained residue was diluted with water (20 mL), extracted with EtOAc (2×15 mL), combined organic layers were washed with water (20 mL) followed by brine (20 mL), dried over Na2SO4, concentrated under reduced pressure afforded crude product. The obtained crude was purified by silica gel chromatography with 30% Ethyl acetate in hexane as eluent to afford (methyl 2-vinylnicotinate (300 mg, 1.84 mmol, 80%) as an off white solid. LCMS: 96%, RT: 1.11 min, m/z=164.0 (M+H)⁺.

### 2-vinylnicotinic acid (4)

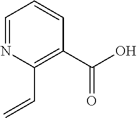

In a 50 ml single necked round bottom flask methyl 2-vinylisonicotinate (300 mg, 1.84 mmol) was dissolved in Tetrahydrofuran (10 mL) and the reaction mixture cooled to 0 °C. To this reaction mixture NaOH (1M), MeOH, H₂O (1 mL, 0.25 mL, 1 mL) were introduced at the same temperature and the mixture was stirred for 30 min. The progress of the reaction was monitored by TLC and LCMS. Volatiles were removed under reduced pressure and reaction mixture was acidified with 1.5 N HCl a pH of 3 to 4 and the solid collected by filtration. The obtained solid was dried under vacuum to afford 2-vinylisonicotinic acid (220 mg, 1.48 mmol, 80%). LCMS: 95%, RT: 0.42 min, m/z: 149.9 (M+H⁺).

### Example A114 (4-((2-methoxyethyl)(2-(phenylamino)pyrimidin-4-yl)amino)piperidin-1-yl)(2-vinylpyridin-3-yl)methanone

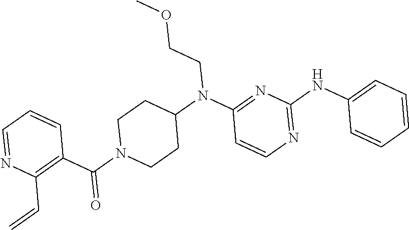 [SMILES: C=Cc1ncccc1C(=O)N1CCC(N(CCOC)c2ccnc(Nc3ccccc3)n2)CC1]

To an oven dried 50 mL two-neck round-bottom flask equipped with N₂ balloon, 2-vinylnicotinic acid (220 mg, 1.48 mmol), DMF (6 mL), PyBOP (536 mg, 1.62 mmol), N⁴-(2-methoxyethyl)-N²-phenyl-N⁴-(piperidin-4-yl)pyrimidine-2,4-diamine hydrochloride (530 mg, 1.48 mmol) and DIPEA (266 mg, 2.061 mmol) were added at 0 °C. The resulting reaction mixture stirred for 4 h at room temperature. The progress of the reaction was monitored by TLC, quenched with ice cold water (30 mL) and extracted with EtOAc (3×50 mL). The combined organic layers were dried over anhydrous sodium sulphate, filtered and concentrated under reduced pressure to afford crude product. The crude mass was purified by column chromatography by using silica gel 100-200 and eluted with 100% EtOAc to afford (4-((2-methoxyethyl)(2-(phenylamino)pyrimidin-4-yl)amino)piperidin-1-yl)(2-vinylpyridin-3-yl)methanone (60 mg, 0.130 mmol, 9%) as an off white solid. 1H-NMR (400 MHz, DMSO-d6): δ 8.70 (s, 1H), 8.63-8.61 (m, 1H), 7.96 (d, J=6.00 Hz, 1H), 7.74 (d, J=7.2 Hz, 1H), 7.71 (d, J=7.60 Hz, 2H), 7.36 (dd, J=8.00, 4.80 Hz, 1H), 7.24 (t, J=8.40 Hz, 2H), 6.90 (t, J=7.60 Hz, 1H), 6.84-6.77 (m, 1H), 6.39 (dd, J=17.2, 2.4 Hz, 1H), 6.19 (d, J=6.00 Hz, 1H), 5.55 (dd, J=10.8, 2.4 Hz, 1H), 4.74 (bs, 1H), 4.50 (bs, 1H), 3.58-3.47 (m, 4H), 3.38-3.35 (m, 1H), 3.31 (s, 3H), 3.17-3.14 (m, 1H), 2.93-2.90 (m, 1H), 1.84-1.65 (m, 4H) ppm. LCMS (Method 2): RT: 1.22, m/z=459.2 (M+H)+.

### Example A115 (4-((2-methoxyethyl)(2-(phenylamino)pyrimidin-4-yl)amino)piperidin-1-yl)(4-vinylpyridin-2-yl)methanone

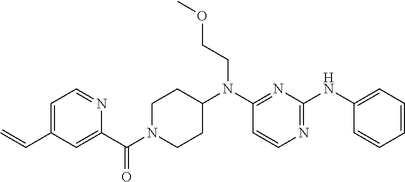 [SMILES: C=Cc1ccnc(C(=O)N2CCC(N(CCOC)c3ccnc(Nc4ccccc4)n3)CC2)c1]

The title compound was prepared in a similar manner to A114. 1H-NMR (400 MHz, DMSO-d6): δ 9.05 (s, 1H), 8.56 (d, J=4.80 Hz, 1H), 7.96 (d, J=6.00 Hz, 1H), 7.71 (t, J=7.60 Hz, 3H), 7.56-7.55 (m, 1H), 7.24 (t, J=8.00 Hz, 2H), 6.90-6.78 (m, 2H), 6.23 (d, J=18.00 Hz, 2H), 5.61 (d, J=11.20 Hz, 1H), 4.62 (d, J=70.80 Hz, 2H), 3.80 (d, J=12.80 Hz, 1H), 3.54-3.47 (m, 4H), 3.28 (s, 3H), 3.20-3.14 (m, 1H), 2.91-2.85 (m, 1H), 1.84-1.63 (m, 4H).

LCMS (Method 3): RT: 2.23, m/z=459.2 (M+H)+.

### A121 (4-((2-methoxyethyl)(2-(phenylamino)pyrimidin-4-yl)amino)piperidin-1-yl)(6-vinylpyridin-2-yl)methanone

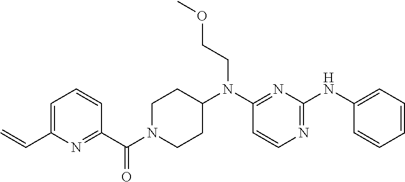 [SMILES: C=Cc1cccc(C(=O)N2CCC(N(CCOC)c3ccnc(Nc4ccccc4)n3)CC2)n1]

The title compound was prepared in a similar manner to A114. LCMS (Method 2): RT: 2.25, m/z=459.2[M+H]+.

### A122 (4-((2-methoxyethyl)(2-(phenylamino)pyrimidin-4-yl)amino)piperidin-1-yl)(4-vinylpyridin-3-yl)methanone

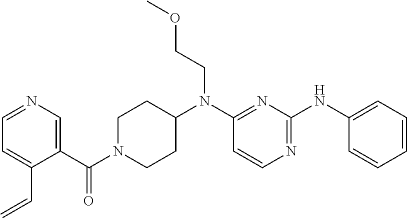 [SMILES: C=Cc1ccncc1C(=O)N1CCC(N(CCOC)c2ccnc(Nc3ccccc3)n2)CC1]

The title compound was prepared in a similar manner to A114. LCMS (Method 4): RT: 1.12, m/z=459.2[M+H]+.

### A124 (4-((2-methoxyethyl)(2-(phenylamino)pyrimidin-4-yl)amino)piperidin-1-yl)(6-vinylpyridin-3-yl)methanone

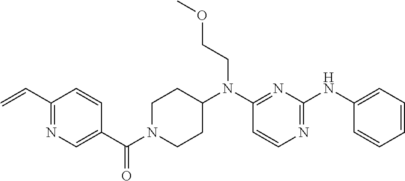 [SMILES: C=Cc1ccc(C(=O)N2CCC(N(CCOC)c3ccnc(Nc4ccccc4)n3)CC2)cn1]

The title compound was prepared in a similar manner to A114. LCMS (Method 1): RT: 1.26, m/z=459.2[M+H]+.

### A133 (4-((2-methoxyethyl)(2-(phenylamino)pyrimidin-4-yl)amino)piperidin-1-yl)(2-vinylphenyl)methanone

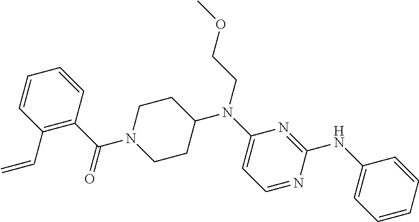 [SMILES: C=Cc1ccccc1C(=O)N1CCC(N(CCOC)c2ccnc(Nc3ccccc3)n2)CC1]

The title compound was prepared in a similar manner to A114. LCMS (Method 1): RT: 1.48, m/z=458.2[M+H]+.

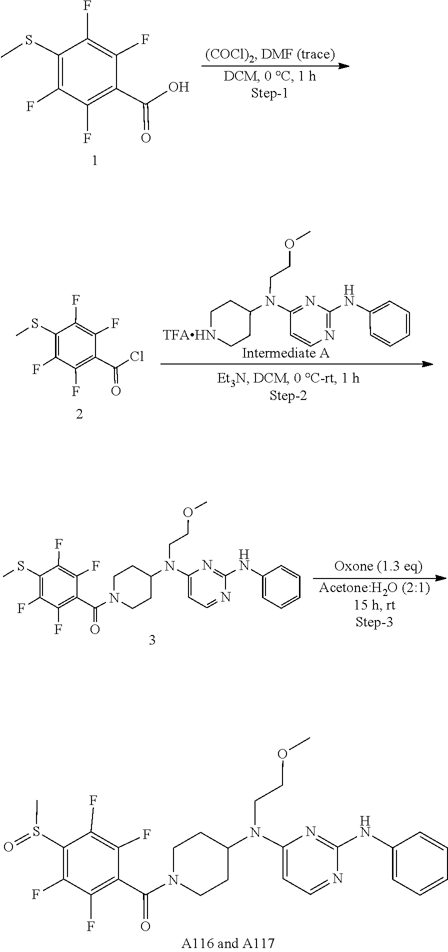

### 2,3,5,6-tetrafluoro-4-(methylthio)benzoyl chloride (2)

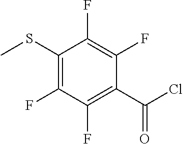

To a stirred solution of 2,3,5,6-tetrafluoro-4-(methylthio)benzoic acid (500 mg, 2.082 mmol) and DCM (5 mL) was added oxalyl chloride (0.357 mL, 4.16 mmol) and 1 drop of DMF (0.016 mL, 0.208 mmol) at 0 °C. Then the reaction mixture was stirred at 25 °C or 1 h. Progress of the reaction was monitored by TLC. Volatiles were removed under reduced pressure to afford the desired product 2,3,5,6-tetrafluoro-4-(methylthio)benzoyl chloride (538 mg, 2.080 mmol, 100%) as colorless liquid which was used for further reaction without further purification.

### (4-((2-methoxyethyl)(2-(phenylamino)pyrimidin-4-yl)amino)piperidin-1-yl)(2,3,5,6-tetrafluoro-4-(methylthio)phenyl)methanone (3)

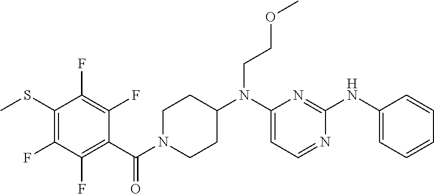 [SMILES: COCCN(c1ccnc(Nc2ccccc2)n1)C1CCN(C(=O)c2c(F)c(F)c(SC)c(F)c2F)CC1]

To a stirred solution of 2,2,2-trifluoro-1-(4-((2-methoxyethyl)(2-(phenylamino)pyrimidin-4-yl)amino)-114-piperidin-1-yl)ethan-1-one (905 mg, 2.049 mmol) in DCM (10 mL) was added triethylamine (1.428 mL, 10.25 mmol) at 0 °C and the mixture was stirred for 5 min followed by addition of a 2,3,5,6-tetrafluoro-4-(methylthio)benzoyl chloride (530 mg, 2.049 mmol) solution in DCM (2 mL). The reaction mixture was stirred at the same temperature for 1 h. The progress of the reaction was monitored by LCMS, quenched with ice cold water (5 mL) and extracted with DCM (3×50 mL). The combined organic layers were dried over anhydrous sodium sulphate, filtered and concentrated under reduced pressure to get crude product. The obtained crude product was purified by chromatography using 100-200 mesh silica gel with 0-100% EtOAc in hexane to get (4-((2-methoxyethyl)(2-(phenylamino)pyrimidin-4-yl)amino)piperidin-1-yl)(2,3,5,6-tetrafluoro-4-(methylthio)phenyl)methanone (380 mg, 0.636 mmol, 31.0%). LCMS: 92.2%, RT: 2.58, m/z=550.2 (M+H)⁺.

### Examples A116 and A117 (4-((2-methoxyethyl)(2-(phenylamino)pyrimidin-4-yl)amino)piperidin-1-yl)(2,3,5,6-tetrafluoro-4-(methylsulfinyl)phenyl)methanone

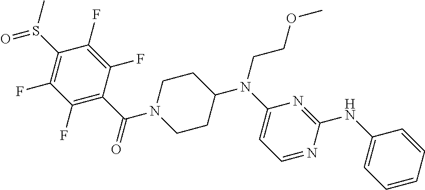 [SMILES: COCCN(c1ccnc(Nc2ccccc2)n1)C1CCN(C(=O)c2c(F)c(F)c(S(C)=O)c(F)c2F)CC1]

To a stirred solution of (4-((2-methoxyethyl)(2-(phenylamino)pyrimidin-4-yl)amino)piperidin-1-yl)(2,3,5,6-tetrafluoro-4-(methylthio)phenyl)methanone (330 mg, 0.600 mmol) in mixture of acetone (5 mL) and water (5 mL) was added Oxone (240 mg, 0.781 mmol). Then the reaction mixture stirred at rt for 15 h. Progress of the reaction was monitored by TLC, quenched with ice cold water and extracted with EtOAc (3×50 mL).The combined organic layers were dried over Na₂SO₄ and concentrated at reduced pressure. The crude residue was purified by chromatography using 100-200 mesh silica gel with 0-50% EtOAc in hexane as eluent to get 270 mg of pure compound. Then the enantiomers were separated by chiral HPLC using column IZ-[250*30] mm, Sum and mobile phase: CO₂: Methanol. First eluting isomer A116-(4-((2-methoxyethyl)(2-(phenylamino)pyrimidin-4-yl)amino)piperidin-1-yl)(2,3,5,6-tetrafluoro-4-(methylsulfinyl)phenyl)methanone (90 mg, 0.158 mmol, 26.2%) and second eluting isomer A117 (R)-(4-((2-methoxyethyl)(2-(phenylamino)pyrimidin-4-yl)amino)piperidin-1-yl)(2,3,5,6-tetrafluoro-4-(methylsulfinyl)phenyl)methanone (100 mg, 0.175 mmol, 29.2%) were isolated as off white solids.

19F NMR (376 MHz, DMSO-d6): δ −139.27 to −138.91 (1F), −140.25 to −139.81 (1F), −141.29 to −141.03 (2F) 1H-VT-NMR (400 MHz, DMSO-d6): δ 8.71 (s, 1H), 7.97 (d, J=6.00 Hz, 1H), 7.70 (d, J=7.60 Hz, 2H), 7.25 (t, J=8.40 Hz, 2H), 6.91 (t, J=7.20 Hz, 1H), 6.21 (d, J=6.00 Hz, 1H), 4.67 (d, J=13.20 Hz, 1H), 4.54-4.51 (m, 1H), 3.72 (d, J=13.20 Hz, 1H), 3.57-3.48 (m, 4H), 3.32-3.29 (m, 4H), 3.22 (s, 3H), 2.99 (t, J=10.80 Hz, 1H), 1.92-1.88 (m, 1H), 1.80-1.65 (m, 3H), LCMS(Method 2): RT: 2.23, m/z=566.2 (M+H)+.

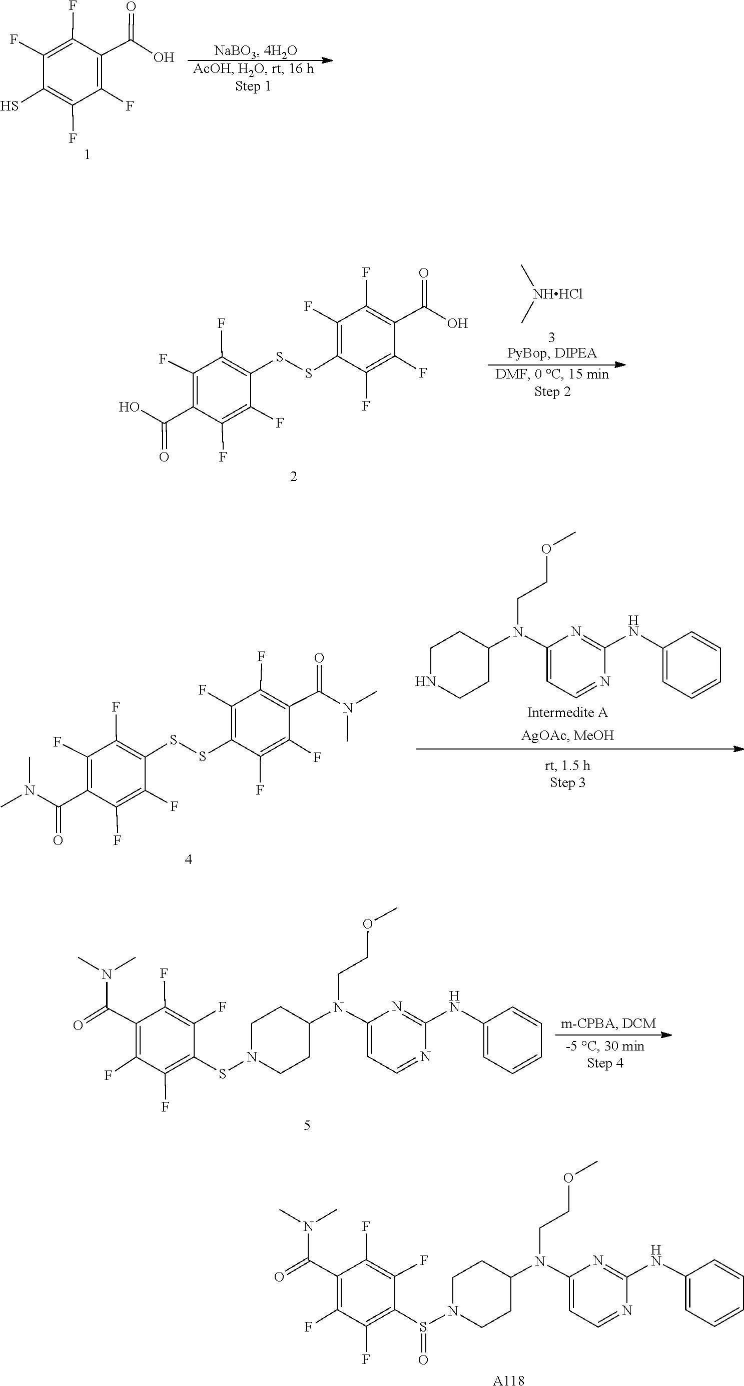

### Synthesis of 4,4′-disulfanediylbis(2,3,5,6-tetrafluorobenzoic acid) (2)

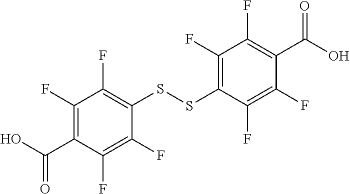 [SMILES: O=C(O)c1c(F)c(F)c(SSc2c(F)c(F)c(C(=O)O)c(F)c2F)c(F)c1F]

To a stirred solution of 2,3,5,6-tetrafluoro-4-mercaptobenzoic acid (1 g, 4.42 mmol) in acetic acid (15 mL) and water (6 mL), sodium perborate tetrahydrate (1.361 g, 8.84 mmol) was added. The resulting reaction mixture was stirred at RT for 16 h. The progress of the reaction was monitored by UPLC. After completion, the reaction mixture was diluted with water (50 mL) and extracted with Ethyl acetate (50 mL×3); the combined organic extracts were washed with brine (20 mL), dried over anhydrous sodium sulphate, filtered and concentrated under reduced pressure (bath temperature: 45 °C) to afford 4,4′-disulfanediylbis(2,3,5,6-tetrafluorobenzoic acid) (850 mg, 1.850 mmol, 41.8%) as an off white solid. LCMS: 98.57%, RT: 1.37 min, m/z=448.97 (M−H)−.

### Synthesis of 4,4′-disulfanediylbis(2,3,5,6-tetrafluoro-N,N-dimethylbenzamide) (4)

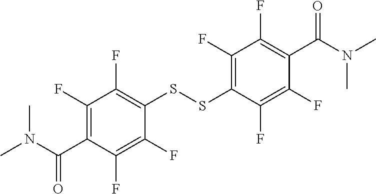

To a stirred solution of 4,4′-disulfanediylbis(2,3,5,6-tetrafluorobenzoic acid) (1.2 g, 2.67 mmol) and dimethylamine hydrochloride (0.456 g, 5.60 mmol) in DMF (20 mL) DIPEA (1.857 mL, 10.66 mmol) was added followed by PyBOP (3.33 g, 6.40 mmol) at 0 °C. The resulting reaction mixture was stirred at 0 °C for 15 min. The progress of the reaction was monitored by LCMS. After completion, the reaction was quenched with water and extracted with EtOAc (2×30 mL), the combined organics were dried over anhydrous Na₂SO₄, filtered and concentrated to afford a crude residue. The obtained crude was purified by neutral alumina using 30% EtOAc/hexane as eluent to afford 4,4′-disulfanediylbis(2,3,5,6-tetrafluoro-N,N-dimethylbenzamide) (270 mg, 0.401 mmol, 15.06%) as a pale yellow gum.

LCMS: 75.67%, RT: 2.31, m/z=505.0 (M+H)⁺.

### Synthesis of 2,3,5,6-tetrafluoro-4-((4-((2-methoxyethyl)(2-(phenylamino)pyrimidin-4-yl)amino)piperidin-1-yl)thio)-N,N-dimethylbenzamide (6)

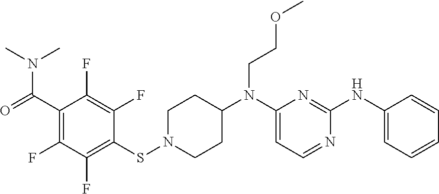 [SMILES: COCCN(c1ccnc(Nc2ccccc2)n1)C1CCN(Sc2c(F)c(F)c(C(=O)N(C)C)c(F)c2F)CC1]

To a solution of N⁴-(2-methoxyethyl)-N²-phenyl-N⁴-(piperidin-4-yl)pyrimidine-2,4-diamine (234 mg, 0.714 mmol) and 4,4′-disulfanediylbis(2,3,5,6-tetrafluoro-N,N-dimethylbenzamide) (240 mg, 0.476 mmol) in MeOH (2.5 mL) was added silver acetate (159 mg, 0.952 mmol) and the mixture was stirred at rt for 1.5 h. The progress of the reaction was monitored by LCMS After completion, the reaction was diluted with MeOH (5 mL), filtered and concentrated under reduced pressure. The crude was purified by neutral alumina using 40% EtOAc/hexane as eluent to afford 2,3,5,6-tetrafluoro-4-((4-((2-methoxyethyl)(2-(phenylamino)pyrimidin-4-yl)amino)piperidin-1-yl)thio)-N,N-dimethylbenzamide (160 mg, 0.235 mmol, 49.4%) as a pale yellow gum. LCMS: 85.58%, RT: 1.75, m/z=579.32 (M+H)⁺.

### Example A118 2,3,5,6-tetrafluoro-4-((4-((2-methoxyethyl)(2-(phenylamino)pyrimidin-4-yl)amino)piperidin-1-yl)sulfinyl)-N,N-dimethylbenzamide

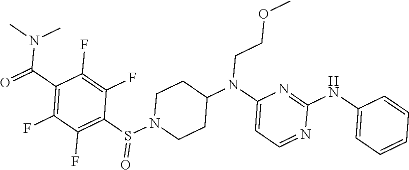 [SMILES: COCCN(c1ccnc(Nc2ccccc2)n1)C1CCN(S(=O)c2c(F)c(F)c(C(=O)N(C)C)c(F)c2F)CC1]

To a solution of 2,3,5,6-tetrafluoro-4-((4-((2-methoxyethyl)(2-(phenylamino)pyrimidin-4-yl)amino)piperidin-1-yl)thio)-N,N-dimethylbenzamide (90 mg, 0.156 mmol) in DCM (5 mL) was added mCPBA (53.7 mg, 0.311 mmol) at −5 °C and the reaction was stirred at the same temperature for 30 min. The progress of the reaction was monitored by LCMS. After completion, the reaction was quenched with water and extracted with DCM (2×20 mL). The combined organic layers were dried over anhydrous Na₂SO₄, filtered and concentrated. The crude was purified by prep-HPLC using 0.1% ABC in water/ACN as buffer to afford 2,3,5,6-tetrafluoro-4-((4-((2-methoxyethyl)(2-(phenylamino)pyrimidin-4-yl)amino)piperidin-1-yl)sulfinyl)-N,N-dimethylbenzamide (9 mg, 0.015 mmol, 9.63%).

¹⁹F NMR (376 MHz, DMSO-d6): δ −141.51 to −140.56 (4F). ¹H-VT-NMR (400 MHz, DMSO-d6): δ 8.72 (s, 1H), 7.95 (d, J=6.00 Hz, 1H), 7.68 (d, J=8.00 Hz, 2H), 7.16 (t, J=8.00 Hz, 2H), 6.88 (t, J=7.20 Hz, 1H), 6.18 (d, J=6.00 Hz, 1H), 4.378-4.34 (m, 1H), 3.86 (d, J=11.20 Hz, 1H), 3.66 (d, J=12.80 Hz, 1H), 3.52-3.47 (m, 4H), 3.29 (s, 3H), 3.09 (s, 3H), 2.97 (s, 3H), 2.88-2.75 (m, 2H), 1.92-1.82 (m, 4H). LCMS (Method 2): RT: 1.40, m/z=595.2 (M+H)+.

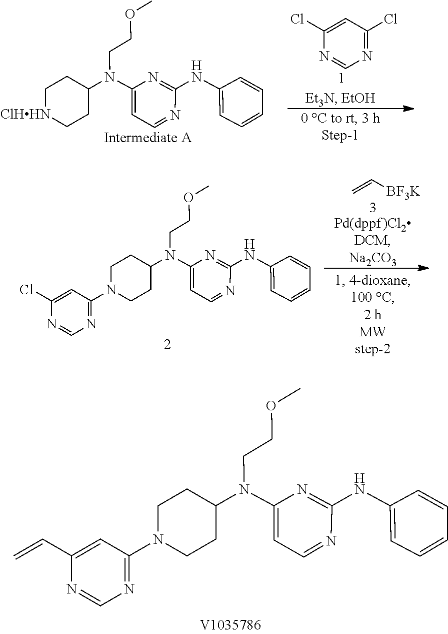

### Synthesis of N4-(1-(6-chloropyrimidin-4-yl)piperidin-4-yl)-N4-(2-methoxyethyl)-N2-phenylpyrimidine-2,4-diamine (2)

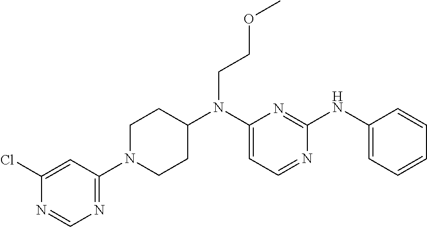 [SMILES: COCCN(c1ccnc(Nc2ccccc2)n1)C1CCN(c2cc(Cl)ncn2)CC1]

In an oven dried 40 mL reaction vial, N⁴-(2-methoxyethyl)-N²-phenyl-N⁴-(piperidin-4-yl)pyrimidine-2,4-diamine Hydrochloride (100 mg, 0.275 mmol), ethanol (5 mL), 2,4-dichloropyrimidine (40.9 mg, 0.275 mmol) and triethylamine (0.077 mL, 0.550 mmol) were combined at 0 °C and stirred for 3 h at rt. The progress of the reaction was monitored by TLC and LCMS. After completion, the volatiles were removed under reduced pressure, the residue was diluted with water (10 mL) and extracted with EtOAc (2×10 mL). The combined organics were dried over Na₂SO₄ and concentrated under reduced pressure to afford N4-(1-(2-chloropyrimidin-4-yl)piperidin-4-yl)-N4-(2-methoxyethyl)-N2-phenylpyrimidine-2,4-diamine (90 mg, 0.170 mmol, 61.8%) as an off white solid. LCMS: 83.88%, RT: 1.325 min, m/z=440.1 (M+H)⁺.

### Example A119 N4-(2-methoxyethyl)-N2-phenyl-N4-(1-(6-vinylpyrimidine-4-yl)piperidin-4-yl)pyrimidine-2,4-diamine

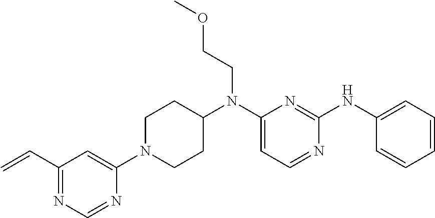 [SMILES: C=Cc1cc(N2CCC(N(CCOC)c3ccnc(Nc4ccccc4)n3)CC2)ncn1]

To a solution of N⁴-(1-(6-chloropyrimidin-4-yl)piperidin-4-yl)-N⁴-(2-methoxyethyl)-N²-phenylpyrimidine-2,4-diamine (200 mg, 0.455 mmol) and potassium vinyltrifluoroborate (122 mg, 0.909 mmol) in 1,4-Dioxane (5 mL) Na₂CO₃ (145 mg, 1.364 mmol) was added. The reaction mixture was degassed with nitrogen for 2 min. To this solution, PdCl2(dppf)-CH2Cl2 adduct (37.1 mg, 0.045 mmol) was added and the mixture was heated at 100 °C for 2 h in microwave reactor. The progress of the reaction was monitored by LCMS, quenched with water and extracted with EtOAc (2×20 mL). The combined organic portions were dried over Na₂SO₄, filtered and concentrated under reduced pressure. The obtained crude was purified by Prep-HPLC to afford N⁴-(2-methoxyethyl)-N²-phenyl-N⁴-(1-(6-vinylpyrimidin-4-yl)piperidin-4-yl)pyrimidine-2,4-diamine (35.5 mg, 0.082 mmol, 18.08%) as an off-white solid.

1H-VT-NMR (400 MHz, DMSO-d6): δ 8.71 (s, 1H), 8.47 (s, 1H), 7.96 (d, J=6.00 Hz, 1H), 7.72 (d, J=8.00 Hz, 2H), 7.23 (t, J=8.00 Hz, 2H), 6.91-6.87 (m, 2H), 6.65 (q, J=10.40 Hz, 1H), 6.37 (dd, J=17.20, 1.60 Hz, 1H), 6.21 (d, J=6.40 Hz, 1H), 5.54 (dd, J=2.00, 10.60 Hz, 1H), 4.63-4.56 (m, 3H), 3.53-3.46 (m, 4H), 3.26 (s, 3H), 3.01-2.98 (m, 2H), 1.81-1.69 (m, 4H) LCMS(Method 2): RT: 1.32, m/z=432.2 (M+H)+.

### Example A126 N⁴-(2-methoxyethyl)-N²-phenyl-N⁴-(1-(4-vinylpyridin-2-yl)piperidin-4-yl)pyrimidine-2,4-diamine

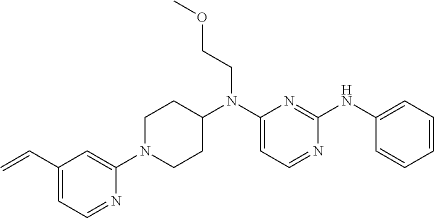 [SMILES: C=Cc1ccnc(N2CCC(N(CCOC)c3ccnc(Nc4ccccc4)n3)CC2)c1]

### Example A127 N⁴-(2-methoxyethyl)-N²-phenyl-N⁴-(1-(2-vinylpyridin-4-yl)piperidin-4-yl)pyrimidine-2,4-diamine

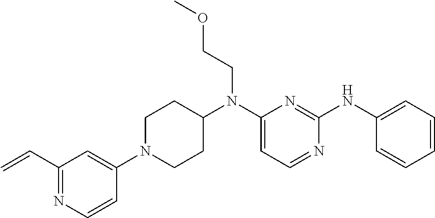 [SMILES: C=Cc1cc(N2CCC(N(CCOC)c3ccnc(Nc4ccccc4)n3)CC2)ccn1]

### Example A129 N⁴-(2-methoxyethyl)-N²-phenyl-N⁴-(1-(2-vinylpyriimidin-4-yl)piperidin-4-yl)pyrimidine-2,4-diamine

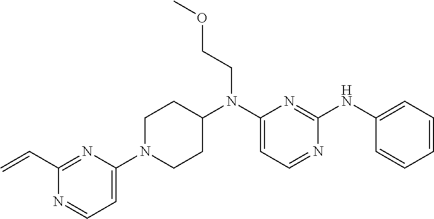 [SMILES: C=Cc1nccc(N2CCC(N(CCOC)c3ccnc(Nc4ccccc4)n3)CC2)n1]

### Example A130 N⁴-(2-methoxyethyl)-N²-phenyl-N⁴-(1-(4-vinylpyrimidin-2-yl)piperidin-4-yl)pyrimidine-2,4-diamine

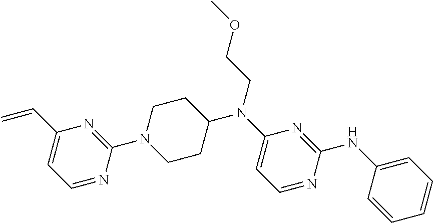 [SMILES: C=Cc1ccnc(N2CCC(N(CCOC)c3ccnc(Nc4ccccc4)n3)CC2)n1]

### Example A131 N⁴-(1-(5-fluoro-2-vinylpyridin-4-yl)piperidin-4-yl)-N⁴-(2-methoxyethyl)-N²-phenylpyrimidine-2,4-diamine

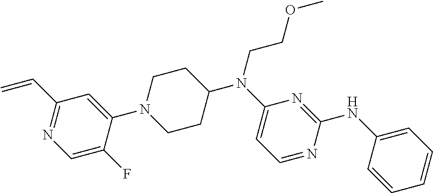 [SMILES: C=Cc1cc(N2CCC(N(CCOC)c3ccnc(Nc4ccccc4)n3)CC2)c(F)cn1]

### Example A134 N⁴-(2-methoxyethyl)-N²-phenyl-N⁴-(1-(6-vinylpyridin-3-yl)piperidin-4-yl)pyrimidine-2,4-diamine

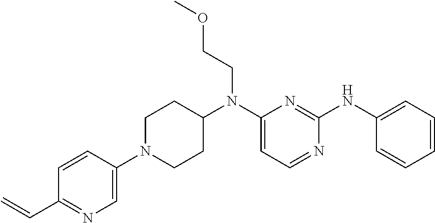 [SMILES: C=Cc1ccc(N2CCC(N(CCOC)c3ccnc(Nc4ccccc4)n3)CC2)cn1]

Table 5 provides further compounds according to the invention. The compounds in the following Table 5 were prepared in the same manner as described herein from the appropriate starting materials. Additional synthesis schemes for examples A613, A614, A618, A619, A621, and A625 are provided following Table 5.

TABLE 5

| Structure | Example |
|---|---|
| 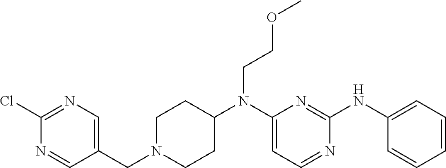 [SMILES: COCCN(c1ccnc(Nc2ccccc2)n1)C1CCN(Cc2cnc(Cl)nc2)CC1] | A135 |
| C₂₃H₂₈ClN₇O | |
| 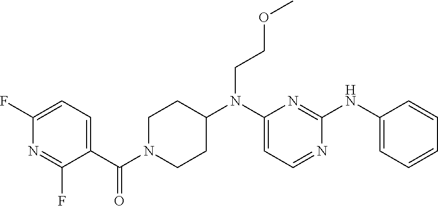 [SMILES: COCCN(c1ccnc(Nc2ccccc2)n1)C1CCN(C(=O)c2ccc(F)nc2F)CC1] | A136 |
| C₂₄H₂₆F₂N₆O₂ | |
| 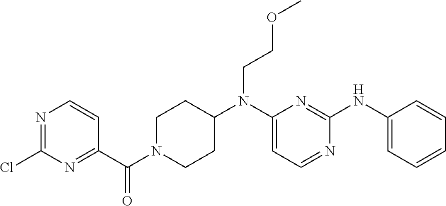 [SMILES: COCCN(c1ccnc(Nc2ccccc2)n1)C1CCN(C(=O)c2ccnc(Cl)n2)CC1] | A137 |
| C₂₃H₂₆ClN₇O₂ | |
| 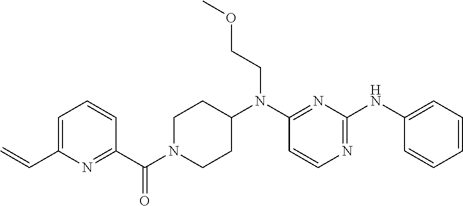 [SMILES: C=Cc1cccc(C(=O)N2CCC(N(CCOC)c3ccnc(Nc4ccccc4)n3)CC2)n1] | A138 |
| C₂₆H₃₀N₆O₂ | |
| 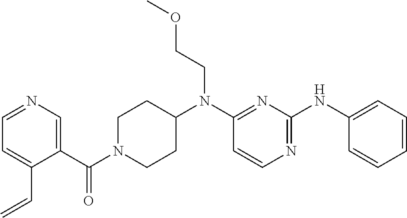 [SMILES: C=Cc1ccncc1C(=O)N1CCC(N(CCOC)c2ccnc(Nc3ccccc3)n2)CC1] | A139 |
| C₂₆H₃₀N₆O₂ | |
| 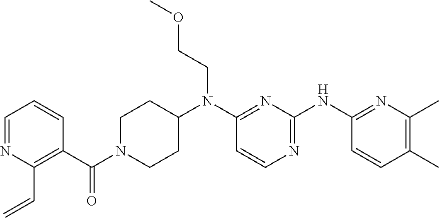 [SMILES: C=Cc1ncccc1C(=O)N1CCC(N(CCOC)c2ccnc(Nc3ccc(C)c(C)n3)n2)CC1] | A140 |
| C₂₇H₃₃N₇O₂ | |
| 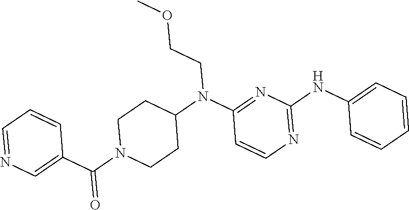 [SMILES: COCCN(c1ccnc(Nc2ccccc2)n1)C1CCN(C(=O)c2cccnc2)CC1] | A141 |
| C₂₄H₂₈N₆O₂ | |
| 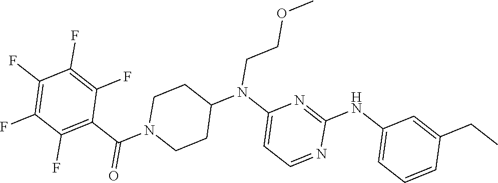 [SMILES: CCc1cccc(Nc2nccc(N(CCOC)C3CCN(C(=O)c4c(F)c(F)c(F)c(F)c4F)CC3)n2)c1] | A142 |
| C₂₇H₂₈F₅N₅O₂ | |
| 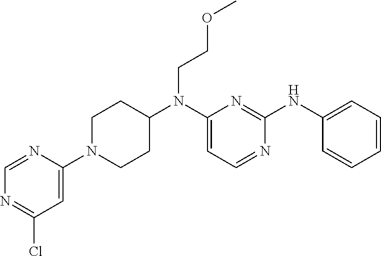 [SMILES: COCCN(c1ccnc(Nc2ccccc2)n1)C1CCN(c2cc(Cl)ncn2)CC1] | A143 |
| C₂₂H₂₆ClN₇O | |
| 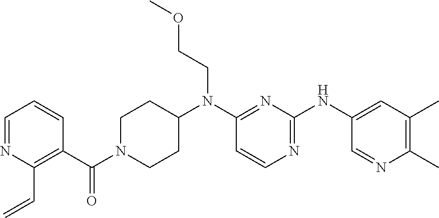 [SMILES: C=Cc1ncccc1C(=O)N1CCC(N(CCOC)c2ccnc(Nc3cnc(C)c(C)c3)n2)CC1] | A144 |
| C₂₇H₃₃N₇O₂ | |
| 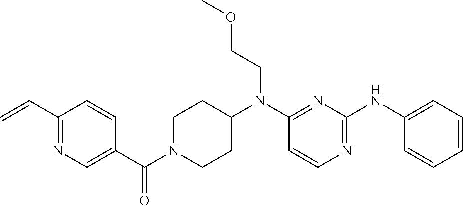 [SMILES: C=Cc1ccc(C(=O)N2CCC(N(CCOC)c3ccnc(Nc4ccccc4)n3)CC2)cn1] | A145 |
| C₂₆H₃₀N₆O₂ | |
| 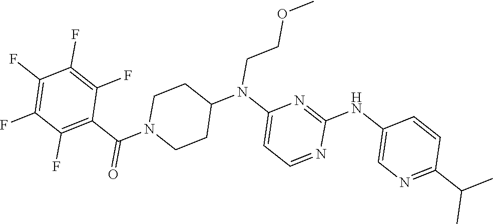 [SMILES: COCCN(c1ccnc(Nc2ccc(C(C)C)nc2)n1)C1CCN(C(=O)c2c(F)c(F)c(F)c(F)c2F)CC1] | A146 |
| C₂₇H₂₉F₅N₆O₂ | |
| 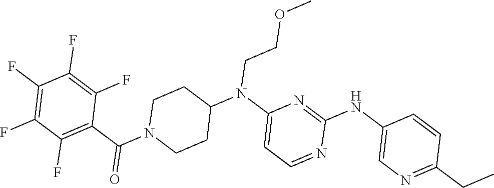 [SMILES: CCc1ccc(Nc2nccc(N(CCOC)C3CCN(C(=O)c4c(F)c(F)c(F)c(F)c4F)CC3)n2)cn1] | A147 |
| C₂₆H₂₇F₅N₆O₂ | |
| 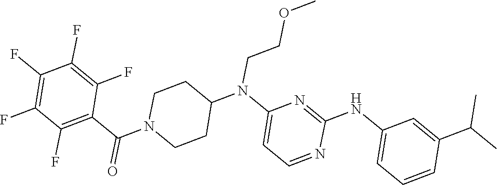 [SMILES: COCCN(c1ccnc(Nc2cccc(C(C)C)c2)n1)C1CCN(C(=O)c2c(F)c(F)c(F)c(F)c2F)CC1] | A148 |
| C₂₈H₃₀F₅N₅O₂ | |
| 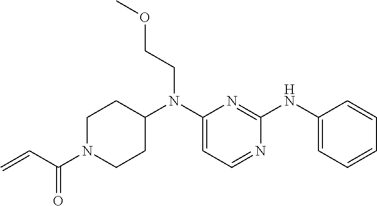 [SMILES: C=CC(=O)N1CCC(N(CCOC)c2ccnc(Nc3ccccc3)n2)CC1] | A149 |
| C₂₁H₂₇N₅O₂ | |
| 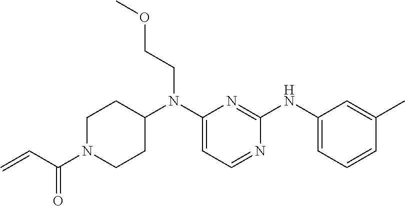 [SMILES: C=CC(=O)N1CCC(N(CCOC)c2ccnc(Nc3cccc(C)c3)n2)CC1] | A150 |
| C₂₂H₂₉N₅O₂ | |
| 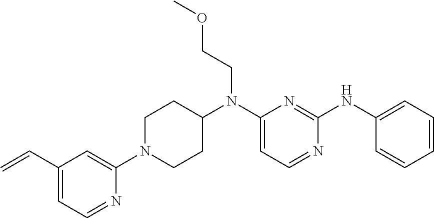 [SMILES: C=Cc1ccnc(N2CCC(N(CCOC)c3ccnc(Nc4ccccc4)n3)CC2)c1] | A151 |
| C₂₅H₃₀N₆O | |
| 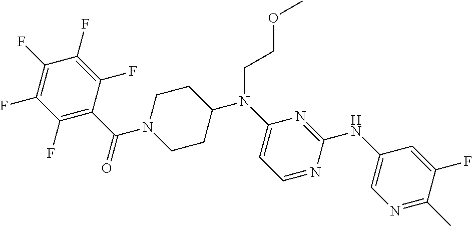 [SMILES: COCCN(c1ccnc(Nc2cnc(C)c(F)c2)n1)C1CCN(C(=O)c2c(F)c(F)c(F)c(F)c2F)CC1] | A152 |
| C₂₅H₂₄F₆N₆O₂ | |
| 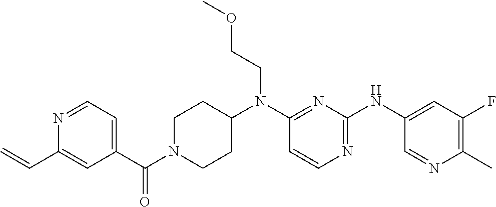 [SMILES: C=Cc1cc(C(=O)N2CCC(N(CCOC)c3ccnc(Nc4cnc(C)c(F)c4)n3)CC2)ccn1] | A153 |
| C₂₆H₃₀FN₇O₂ | |
| 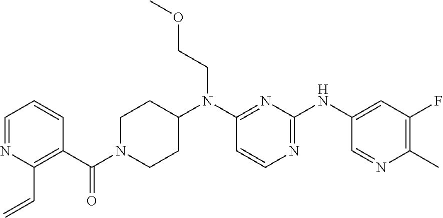 [SMILES: C=Cc1ncccc1C(=O)N1CCC(N(CCOC)c2ccnc(Nc3cnc(C)c(F)c3)n2)CC1] | A154 |
| C₂₆H₃₀FN₇O₂ | |
| 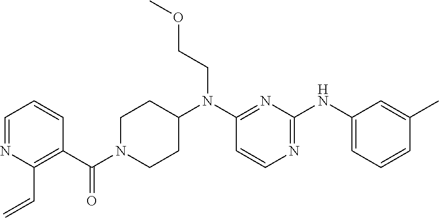 [SMILES: C=Cc1ncccc1C(=O)N1CCC(N(CCOC)c2ccnc(Nc3cccc(C)c3)n2)CC1] | A155 |
| C₂₇H₃₂N₆O₂ | |
| 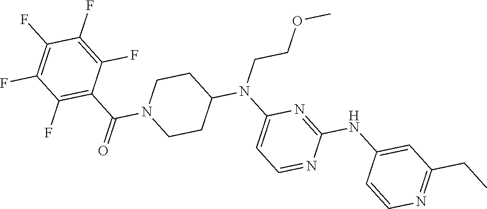 [SMILES: CCc1cc(Nc2nccc(N(CCOC)C3CCN(C(=O)c4c(F)c(F)c(F)c(F)c4F)CC3)n2)ccn1] | A156 |
| C₂₆H₇₂F₅N₆O₂ | |
| 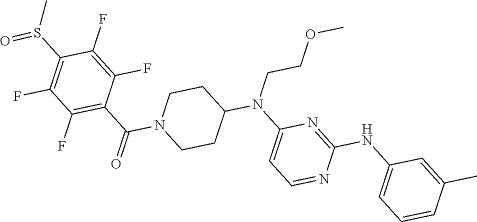 [SMILES: COCCN(c1ccnc(Nc2cccc(C)c2)n1)C1CCN(C(=O)c2c(F)c(F)c(S(C)=O)c(F)c2F)CC1] | A157 |
| C₂₇H₂₉F₄N₅O₃S | |
| 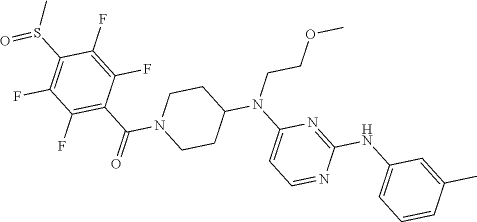 [SMILES: COCCN(c1ccnc(Nc2cccc(C)c2)n1)C1CCN(C(=O)c2c(F)c(F)c(S(C)=O)c(F)c2F)CC1] | A158 |
| C₂₇H₂₉F₄N₅O₃S | |
| 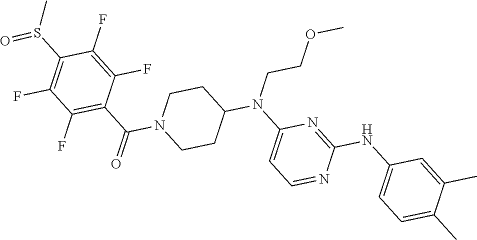 [SMILES: COCCN(c1ccnc(Nc2ccc(C)c(C)c2)n1)C1CCN(C(=O)c2c(F)c(F)c(S(C)=O)c(F)c2F)CC1] | A159 |
| C₂₈H₃₁F₄N₅O₃S | |
| 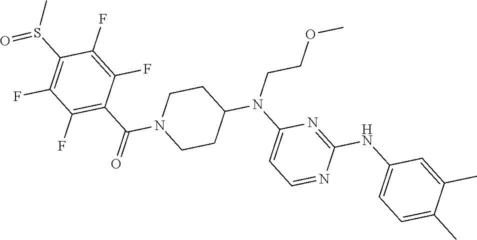 [SMILES: COCCN(c1ccnc(Nc2ccc(C)c(C)c2)n1)C1CCN(C(=O)c2c(F)c(F)c(S(C)=O)c(F)c2F)CC1] | A160 |
| C₂₈H₃₁F₄N₅O₃S | |
| 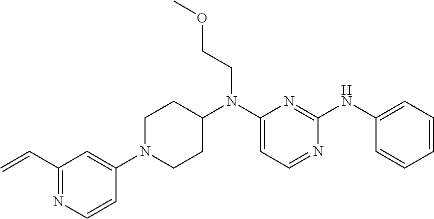 [SMILES: C=Cc1cc(N2CCC(N(CCOC)c3ccnc(Nc4ccccc4)n3)CC2)ccn1] | A161 |
| C₂₅H₃₀N₆O | |
| 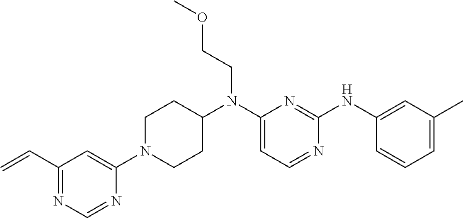 [SMILES: C=Cc1cc(N2CCC(N(CCOC)c3ccnc(Nc4cccc(C)c4)n3)CC2)ncn1] | A162 |
| C₂₅H₃₁N₇O | |
| 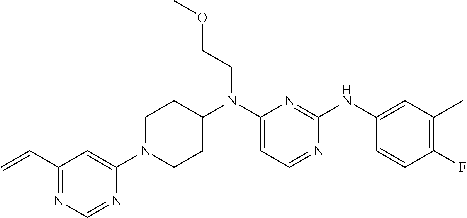 [SMILES: C=Cc1cc(N2CCC(N(CCOC)c3ccnc(Nc4ccc(F)c(C)c4)n3)CC2)ncn1] | A163 |
| C₂₅H₃₀FN₇O | |
| 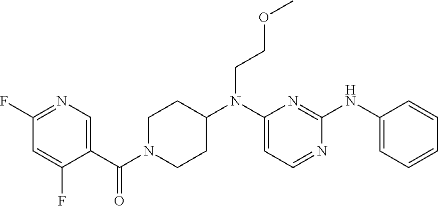 [SMILES: COCCN(c1ccnc(Nc2ccccc2)n1)C1CCN(C(=O)c2cnc(F)cc2F)CC1] | A164 |
| C₂₄H₂₆F₂N₆O₂ | |
| 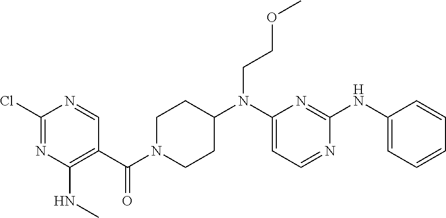 [SMILES: CNc1nc(Cl)ncc1C(=O)N1CCC(N(CCOC)c2ccnc(Nc3ccccc3)n2)CC1] | A165 |
| C₂₄H₂₉ClN₈O₂ | |
| 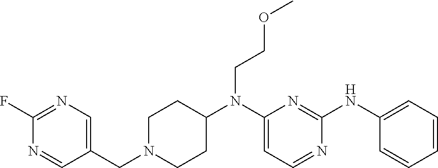 [SMILES: COCCN(c1ccnc(Nc2ccccc2)n1)C1CCN(Cc2cnc(F)nc2)CC1] | A166 |
| C₂₃H₂₈FN₇O | |
| 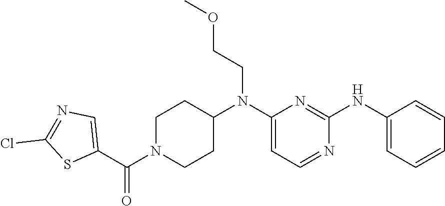 [SMILES: COCCN(c1ccnc(Nc2ccccc2)n1)C1CCN(C(=O)c2cnc(Cl)s2)CC1] | A167 |
| C₂₂H₂₅ClN₆O₂S | |
| 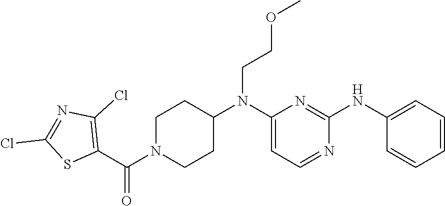 [SMILES: COCCN(c1ccnc(Nc2ccccc2)n1)C1CCN(C(=O)c2sc(Cl)nc2Cl)CC1] | A168 |
| C₂₂H₂₄Cl₂N₆O₂S | |
| 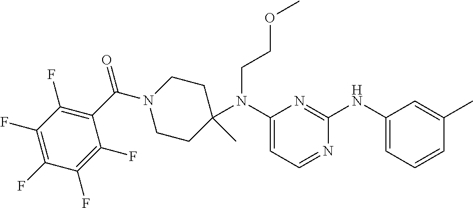 [SMILES: COCCN(c1ccnc(Nc2cccc(C)c2)n1)C1(C)CCN(C(=O)c2c(F)c(F)c(F)c(F)c2F)CC1] | A169 |
| C₂₇H₂₈F₅N₅O₂ | |
| 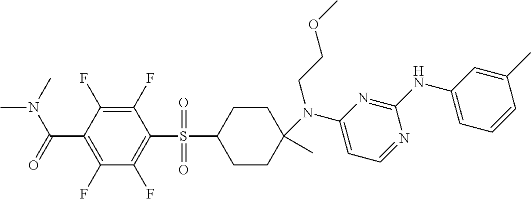 | A170 |
| C₂₉H₃₄F₄N₆O₄S | |
| 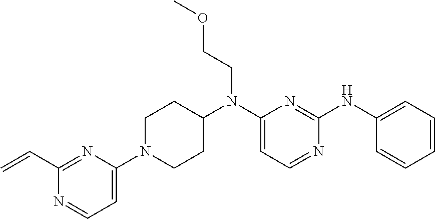 [SMILES: C=Cc1nccc(N2CCC(N(CCOC)c3ccnc(Nc4ccccc4)n3)CC2)n1] | A171 |
| C₂₄H₂₉N₇O | |
| 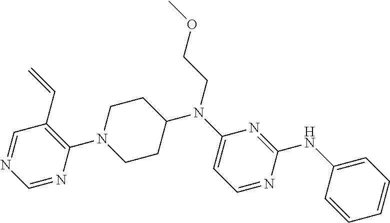 [SMILES: C=Cc1cncnc1N1CCC(N(CCOC)c2ccnc(Nc3ccccc3)n2)CC1] | A172 |
| C₂₄H₂₉N₇O | |
| 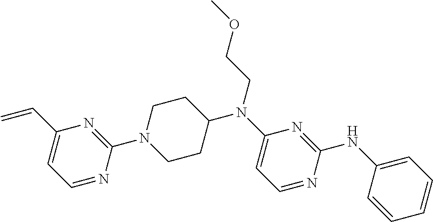 [SMILES: C=Cc1ccnc(N2CCC(N(CCOC)c3ccnc(Nc4ccccc4)n3)CC2)n1] | A173 |
| C₂₄H₂₉N₇O | |
| 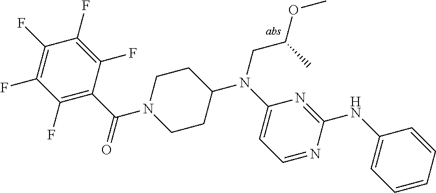 [SMILES: CO[C@H](C)CN(c1ccnc(Nc2ccccc2)n1)C1CCN(C(=O)c2c(F)c(F)c(F)c(F)c2F)CC1] | A174 |
| C₂₆H₂₆F₅N₅O₂ | |
| 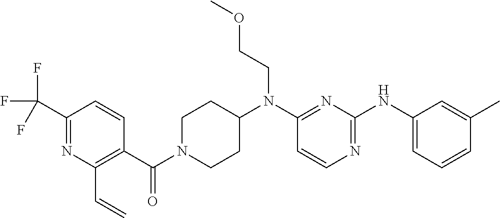 [SMILES: C=Cc1nc(C(F)(F)F)ccc1C(=O)N1CCC(N(CCOC)c2ccnc(Nc3cccc(C)c3)n2)CC1] | A175 |
| C₂₈H₃₁F₃N₆O₂ | |
| 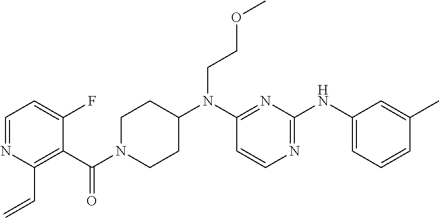 [SMILES: C=Cc1nccc(F)c1C(=O)N1CCC(N(CCOC)c2ccnc(Nc3cccc(C)c3)n2)CC1] | A176 |
| C₂₇H₃₁FN₆O₂ | |
| 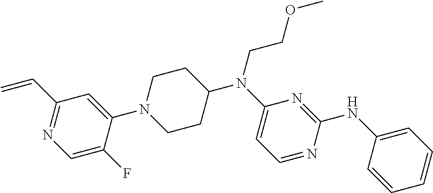 [SMILES: C=Cc1cc(N2CCC(N(CCOC)c3ccnc(Nc4ccccc4)n3)CC2)c(F)cn1] | A177 |
| C₂₅H₂₉FN₆O | |
| 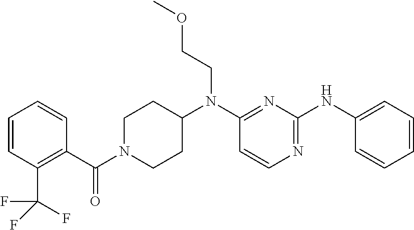 [SMILES: COCCN(c1ccnc(Nc2ccccc2)n1)C1CCN(C(=O)c2ccccc2C(F)(F)F)CC1] | A178 |
| C₂₆H₂₈F₃N₅O₂ | |
| 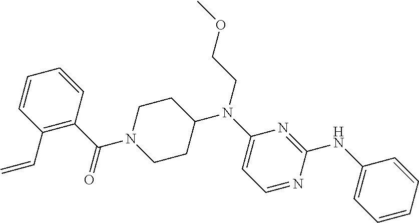 [SMILES: C=Cc1ccccc1C(=O)N1CCC(N(CCOC)c2ccnc(Nc3ccccc3)n2)CC1] | A179 |
| C₂₇H₃₁N₅O₂ | |
| 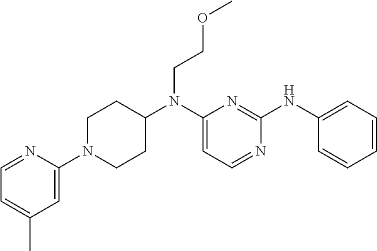 [SMILES: COCCN(c1ccnc(Nc2ccccc2)n1)C1CCN(c2cc(C)ccn2)CC1] | A180 |
| C₂₄H₃₀N₆O | |
| 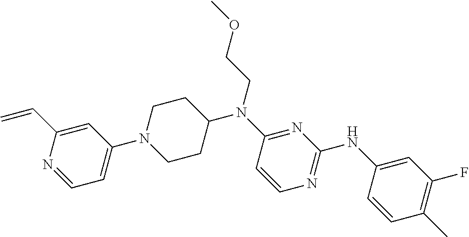 [SMILES: C=Cc1cc(N2CCC(N(CCOC)c3ccnc(Nc4ccc(C)c(F)c4)n3)CC2)ccn1] | A181 |
| C₂₅H₃₀FN₇O | |
| 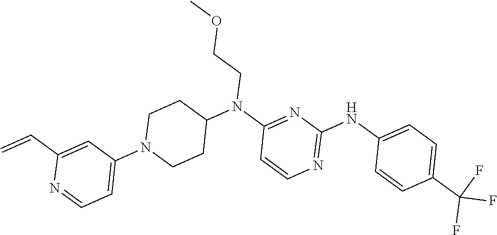 [SMILES: C=Cc1cc(N2CCC(N(CCOC)c3ccnc(Nc4ccc(C(F)(F)F)cc4)n3)CC2)ccn1] | A182 |
| C₂₆H₂₉F₃N₆O | |
| 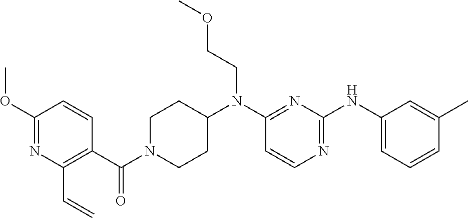 [SMILES: C=Cc1nc(OC)ccc1C(=O)N1CCC(N(CCOC)c2ccnc(Nc3cccc(C)c3)n2)CC1] | A183 |
| C₂₈H₃₄N₆O₃ | |
| 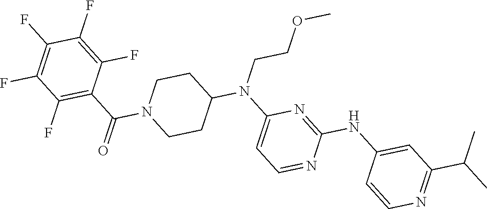 [SMILES: COCCN(c1ccnc(Nc2ccnc(C(C)C)c2)n1)C1CCN(C(=O)c2c(F)c(F)c(F)c(F)c2F)CC1] | A184 |
| C₂₇H₂₉F₅N₆O₂ | |
| 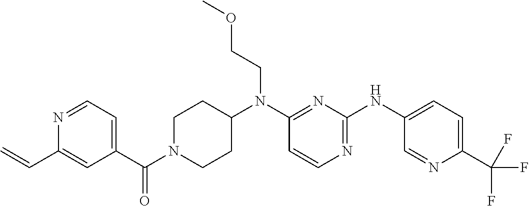 [SMILES: C=Cc1cc(C(=O)N2CCC(N(CCOC)c3ccnc(Nc4ccc(C(F)(F)F)nc4)n3)CC2)ccn1] | A185 |
| C₂₆H₂₈F₃N₇O₂ | |
| 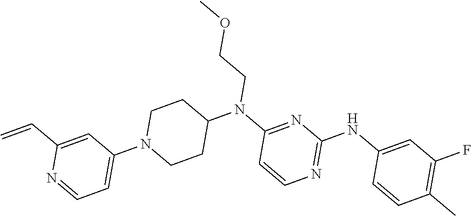 [SMILES: C=Cc1cc(N2CCC(N(CCOC)c3ccnc(Nc4ccc(C)c(F)c4)n3)CC2)ccn1] | A186 |
| C₂₆H₃₁FN₆O | |
| 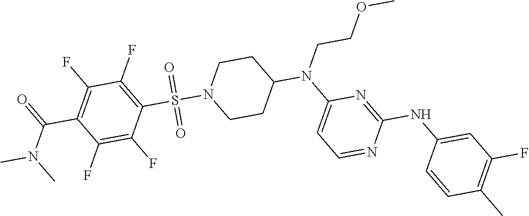 [SMILES: COCCN(c1ccnc(Nc2ccc(C)c(F)c2)n1)C1CCN(S(=O)(=O)c2c(F)c(F)c(C(=O)N(C)C)c(F)c2F)CC1] | A187 |
| C₂₈H₃₁F₅N₆O₄S | |
| 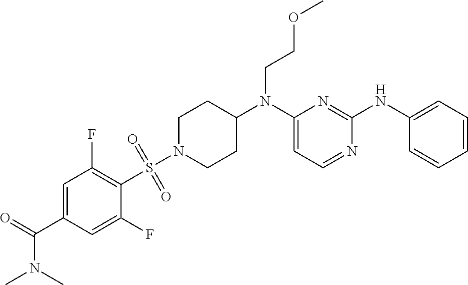 [SMILES: COCCN(c1ccnc(Nc2ccccc2)n1)C1CCN(S(=O)(=O)c2c(F)cc(C(=O)N(C)C)cc2F)CC1] | A188 |
| C₂₇H₃₂F₂N₆O₄S | |
| 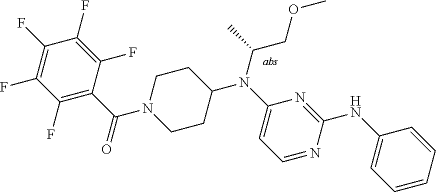 [SMILES: COC[C@@H](C)N(c1ccnc(Nc2ccccc2)n1)C1CCN(C(=O)c2c(F)c(F)c(F)c(F)c2F)CC1] | A189 |
| C₂₆H₂₆F₅N₅O₂ | |
| 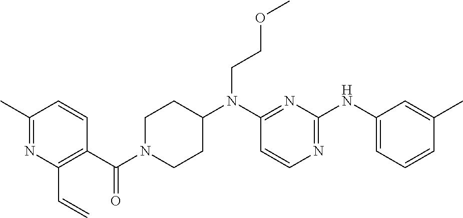 | A190 |
| C₂₈H₃₄N₆O₂ | |
| 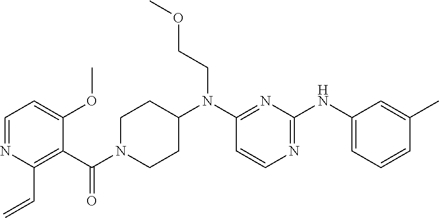 [SMILES: C=Cc1nccc(OC)c1C(=O)N1CCC(N(CCOC)c2ccnc(Nc3cccc(C)c3)n2)CC1] | A191 |
| C₂₈H₃₄N₆O₃ | |
| 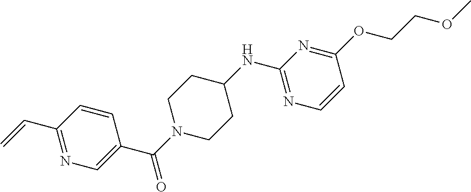 [SMILES: C=Cc1ccc(C(=O)N2CCC(Nc3nccc(OCCOC)n3)CC2)cn1] | A192 |
| C₂₀H₂₅N₅O₃ | |
| 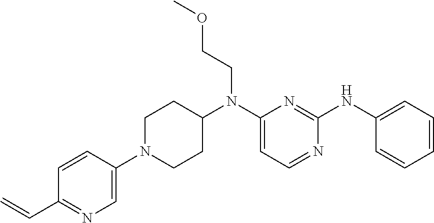 [SMILES: C=Cc1ccc(N2CCC(N(CCOC)c3ccnc(Nc4ccccc4)n3)CC2)cn1] | A193 |
| C₂₅H₃₀N₆O | |
| 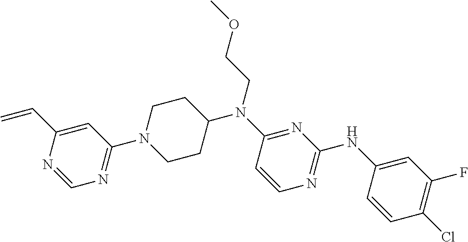 [SMILES: C=Cc1cc(N2CCC(N(CCOC)c3ccnc(Nc4ccc(Cl)c(F)c4)n3)CC2)ncn1] | A194 |
| C₂₄H₂₇ClFN₇O | |
| 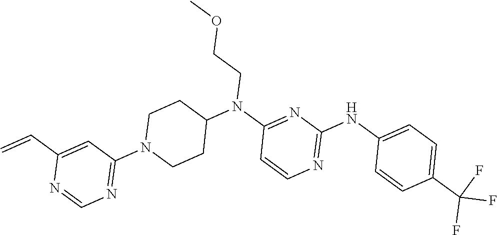 [SMILES: C=Cc1cc(N2CCC(N(CCOC)c3ccnc(Nc4ccc(C(F)(F)F)cc4)n3)CC2)ncn1] | A195 |
| C₂₅H₂₈F₃N₇O | |
| 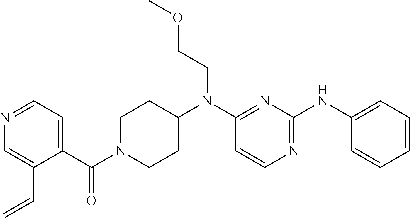 [SMILES: C=Cc1cnccc1C(=O)N1CCC(N(CCOC)c2ccnc(Nc3ccccc3)n2)CC1] | A196 |
| C₂₆H₃₀N₆O₂ | |
| 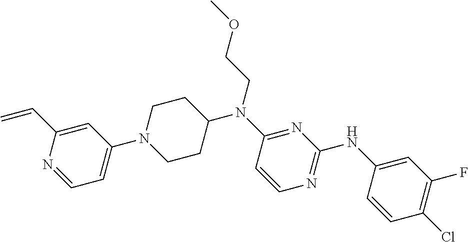 [SMILES: C=Cc1cc(N2CCC(N(CCOC)c3ccnc(Nc4ccc(Cl)c(F)c4)n3)CC2)ccn1] | A197 |
| C₂₅H₂₈ClFN₆O | |
| 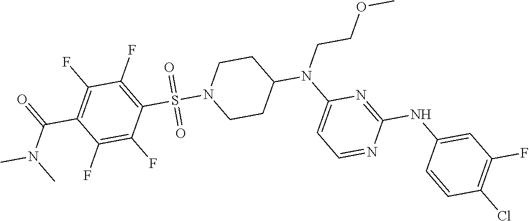 [SMILES: COCCN(c1ccnc(Nc2ccc(Cl)c(F)c2)n1)C1CCN(S(=O)(=O)c2c(F)c(F)c(C(=O)N(C)C)c(F)c2F)CC1] | A198 |
| C₂₇H₂₈ClF₅N₆O₄S | |
| 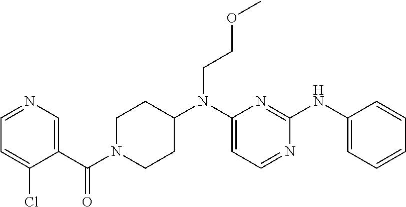 [SMILES: COCCN(c1ccnc(Nc2ccccc2)n1)C1CCN(C(=O)c2cnccc2Cl)CC1] | A199 |
| C₂₄H₂₇ClN₆O₂ | |
| 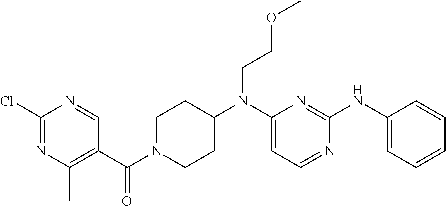 [SMILES: COCCN(c1ccnc(Nc2ccccc2)n1)C1CCN(C(=O)c2cnc(Cl)nc2C)CC1] | A200 |
| C₂₄H₂₈ClN₇O₂ | |
| 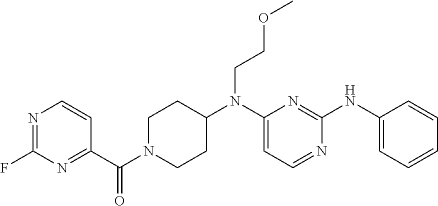 [SMILES: COCCN(c1ccnc(Nc2ccccc2)n1)C1CCN(C(=O)c2ccnc(F)n2)CC1] | A201 |
| C₂₃H₂₆FN₇O₂ | |
| 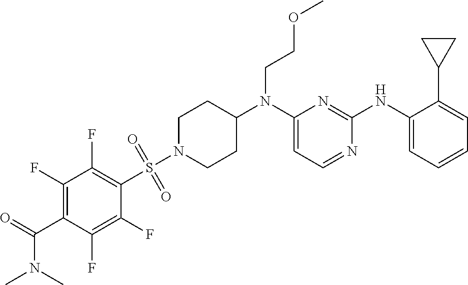 [SMILES: COCCN(c1ccnc(Nc2ccccc2C2CC2)n1)C1CCN(S(=O)(=O)c2c(F)c(F)c(C(=O)N(C)C)c(F)c2F)CC1] | A202 |
| C₃₀H₃₄F₄N₆O₄S | |
| 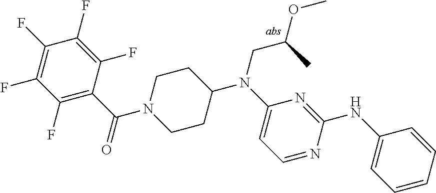 [SMILES: CO[C@@H](C)CN(c1ccnc(Nc2ccccc2)n1)C1CCN(C(=O)c2c(F)c(F)c(F)c(F)c2F)CC1] | A203 |
| C₂₆H₂₆F₅N₅O₂ | |
| 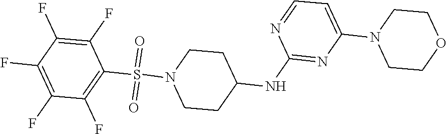 [SMILES: O=S(=O)(c1c(F)c(F)c(F)c(F)c1F)N1CCC(Nc2nccc(N3CCOCC3)n2)CC1] | A204 |
| C₁₉H₂₀F₅N₅O₃S | |
| 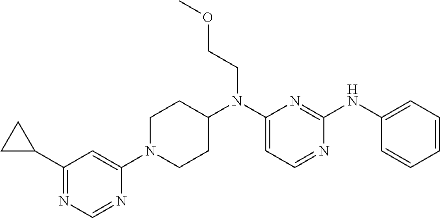 [SMILES: COCCN(c1ccnc(Nc2ccccc2)n1)C1CCN(c2cc(C3CC3)ncn2)CC1] | A205 |
| C₂₅H₃₁N₇O | |
| 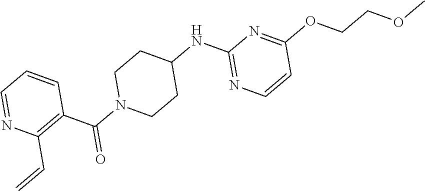 [SMILES: C=Cc1ncccc1C(=O)N1CCC(Nc2nccc(OCCOC)n2)CC1] | A206 |
| C₂₀H₂₅N₅O₃ | |
| 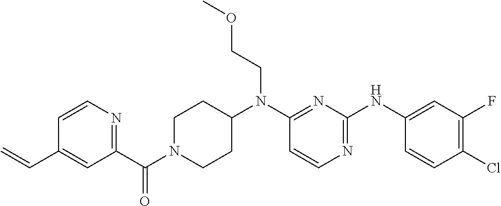 [SMILES: C=Cc1ccnc(C(=O)N2CCC(N(CCOC)c3ccnc(Nc4ccc(Cl)c(F)c4)n3)CC2)c1] | A207 |
| C₂₆H₂₈ClFN₆O₂ | |
| 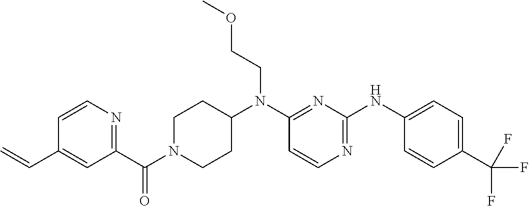 [SMILES: C=Cc1ccnc(C(=O)N2CCC(N(CCOC)c3ccnc(Nc4ccc(C(F)(F)F)cc4)n3)CC2)c1] | A208 |
| C₂₇H₂₉F₃N₆O₂ | |
| 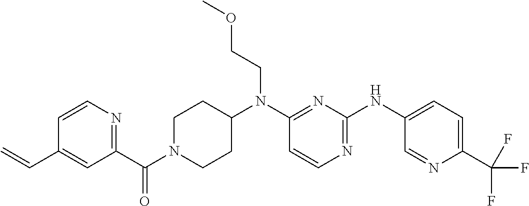 [SMILES: C=Cc1ccnc(C(=O)N2CCC(N(CCOC)c3ccnc(Nc4ccc(C(F)(F)F)nc4)n3)CC2)c1] | A209 |
| C₂₆H₂₈F₃N₇O₂ | |
| 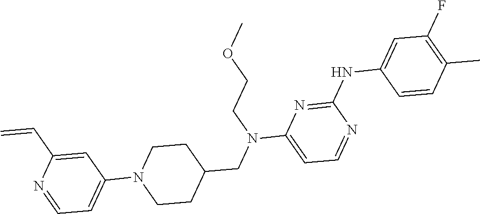 [SMILES: C=Cc1cc(N2CCC(CN(CCOC)c3ccnc(Nc4ccc(C)c(F)c4)n3)CC2)ccn1] | A210 |
| C₂₇H₃₃FN₆O | |
| 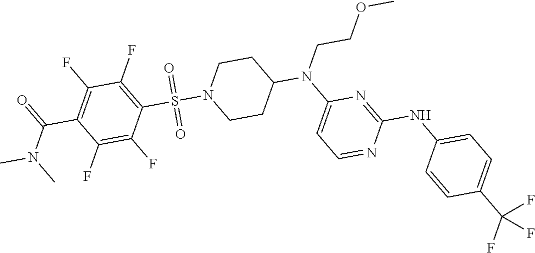 [SMILES: COCCN(c1ccnc(Nc2ccc(C(F)(F)F)cc2)n1)C1CCN(S(=O)(=O)c2c(F)c(F)c(C(=O)N(C)C)c(F)c2F)CC1] | A211 |
| C₂₈H₂₉F₇N₆O₄S | |
| 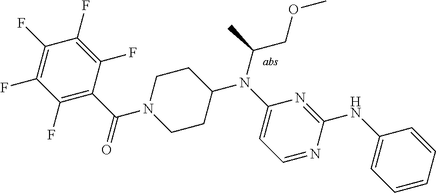 [SMILES: COC[C@H](C)N(c1ccnc(Nc2ccccc2)n1)C1CCN(C(=O)c2c(F)c(F)c(F)c(F)c2F)CC1] | A212 |
| C₂₆H₂₆F₅N₅O₂ | |
| 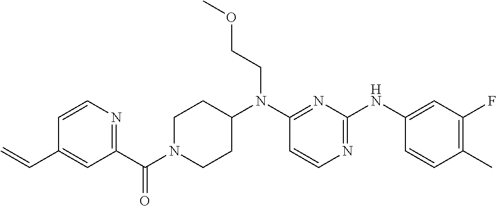 [SMILES: C=Cc1ccnc(C(=O)N2CCC(N(CCOC)c3ccnc(Nc4ccc(C)c(F)c4)n3)CC2)c1] | A213 |
| C₂₇H₃₁FN₆O₂ | |
| 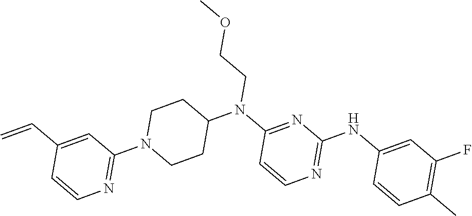 [SMILES: C=Cc1ccnc(N2CCC(N(CCOC)c3ccnc(Nc4ccc(C)c(F)c4)n3)CC2)c1] | A214 |
| C₂₆H₃₁FN₆O | |
| 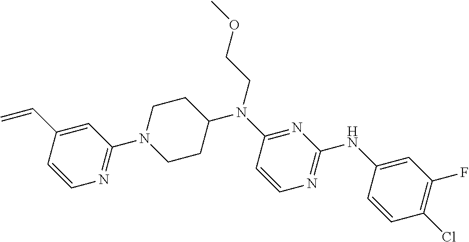 [SMILES: C=Cc1ccnc(N2CCC(N(CCOC)c3ccnc(Nc4ccc(Cl)c(F)c4)n3)CC2)c1] | A215 |
| C₂₅H₂₈ClFN₆O | |
| 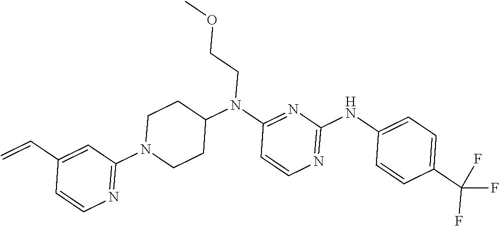 [SMILES: C=Cc1ccnc(N2CCC(N(CCOC)c3ccnc(Nc4ccc(C(F)(F)F)cc4)n3)CC2)c1] | A216 |
| C₂₆H₂₉F₃N₆O | |
| 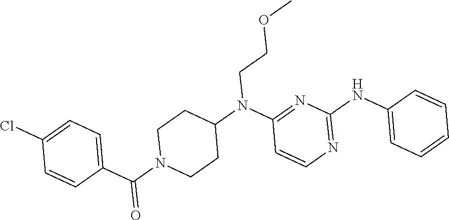 [SMILES: COCCN(c1ccnc(Nc2ccccc2)n1)C1CCN(C(=O)c2ccc(Cl)cc2)CC1] | A217 |
| C₂₅H₂₈ClN₅O₂ | |
| 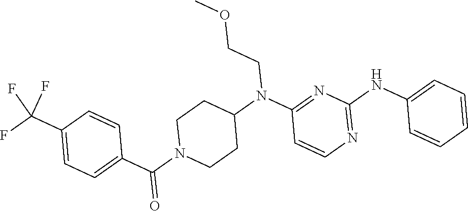 [SMILES: COCCN(c1ccnc(Nc2ccccc2)n1)C1CCN(C(=O)c2ccc(C(F)(F)F)cc2)CC1] | A218 |
| C₂₆H₂₈F₃N₅O₂ | |
| 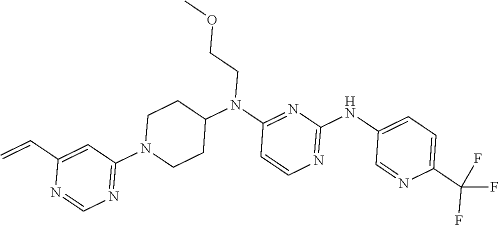 [SMILES: C=Cc1cc(N2CCC(N(CCOC)c3ccnc(Nc4ccc(C(F)(F)F)nc4)n3)CC2)ncn1] | A219 |
| C₂₄H₂₇F₃N₈O | |
| 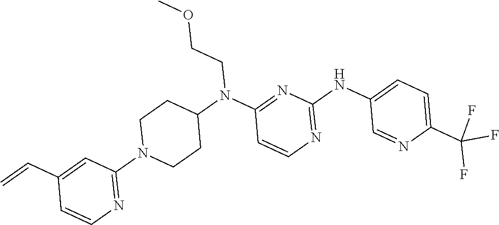 [SMILES: C=Cc1ccnc(N2CCC(N(CCOC)c3ccnc(Nc4ccc(C(F)(F)F)nc4)n3)CC2)c1] | A220 |
| C₂₅H₂₈F₃N₇O | |
| 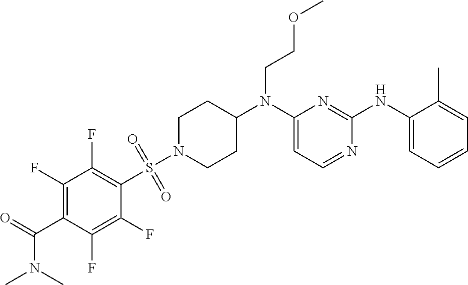 [SMILES: COCCN(c1ccnc(Nc2ccccc2C)n1)C1CCN(S(=O)(=O)c2c(F)c(F)c(C(=O)N(C)C)c(F)c2F)CC1] | A221 |
| C₂₈H₃₂F₄N₆O₄S | |
| 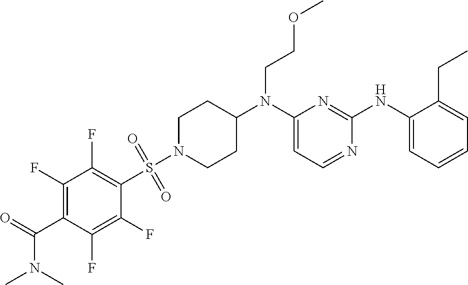 [SMILES: CCc1ccccc1Nc1nccc(N(CCOC)C2CCN(S(=O)(=O)c3c(F)c(F)c(C(=O)N(C)C)c(F)c3F)CC2)n1] | A222 |
| C₂₉H₃₄F₄N₆O₄S | |
| 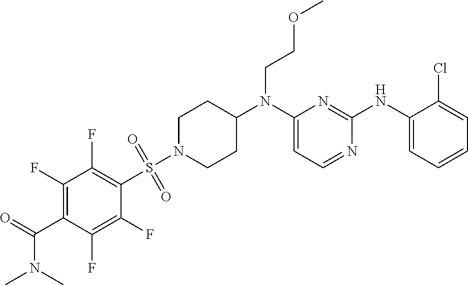 [SMILES: COCCN(c1ccnc(Nc2ccccc2Cl)n1)C1CCN(S(=O)(=O)c2c(F)c(F)c(C(=O)N(C)C)c(F)c2F)CC1] | A223 |
| C₂₇H₂₉ClF₄N₆O₄S | |
| 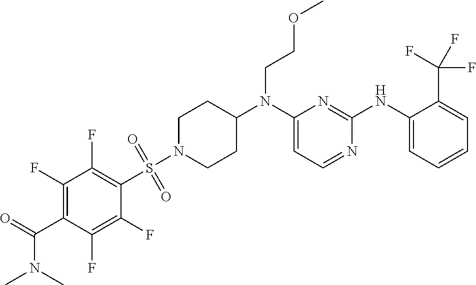 [SMILES: COCCN(c1ccnc(Nc2ccccc2C(F)(F)F)n1)C1CCN(S(=O)(=O)c2c(F)c(F)c(C(=O)N(C)C)c(F)c2F)CC1] | A224 |
| C₂₈H₂₉F₇N₆O₄S | |
| 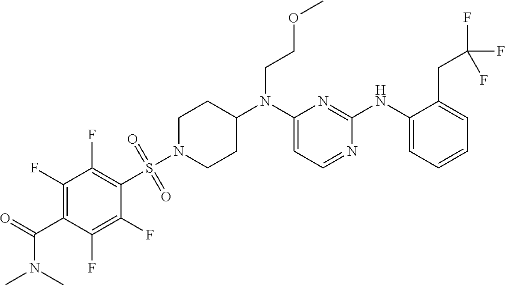 [SMILES: COCCN(c1ccnc(Nc2ccccc2CC(F)(F)F)n1)C1CCN(S(=O)(=O)c2c(F)c(F)c(C(=O)N(C)C)c(F)c2F)CC1] | A225 |
| C₂₉H₃₁F₇N₆O₄S | |
| 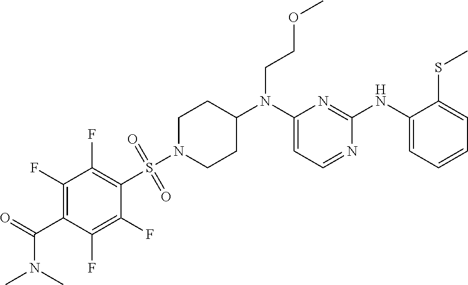 [SMILES: COCCN(c1ccnc(Nc2ccccc2SC)n1)C1CCN(S(=O)(=O)c2c(F)c(F)c(C(=O)N(C)C)c(F)c2F)CC1] | A226 |
| C₂₈H₃₂F₄N₆O₄S₂ | |
| 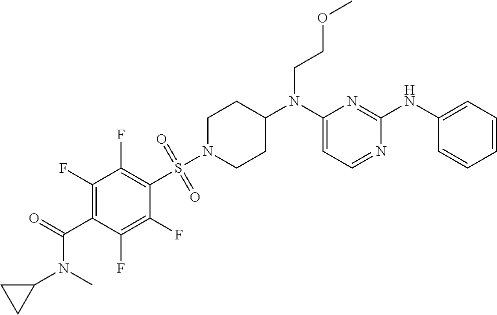 [SMILES: COCCN(c1ccnc(Nc2ccccc2)n1)C1CCN(S(=O)(=O)c2c(F)c(F)c(C(=O)N(C)C3CC3)c(F)c2F)CC1] | A227 |
| C₂₉H₃₂F₄N₆O₄S | |
| 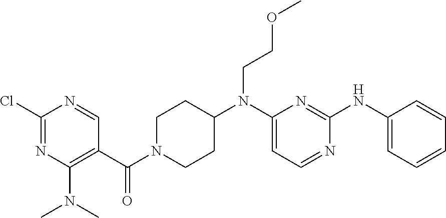 [SMILES: COCCN(c1ccnc(Nc2ccccc2)n1)C1CCN(C(=O)c2cnc(Cl)nc2N(C)C)CC1] | A228 |
| C₂₅H₃₁ClN₈O₂ | |
| 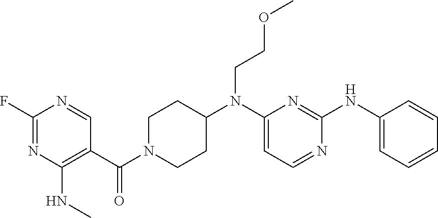 [SMILES: CNc1nc(F)ncc1C(=O)N1CCC(N(CCOC)c2ccnc(Nc3ccccc3)n2)CC1] | A229 |
| C₂₄H₂₉FN₈O₂ | |
| 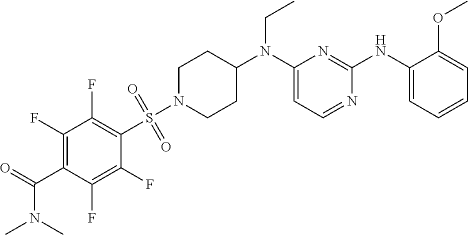 [SMILES: CCN(c1ccnc(Nc2ccccc2OC)n1)C1CCN(S(=O)(=O)c2c(F)c(F)c(C(=O)N(C)C)c(F)c2F)CC1] | A230 |
| C₂₇H₃₀F₄N₆O₄S | |
| 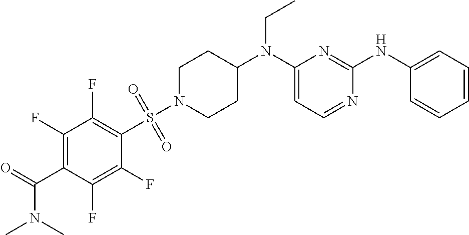 [SMILES: CCN(c1ccnc(Nc2ccccc2)n1)C1CCN(S(=O)(=O)c2c(F)c(F)c(C(=O)N(C)C)c(F)c2F)CC1] | A231 |
| C₂₆H₂₈F₄N₆O₃S | |
| 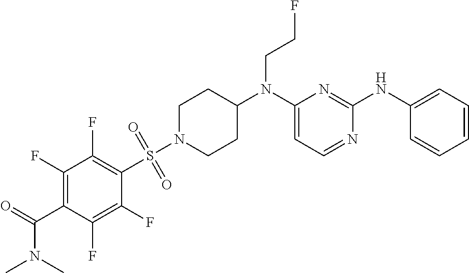 [SMILES: CN(C)C(=O)c1c(F)c(F)c(S(=O)(=O)N2CCC(N(CCF)c3ccnc(Nc4ccccc4)n3)CC2)c(F)c1F] | A232 |
| C₂₆H₂₇F₅N₆O₃S | |
| 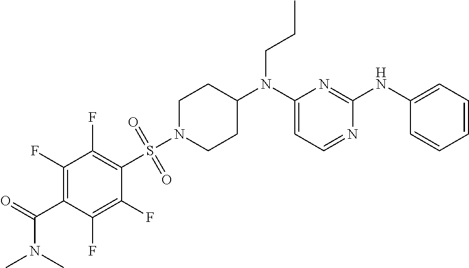 [SMILES: CCCN(c1ccnc(Nc2ccccc2)n1)C1CCN(S(=O)(=O)c2c(F)c(F)c(C(=O)N(C)C)c(F)c2F)CC1] | A233 |
| C₂₇H₃₀F₄N₆O₃S | |
| 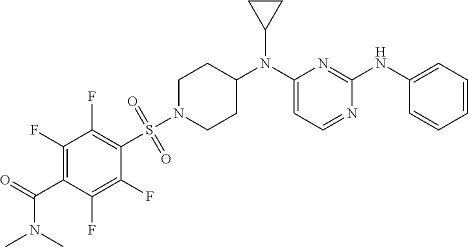 [SMILES: CN(C)C(=O)c1c(F)c(F)c(S(=O)(=O)N2CCC(N(c3ccnc(Nc4ccccc4)n3)C3CC3)CC2)c(F)c1F] | A234 |
| C₂₇H₂₈F₄N₆O₃S | |
| 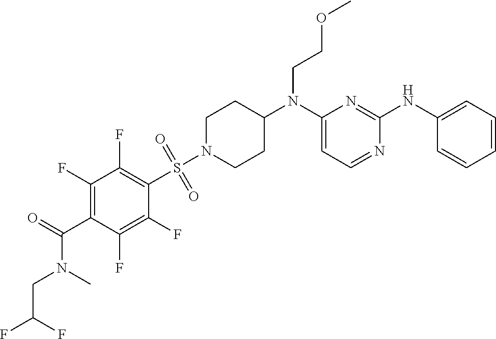 [SMILES: COCCN(c1ccnc(Nc2ccccc2)n1)C1CCN(S(=O)(=O)c2c(F)c(F)c(C(=O)N(C)CC(F)F)c(F)c2F)CC1] | A235 |
| C₂₈H₃₀F₆N₆O₄S | |
| 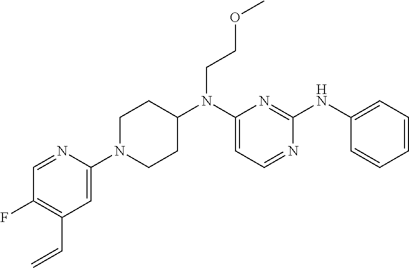 [SMILES: C=Cc1cc(N2CCC(N(CCOC)c3ccnc(Nc4ccccc4)n3)CC2)ncc1F] | A236 |
| C₂₅H₂₉FN₆O | |
| 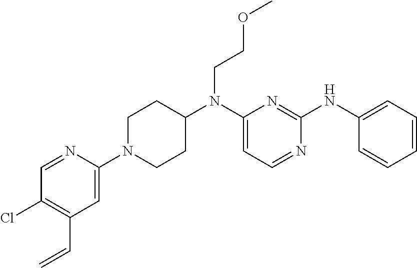 [SMILES: C=Cc1cc(N2CCC(N(CCOC)c3ccnc(Nc4ccccc4)n3)CC2)ncc1Cl] | A237 |
| C₂₅H₂₉ClN₆O | |
| 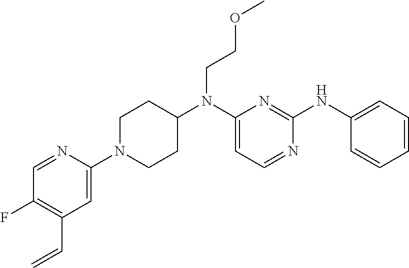 [SMILES: C=Cc1cc(N2CCC(N(CCOC)c3ccnc(Nc4ccccc4)n3)CC2)ncc1F] | A238 |
| C₂₆H₃₂N₆O | |
| 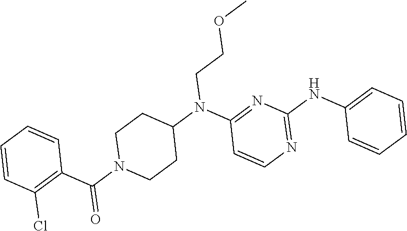 [SMILES: COCCN(c1ccnc(Nc2ccccc2)n1)C1CCN(C(=O)c2ccccc2Cl)CC1] | A239 |
| C₂₅H₂₈ClN₅O₂ | |
| 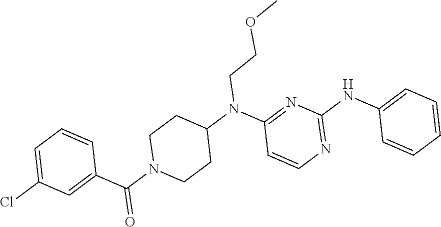 [SMILES: COCCN(c1ccnc(Nc2ccccc2)n1)C1CCN(C(=O)c2cccc(Cl)c2)CC1] | A240 |
| C₂₅H₂₈ClN₅O₂ | |
| 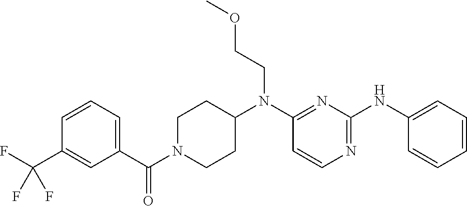 [SMILES: COCCN(c1ccnc(Nc2ccccc2)n1)C1CCN(C(=O)c2cccc(C(F)(F)F)c2)CC1] | A241 |
| C₂₆H₂₈F₃N₅O₂ | |
| 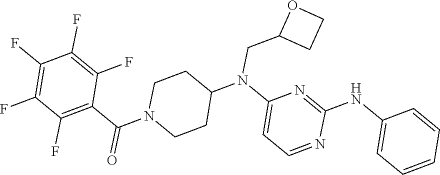 [SMILES: O=C(c1c(F)c(F)c(F)c(F)c1F)N1CCC(N(CC2CCO2)c2ccnc(Nc3ccccc3)n2)CC1] | A242 |
| C₂₆H₂₄F₅N₅O₂ | |
| 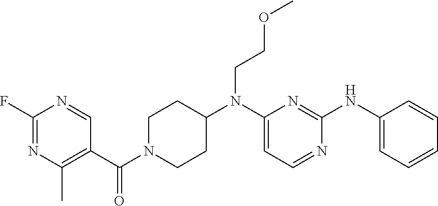 [SMILES: COCCN(c1ccnc(Nc2ccccc2)n1)C1CCN(C(=O)c2cnc(F)nc2C)CC1] | A243 |
| C₂₄H₂₈FN₇O₂ | |
| 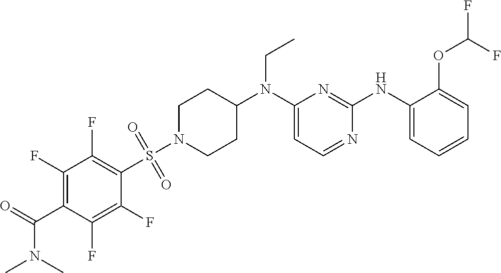 [SMILES: CCN(c1ccnc(Nc2ccccc2OC(F)F)n1)C1CCN(S(=O)(=O)c2c(F)c(F)c(C(=O)N(C)C)c(F)c2F)CC1] | A244 |
| C₂₇H₂₈F₆N₆O₄S | |
| 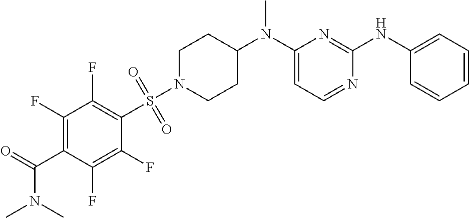 [SMILES: CN(C)C(=O)c1c(F)c(F)c(S(=O)(=O)N2CCC(N(C)c3ccnc(Nc4ccccc4)n3)CC2)c(F)c1F] | A245 |
| C₂₅H₂₆F₄N₆O₃S | |
| 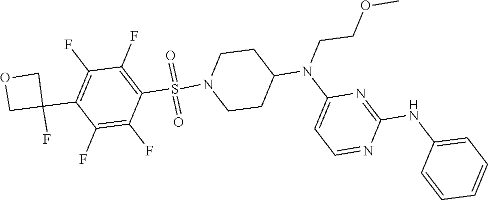 [SMILES: COCCN(c1ccnc(Nc2ccccc2)n1)C1CCN(S(=O)(=O)c2c(F)c(F)c(C3(F)COC3)c(F)c2F)CC1] | A246 |
| C₂₇H₂₈F₅N₅O₄S | |
| 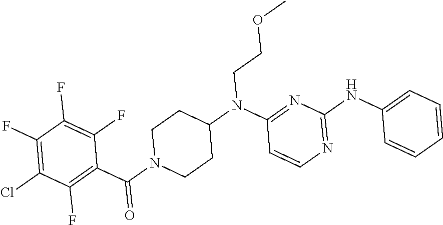 [SMILES: COCCN(c1ccnc(Nc2ccccc2)n1)C1CCN(C(=O)c2c(F)c(F)c(F)c(Cl)c2F)CC1] | A247 |
| C₂₅H₂₄ClF₄N₅O₂ | |
| 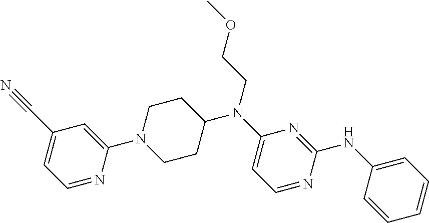 [SMILES: COCCN(c1ccnc(Nc2ccccc2)n1)C1CCN(c2cc(C#N)ccn2)CC1] | A248 |
| C₂₅H₂₈N₆O | |
| 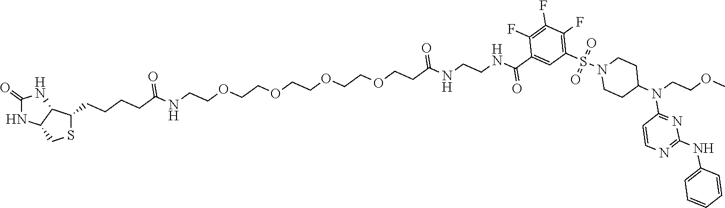 [SMILES: COCCN(c1ccnc(Nc2ccccc2)n1)C1CCN(S(=O)(=O)c2cc(C(=O)NCCNC(=O)CCOCCOCCOCCOCCNC(=O)CCCC[C@@H]3SC[C@@H]4NC(=O)N[C@@H]43)c(F)c(F)c2F)CC1] | A249 |
| C₄₈H₆₇F₃N₁₀O₁₁S₂ | |
| 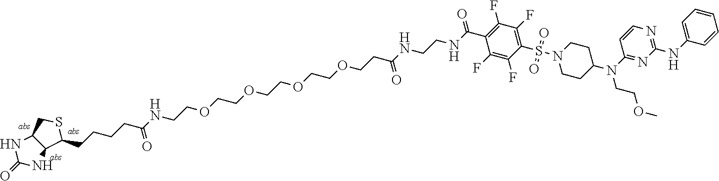 [SMILES: COCCN(c1ccnc(Nc2ccccc2)n1)C1CCN(S(=O)(=O)c2c(F)c(F)c(C(=O)NCCNC(=O)CCOCCOCCOCCOCCNC(=O)CCCC[C@@H]3SC[C@@H]4NC(=O)N[C@@H]43)c(F)c2F)CC1] | A250 |
| C₄₈H₆₆F₄N₁₀O₁₁S₂ | |
| 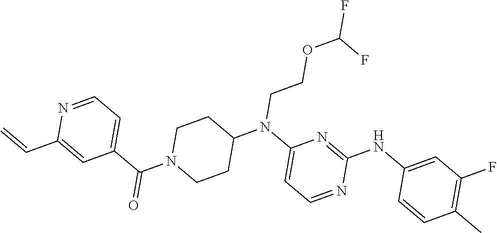 [SMILES: C=Cc1cc(C(=O)N2CCC(N(CCOC(F)F)c3ccnc(Nc4ccc(C)c(F)c4)n3)CC2)ccn1] | A251 |
| C₂₇H₂₉F₃N₆O₂ | |
| 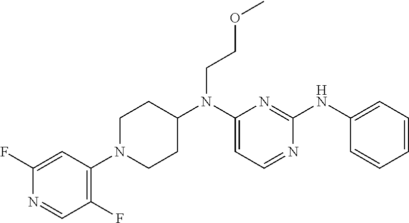 [SMILES: COCCN(c1ccnc(Nc2ccccc2)n1)C1CCN(c2cc(F)ncc2F)CC1] | A252 |
| C₂₃H₂₆F₂N₆O | |
| 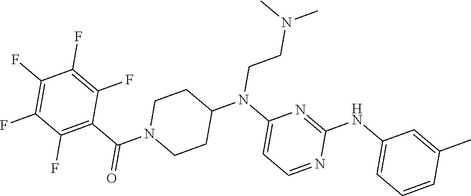 [SMILES: Cc1cccc(Nc2nccc(N(CCN(C)C)C3CCN(C(=O)c4c(F)c(F)c(F)c(F)c4F)CC3)n2)c1] | A253 |
| C₂₇H₂₉F₅N₆O | |
| 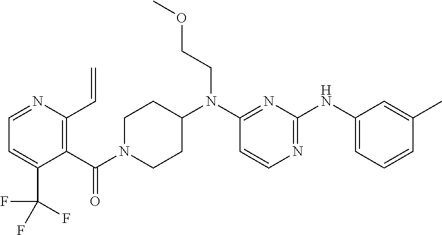 [SMILES: C=Cc1nccc(C(F)(F)F)c1C(=O)N1CCC(N(CCOC)c2ccnc(Nc3cccc(C)c3)n2)CC1] | A254 |
| C₂₈H₃₁F₃N₆O₂ | |
| 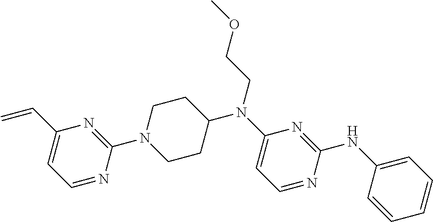 [SMILES: C=Cc1ccnc(N2CCC(N(CCOC)c3ccnc(Nc4ccccc4)n3)CC2)n1] | A255 |
| C₂₅H₃₀N₆O | |
| 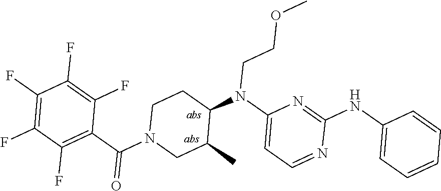 [SMILES: COCCN(c1ccnc(Nc2ccccc2)n1)[C@@H]1CCN(C(=O)c2c(F)c(F)c(F)c(F)c2F)C[C@@H]1C] | A256 |
| C₂₆H₂₆F₅N₅O₂ | |
| 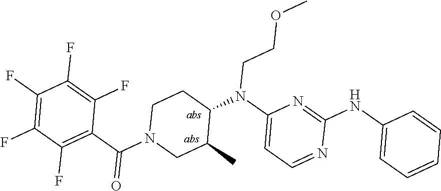 [SMILES: COCCN(c1ccnc(Nc2ccccc2)n1)[C@H]1CCN(C(=O)c2c(F)c(F)c(F)c(F)c2F)C[C@@H]1C] | A257 |
| C₂₆H₂₆F₅N₅O₂ | |
| 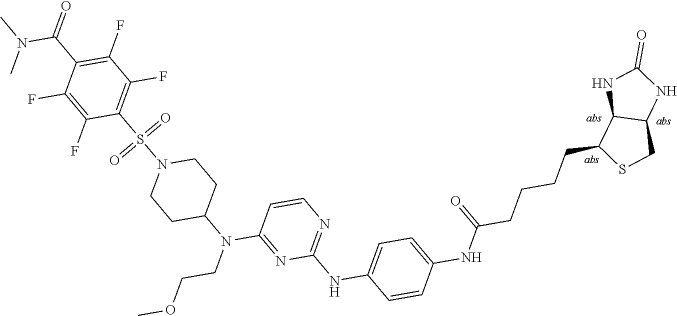 [SMILES: COCCN(c1ccnc(Nc2ccc(NC(=O)CCCC[C@@H]3SC[C@@H]4NC(=O)N[C@@H]43)cc2)n1)C1CCN(S(=O)(=O)c2c(F)c(F)c(C(=O)N(C)C)c(F)c2F)CC1] | A258 |
| C₃₇H₄₅F₄N₉O₆S₂ | |
| 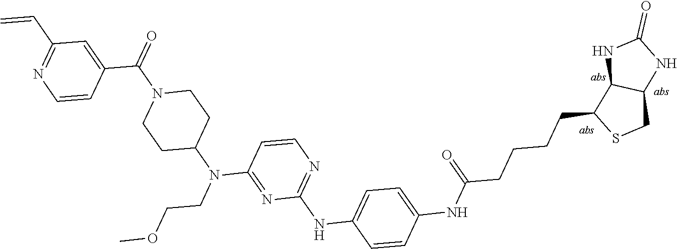 [SMILES: C=Cc1cc(C(=O)N2CCC(N(CCOC)c3ccnc(Nc4ccc(NC(=O)CCCC[C@@H]5SC[C@@H]6NC(=O)N[C@@H]65)cc4)n3)CC2)ccn1] | A259 |
| C₃₆H₄₅N₉O₄S | |
| 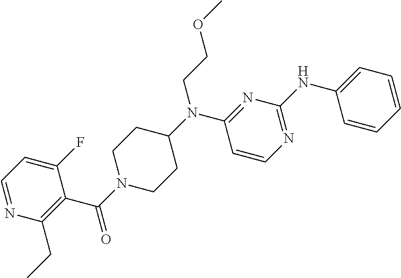 [SMILES: CCc1nccc(F)c1C(=O)N1CCC(N(CCOC)c2ccnc(Nc3ccccc3)n2)CC1] | A260 |
| C₂₆H₃₁FN₆O₂ | |
| 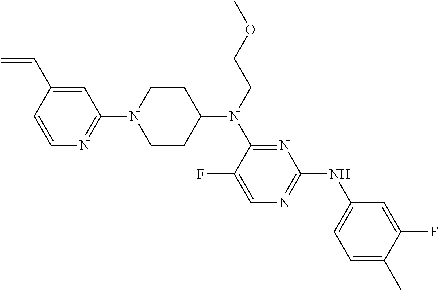 [SMILES: C=Cc1ccnc(N2CCC(N(CCOC)c3nc(Nc4ccc(C)c(F)c4)ncc3F)CC2)c1] | A261 |
| C₂₆H₃₀F₂N₆O | |
| 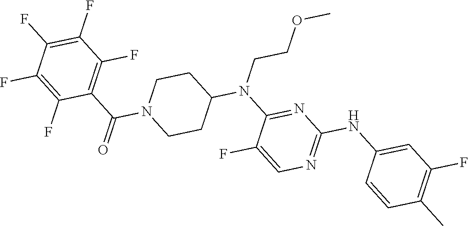 [SMILES: COCCN(c1nc(Nc2ccc(C)c(F)c2)ncc1F)C1CCN(C(=O)c2c(F)c(F)c(F)c(F)c2F)CC1] | A262 |
| C₂₆H₂₄F₇N₅O₂ | |
| 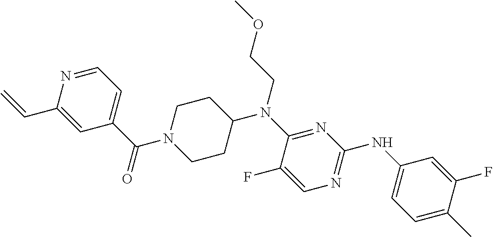 [SMILES: C=Cc1cc(C(=O)N2CCC(N(CCOC)c3nc(Nc4ccc(C)c(F)c4)ncc3F)CC2)ccn1] | A263 |
| C₂₇H₃₀F₂N₆O2 | |
| 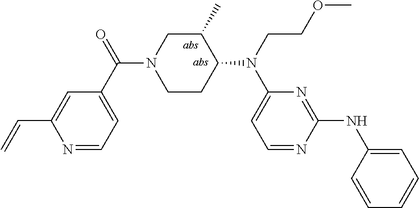 [SMILES: C=Cc1cc(C(=O)N2CC[C@@H](N(CCOC)c3ccnc(Nc4ccccc4)n3)[C@@H](C)C2)ccn1] | A264 |
| C₂₇H₃₂N₆O₂ | |
| 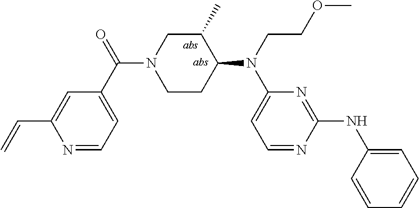 [SMILES: C=Cc1cc(C(=O)N2CC[C@H](N(CCOC)c3ccnc(Nc4ccccc4)n3)[C@@H](C)C2)ccn1] | A265 |
| C₂₇H₃₂N₆O₂ | |
| 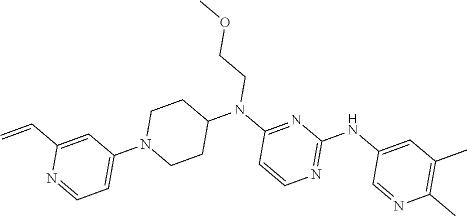 [SMILES: C=Cc1cc(N2CCC(N(CCOC)c3ccnc(Nc4cnc(C)c(C)c4)n3)CC2)ccn1] | A266 |
| C₂₆H₃₃N₇O | |
| 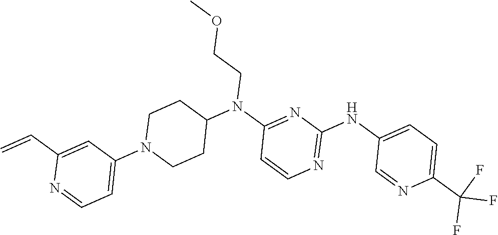 [SMILES: C=Cc1cc(N2CCC(N(CCOC)c3ccnc(Nc4ccc(C(F)(F)F)nc4)n3)CC2)ccn1] | A267 |
| C₂₅H₂₈F₃N₇O | |
| 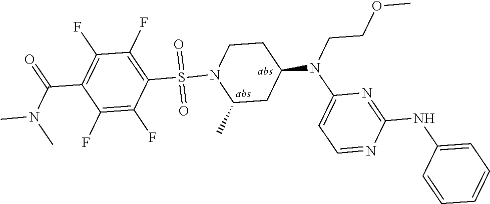 [SMILES: COCCN(c1ccnc(Nc2ccccc2)n1)[C@@H]1CCN(S(=O)(=O)c2c(F)c(F)c(C(=O)N(C)C)c(F)c2F)[C@@H](C)C1] | A268 |
| C₂₈H₃₂F₄N₆O₄S | |
| 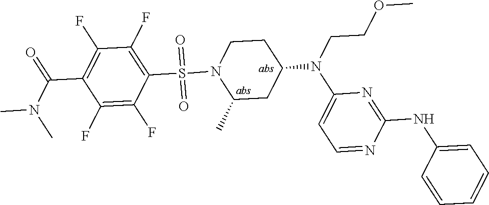 [SMILES: COCCN(c1ccnc(Nc2ccccc2)n1)[C@H]1CCN(S(=O)(=O)c2c(F)c(F)c(C(=O)N(C)C)c(F)c2F)[C@@H](C)C1] | A269 |
| C₂₈H₃₂F₄N₆O₄S | |
| 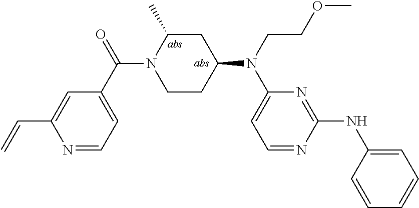 [SMILES: C=Cc1cc(C(=O)N2CC[C@H](N(CCOC)c3ccnc(Nc4ccccc4)n3)C[C@H]2C)ccn1] | A270 |
| C₂₇H₃₂N₆O₂ | |
| 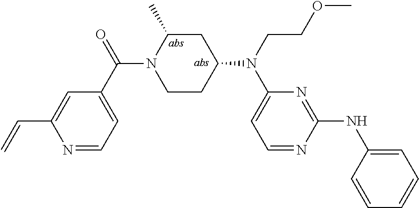 [SMILES: C=Cc1cc(C(=O)N2CC[C@@H](N(CCOC)c3ccnc(Nc4ccccc4)n3)C[C@H]2C)ccn1] | A271 |
| C₂₇H₃₂N₆O₂ | |
| 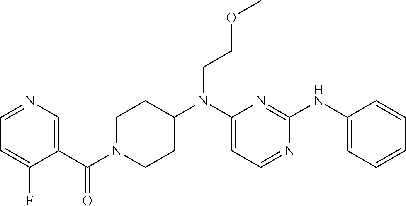 [SMILES: COCCN(c1ccnc(Nc2ccccc2)n1)C1CCN(C(=O)c2cnccc2F)CC1] | A272 |
| C₂₄H₂₇FN₆O₂ | |
| 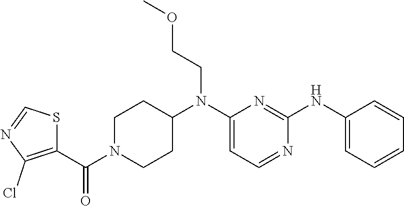 [SMILES: COCCN(c1ccnc(Nc2ccccc2)n1)C1CCN(C(=O)c2scnc2Cl)CC1] | A273 |
| C₂₂H₂₅ClN₆O₂S | |
| 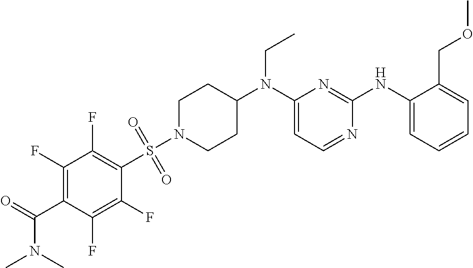 [SMILES: CCN(c1ccnc(Nc2ccccc2COC)n1)C1CCN(S(=O)(=O)c2c(F)c(F)c(C(=O)N(C)C)c(F)c2F)CC1] | A274 |
| C₂₈H₃₂F₄N₆O₄S | |
| 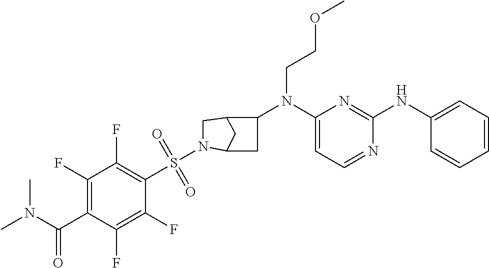 [SMILES: COCCN(c1ccnc(Nc2ccccc2)n1)C1CC2CC1CN2S(=O)(=O)c1c(F)c(F)c(C(=O)N(C)C)c(F)c1F] | A275 |
| C₂₈H₃₀F₄N₆O₄S | |
| 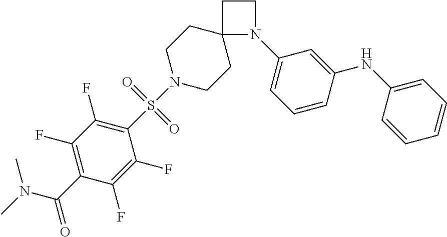 [SMILES: CN(C)C(=O)c1c(F)c(F)c(S(=O)(=O)N2CCC3(CCN3c3cccc(Nc4ccccc4)c3)CC2)c(F)c1F] | A276 |
| C₂₆H₂₆F₄N₆O₃S | |
| 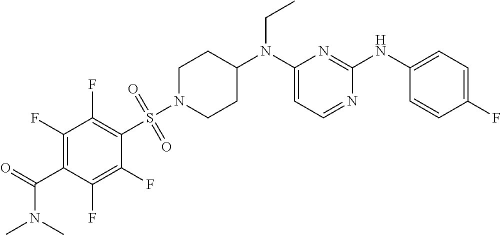 [SMILES: CCN(c1ccnc(Nc2ccc(F)cc2)n1)C1CCN(S(=O)(=O)c2c(F)c(F)c(C(=O)N(C)C)c(F)c2F)CC1] | A277 |
| C₂₆H₂₇F₅N₆O₃S | |
| 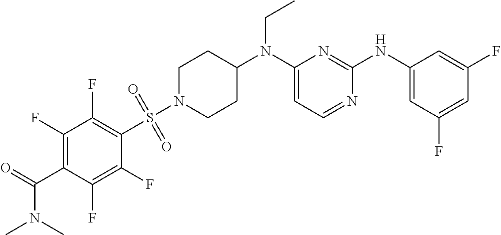 [SMILES: CCN(c1ccnc(Nc2cc(F)cc(F)c2)n1)C1CCN(S(=O)(=O)c2c(F)c(F)c(C(=O)N(C)C)c(F)c2F)CC1] | A278 |
| C₂₆H₂₆F₆N₆O₃S | |
| 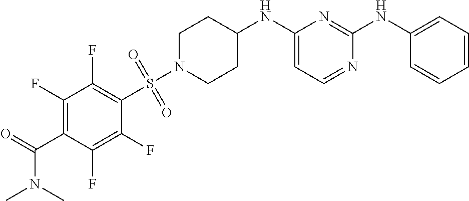 [SMILES: CN(C)C(=O)c1c(F)c(F)c(S(=O)(=O)N2CCC(Nc3ccnc(Nc4ccccc4)n3)CC2)c(F)c1F] | A279 |
| C₂₄H₂₄F₄N₆O₃S | |
| 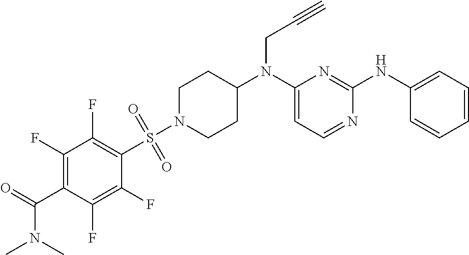 [SMILES: C#CCN(c1ccnc(Nc2ccccc2)n1)C1CCN(S(=O)(=O)c2c(F)c(F)c(C(=O)N(C)C)c(F)c2F)CC1] | A280 |
| C₂₇H₂₆F₄N₆O₃S | |
| 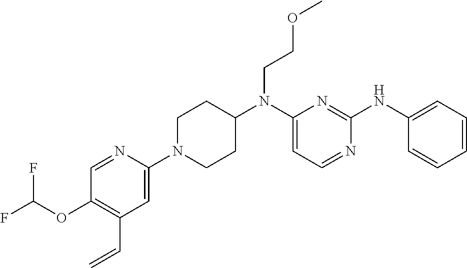 [SMILES: C=Cc1cc(N2CCC(N(CCOC)c3ccnc(Nc4ccccc4)n3)CC2)ncc1OC(F)F] | A281 |
| C₂₆H₃₀F₂N₆O₂ | |
| 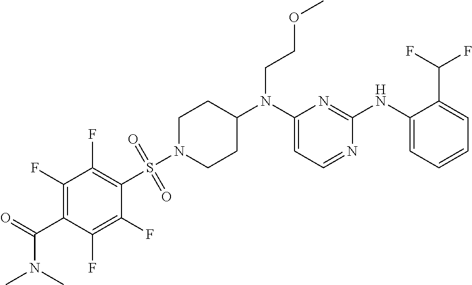 [SMILES: COCCN(c1ccnc(Nc2ccccc2C(F)F)n1)C1CCN(S(=O)(=O)c2c(F)c(F)c(C(=O)N(C)C)c(F)c2F)CC1] | A282 |
| C₂₈H₃₀F₆N₆O₄S | |
| 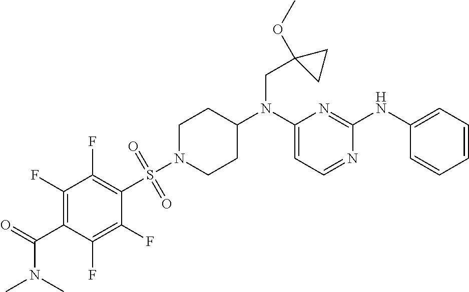 [SMILES: COC1(CN(c2ccnc(Nc3ccccc3)n2)C2CCN(S(=O)(=O)c3c(F)c(F)c(C(=O)N(C)C)c(F)c3F)CC2)CC1] | A283 |
| C₂₉H₃₂F₄N₆O₄S | |
| 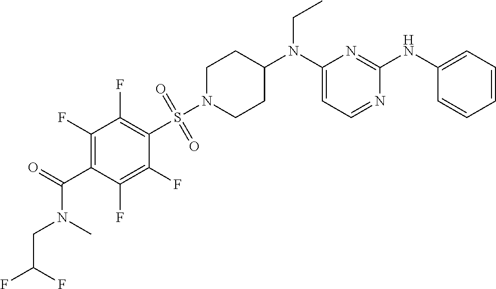 [SMILES: CCN(c1ccnc(Nc2ccccc2)n1)C1CCN(S(=O)(=O)c2c(F)c(F)c(C(=O)N(C)CC(F)F)c(F)c2F)CC1] | A284 |
| C₂₇H₂₈F₆N₆O₃S | |
| 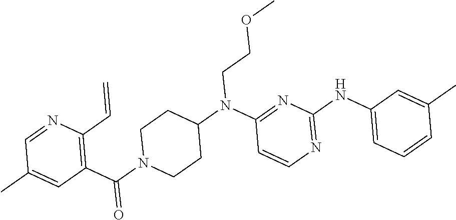 | A285 |
| C₂₈H₃₄N₆O₂ | |
| 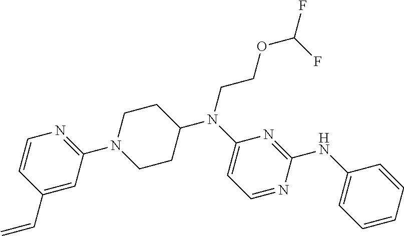 | A286 |
| C₂₅H₂₈F₂N₆O | |
| 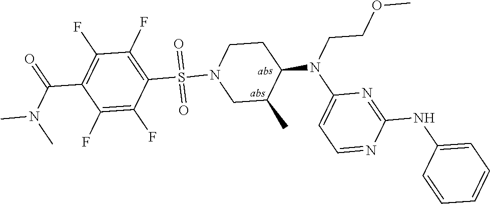 [SMILES: COCCN(c1ccnc(Nc2ccccc2)n1)[C@@H]1CCN(S(=O)(=O)c2c(F)c(F)c(C(=O)N(C)C)c(F)c2F)C[C@@H]1C] | A287 |
| C₂₈H₃₂F₄N₆O4S | |
| 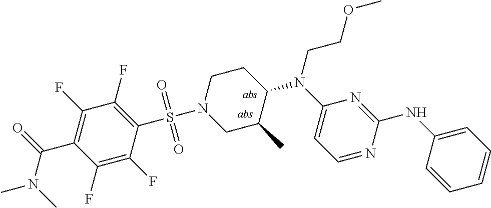 [SMILES: COCCN(c1ccnc(Nc2ccccc2)n1)[C@H]1CCN(S(=O)(=O)c2c(F)c(F)c(C(=O)N(C)C)c(F)c2F)C[C@@H]1C] | A288 |
| C₂₈H₃₂F₄N₆O₄S | |
| 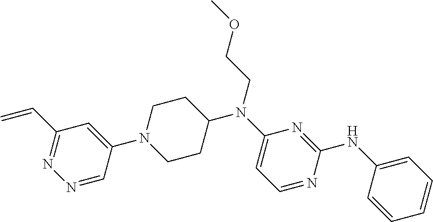 [SMILES: C=Cc1cc(N2CCC(N(CCOC)c3ccnc(Nc4ccccc4)n3)CC2)cnn1] | A289 |
| C₂₄H₂₉N₇O | |
| 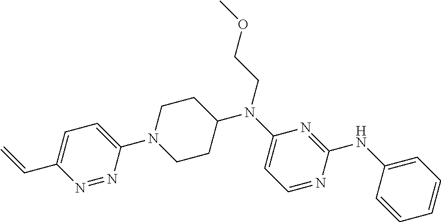 [SMILES: C=Cc1ccc(N2CCC(N(CCOC)c3ccnc(Nc4ccccc4)n3)CC2)nn1] | A290 |
| C₂₄H₂₉N₇O | |
| 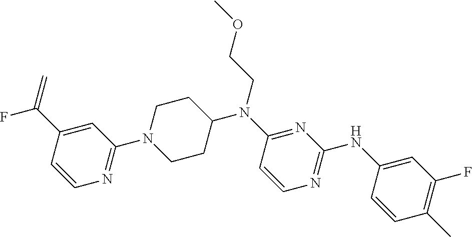 [SMILES: C=C(F)c1ccnc(N2CCC(N(CCOC)c3ccnc(Nc4ccc(C)c(F)c4)n3)CC2)c1] | A291 |
| C₂₆H₃₀F₂N₆O | |
| 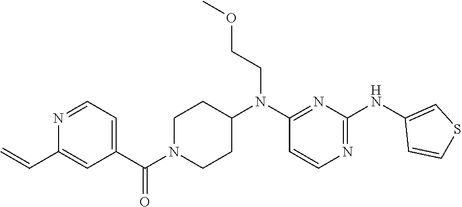 [SMILES: C=Cc1cc(C(=O)N2CCC(N(CCOC)c3ccnc(Nc4ccsc4)n3)CC2)ccn1] | A292 |
| C₂₄H₂₈N₆O₂S | |
| 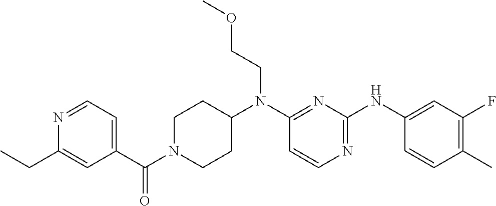 [SMILES: CCc1cc(C(=O)N2CCC(N(CCOC)c3ccnc(Nc4ccc(C)c(F)c4)n3)CC2)ccn1] | A293 |
| C₂₇H₃₃FN₆O₂ | |
| 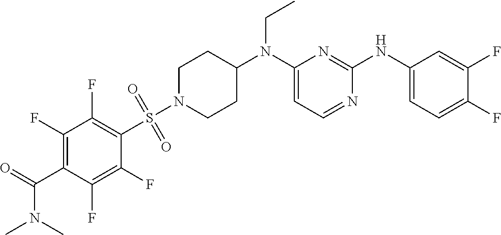 [SMILES: CCN(c1ccnc(Nc2ccc(F)c(F)c2)n1)C1CCN(S(=O)(=O)c2c(F)c(F)c(C(=O)N(C)C)c(F)c2F)CC1] | A294 |
| C₂₆H₂₆F₆N₆O₃S | |
| 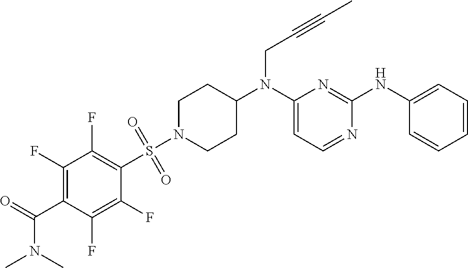 [SMILES: CC#CCN(c1ccnc(Nc2ccccc2)n1)C1CCN(S(=O)(=O)c2c(F)c(F)c(C(=O)N(C)C)c(F)c2F)CC1] | A295 |
| C₂₈H₂₈F₄N₆O₃S | |
| 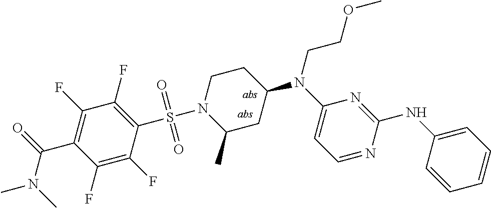 [SMILES: COCCN(c1ccnc(Nc2ccccc2)n1)[C@@H]1CCN(S(=O)(=O)c2c(F)c(F)c(C(=O)N(C)C)c(F)c2F)[C@H](C)C1] | A296 |
| C₂₈H₃₂F₄N₆O₄S | |
| 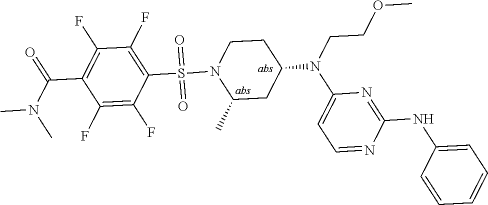 [SMILES: COCCN(c1ccnc(Nc2ccccc2)n1)[C@H]1CCN(S(=O)(=O)c2c(F)c(F)c(C(=O)N(C)C)c(F)c2F)[C@@H](C)C1] | A297 |
| C₂₈H₃₂F₄N₆O₄S | |
| 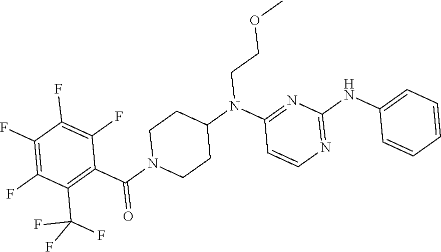 [SMILES: COCCN(c1ccnc(Nc2ccccc2)n1)C1CCN(C(=O)c2c(F)c(F)c(F)c(F)c2C(F)(F)F)CC1] | A298 |
| C₂₆H₂₄F₇N₅O₂ | |
| 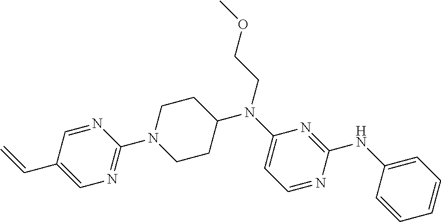 [SMILES: C=Cc1cnc(N2CCC(N(CCOC)c3ccnc(Nc4ccccc4)n3)CC2)nc1] | A299 |
| C₂₄H₂₉N₇O | |
| 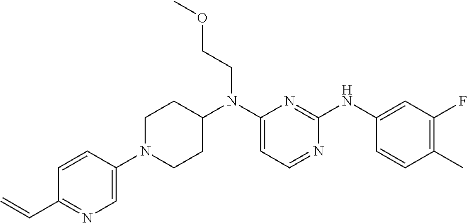 [SMILES: C=Cc1ccc(N2CCC(N(CCOC)c3ccnc(Nc4ccc(C)c(F)c4)n3)CC2)cn1] | A300 |
| C₂₆H₃₁FN₆O | |
| 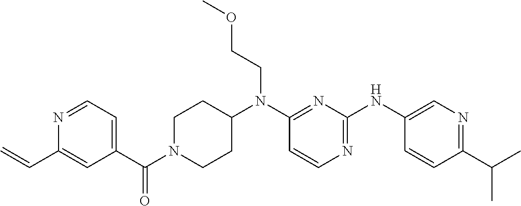 [SMILES: C=Cc1cc(C(=O)N2CCC(N(CCOC)c3ccnc(Nc4ccc(C(C)C)nc4)n3)CC2)ccn1] | A301 |
| C₂₈H₃₅N₇O₂ | |
| 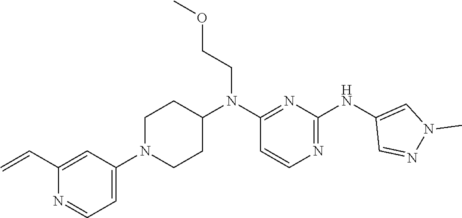 [SMILES: C=Cc1cc(N2CCC(N(CCOC)c3ccnc(Nc4cnn(C)c4)n3)CC2)ccn1] | A302 |
| C₂₃H₃₀N₈O | |
| 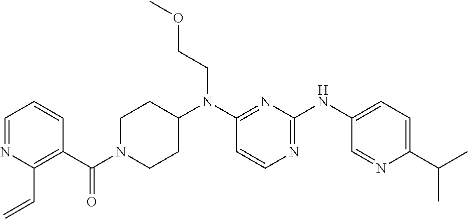 [SMILES: C=Cc1ncccc1C(=O)N1CCC(N(CCOC)c2ccnc(Nc3ccc(C(C)C)nc3)n2)CC1] | A303 |
| C₂₈H₃₅N₇O₂ | |
| 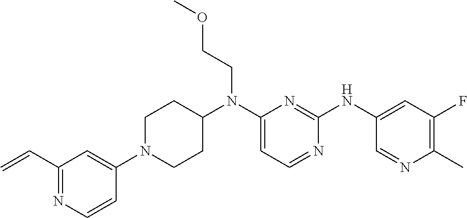 [SMILES: C=Cc1cc(N2CCC(N(CCOC)c3ccnc(Nc4cnc(C)c(F)c4)n3)CC2)ccn1] | A304 |
| C₂₅H₃₀FN₇O | |
| 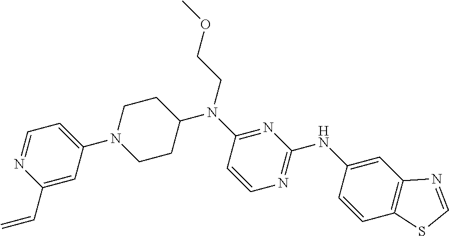 [SMILES: C=Cc1cc(N2CCC(N(CCOC)c3ccnc(Nc4ccc5scnc5c4)n3)CC2)ccn1] | A305 |
| C₂₆H₂₉N₇OS | |
| 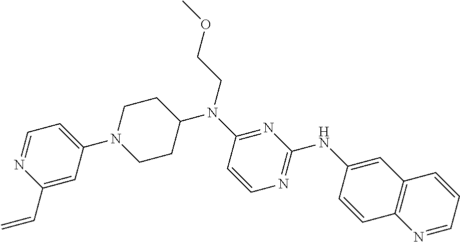 [SMILES: C=Cc1cc(N2CCC(N(CCOC)c3ccnc(Nc4ccc5ncccc5c4)n3)CC2)ccn1] | A306 |
| C₂₈H₃₁N₇₈O | |
| 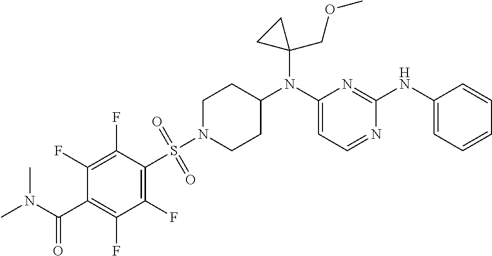 [SMILES: COCC1(N(c2ccnc(Nc3ccccc3)n2)C2CCN(S(=O)(=O)c3c(F)c(F)c(C(=O)N(C)C)c(F)c3F)CC2)CC1] | A307 |
| C₂₉H₃₂F₄N₆O₄S | |
| 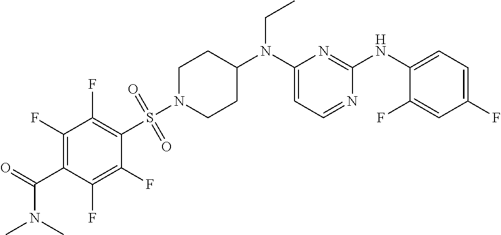 [SMILES: CCN(c1ccnc(Nc2ccc(F)cc2F)n1)C1CCN(S(=O)(=O)c2c(F)c(F)c(C(=O)N(C)C)c(F)c2F)CC1] | A308 |
| C₂₆H₂₆F₆N₆O₃S | |
| 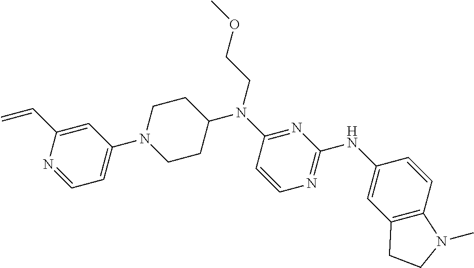 [SMILES: C=Cc1cc(N2CCC(N(CCOC)c3ccnc(Nc4ccc5c(c4)CCN5C)n3)CC2)ccn1] | A309 |
| C₂₈H₃₅N₇O | |
| 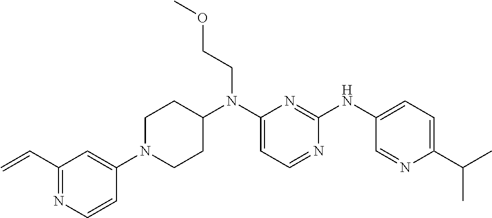 [SMILES: C=Cc1cc(N2CCC(N(CCOC)c3ccnc(Nc4ccc(C(C)C)nc4)n3)CC2)ccn1] | A310 |
| C₂₇H₃₅N₇O | |
| 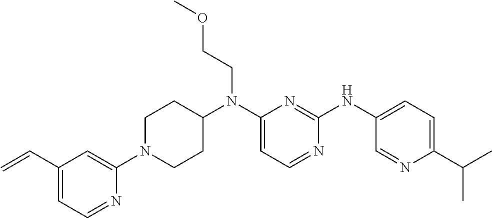 [SMILES: C=Cc1ccnc(N2CCC(N(CCOC)c3ccnc(Nc4ccc(C(C)C)nc4)n3)CC2)c1] | A311 |
| C₂₇H₃₅N₇O | |
| 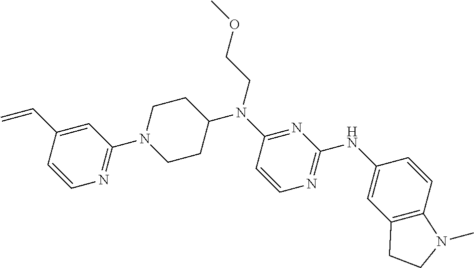 [SMILES: C=Cc1ccnc(N2CCC(N(CCOC)c3ccnc(Nc4ccc5c(c4)CCN5C)n3)CC2)c1] | A312 |
| C₂₈H₃₅N₇O | |
| 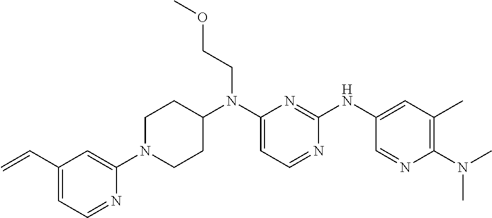 [SMILES: C=Cc1ccnc(N2CCC(N(CCOC)c3ccnc(Nc4cnc(N(C)C)c(C)c4)n3)CC2)c1] | A313 |
| C₂₇H₃₆N₈O | |
| 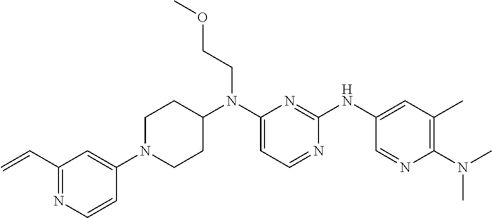 [SMILES: C=Cc1cc(N2CCC(N(CCOC)c3ccnc(Nc4cnc(N(C)C)c(C)c4)n3)CC2)ccn1] | A314 |
| C₂₇H₃₆N₈O | |
| 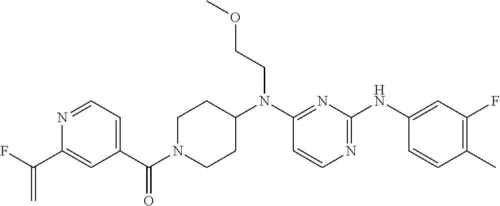 [SMILES: C=C(F)c1cc(C(=O)N2CCC(N(CCOC)c3ccnc(Nc4ccc(C)c(F)c4)n3)CC2)ccn1] | A315 |
| C₂₇H₃₀F₂N₆O2 | |
| 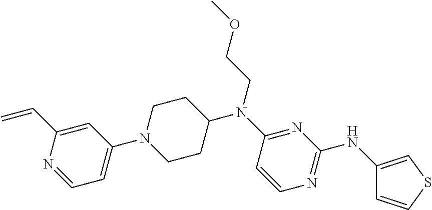 [SMILES: C=Cc1cc(N2CCC(N(CCOC)c3ccnc(Nc4ccsc4)n3)CC2)ccn1] | A316 |
| C₂₃H₂₈N₆OS | |
| 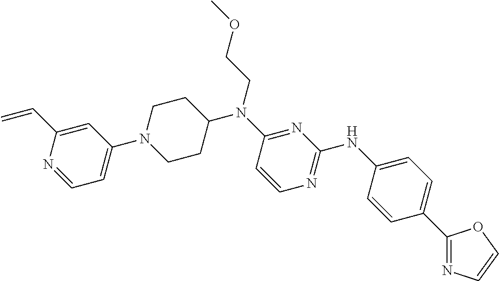 [SMILES: C=Cc1cc(N2CCC(N(CCOC)c3ccnc(Nc4ccc(-c5ncco5)cc4)n3)CC2)ccn1] | A317 |
| C₂₈H₃₁N₇O₂ | |
| 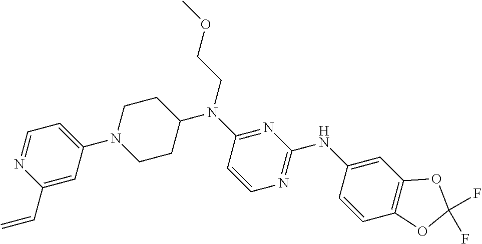 [SMILES: C=Cc1cc(N2CCC(N(CCOC)c3ccnc(Nc4ccc5c(c4)OC(F)(F)O5)n3)CC2)ccn1] | A318 |
| C₂₆H₂₈F₂N₆O₃ | |
| 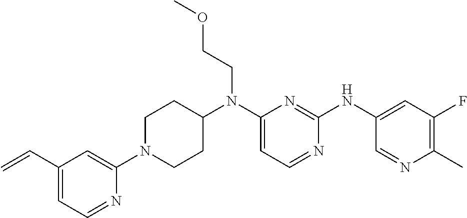 [SMILES: C=Cc1ccnc(N2CCC(N(CCOC)c3ccnc(Nc4cnc(C)c(F)c4)n3)CC2)c1] | A319 |
| C₂₅H₃₀FN₇O | |
| 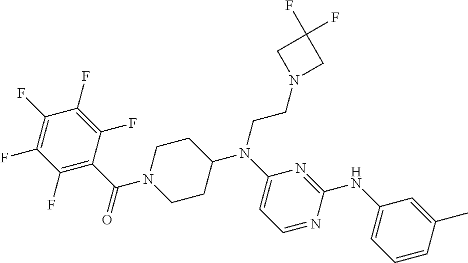 [SMILES: Cc1cccc(Nc2nccc(N(CCN3CC(F)(F)C3)C3CCN(C(=O)c4c(F)c(F)c(F)c(F)c4F)CC3)n2)c1] | A320 |
| C₂₈H₂₇F₇N₆O | |
| 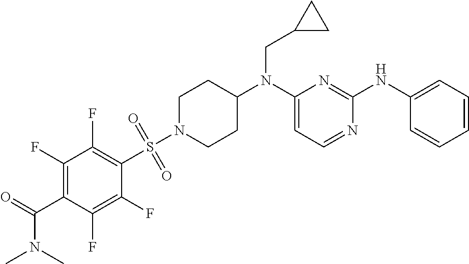 [SMILES: CN(C)C(=O)c1c(F)c(F)c(S(=O)(=O)N2CCC(N(CC3CC3)c3ccnc(Nc4ccccc4)n3)CC2)c(F)c1F] | A321 |
| C₂₈H₃₀F₄N₆O₃S | |
| 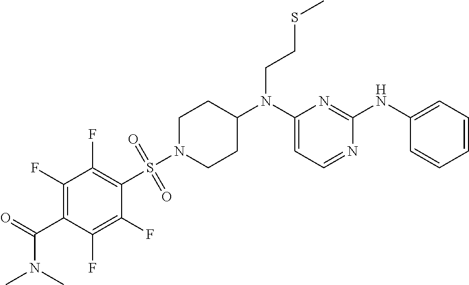 [SMILES: CSCCN(c1ccnc(Nc2ccccc2)n1)C1CCN(S(=O)(=O)c2c(F)c(F)c(C(=O)N(C)C)c(F)c2F)CC1] | A322 |
| C₂₇H₃₀F₄N₆O₃S₂ | |
| 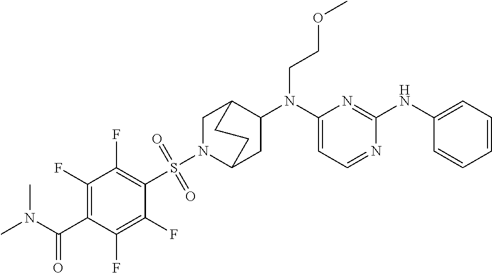 [SMILES: COCCN(c1ccnc(Nc2ccccc2)n1)C1CC2CCC1CN2S(=O)(=O)c1c(F)c(F)c(C(=O)N(C)C)c(F)c1F] | A323 |
| C₂₉H₃₂F₄N₆O₄S | |
| 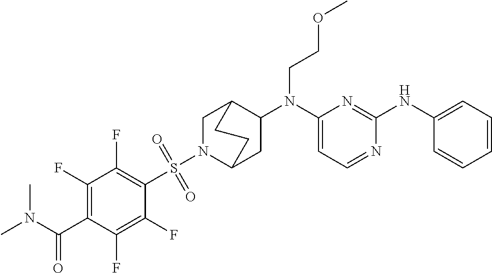 [SMILES: COCCN(c1ccnc(Nc2ccccc2)n1)C1CC2CCC1CN2S(=O)(=O)c1c(F)c(F)c(C(=O)N(C)C)c(F)c1F] | A324 |
| C₂₉H₃₂F₄N₆O₄S | |
| 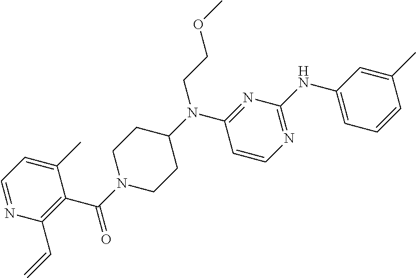 [SMILES: C=Cc1nccc(C)c1C(=O)N1CCC(N(CCOC)c2ccnc(Nc3cccc(C)c3)n2)CC1] | A325 |
| C₂₈H₃₄N₆O₂ | |
| 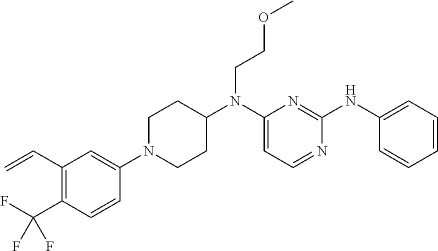 [SMILES: C=Cc1cc(N2CCC(N(CCOC)c3ccnc(Nc4ccccc4)n3)CC2)ccc1C(F)(F)F] | A326 |
| C₂₆H₂₉F₃N₆O | |
| 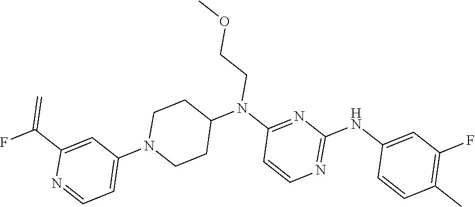 [SMILES: C=C(F)c1cc(N2CCC(N(CCOC)c3ccnc(Nc4ccc(C)c(F)c4)n3)CC2)ccn1] | A327 |
| C₂₆H₃₀F₂N₆O | |
| 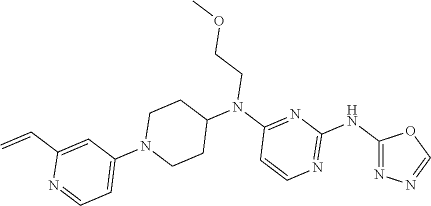 [SMILES: C=Cc1cc(N2CCC(N(CCOC)c3ccnc(Nc4nnco4)n3)CC2)ccn1] | A328 |
| C₂₁H₂₆N₈O₂ | |
| 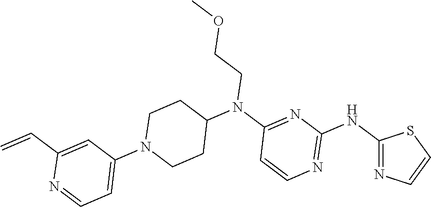 [SMILES: C=Cc1cc(N2CCC(N(CCOC)c3ccnc(Nc4nccs4)n3)CC2)ccn1] | A329 |
| C₂₂H₂₇N₇OS | |
| 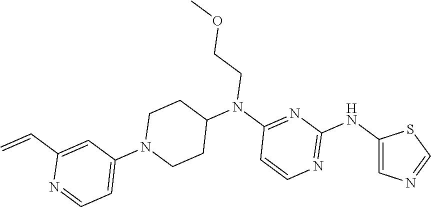 [SMILES: C=Cc1cc(N2CCC(N(CCOC)c3ccnc(Nc4cncs4)n3)CC2)ccn1] | A330 |
| C₂₂H₂₇N₇OS | |
| 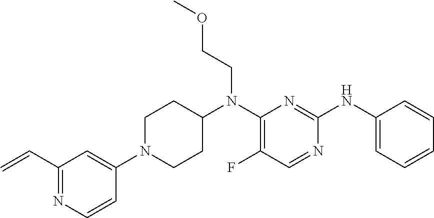 [SMILES: C=Cc1cc(N2CCC(N(CCOC)c3nc(Nc4ccccc4)ncc3F)CC2)ccn1] | A331 |
| C₂₅H₂₉FN₆O | |
| 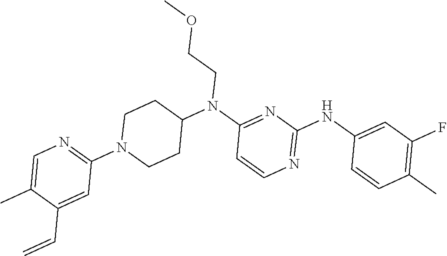 [SMILES: C=Cc1cc(N2CCC(N(CCOC)c3ccnc(Nc4ccc(C)c(F)c4)n3)CC2)ncc1C] | A332 |
| C₂₇H₃₃FN₆O | |
| 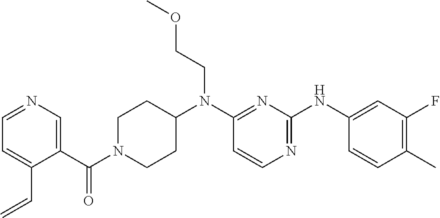 [SMILES: C=Cc1ccncc1C(=O)N1CCC(N(CCOC)c2ccnc(Nc3ccc(C)c(F)c3)n2)CC1] | A333 |
| C₂₇H₃₁FN₆O2 | |
| 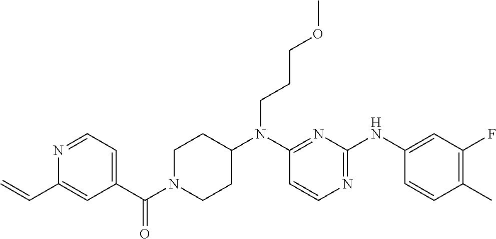 [SMILES: C=Cc1cc(C(=O)N2CCC(N(CCCOC)c3ccnc(Nc4ccc(C)c(F)c4)n3)CC2)ccn1] | A334 |
| C₂₈H₃₃FN₆O₂ | |
| 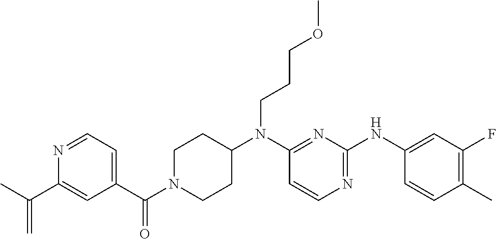 [SMILES: C=C(C)c1cc(C(=O)N2CCC(N(CCCOC)c3ccnc(Nc4ccc(C)c(F)c4)n3)CC2)ccn1] | A335 |
| C₂₉H₃₅FN₆O₂ | |
| 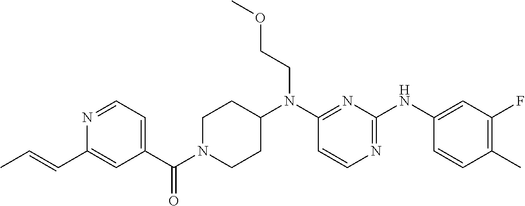 [SMILES: C/C=C/c1cc(C(=O)N2CCC(N(CCOC)c3ccnc(Nc4ccc(C)c(F)c4)n3)CC2)ccn1] | A336 |
| C₂₈H₃₃FN₆O₂ | |
| 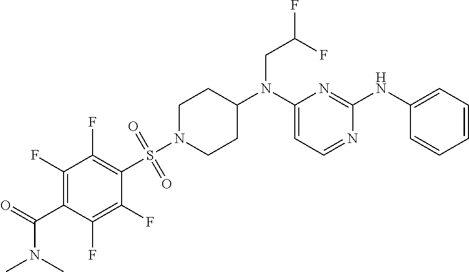 [SMILES: CN(C)C(=O)c1c(F)c(F)c(S(=O)(=O)N2CCC(N(CC(F)F)c3ccnc(Nc4ccccc4)n3)CC2)c(F)c1F] | A337 |
| C₂₆H₂₆F₆N₆O₃S | |
| 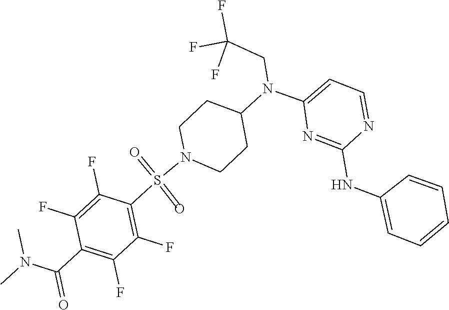 | A338 |
| C₂₆H₂₅F₇N₆O₃S | |
| 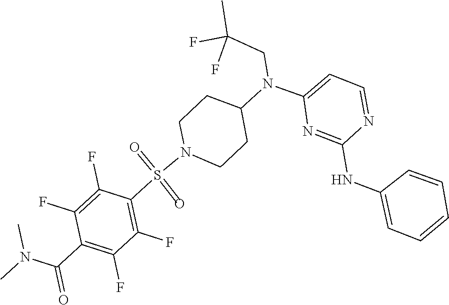 | A339 |
| C₂₇H₂₈F₆N₆O₃S | |
| 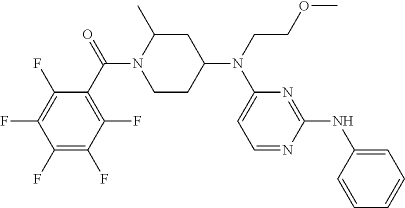 [SMILES: COCCN(c1ccnc(Nc2ccccc2)n1)C1CCN(C(=O)c2c(F)c(F)c(F)c(F)c2F)C(C)C1] | A340 |
| C₂₆H₂₆F₅N₅O₂ | |
| 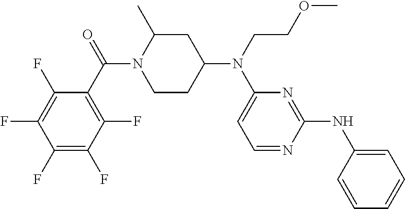 [SMILES: COCCN(c1ccnc(Nc2ccccc2)n1)C1CCN(C(=O)c2c(F)c(F)c(F)c(F)c2F)C(C)C1] | A341 |
| C₂₆H₂₆F₅N₅O₂ | |
| 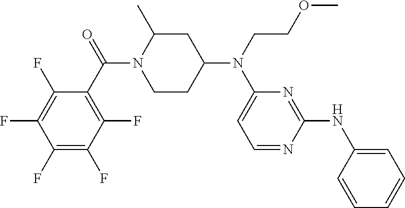 [SMILES: COCCN(c1ccnc(Nc2ccccc2)n1)C1CCN(C(=O)c2c(F)c(F)c(F)c(F)c2F)C(C)C1] | A342 |
| C₂₆H₂₆F₅N₅O₂ | |
| 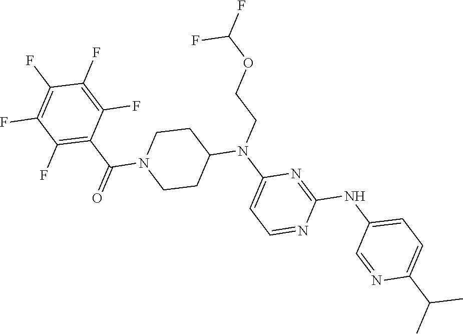 [SMILES: CC(C)c1ccc(Nc2nccc(N(CCOC(F)F)C3CCN(C(=O)c4c(F)c(F)c(F)c(F)c4F)CC3)n2)cn1] | A343 |
| C₂₇H₂₇F₇N₆O₂ | |
| 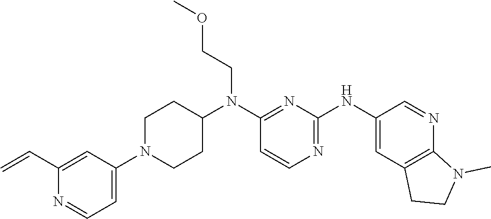 [SMILES: C=Cc1cc(N2CCC(N(CCOC)c3ccnc(Nc4cnc5c(c4)CCN5C)n3)CC2)ccn1] | A344 |
| C₂₇H₃₄N₈O | |
| 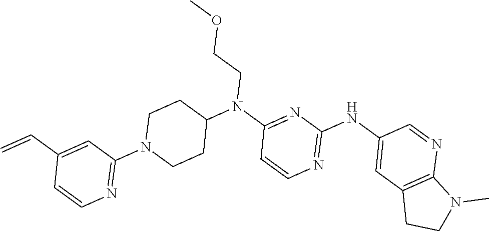 [SMILES: C=Cc1ccnc(N2CCC(N(CCOC)c3ccnc(Nc4cnc5c(c4)CCN5C)n3)CC2)c1] | A345 |
| C₂₇H₃₄N₈O | |
| 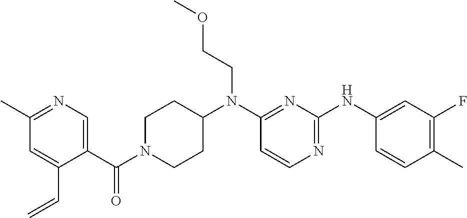 | A346 |
| C₂₈H₃₃FN₆O₂ | |
| 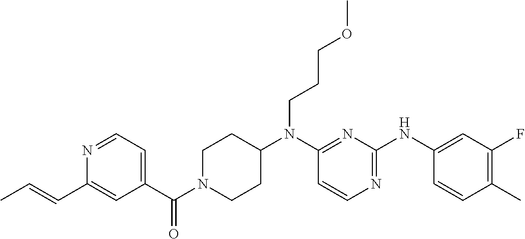 [SMILES: C/C=C/c1cc(C(=O)N2CCC(N(CCCOC)c3ccnc(Nc4ccc(C)c(F)c4)n3)CC2)ccn1] | A347 |
| C₂₉H₃₅FN₆O₂ | |
| 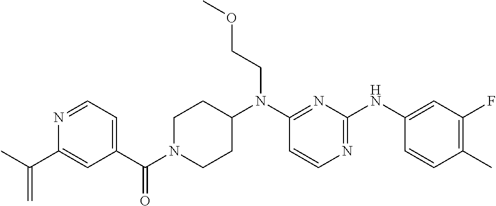 [SMILES: C=C(C)c1cc(C(=O)N2CCC(N(CCOC)c3ccnc(Nc4ccc(C)c(F)c4)n3)CC2)ccn1] | A348 |
| C₂₈H₃₃FN₆O2 | |
| 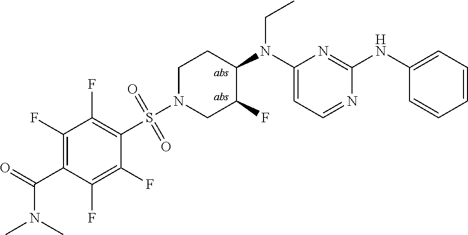 [SMILES: CCN(c1ccnc(Nc2ccccc2)n1)[C@@H]1CCN(S(=O)(=O)c2c(F)c(F)c(C(=O)N(C)C)c(F)c2F)C[C@@H]1F] | A349 |
| C₂₆H₂₇F₅N₆O₃S | |
| 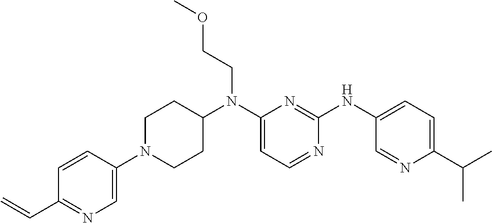 [SMILES: C=Cc1ccc(N2CCC(N(CCOC)c3ccnc(Nc4ccc(C(C)C)nc4)n3)CC2)cn1] | A350 |
| C₂₇H₃₅N₇O | |
| 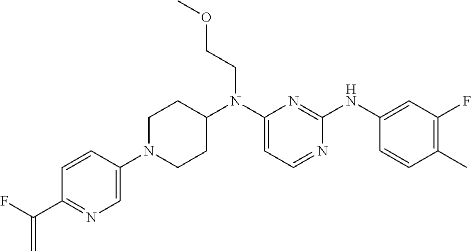 [SMILES: C=C(F)c1ccc(N2CCC(N(CCOC)c3ccnc(Nc4ccc(C)c(F)c4)n3)CC2)cn1] | A351 |
| C₂₆H₃₀F₂N₆O | |
| 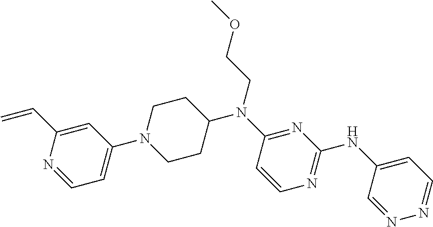 [SMILES: C=Cc1cc(N2CCC(N(CCOC)c3ccnc(Nc4ccnnc4)n3)CC2)ccn1] | A352 |
| C₂₃H₂₈N₈O | |
| 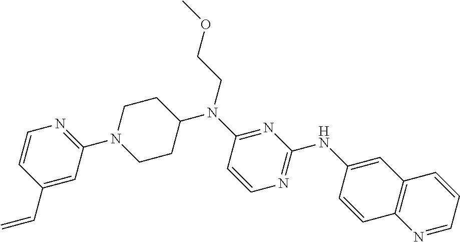 [SMILES: C=Cc1ccnc(N2CCC(N(CCOC)c3ccnc(Nc4ccc5ncccc5c4)n3)CC2)c1] | A353 |
| C₂₈H₃₁N₇O | |
| 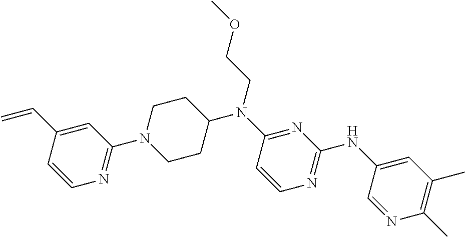 [SMILES: C=Cc1ccnc(N2CCC(N(CCOC)c3ccnc(Nc4cnc(C)c(C)c4)n3)CC2)c1] | A354 |
| C₂₆H₃₃N₇O | |
| 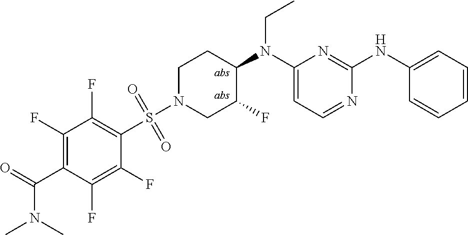 [SMILES: CCN(c1ccnc(Nc2ccccc2)n1)[C@@H]1CCN(S(=O)(=O)c2c(F)c(F)c(C(=O)N(C)C)c(F)c2F)C[C@H]1F] | A355 |
| C₂₆H₂₇F₅N₆O₃S | |
| 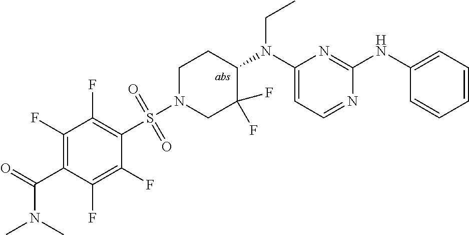 [SMILES: CCN(c1ccnc(Nc2ccccc2)n1)[C@H]1CCN(S(=O)(=O)c2c(F)c(F)c(C(=O)N(C)C)c(F)c2F)CC1(F)F] | A356 |
| C₂₆H₂₆F₆N₆O₃S | |
| 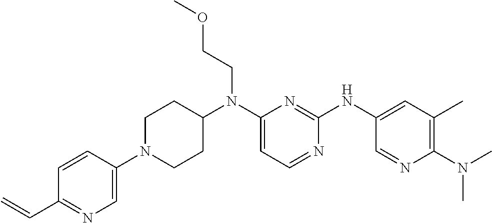 [SMILES: C=Cc1ccc(N2CCC(N(CCOC)c3ccnc(Nc4cnc(N(C)C)c(C)c4)n3)CC2)cn1] | A357 |
| C₂₇H₃₆N₈O | |
| 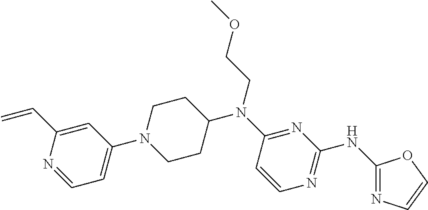 [SMILES: C=Cc1cc(N2CCC(N(CCOC)c3ccnc(Nc4ncco4)n3)CC2)ccn1] | A358 |
| C₂₂H₂₇N₇O₂ | |
| 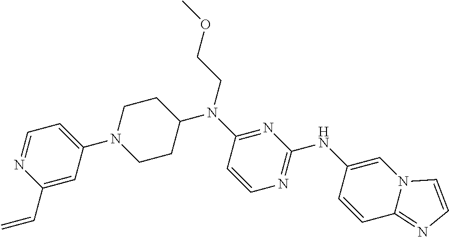 [SMILES: C=Cc1cc(N2CCC(N(CCOC)c3ccnc(Nc4ccc5nccn5c4)n3)CC2)ccn1] | A359 |
| C₂₆H₃₀N₈O | |
| 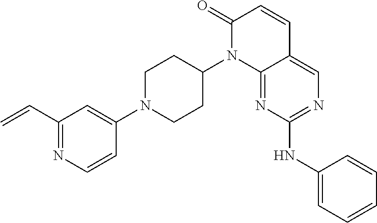 [SMILES: C=Cc1cc(N2CCC(n3c(=O)ccc4cnc(Nc5ccccc5)nc43)CC2)ccn1] | A360 |
| C₂₅H₂₄N₆O | |
| 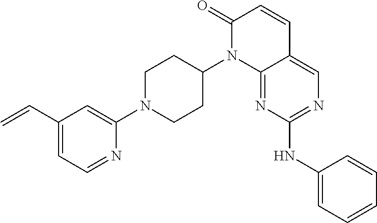 [SMILES: C=Cc1ccnc(N2CCC(n3c(=O)ccc4cnc(Nc5ccccc5)nc43)CC2)c1] | A361 |
| C₂₅H₂₄N₆O | |
| 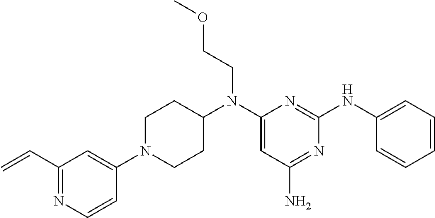 [SMILES: C=Cc1cc(N2CCC(N(CCOC)c3cc(N)nc(Nc4ccccc4)n3)CC2)ccn1] | A362 |
| C₂₅H₃₁N₇O | |
| 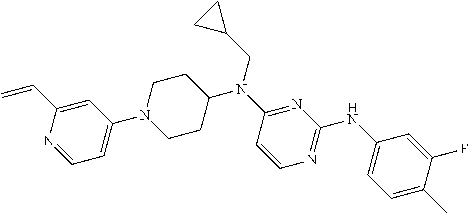 [SMILES: C=Cc1cc(N2CCC(N(CC3CC3)c3ccnc(Nc4ccc(C)c(F)c4)n3)CC2)ccn1] | A363 |
| C₂₇H₃₁FN₆ | |
| 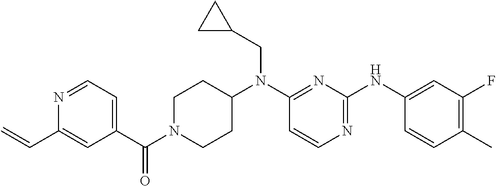 [SMILES: C=Cc1cc(C(=O)N2CCC(N(CC3CC3)c3ccnc(Nc4ccc(C)c(F)c4)n3)CC2)ccn1] | A364 |
| C₂₈H₃₁FN₆O | |
| 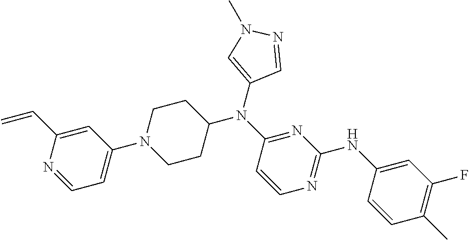 [SMILES: C=Cc1cc(N2CCC(N(c3cnn(C)c3)c3ccnc(Nc4ccc(C)c(F)c4)n3)CC2)ccn1] | A365 |
| C₂₇H₂₉FN₈ | |
| 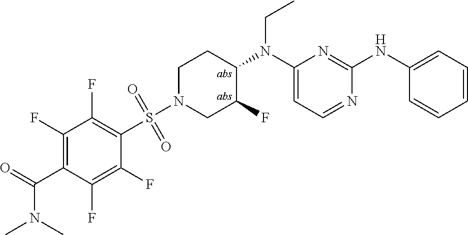 [SMILES: CCN(c1ccnc(Nc2ccccc2)n1)[C@H]1CCN(S(=O)(=O)c2c(F)c(F)c(C(=O)N(C)C)c(F)c2F)C[C@@H]1F] | A366 |
| C₂₆H₂₇F₅N₆O₃S | |
| 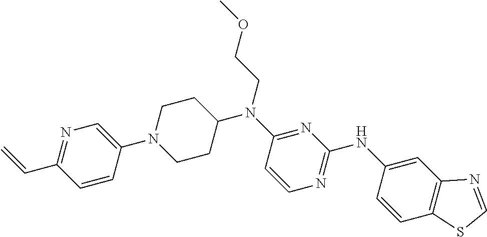 [SMILES: C=Cc1ccc(N2CCC(N(CCOC)c3ccnc(Nc4ccc5scnc5c4)n3)CC2)cn1] | A367 |
| C₂₆H₂₉N₇OS | |
| 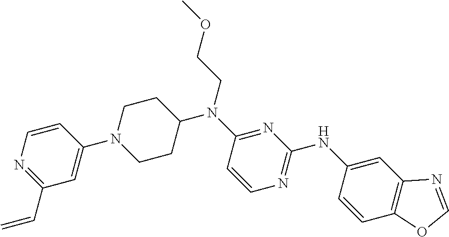 [SMILES: C=Cc1cc(N2CCC(N(CCOC)c3ccnc(Nc4ccc5ocnc5c4)n3)CC2)ccn1] | A368 |
| C₂₆H₂₉N₇O₂ | |
| 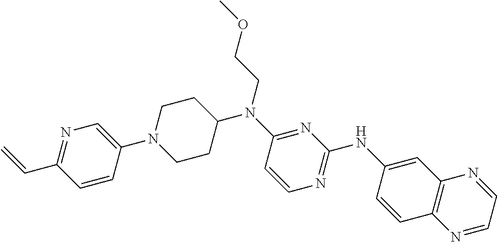 [SMILES: C=Cc1ccc(N2CCC(N(CCOC)c3ccnc(Nc4ccc5nccnc5c4)n3)CC2)cn1] | A369 |
| C₂₇H₃₀N₈O | |
| 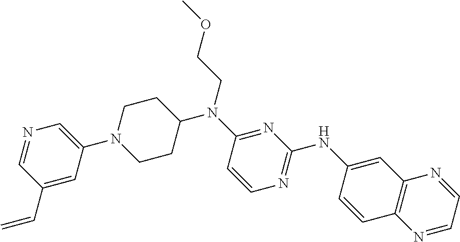 [SMILES: C=Cc1cncc(N2CCC(N(CCOC)c3ccnc(Nc4ccc5nccnc5c4)n3)CC2)c1] | A370 |
| C₂₇H₃₀N₈O | |
| 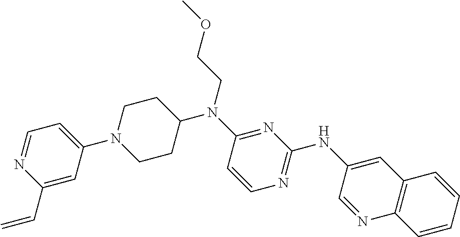 [SMILES: C=Cc1cc(N2CCC(N(CCOC)c3ccnc(Nc4cnc5ccccc5c4)n3)CC2)ccn1] | A371 |
| C₂₈H₃₁N₇O | |
| 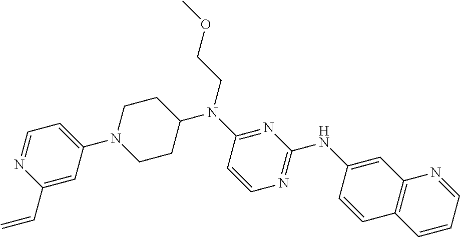 [SMILES: C=Cc1cc(N2CCC(N(CCOC)c3ccnc(Nc4ccc5cccnc5c4)n3)CC2)ccn1] | A372 |
| C₂₈H₃₁N₇O | |
| 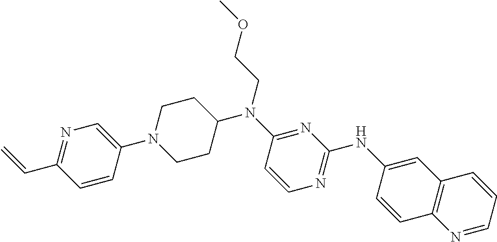 [SMILES: C=Cc1ccc(N2CCC(N(CCOC)c3ccnc(Nc4ccc5ncccc5c4)n3)CC2)cn1] | A373 |
| C₂₈H₃₁N₇O | |
| 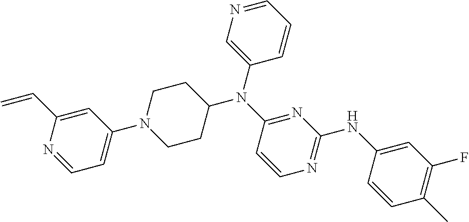 [SMILES: C=Cc1cc(N2CCC(N(c3cccnc3)c3ccnc(Nc4ccc(C)c(F)c4)n3)CC2)ccn1] | A374 |
| C₂₈H₂₈FN₇ | |
| 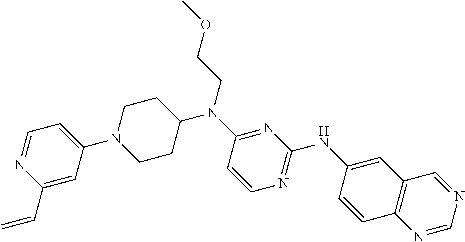 [SMILES: C=Cc1cc(N2CCC(N(CCOC)c3ccnc(Nc4ccc5ncncc5c4)n3)CC2)ccn1] | A375 |
| C₂₇H₃₀N₈O | |
| 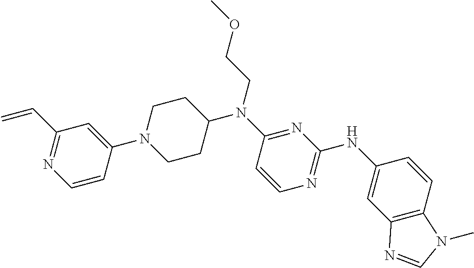 [SMILES: C=Cc1cc(N2CCC(N(CCOC)c3ccnc(Nc4ccc5c(c4)ncn5C)n3)CC2)ccn1] | A376 |
| C₂₇H₃₂N₇O | |
| 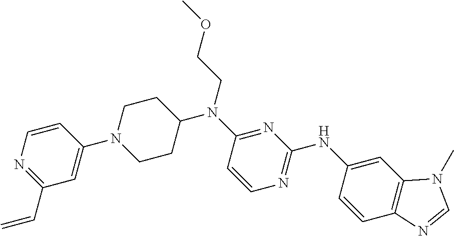 [SMILES: C=Cc1cc(N2CCC(N(CCOC)c3ccnc(Nc4ccc5ncn(C)c5c4)n3)CC2)ccn1] | A377 |
| C₂₇H₃₂N₈O | |
| 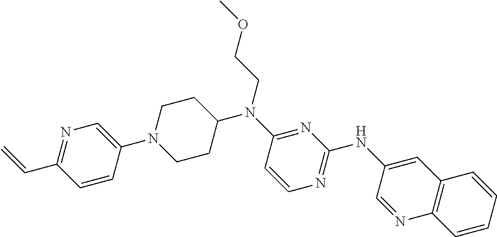 [SMILES: C=Cc1ccc(N2CCC(N(CCOC)c3ccnc(Nc4cnc5ccccc5c4)n3)CC2)cn1] | A378 |
| C₂₈H₃₁N₇O | |
| 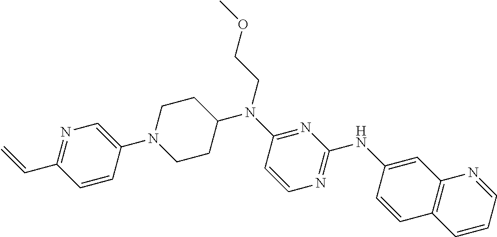 [SMILES: C=Cc1ccc(N2CCC(N(CCOC)c3ccnc(Nc4ccc5cccnc5c4)n3)CC2)cn1] | A379 |
| C₂₈H₃₁N₇O | |
| 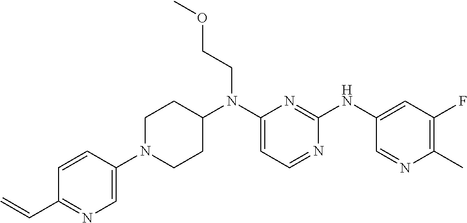 [SMILES: C=Cc1ccc(N2CCC(N(CCOC)c3ccnc(Nc4cnc(C)c(F)c4)n3)CC2)cn1] | A380 |
| C₂₅H₃₀FN₇O | |
| 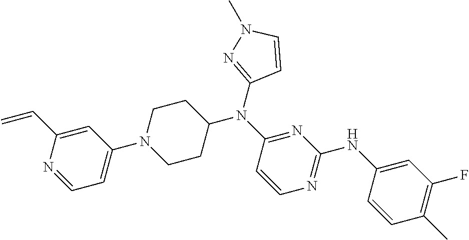 [SMILES: C=Cc1cc(N2CCC(N(c3ccnc(Nc4ccc(C)c(F)c4)n3)c3ccn(C)n3)CC2)ccn1] | A381 |
| C₂₇H₂₉FN₈ | |
| 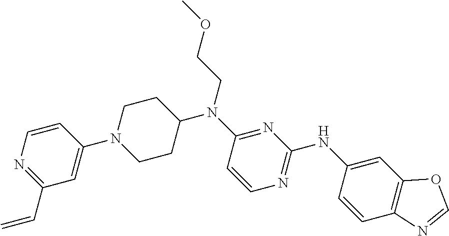 [SMILES: C=Cc1cc(N2CCC(N(CCOC)c3ccnc(Nc4ccc5ncoc5c4)n3)CC2)ccn1] | A382 |
| C₂₆H₂₉N₇O₂ | |
| 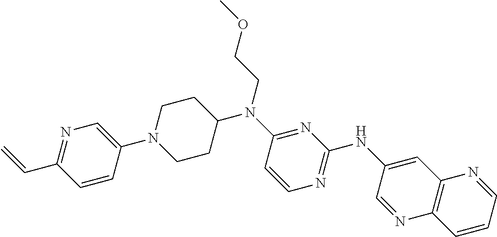 [SMILES: C=Cc1ccc(N2CCC(N(CCOC)c3ccnc(Nc4cnc5cccnc5c4)n3)CC2)cn1] | A383 |
| C₂₇H₃₀N₈O | |
| 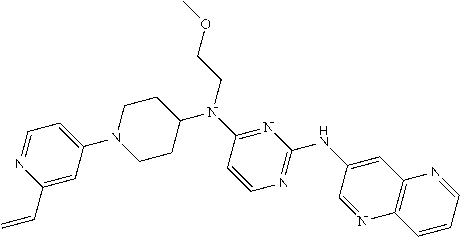 [SMILES: C=Cc1cc(N2CCC(N(CCOC)c3ccnc(Nc4cnc5cccnc5c4)n3)CC2)ccn1] | A384 |
| C₂₇H₃₀N₈O | |
| 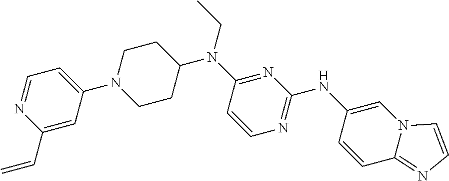 [SMILES: C=Cc1cc(N2CCC(N(CC)c3ccnc(Nc4ccc5nccn5c4)n3)CC2)ccn1] | A385 |
| C₂₅H₂₈N₈ | |
| 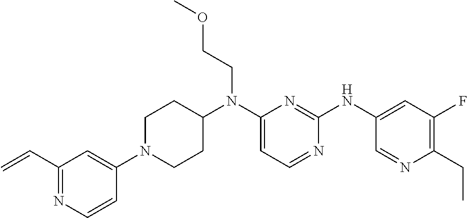 [SMILES: C=Cc1cc(N2CCC(N(CCOC)c3ccnc(Nc4cnc(CC)c(F)c4)n3)CC2)ccn1] | A386 |
| C₂₆H₃₂FN₇O | |
| 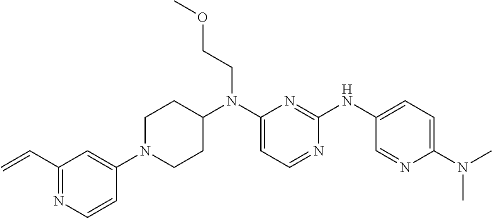 [SMILES: C=Cc1cc(N2CCC(N(CCOC)c3ccnc(Nc4ccc(N(C)C)nc4)n3)CC2)ccn1] | A387 |
| C₂₆H₃₄N₈O | |
| 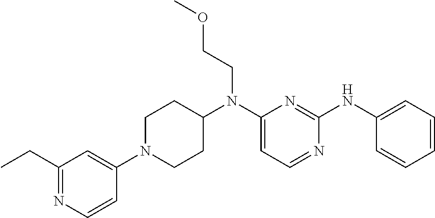 [SMILES: CCc1cc(N2CCC(N(CCOC)c3ccnc(Nc4ccccc4)n3)CC2)ccn1] | A388 |
| C₂₅H₃₂N₆O | |
| 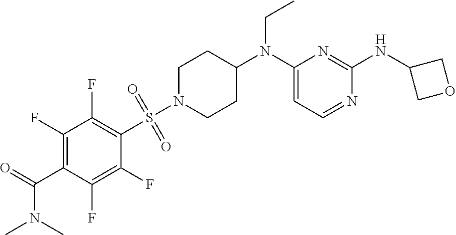 [SMILES: CCN(c1ccnc(NC2COC2)n1)C1CCN(S(=O)(=O)c2c(F)c(F)c(C(=O)N(C)C)c(F)c2F)CC1] | A389 |
| C₂₃H₂₈F₄N₆O₄S | |
| 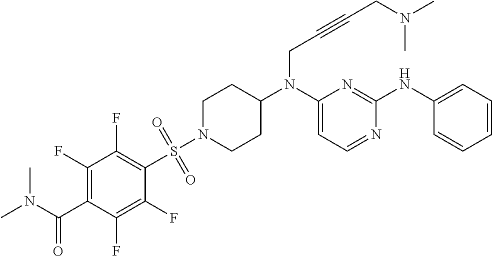 [SMILES: CN(C)CC#CCN(c1ccnc(Nc2ccccc2)n1)C1CCN(S(=O)(=O)c2c(F)c(F)c(C(=O)N(C)C)c(F)c2F)CC1] | A390 |
| C₃₀H₃₃F₄N₇O₃S | |
| 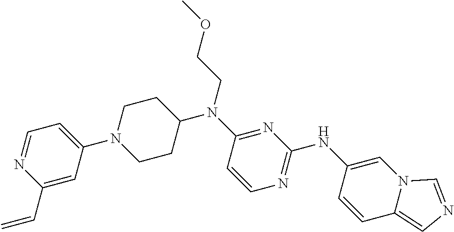 [SMILES: C=Cc1cc(N2CCC(N(CCOC)c3ccnc(Nc4ccc5cncn5c4)n3)CC2)ccn1] | A391 |
| C₂₆H₃₀N₈O | |
| 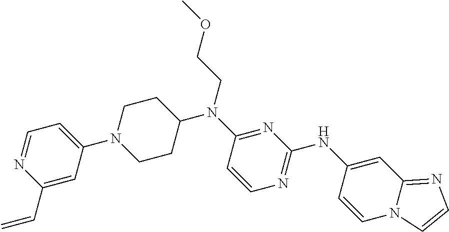 [SMILES: C=Cc1cc(N2CCC(N(CCOC)c3ccnc(Nc4ccn5ccnc5c4)n3)CC2)ccn1] | A392 |
| C₂₆H₃₀N₈O | |
| 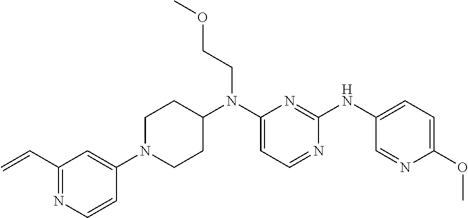 [SMILES: C=Cc1cc(N2CCC(N(CCOC)c3ccnc(Nc4ccc(OC)nc4)n3)CC2)ccn1] | A393 |
| C₂₅H₃₁N₇O₂ | |
| 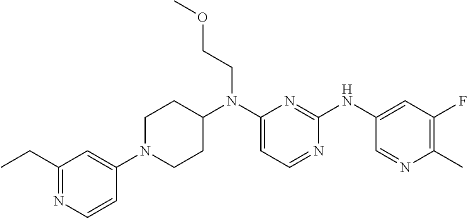 [SMILES: CCc1cc(N2CCC(N(CCOC)c3ccnc(Nc4cnc(C)c(F)c4)n3)CC2)ccn1] | A394 |
| C₂₅H₃₂FN₇O | |
| 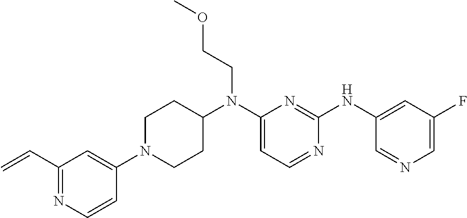 [SMILES: C=Cc1cc(N2CCC(N(CCOC)c3ccnc(Nc4cncc(F)c4)n3)CC2)ccn1] | A395 |
| C₂₄H₂₈FN₇O | |
| 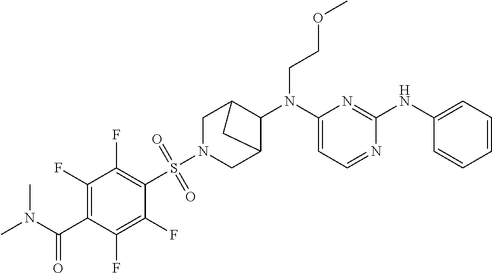 [SMILES: COCCN(c1ccnc(Nc2ccccc2)n1)C1C2CC1CN(S(=O)(=O)c1c(F)c(F)c(C(=O)N(C)C)c(F)c1F)C2] | A396 |
| C₂₈H₃₀F₄N₆O₄S | |
| 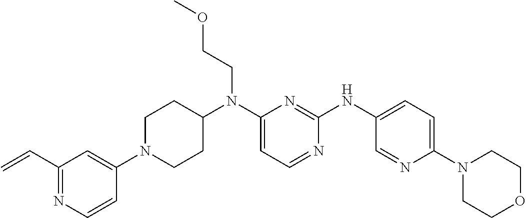 [SMILES: C=Cc1cc(N2CCC(N(CCOC)c3ccnc(Nc4ccc(N5CCOCC5)nc4)n3)CC2)ccn1] | A397 |
| C₂₈H₃₆N₈O₂ | |
| 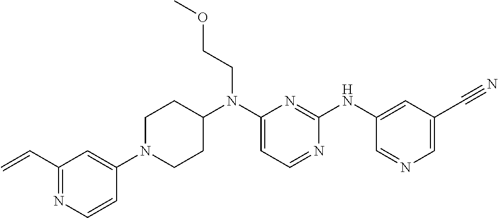 [SMILES: C=Cc1cc(N2CCC(N(CCOC)c3ccnc(Nc4cncc(C#N)c4)n3)CC2)ccn1] | A398 |
| C₂₅H₂₈N₈O | |
| 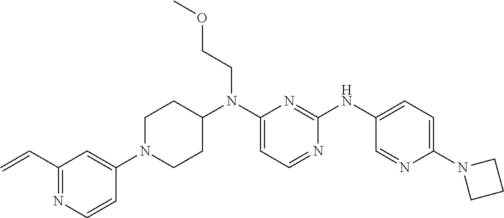 [SMILES: C=Cc1cc(N2CCC(N(CCOC)c3ccnc(Nc4ccc(N5CCC5)nc4)n3)CC2)ccn1] | A399 |
| C₂₇H₃₄N₈O | |
| 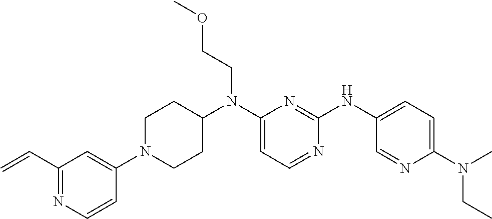 [SMILES: C=Cc1cc(N2CCC(N(CCOC)c3ccnc(Nc4ccc(N(C)CC)nc4)n3)CC2)ccn1] | A400 |
| C₂₇H₃₆N₈O | |
| 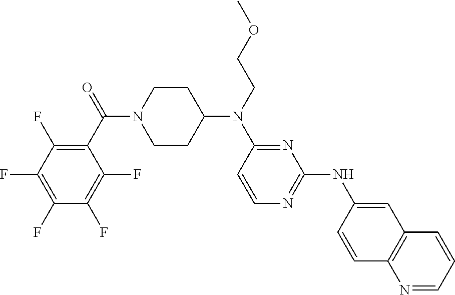 [SMILES: COCCN(c1ccnc(Nc2ccc3ncccc3c2)n1)C1CCN(C(=O)c2c(F)c(F)c(F)c(F)c2F)CC1] | A401 |
| C₂₈H₂₅F₅N₆O2 | |
| 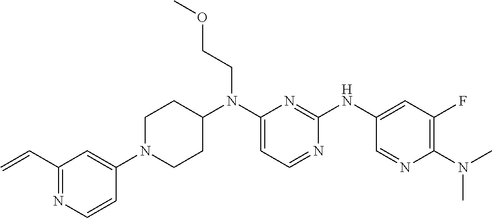 [SMILES: C=Cc1cc(N2CCC(N(CCOC)c3ccnc(Nc4cnc(N(C)C)c(F)c4)n3)CC2)ccn1] | A402 |
| C₂₆H₃₃FN₈O | |
| 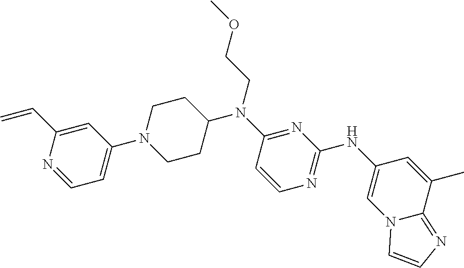 [SMILES: C=Cc1cc(N2CCC(N(CCOC)c3ccnc(Nc4cc(C)c5nccn5c4)n3)CC2)ccn1] | A403 |
| C₂₇H₃₂N₈O | |
| 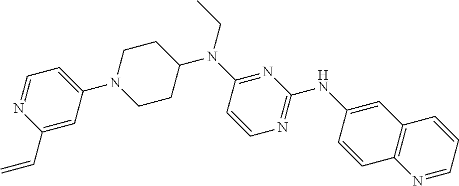 [SMILES: C=Cc1cc(N2CCC(N(CC)c3ccnc(Nc4ccc5ncccc5c4)n3)CC2)ccn1] | A404 |
| C₂₇H₂₉N₇ | |
| 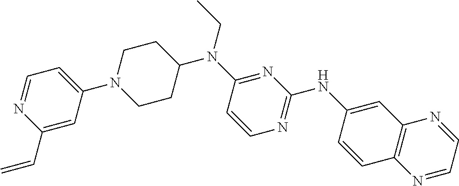 [SMILES: C=Cc1cc(N2CCC(N(CC)c3ccnc(Nc4ccc5nccnc5c4)n3)CC2)ccn1] | A405 |
| C₂₆H₂₈N₈ | |
| 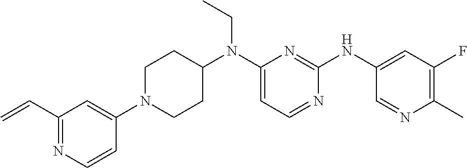 [SMILES: C=Cc1cc(N2CCC(N(CC)c3ccnc(Nc4cnc(C)c(F)c4)n3)CC2)ccn1] | A406 |
| C₂₄H₂₈FN₇ | |
| 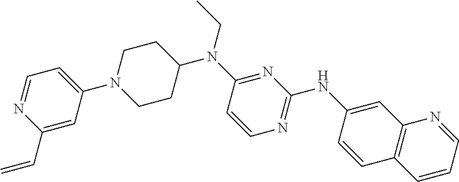 [SMILES: C=Cc1cc(N2CCC(N(CC)c3ccnc(Nc4ccc5cccnc5c4)n3)CC2)ccn1] | A407 |
| C₂₇H₂₉N₇ | |
| 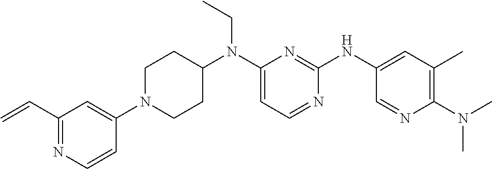 [SMILES: C=Cc1cc(N2CCC(N(CC)c3ccnc(Nc4cnc(N(C)C)c(C)c4)n3)CC2)ccn1] | A408 |
| C₂₆H₃₄N₈ | |
| 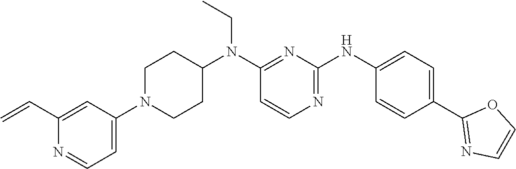 [SMILES: C=Cc1cc(N2CCC(N(CC)c3ccnc(Nc4ccc(-c5ncco5)cc4)n3)CC2)ccn1] | A409 |
| C₂₇H₂₉N₇O | |
| 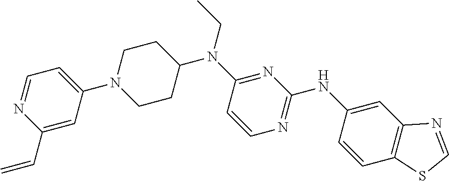 [SMILES: C=Cc1cc(N2CCC(N(CC)c3ccnc(Nc4ccc5scnc5c4)n3)CC2)ccn1] | A410 |
| C₂₅H₂₇N₇S | |
| 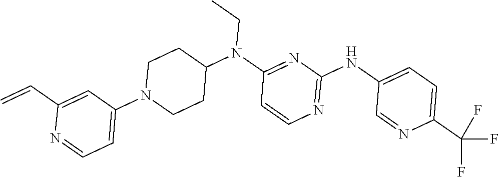 [SMILES: C=Cc1cc(N2CCC(N(CC)c3ccnc(Nc4ccc(C(F)(F)F)nc4)n3)CC2)ccn1] | A411 |
| C₂₄H₂₆F₃N₇ | |
| 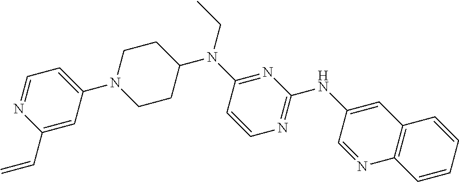 [SMILES: C=Cc1cc(N2CCC(N(CC)c3ccnc(Nc4cnc5ccccc5c4)n3)CC2)ccn1] | A412 |
| C₂₇H₂₉N₇ | |
| 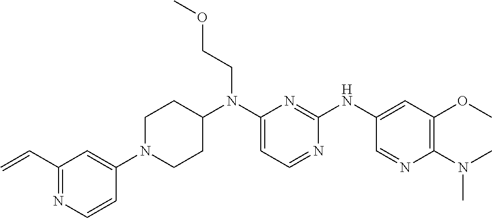 [SMILES: C=Cc1cc(N2CCC(N(CCOC)c3ccnc(Nc4cnc(N(C)C)c(OC)c4)n3)CC2)ccn1] | A413 |
| C₂₇H₃₆N₈O₂ | |
| 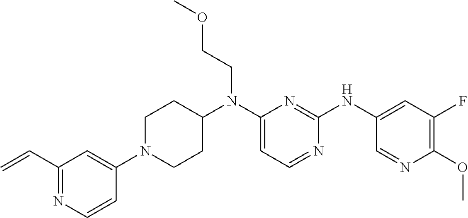 [SMILES: C=Cc1cc(N2CCC(N(CCOC)c3ccnc(Nc4cnc(OC)c(F)c4)n3)CC2)ccn1] | A414 |
| C₂₅H₃₀FN₇O₂ | |
| 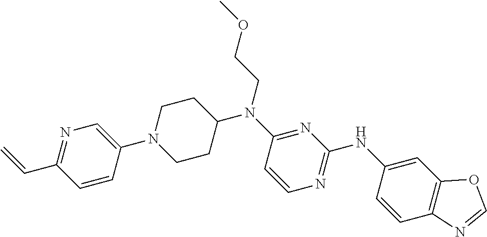 [SMILES: C=Cc1ccc(N2CCC(N(CCOC)c3ccnc(Nc4ccc5ncoc5c4)n3)CC2)cn1] | A415 |
| C₂₆H₂₉N₇O₂ | |
| 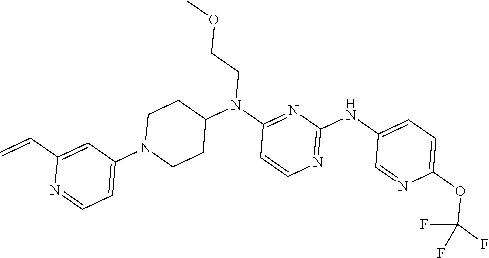 [SMILES: C=Cc1cc(N2CCC(N(CCOC)c3ccnc(Nc4ccc(OC(F)(F)F)nc4)n3)CC2)ccn1] | A416 |
| C₂₅H₂₈F₃N₇O₂ | |
| 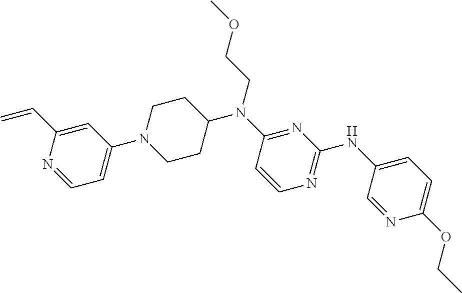 [SMILES: C=Cc1cc(N2CCC(N(CCOC)c3ccnc(Nc4ccc(OCC)nc4)n3)CC2)ccn1] | A417 |
| C₂₆H₃₃N₇O₂ | |
| 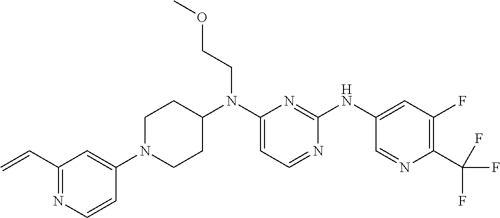 [SMILES: C=Cc1cc(N2CCC(N(CCOC)c3ccnc(Nc4cnc(C(F)(F)F)c(F)c4)n3)CC2)ccn1] | A418 |
| C₂₅H₂₇F₄N₇O | |
| 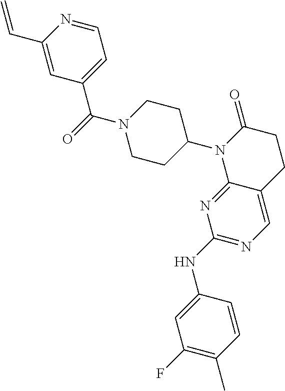 [SMILES: C=Cc1cc(C(=O)N2CCC(N3C(=O)CCc4cnc(Nc5ccc(C)c(F)c5)nc43)CC2)ccn1] | A419 |
| C₂₇H₂₇FN₆O₂ | |
| 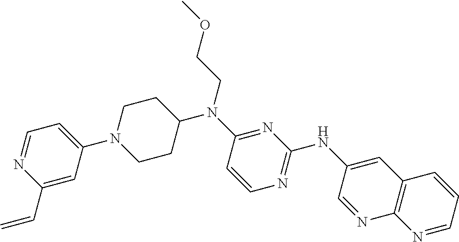 [SMILES: C=Cc1cc(N2CCC(N(CCOC)c3ccnc(Nc4cnc5ncccc5c4)n3)CC2)ccn1] | A420 |
| C₂₇H₃₀N₈O | |
| 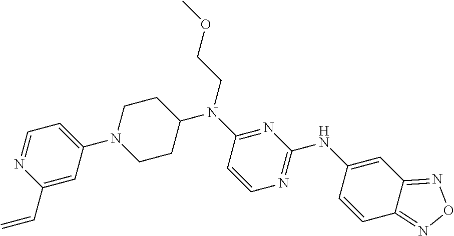 [SMILES: C=Cc1cc(N2CCC(N(CCOC)c3ccnc(Nc4ccc5nonc5c4)n3)CC2)ccn1] | A421 |
| C₂₅H₂₈N₈O₂ | |
| 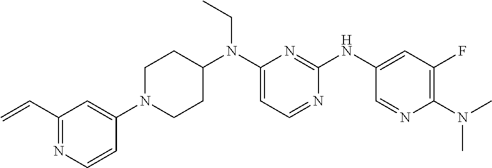 [SMILES: C=Cc1cc(N2CCC(N(CC)c3ccnc(Nc4cnc(N(C)C)c(F)c4)n3)CC2)ccn1] | A422 |
| C₂₅H₃₁FN₈ | |
| 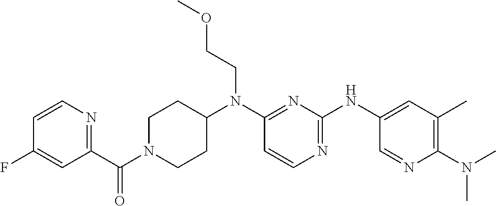 [SMILES: COCCN(c1ccnc(Nc2cnc(N(C)C)c(C)c2)n1)C1CCN(C(=O)c2cc(F)ccn2)CC1] | A423 |
| C₂₆H₃₃FN₈O₂ | |
| 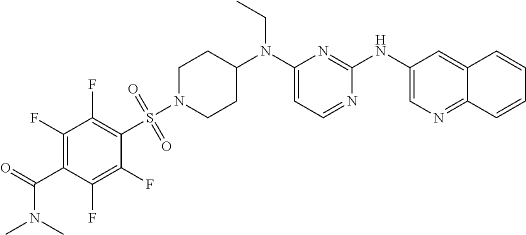 [SMILES: CCN(c1ccnc(Nc2cnc3ccccc3c2)n1)C1CCN(S(=O)(=O)c2c(F)c(F)c(C(=O)N(C)C)c(F)c2F)CC1] | A424 |
| C₂₉H₂₉F₄N₇O₃S | |
| 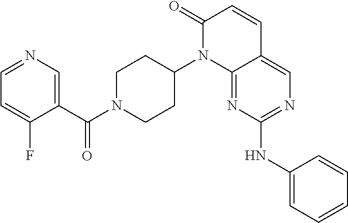 [SMILES: O=C(c1cnccc1F)N1CCC(n2c(=O)ccc3cnc(Nc4ccccc4)nc32)CC1] | A425 |
| C₂₄H₂₁FN₆O₂ | |
| 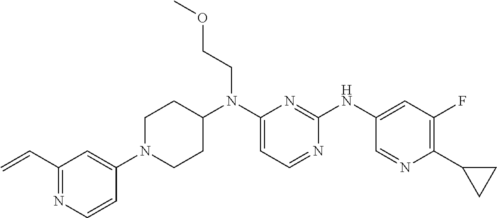 [SMILES: C=Cc1cc(N2CCC(N(CCOC)c3ccnc(Nc4cnc(C5CC5)c(F)c4)n3)CC2)ccn1] | A426 |
| C₂₇H₃₂FN₇O | |
| 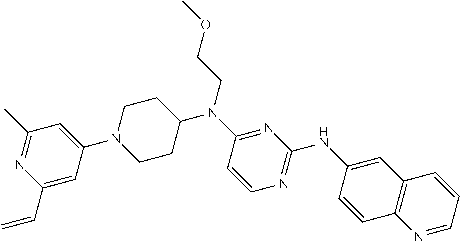 [SMILES: C=Cc1cc(N2CCC(N(CCOC)c3ccnc(Nc4ccc5ncccc5c4)n3)CC2)cc(C)n1] | A427 |
| C₂₉H₃₃N₇O | |
| 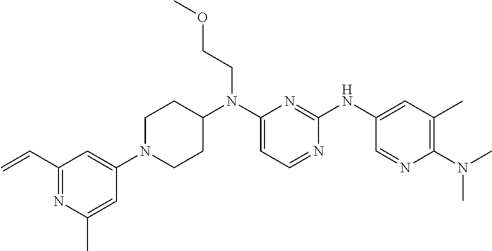 [SMILES: C=Cc1cc(N2CCC(N(CCOC)c3ccnc(Nc4cnc(N(C)C)c(C)c4)n3)CC2)cc(C)n1] | A428 |
| C₂₈H₃₈N₈O | |
| 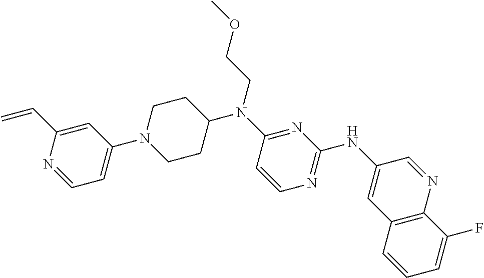 [SMILES: C=Cc1cc(N2CCC(N(CCOC)c3ccnc(Nc4cnc5c(F)cccc5c4)n3)CC2)ccn1] | A429 |
| C₂₈H₃₀FN₇O | |
| 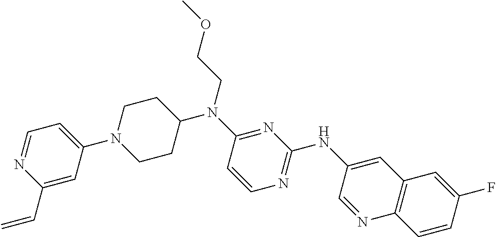 [SMILES: C=Cc1cc(N2CCC(N(CCOC)c3ccnc(Nc4cnc5ccc(F)cc5c4)n3)CC2)ccn1] | A430 |
| C₂₈H₃₀FN₇O | |
| 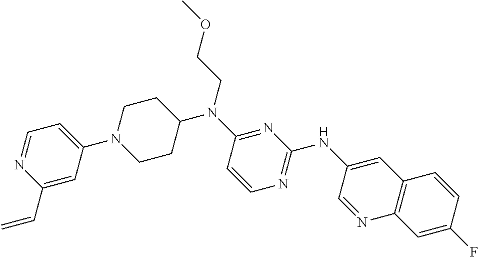 [SMILES: C=Cc1cc(N2CCC(N(CCOC)c3ccnc(Nc4cnc5cc(F)ccc5c4)n3)CC2)ccn1] | A431 |
| C₂₈H₃₀FN₇O | |
| 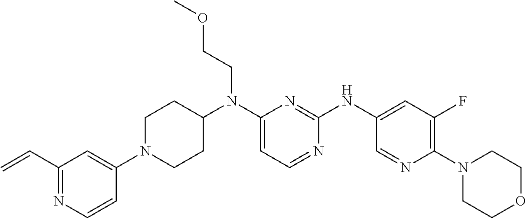 [SMILES: C=Cc1cc(N2CCC(N(CCOC)c3ccnc(Nc4cnc(N5CCOCC5)c(F)c4)n3)CC2)ccn1] | A432 |
| C₂₈H₃₅FN₈O₂ | |
| 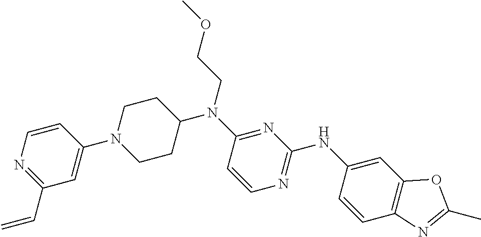 [SMILES: C=Cc1cc(N2CCC(N(CCOC)c3ccnc(Nc4ccc5nc(C)oc5c4)n3)CC2)ccn1] | A433 |
| C₂₇H₃₁N₇O₂ | |
| 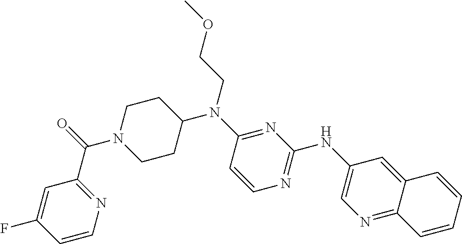 [SMILES: COCCN(c1ccnc(Nc2cnc3ccccc3c2)n1)C1CCN(C(=O)c2cc(F)ccn2)CC1] | A434 |
| C₂₇H₂₈FN₇O₂ | |
| 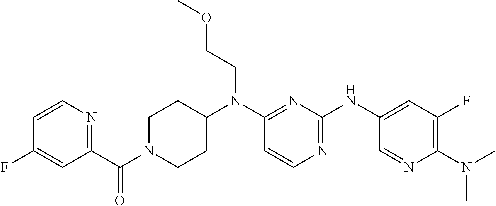 [SMILES: COCCN(c1ccnc(Nc2cnc(N(C)C)c(F)c2)n1)C1CCN(C(=O)c2cc(F)ccn2)CC1] | A435 |
| C₂₅H₃₀F₂N₈O₂ | |
| 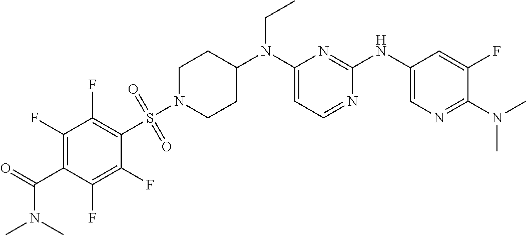 [SMILES: CCN(c1ccnc(Nc2cnc(N(C)C)c(F)c2)n1)C1CCN(S(=O)(=O)c2c(F)c(F)c(C(=O)N(C)C)c(F)c2F)CC1] | A436 |
| C₂₇H₃₁F₅N₈O₃S | |
| 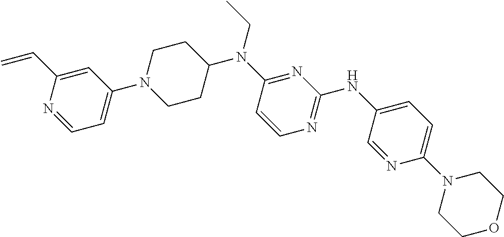 [SMILES: C=Cc1cc(N2CCC(N(CC)c3ccnc(Nc4ccc(N5CCOCC5)nc4)n3)CC2)ccn1] | A437 |
| C₂₇H₃₄N₈O | |
| 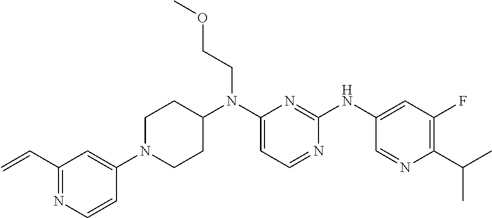 [SMILES: C=Cc1cc(N2CCC(N(CCOC)c3ccnc(Nc4cnc(C(C)C)c(F)c4)n3)CC2)ccn1] | A438 |
| C₂₇H₃₄FN₇O | |
| 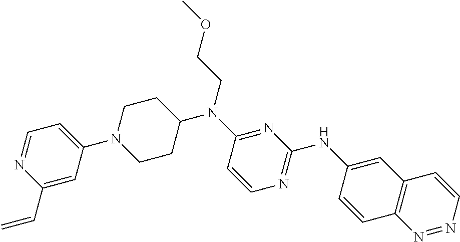 [SMILES: C=Cc1cc(N2CCC(N(CCOC)c3ccnc(Nc4ccc5nnccc5c4)n3)CC2)ccn1] | A439 |
| C₂₇H₃₀N₈O | |
| 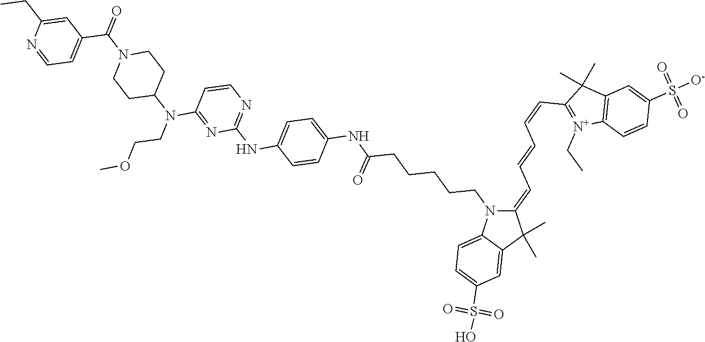 [SMILES: CCc1cc(C(=O)N2CCC(N(CCOC)c3ccnc(Nc4ccc(NC(=O)CCCCCN5\C(=C/C=C/C=C\C6=[N+](CC)c7ccc(S(=O)(=O)[O-])cc7C6(C)C)C(C)(C)c6cc(S(=O)(=O)O)ccc65)cc4)n3)CC2)ccn1] | A440 |
| C₅₉H₇₁N₉O₉S₂ | |
| 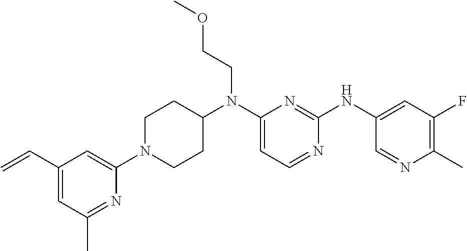 [SMILES: C=Cc1cc(C)nc(N2CCC(N(CCOC)c3ccnc(Nc4cnc(C)c(F)c4)n3)CC2)c1] | A441 |
| C₂₆H₃₂FN₇O | |
| 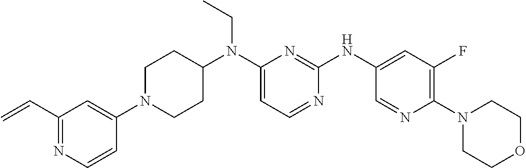 [SMILES: C=Cc1cc(N2CCC(N(CC)c3ccnc(Nc4cnc(N5CCOCC5)c(F)c4)n3)CC2)ccn1] | A442 |
| C₂₇H₃₃FN₈O | |
| 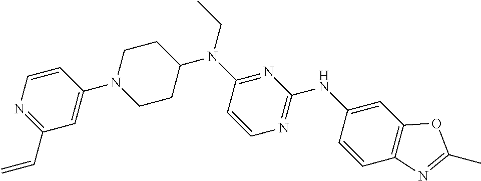 [SMILES: C=Cc1cc(N2CCC(N(CC)c3ccnc(Nc4ccc5nc(C)oc5c4)n3)CC2)ccn1] | A443 |
| C₂₆H₂₉N₇O | |
| 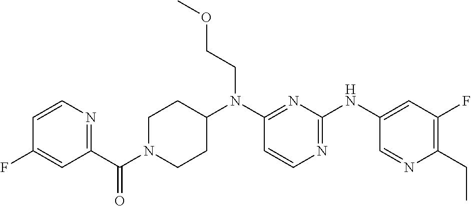 [SMILES: CCc1ncc(Nc2nccc(N(CCOC)C3CCN(C(=O)c4cc(F)ccn4)CC3)n2)cc1F] | A444 |
| C₂₅H₂₉F₂N₇O₂ | |
| 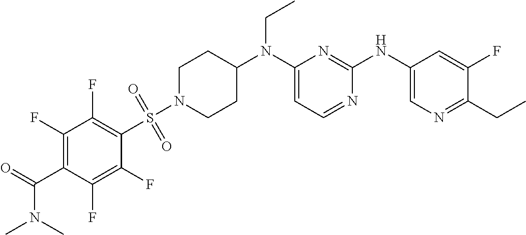 [SMILES: CCc1ncc(Nc2nccc(N(CC)C3CCN(S(=O)(=O)c4c(F)c(F)c(C(=O)N(C)C)c(F)c4F)CC3)n2)cc1F] | A445 |
| C₂₇H₃₀F₅ₗN₇O₃S | |
| 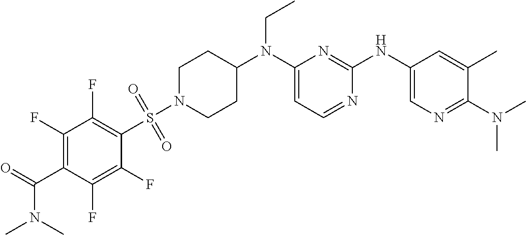 [SMILES: CCN(c1ccnc(Nc2cnc(N(C)C)c(C)c2)n1)C1CCN(S(=O)(=O)c2c(F)c(F)c(C(=O)N(C)C)c(F)c2F)CC1] | A446 |
| C₂₈H₃₄F₄N₈O₃S | |
| 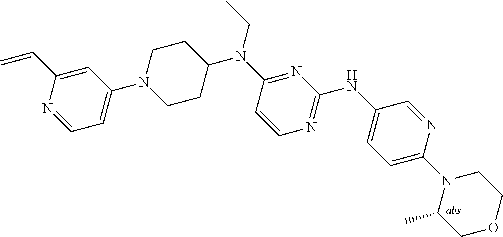 [SMILES: C=Cc1cc(N2CCC(N(CC)c3ccnc(Nc4ccc(N5CCOC[C@@H]5C)nc4)n3)CC2)ccn1] | A447 |
| C₂₈H₃₆N₈O | |
| 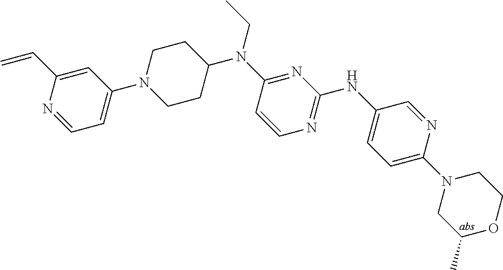 [SMILES: C=Cc1cc(N2CCC(N(CC)c3ccnc(Nc4ccc(N5CCO[C@H](C)C5)nc4)n3)CC2)ccn1] | A448 |
| C₂₈H₃₆N₈O | |
| 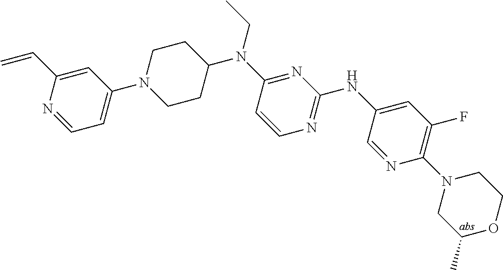 [SMILES: C=Cc1cc(N2CCC(N(CC)c3ccnc(Nc4cnc(N5CCO[C@H](C)C5)c(F)c4)n3)CC2)ccn1] | A449 |
| C₂₈H₃₅FN₈O | |
| 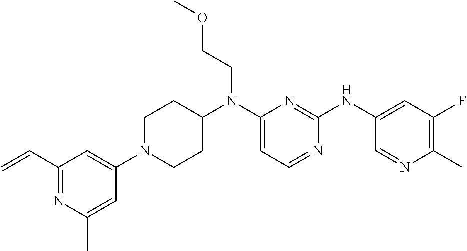 [SMILES: C=Cc1cc(N2CCC(N(CCOC)c3ccnc(Nc4cnc(C)c(F)c4)n3)CC2)cc(C)n1] | A450 |
| C₂₆H₃₂FN₇O | |
| 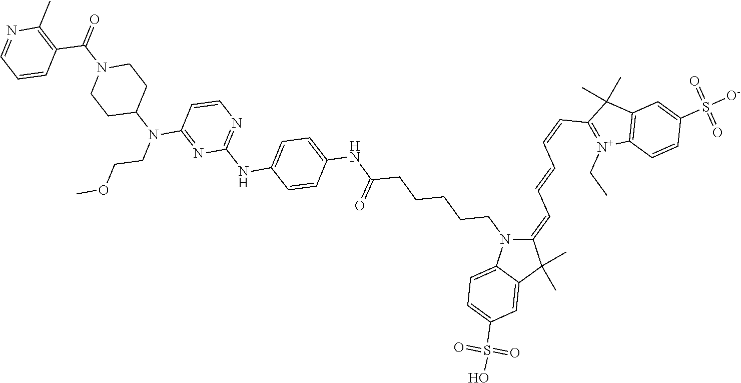 [SMILES: CC[N+]1=C(\C=C/C=C/C=C2\N(CCCCCC(=O)Nc3ccc(Nc4nccc(N(CCOC)C5CCN(C(=O)c6cccnc6C)CC5)n4)cc3)c3ccc(S(=O)(=O)O)cc3C2(C)C)C(C)(C)c2cc(S(=O)(=O)[O-])ccc21] | A451 |
| C₅₈H₆₉N₉O₉S₂ | |
| 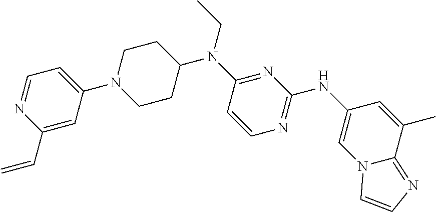 [SMILES: C=Cc1cc(N2CCC(N(CC)c3ccnc(Nc4cc(C)c5nccn5c4)n3)CC2)ccn1] | A452 |
| C₂₆H₃₀N₈ | |
| 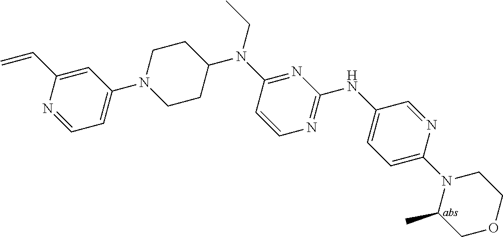 [SMILES: C=Cc1cc(N2CCC(N(CC)c3ccnc(Nc4ccc(N5CCOC[C@H]5C)nc4)n3)CC2)ccn1] | A453 |
| C₂₈H₃₆N₈O | |
| 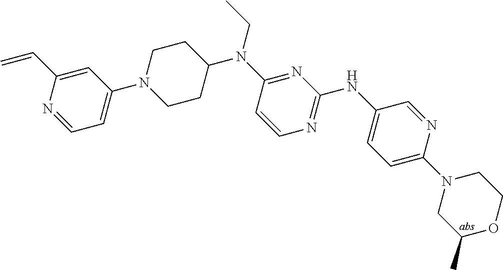 [SMILES: C=Cc1cc(N2CCC(N(CC)c3ccnc(Nc4ccc(N5CCO[C@@H](C)C5)nc4)n3)CC2)ccn1] | A454 |
| C₂₈H₃₆N₈O | |
| 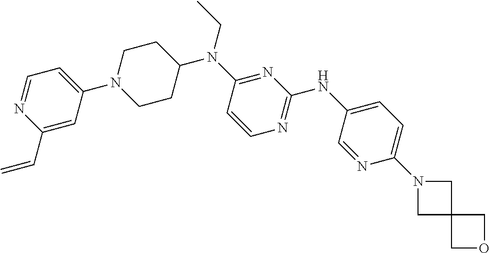 [SMILES: C=Cc1cc(N2CCC(N(CC)c3ccnc(Nc4ccc(N5CC6(COC6)C5)nc4)n3)CC2)ccn1] | A455 |
| C₂₈H₃₄N₈O | |
| 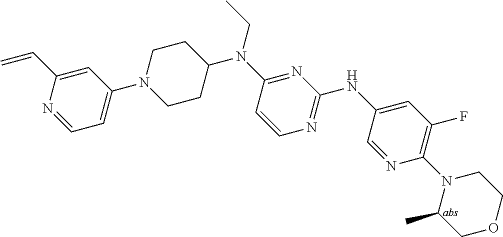 [SMILES: C=Cc1cc(N2CCC(N(CC)c3ccnc(Nc4cnc(N5CCOC[C@H]5C)c(F)c4)n3)CC2)ccn1] | A456 |
| C₂₈H₃₅FN₈O | |
| 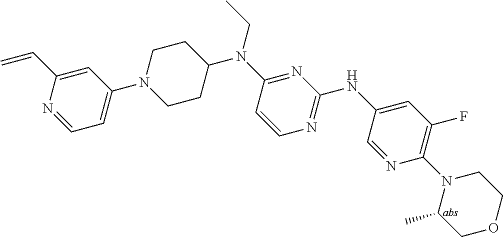 [SMILES: C=Cc1cc(N2CCC(N(CC)c3ccnc(Nc4cnc(N5CCOC[C@@H]5C)c(F)c4)n3)CC2)ccn1] | A457 |
| C₂₈H₃₅FN₈O | |
| 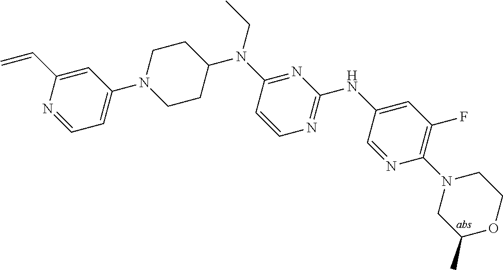 [SMILES: C=Cc1cc(N2CCC(N(CC)c3ccnc(Nc4cnc(N5CCO[C@@H](C)C5)c(F)c4)n3)CC2)ccn1] | A458 |
| C₂₈H₃₅FN₈O | |
| 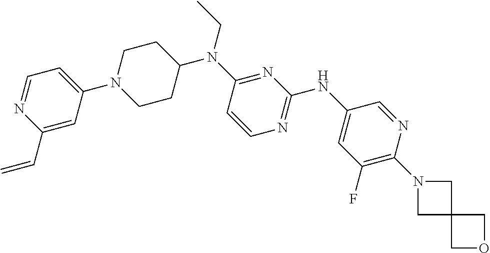 [SMILES: C=Cc1cc(N2CCC(N(CC)c3ccnc(Nc4cnc(N5CC6(COC6)C5)c(F)c4)n3)CC2)ccn1] | A459 |
| C₂₈H₃₃FN₈O | |
| 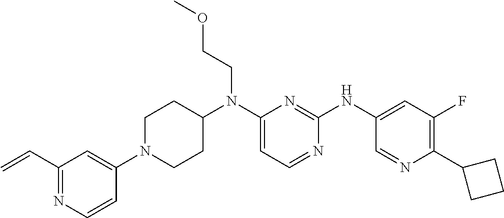 [SMILES: C=Cc1cc(N2CCC(N(CCOC)c3ccnc(Nc4cnc(C5CCC5)c(F)c4)n3)CC2)ccn1] | A460 |
| C₂₈H₃₄FN₇O | |
| 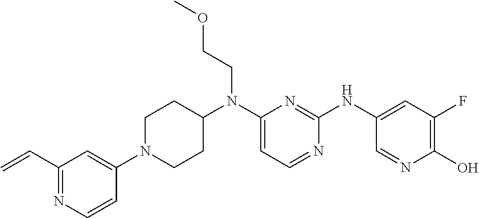 [SMILES: C=Cc1cc(N2CCC(N(CCOC)c3ccnc(Nc4cnc(O)c(F)c4)n3)CC2)ccn1] | A461 |
| C₂₄H₂₈FN₇O₂ | |
| 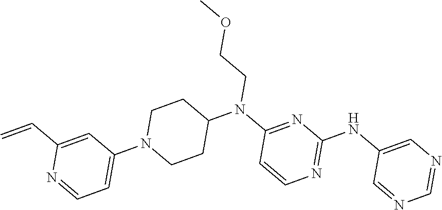 [SMILES: C=Cc1cc(N2CCC(N(CCOC)c3ccnc(Nc4cncnc4)n3)CC2)ccn1] | A462 |
| C₂₃H₂₈N₈O | |
| 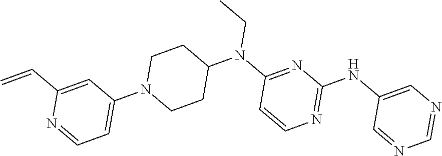 [SMILES: C=Cc1cc(N2CCC(N(CC)c3ccnc(Nc4cncnc4)n3)CC2)ccn1] | A463 |
| C₂₂H₂₆N₈ | |
| 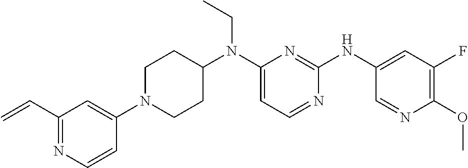 [SMILES: C=Cc1cc(N2CCC(N(CC)c3ccnc(Nc4cnc(OC)c(F)c4)n3)CC2)ccn1] | A464 |
| C₂₄H₂₈FN₇O | |
| 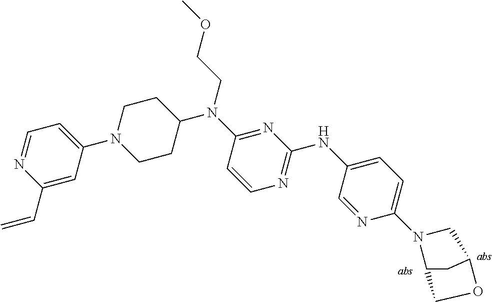 | A465 |
| C₂₉H₃₆N₈O₂ | |
| 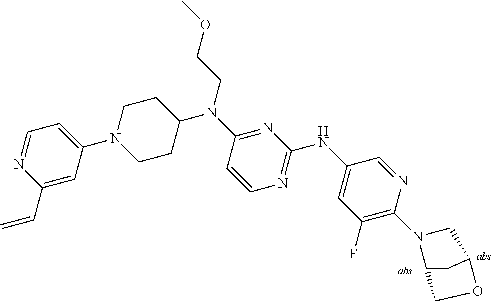 | A466 |
| C₂₉H₃₅FN₈O₂ | |
| 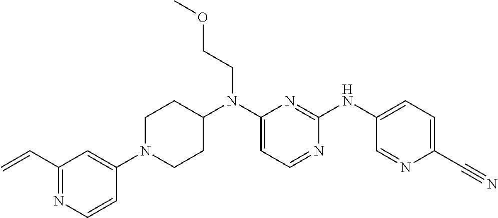 [SMILES: C=Cc1cc(N2CCC(N(CCOC)c3ccnc(Nc4ccc(C#N)nc4)n3)CC2)ccn1] | A467 |
| C₂₅H₂₈N₈O | |
| 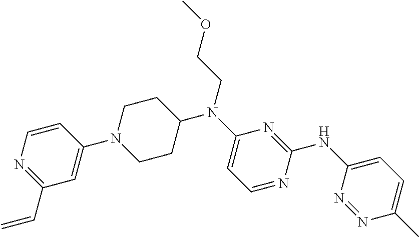 [SMILES: C=Cc1cc(N2CCC(N(CCOC)c3ccnc(Nc4ccc(C)nn4)n3)CC2)ccn1] | A468 |
| C₂₄H₃₀N₈O | |
| 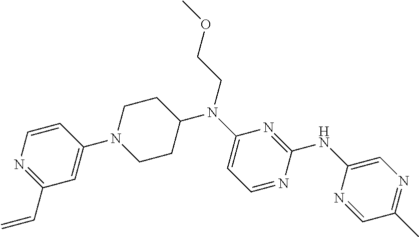 [SMILES: C=Cc1cc(N2CCC(N(CCOC)c3ccnc(Nc4cnc(C)cn4)n3)CC2)ccn1] | A469 |
| C₂₄H₃₀N₈O | |
| 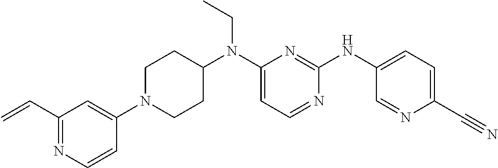 [SMILES: C=Cc1cc(N2CCC(N(CC)c3ccnc(Nc4ccc(C#N)nc4)n3)CC2)ccn1] | A470 |
| C₂₄H₂₆N₈ | |
| 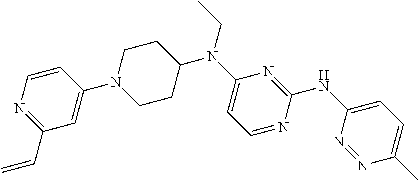 [SMILES: C=Cc1cc(N2CCC(N(CC)c3ccnc(Nc4ccc(C)nn4)n3)CC2)ccn1] | A471 |
| C₂₃H₂₈N₈ | |
| 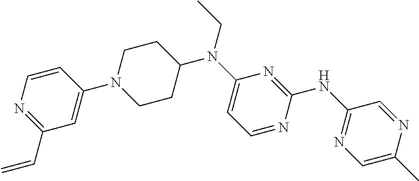 [SMILES: C=Cc1cc(N2CCC(N(CC)c3ccnc(Nc4cnc(C)cn4)n3)CC2)ccn1] | A472 |
| C₂₃H₂₈N₈ | |
| 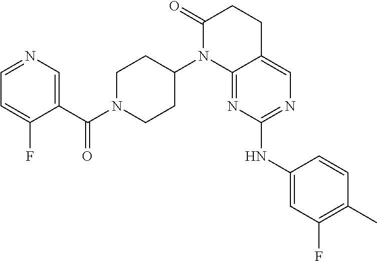 [SMILES: Cc1ccc(Nc2ncc3c(n2)N(C2CCN(C(=O)c4cnccc4F)CC2)C(=O)CC3)cc1F] | A473 |
| C₂₅H₂₄F₂N₆O₂ | |
| 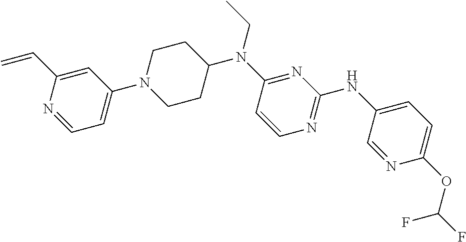 [SMILES: C=Cc1cc(N2CCC(N(CC)c3ccnc(Nc4ccc(OC(F)F)nc4)n3)CC2)ccn1] | A474 |
| C₂₄H₂₇F₂N₇O | |
| 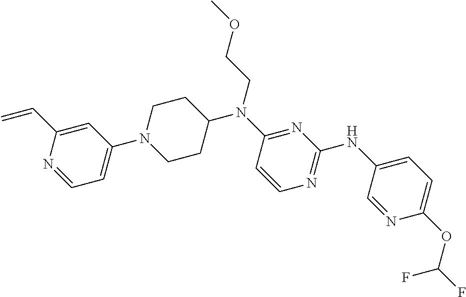 [SMILES: C=Cc1cc(N2CCC(N(CCOC)c3ccnc(Nc4ccc(OC(F)F)nc4)n3)CC2)ccn1] | A475 |
| C₂₅H₂₉F₂N₇O₂ | |
| 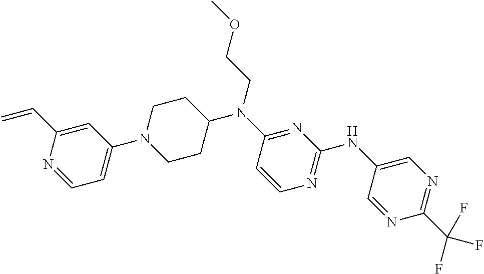 [SMILES: C=Cc1cc(N2CCC(N(CCOC)c3ccnc(Nc4cnc(C(F)(F)F)nc4)n3)CC2)ccn1] | A476 |
| C₂₄H₂₇F₃N₈O | |
| 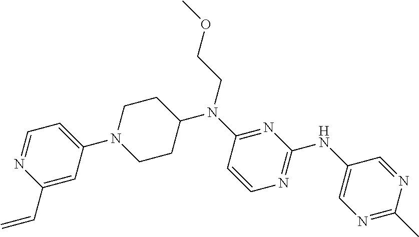 [SMILES: C=Cc1cc(N2CCC(N(CCOC)c3ccnc(Nc4cnc(C)nc4)n3)CC2)ccn1] | A477 |
| C₂₄H₃₀N₈O | |
| 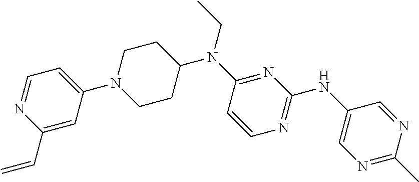 [SMILES: C=Cc1cc(N2CCC(N(CC)c3ccnc(Nc4cnc(C)nc4)n3)CC2)ccn1] | A478 |
| C₂₃H₂₈N₈ | |
| 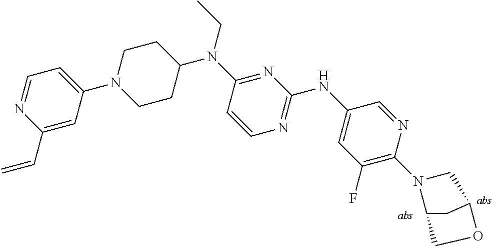 [SMILES: C=Cc1cc(N2CCC(N(CC)c3ccnc(Nc4cnc(N5C[C@H]6C[C@@H]5CO6)c(F)c4)n3)CC2)ccn1] | A479 |
| C₂₈H₃₃FN₈O | |
| 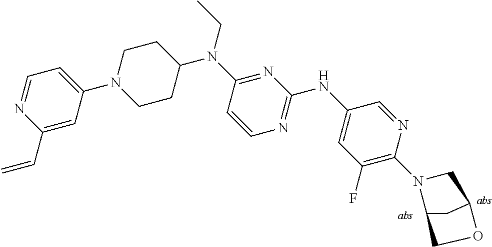 [SMILES: C=Cc1cc(N2CCC(N(CC)c3ccnc(Nc4cnc(N5C[C@@H]6C[C@H]5CO6)c(F)c4)n3)CC2)ccn1] | A480 |
| C₂₈H₃₃FN₈O | |
| 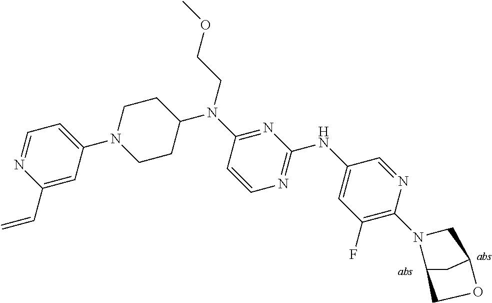 [SMILES: C=Cc1cc(N2CCC(N(CCOC)c3ccnc(Nc4cnc(N5C[C@@H]6C[C@H]5CO6)c(F)c4)n3)CC2)ccn1] | A481 |
| C₂₉H₃₅FN₈O₂ | |
| 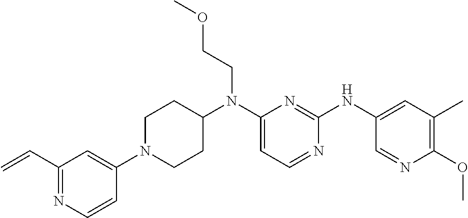 [SMILES: C=Cc1cc(N2CCC(N(CCOC)c3ccnc(Nc4cnc(OC)c(C)c4)n3)CC2)ccn1] | A482 |
| C₂₆H₃₃N₇O₂ | |
| 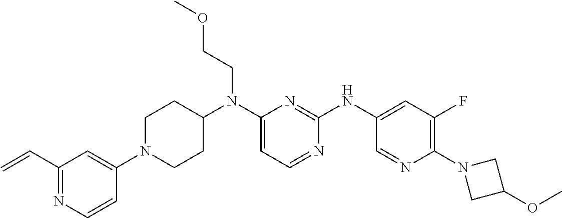 [SMILES: C=Cc1cc(N2CCC(N(CCOC)c3ccnc(Nc4cnc(N5CC(OC)C5)c(F)c4)n3)CC2)ccn1] | A483 |
| C₂₈H₃₅FN₈O₂ | |
| 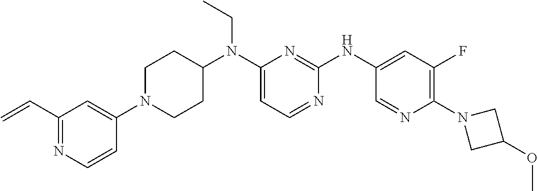 [SMILES: C=Cc1cc(N2CCC(N(CC)c3ccnc(Nc4cnc(N5CC(OC)C5)c(F)c4)n3)CC2)ccn1] | A484 |
| C₂₇H₃₃FN₈O | |
| 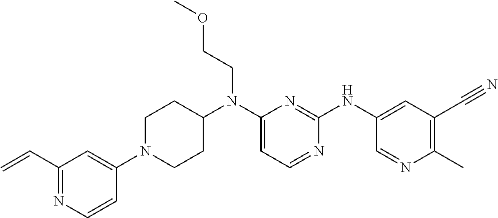 [SMILES: C=Cc1cc(N2CCC(N(CCOC)c3ccnc(Nc4cnc(C)c(C#N)c4)n3)CC2)ccn1] | A485 |
| C₂₆H₃₀N₈O | |
| 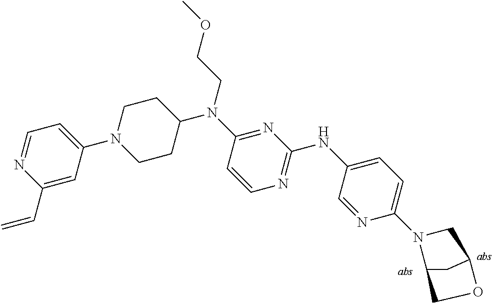 [SMILES: C=Cc1cc(N2CCC(N(CCOC)c3ccnc(Nc4ccc(N5C[C@@H]6C[C@H]5CO6)nc4)n3)CC2)ccn1] | A486 |
| C₂₉H₃₆N₈O₂ | |
| 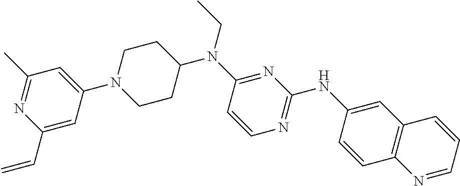 [SMILES: C=Cc1cc(N2CCC(N(CC)c3ccnc(Nc4ccc5ncccc5c4)n3)CC2)cc(C)n1] | A487 |
| C₂₈H₃₁N₇ | |
| 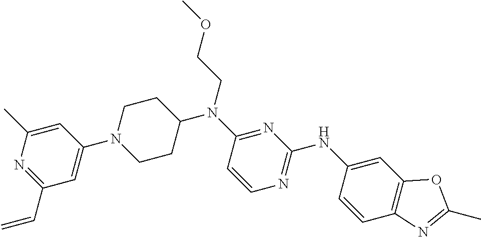 [SMILES: C=Cc1cc(N2CCC(N(CCOC)c3ccnc(Nc4ccc5nc(C)oc5c4)n3)CC2)cc(C)n1] | A488 |
| C₂₈H₃₃N₇O₂ | |
| 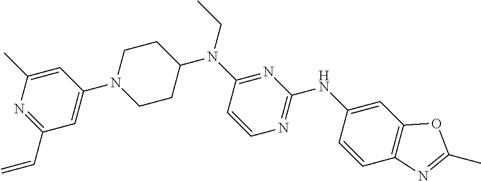 [SMILES: C=Cc1cc(N2CCC(N(CC)c3ccnc(Nc4ccc5nc(C)oc5c4)n3)CC2)cc(C)n1] | A489 |
| C₂₇H₃₁N₇O | |
| 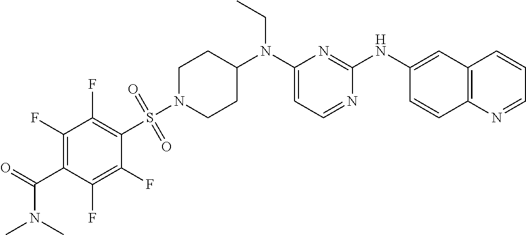 [SMILES: CCN(c1ccnc(Nc2ccc3ncccc3c2)n1)C1CCN(S(=O)(=O)c2c(F)c(F)c(C(=O)N(C)C)c(F)c2F)CC1] | A490 |
| C₂₉H₂₉F₄N₇O₃S | |
| 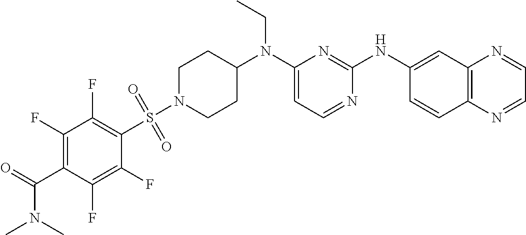 [SMILES: CCN(c1ccnc(Nc2ccc3nccnc3c2)n1)C1CCN(S(=O)(=O)c2c(F)c(F)c(C(=O)N(C)C)c(F)c2F)CC1] | A491 |
| C₂₈H₂₈F₄N₈O₃S | |
| 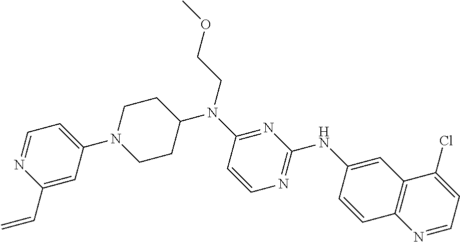 [SMILES: C=Cc1cc(N2CCC(N(CCOC)c3ccnc(Nc4ccc5nccc(Cl)c5c4)n3)CC2)ccn1] | A492 |
| C₂₈H₃₀ClN₇O | |
| 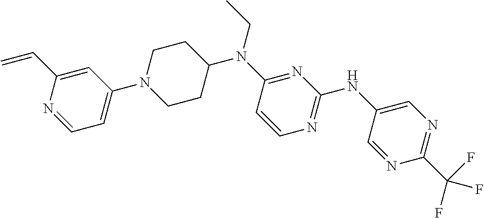 [SMILES: C=Cc1cc(N2CCC(N(CC)c3ccnc(Nc4cnc(C(F)(F)F)nc4)n3)CC2)ccn1] | A493 |
| C₂₃H₂₅F₃N₈ | |
| 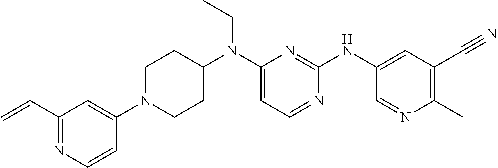 [SMILES: C=Cc1cc(N2CCC(N(CC)c3ccnc(Nc4cnc(C)c(C#N)c4)n3)CC2)ccn1] | A494 |
| C₂₅H₂₈N₈ | |
| 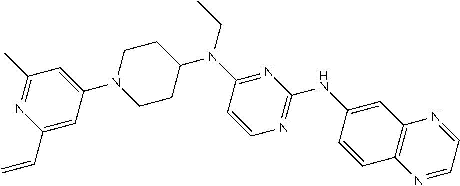 [SMILES: C=Cc1cc(N2CCC(N(CC)c3ccnc(Nc4ccc5nccnc5c4)n3)CC2)cc(C)n1] | A495 |
| C₂₇H₃₀N₈ | |
| 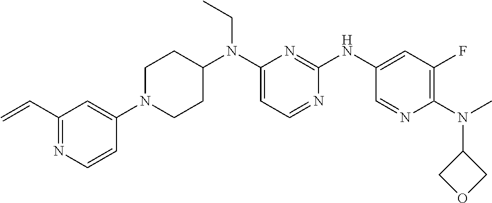 [SMILES: C=Cc1cc(N2CCC(N(CC)c3ccnc(Nc4cnc(N(C)C5COC5)c(F)c4)n3)CC2)ccn1] | A496 |
| C₂₇H₃₃FN₈O | |
| 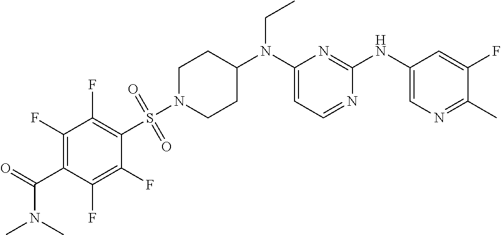 [SMILES: CCN(c1ccnc(Nc2cnc(C)c(F)c2)n1)C1CCN(S(=O)(=O)c2c(F)c(F)c(C(=O)N(C)C)c(F)c2F)CC1] | A497 |
| C₂₆H₂₈F₅N₇O₃S | |
| 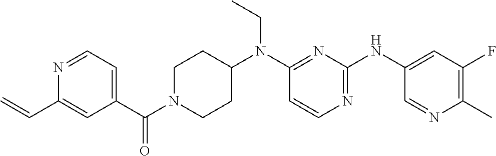 [SMILES: C=Cc1cc(C(=O)N2CCC(N(CC)c3ccnc(Nc4cnc(C)c(F)c4)n3)CC2)ccn1] | A498 |
| C₂₅H₂₈FN₇O | |
| 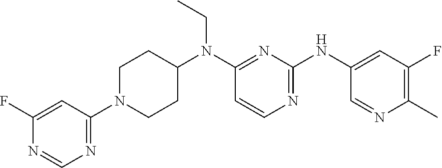 [SMILES: CCN(c1ccnc(Nc2cnc(C)c(F)c2)n1)C1CCN(c2cc(F)ncn2)CC1] | A499 |
| C₂₁H₂₄F₂N₈ | |
| 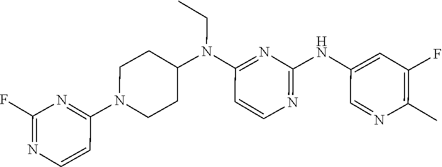 [SMILES: CCN(c1ccnc(Nc2cnc(C)c(F)c2)n1)C1CCN(c2ccnc(F)n2)CC1] | A500 |
| C₂₁H₂₄F₂N₈ | |
| 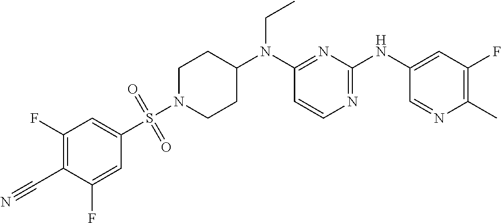 [SMILES: CCN(c1ccnc(Nc2cnc(C)c(F)c2)n1)C1CCN(S(=O)(=O)c2cc(F)c(C#N)c(F)c2)CC1] | A501 |
| C₂₄H₂₄F₃N₇O₂S | |
| 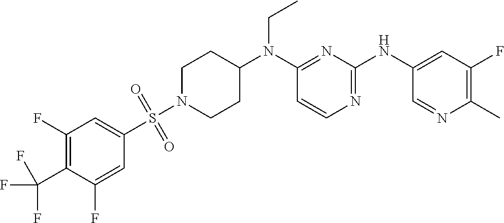 [SMILES: CCN(c1ccnc(Nc2cnc(C)c(F)c2)n1)C1CCN(S(=O)(=O)c2cc(F)c(C(F)(F)F)c(F)c2)CC1] | A502 |
| C₂₄H₂₄F₆N₆O₂S | |
| 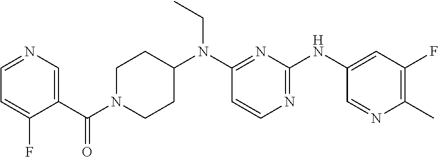 [SMILES: CCN(c1ccnc(Nc2cnc(C)c(F)c2)n1)C1CCN(C(=O)c2cnccc2F)CC1] | A503 |
| C₂₃H₂₅F₂N₇O | |
| 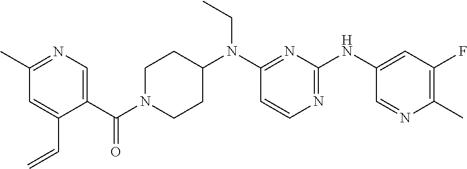 | A504 |
| C₂₆H₃₀FN₇O | |
| 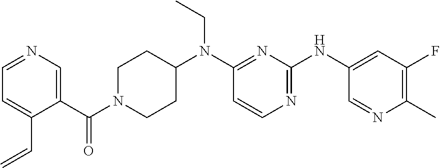 [SMILES: C=Cc1ccncc1C(=O)N1CCC(N(CC)c2ccnc(Nc3cnc(C)c(F)c3)n2)CC1] | A505 |
| C₂₅H₂₈FN₇O | |
| 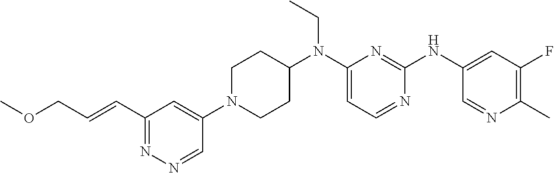 [SMILES: CCN(c1ccnc(Nc2cnc(C)c(F)c2)n1)C1CCN(c2cnnc(/C=C/COC)c2)CC1] | A506 |
| C₂₅H₃₁FN₈O | |
| 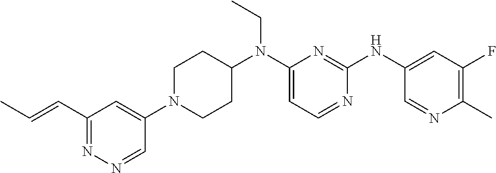 [SMILES: C/C=C/c1cc(N2CCC(N(CC)c3ccnc(Nc4cnc(C)c(F)c4)n3)CC2)cnn1] | A507 |
| C₂₄H₂₉FN₈ | |
| 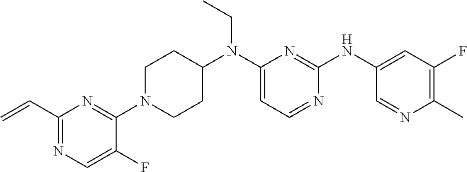 [SMILES: C=Cc1ncc(F)c(N2CCC(N(CC)c3ccnc(Nc4cnc(C)c(F)c4)n3)CC2)n1] | A508 |
| C₂₃H₂₆F₂N₈ | |
| 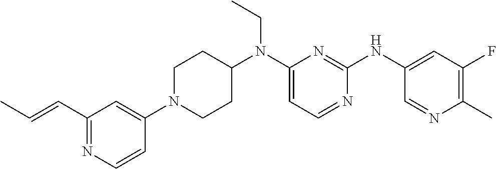 [SMILES: C/C=C/c1cc(N2CCC(N(CC)c3ccnc(Nc4cnc(C)c(F)c4)n3)CC2)ccn1] | A509 |
| C₂₅H₃₀FN₇ | |
| 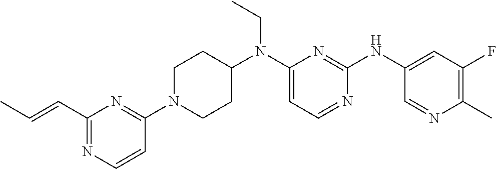 [SMILES: C/C=C/c1nccc(N2CCC(N(CC)c3ccnc(Nc4cnc(C)c(F)c4)n3)CC2)n1] | A510 |
| C₂₄H₂₉FN₈ | |
| 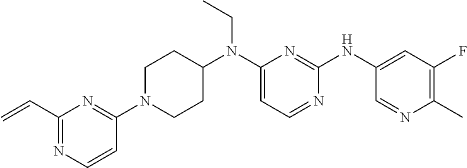 [SMILES: C=Cc1nccc(N2CCC(N(CC)c3ccnc(Nc4cnc(C)c(F)c4)n3)CC2)n1] | A511 |
| C₂₃H₂₇FN₈ | |
| 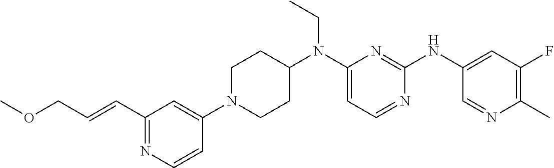 [SMILES: CCN(c1ccnc(Nc2cnc(C)c(F)c2)n1)C1CCN(c2ccnc(/C=C/COC)c2)CC1] | A512 |
| C₂₆H₃₂FN₇O | |
| 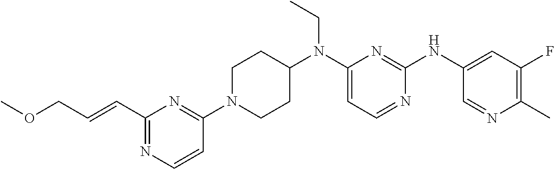 [SMILES: CCN(c1ccnc(Nc2cnc(C)c(F)c2)n1)C1CCN(c2ccnc(/C=C/COC)n2)CC1] | A513 |
| C₂₅H₃₁FN₈O | |
| 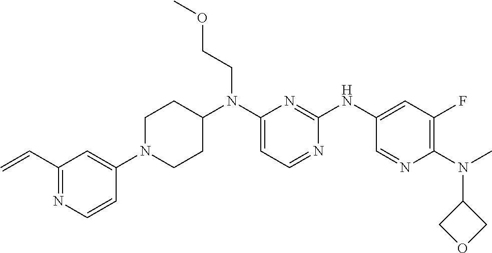 [SMILES: C=Cc1cc(N2CCC(N(CCOC)c3ccnc(Nc4cnc(N(C)C5COC5)c(F)c4)n3)CC2)ccn1] | A514 |
| C₂₈H₃₅FN₈O₂ | |
| 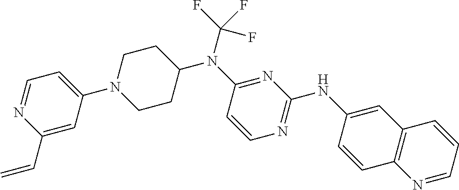 [SMILES: C=Cc1cc(N2CCC(N(c3ccnc(Nc4ccc5ncccc5c4)n3)C(F)(F)F)CC2)ccn1] | A515 |
| C₂₆H₂₄F₃N₇ | |
| 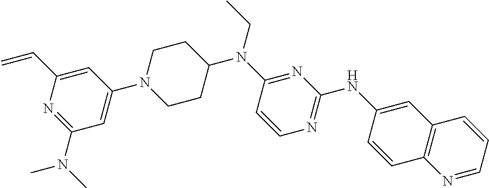 [SMILES: C=Cc1cc(N2CCC(N(CC)c3ccnc(Nc4ccc5ncccc5c4)n3)CC2)cc(N(C)C)n1] | A516 |
| C₂₉H₃₄N₈ | |
| 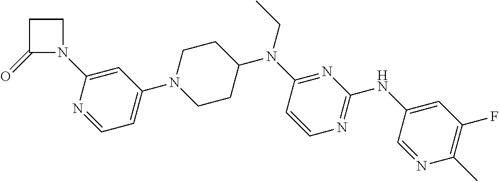 [SMILES: CCN(c1ccnc(Nc2cnc(C)c(F)c2)n1)C1CCN(c2ccnc(N3CCC3=O)c2)CC1] | A517 |
| C₂₅H₂₉FN₈O | |
| 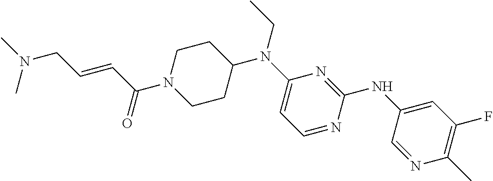 [SMILES: CCN(c1ccnc(Nc2cnc(C)c(F)c2)n1)C1CCN(C(=O)/C=C/CN(C)C)CC1] | A518 |
| C₂₃H₃₂FN₇O | |
| 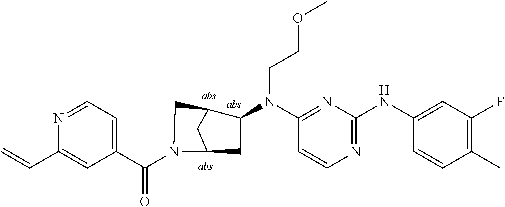 [SMILES: C=Cc1cc(C(=O)N2C[C@@H]3C[C@H]2C[C@@H]3N(CCOC)c2ccnc(Nc3ccc(C)c(F)c3)n2)ccn1] | A519 |
| C₂₈H₃₁FN₆O₂ | |
| 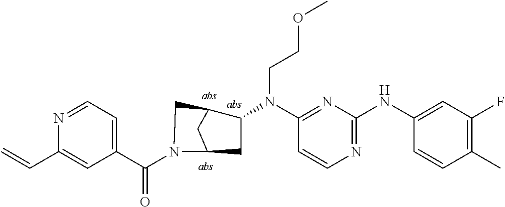 [SMILES: C=Cc1cc(C(=O)N2C[C@@H]3C[C@H]2C[C@H]3N(CCOC)c2ccnc(Nc3ccc(C)c(F)c3)n2)ccn1] | A520 |
| C₂₈H₃₁FN₆O₂ | |
| 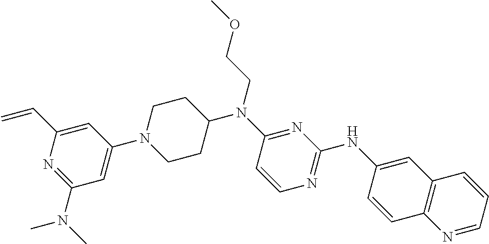 [SMILES: C=Cc1cc(N2CCC(N(CCOC)c3ccnc(Nc4ccc5ncccc5c4)n3)CC2)cc(N(C)C)n1] | A521 |
| C₃₀H₃₆N₈O | |
| 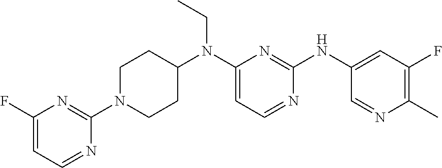 [SMILES: CCN(c1ccnc(Nc2cnc(C)c(F)c2)n1)C1CCN(c2nccc(F)n2)CC1] | A522 |
| C₂₁H₂₄F₂N₈ | |
| 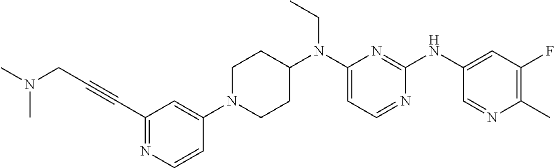 [SMILES: CCN(c1ccnc(Nc2cnc(C)c(F)c2)n1)C1CCN(c2ccnc(C#CCN(C)C)c2)CC1] | A523 |
| C₂₇H₃₃FN₈ | |
| 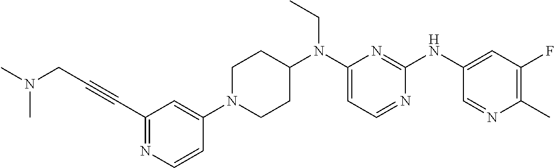 [SMILES: CCN(c1ccnc(Nc2cnc(C)c(F)c2)n1)C1CCN(c2ccnc(C#CCN(C)C)c2)CC1] | A524 |
| C₂₆H₃₂FN₇O | |
| 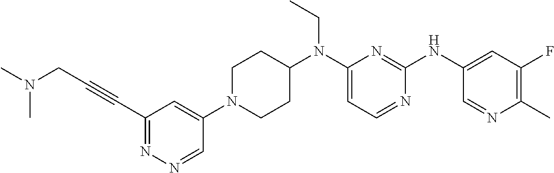 [SMILES: CCN(c1ccnc(Nc2cnc(C)c(F)c2)n1)C1CCN(c2cnnc(C#CCN(C)C)c2)CC1] | A525 |
| C₂₆H₃₂FN₉ | |
| 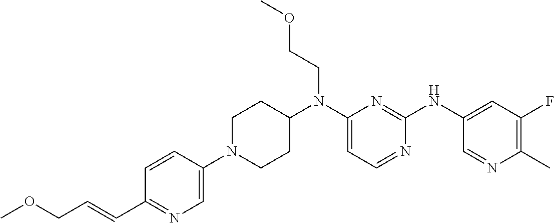 [SMILES: COC/C=C/c1ccc(N2CCC(N(CCOC)c3ccnc(Nc4cnc(C)c(F)c4)n3)CC2)cn1] | A526 |
| C₂₇H₃₄FN₇O₂ | |
| 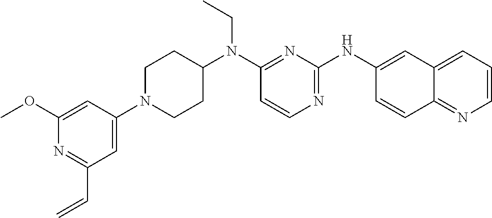 [SMILES: C=Cc1cc(N2CCC(N(CC)c3ccnc(Nc4ccc5ncccc5c4)n3)CC2)cc(OC)n1] | A527 |
| C₂₈H₃₁N₇O | |
| 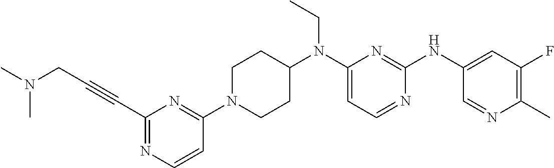 [SMILES: CCN(c1ccnc(Nc2cnc(C)c(F)c2)n1)C1CCN(c2ccnc(C#CCN(C)C)n2)CC1] | A528 |
| C₂₆H₃₂FN₉ | |
| 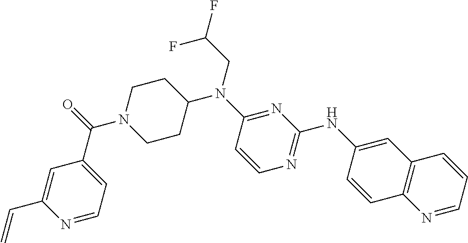 [SMILES: C=Cc1cc(C(=O)N2CCC(N(CC(F)F)c3ccnc(Nc4ccc5ncccc5c4)n3)CC2)ccn1] | A529 |
| C₂₈H₂₇F₂N₇O | |
| 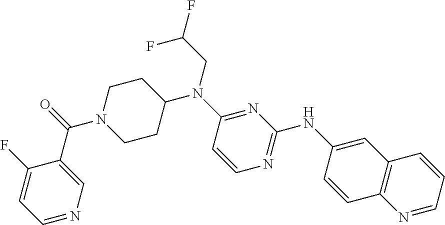 [SMILES: O=C(c1cnccc1F)N1CCC(N(CC(F)F)c2ccnc(Nc3ccc4ncccc4c3)n2)CC1] | A530 |
| C₂₆H₂₄F₃N₇O | |
| 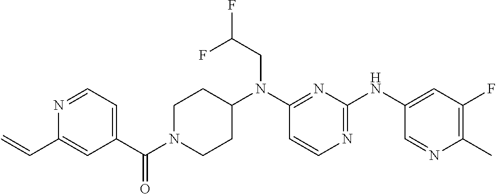 [SMILES: C=Cc1cc(C(=O)N2CCC(N(CC(F)F)c3ccnc(Nc4cnc(C)c(F)c4)n3)CC2)ccn1] | A531 |
| C₂₅H₂₆F₃N₇O | |
| 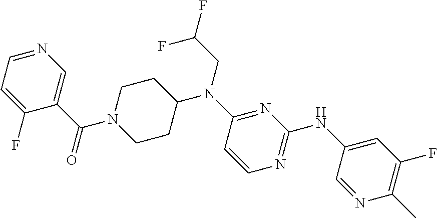 [SMILES: Cc1ncc(Nc2nccc(N(CC(F)F)C3CCN(C(=O)c4cnccc4F)CC3)n2)cc1F] | A532 |
| C₂₃H₂₃F₄N₇O | |
| 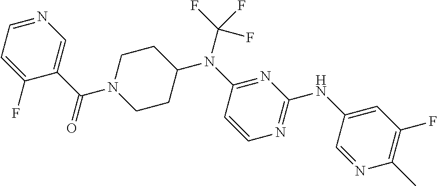 [SMILES: Cc1ncc(Nc2nccc(N(C3CCN(C(=O)c4cnccc4F)CC3)C(F)(F)F)n2)cc1F] | A533 |
| C₂₂H₂₀F₅N₇O | |
| 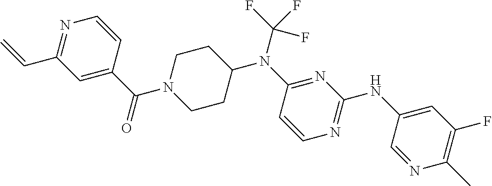 [SMILES: C=Cc1cc(C(=O)N2CCC(N(c3ccnc(Nc4cnc(C)c(F)c4)n3)C(F)(F)F)CC2)ccn1] | A534 |
| C₂₄H₂₃F₄N₇O | |
| 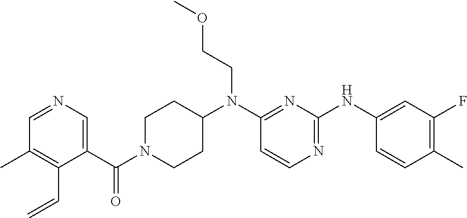 | A535 |
| C₂₈H₃₃FN₆O₂ | |
| 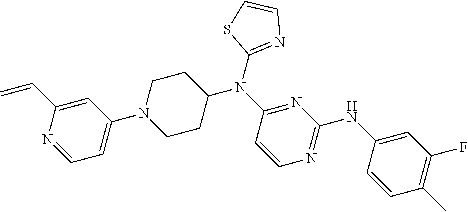 [SMILES: C=Cc1cc(N2CCC(N(c3ccnc(Nc4ccc(C)c(F)c4)n3)c3nccs3)CC2)ccn1] | A536 |
| C₂₆H₂₆FN₇S | |
| 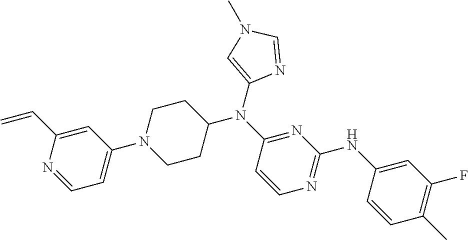 [SMILES: C=Cc1cc(N2CCC(N(c3cn(C)cn3)c3ccnc(Nc4ccc(C)c(F)c4)n3)CC2)ccn1] | A537 |
| C₂₇H₂₉FN₈ | |
| 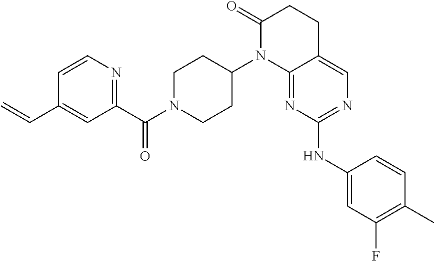 [SMILES: C=Cc1ccnc(C(=O)N2CCC(N3C(=O)CCc4cnc(Nc5ccc(C)c(F)c5)nc43)CC2)c1] | A538 |
| C₂₇H₂₇FN₆O₂ | |
| 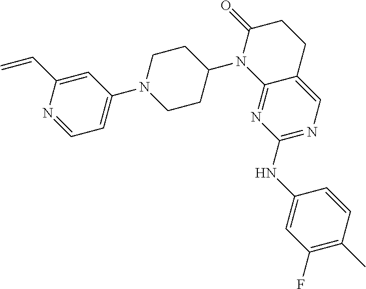 [SMILES: C=Cc1cc(N2CCC(N3C(=O)CCc4cnc(Nc5ccc(C)c(F)c5)nc43)CC2)ccn1] | A539 |
| C₂₆H₂₇FN₆O | |
| 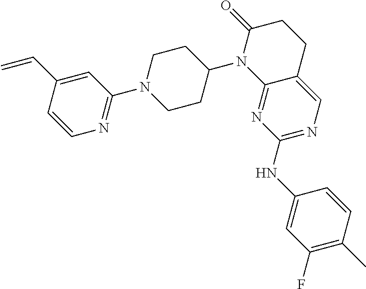 [SMILES: C=Cc1ccnc(N2CCC(N3C(=O)CCc4cnc(Nc5ccc(C)c(F)c5)nc43)CC2)c1] | A540 |
| C₂₆H₂₇FN₆O | |
| 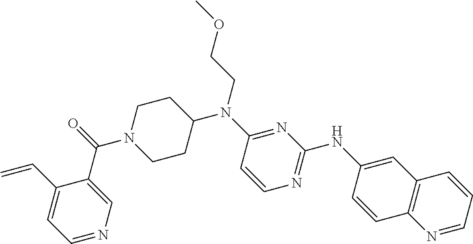 [SMILES: C=Cc1ccncc1C(=O)N1CCC(N(CCOC)c2ccnc(Nc3ccc4ncccc4c3)n2)CC1] | A541 |
| C₂₉H₃₁N₇O₂ | |
| 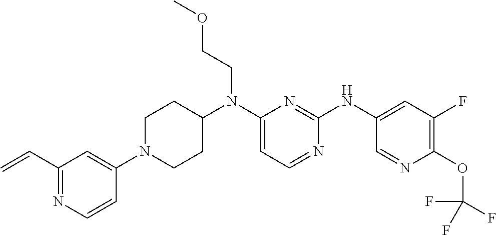 [SMILES: C=Cc1cc(N2CCC(N(CCOC)c3ccnc(Nc4cnc(OC(F)(F)F)c(F)c4)n3)CC2)ccn1] | A542 |
| C₂₅H₂₇F₄N₇O₂ | |
| 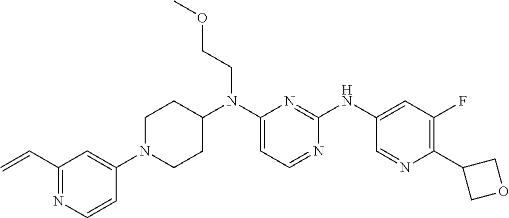 [SMILES: C=Cc1cc(N2CCC(N(CCOC)c3ccnc(Nc4cnc(C5COC5)c(F)c4)n3)CC2)ccn1] | A543 |
| C₂₇H₃₂FN₇O₂ | |
| 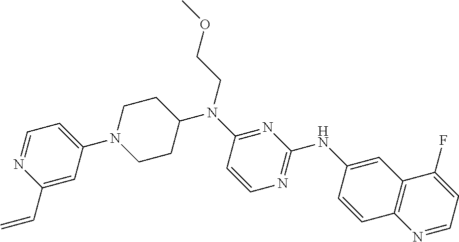 [SMILES: C=Cc1cc(N2CCC(N(CCOC)c3ccnc(Nc4ccc5nccc(F)c5c4)n3)CC2)ccn1] | A544 |
| C₂₈H₃₀FN₇O | |
| 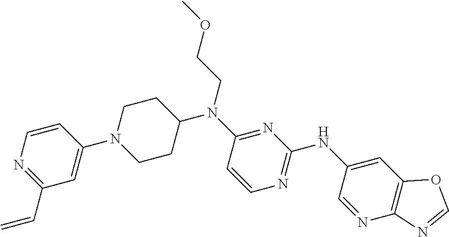 [SMILES: C=Cc1cc(N2CCC(N(CCOC)c3ccnc(Nc4cnc5ncoc5c4)n3)CC2)ccn1] | A545 |
| C₂₅H₂₈N₈O₂ | |
| 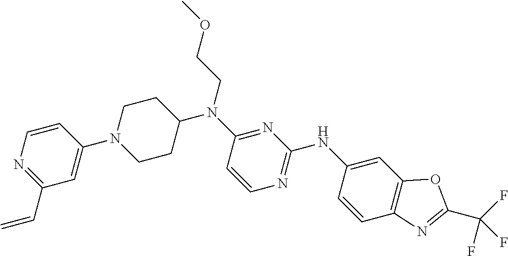 [SMILES: C=Cc1cc(N2CCC(N(CCOC)c3ccnc(Nc4ccc5nc(C(F)(F)F)oc5c4)n3)CC2)ccn1] | A546 |
| C₂₇H₂₈F₃N₇O₂ | |
| 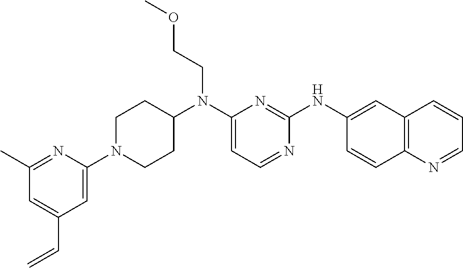 [SMILES: C=Cc1cc(C)nc(N2CCC(N(CCOC)c3ccnc(Nc4ccc5ncccc5c4)n3)CC2)c1] | A547 |
| C₂₉H₃₃N₇O | |
| 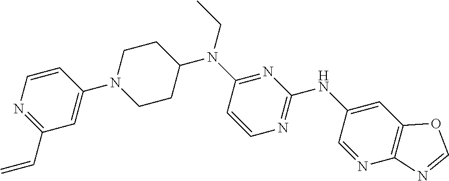 [SMILES: C=Cc1cc(N2CCC(N(CC)c3ccnc(Nc4cnc5ncoc5c4)n3)CC2)ccn1] | A548 |
| C₂₄H₂₆N₈O | |
| 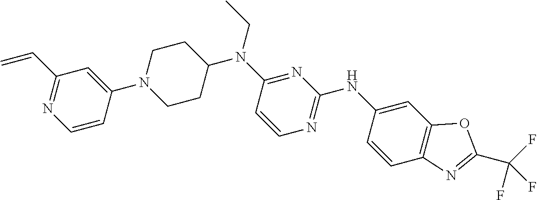 [SMILES: C=Cc1cc(N2CCC(N(CC)c3ccnc(Nc4ccc5nc(C(F)(F)F)oc5c4)n3)CC2)ccn1] | A549 |
| C₂₆H₂₆F₃N₇O | |
| 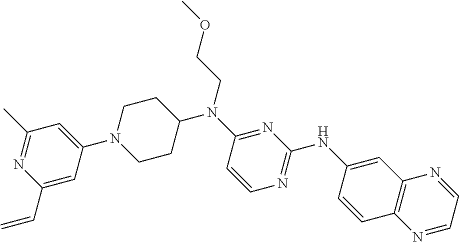 [SMILES: C=Cc1cc(N2CCC(N(CCOC)c3ccnc(Nc4ccc5nccnc5c4)n3)CC2)cc(C)n1] | A550 |
| C₂₈H₃₂N₈O | |
| 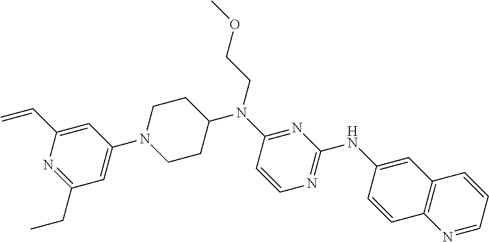 [SMILES: C=Cc1cc(N2CCC(N(CCOC)c3ccnc(Nc4ccc5ncccc5c4)n3)CC2)cc(CC)n1] | A551 |
| C₃₀H₃₅N₇O | |
| 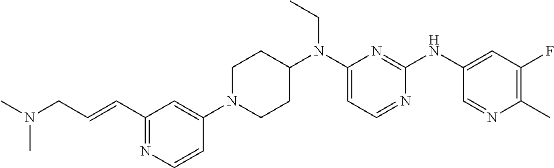 [SMILES: CCN(c1ccnc(Nc2cnc(C)c(F)c2)n1)C1CCN(c2ccnc(/C=C/CN(C)C)c2)CC1] | A552 |
| C₂₇H₃₅FN₈ | |
| 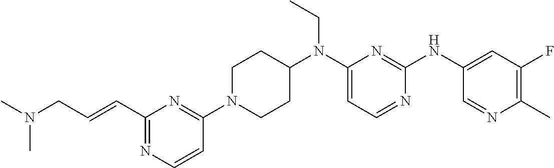 [SMILES: CCN(c1ccnc(Nc2cnc(C)c(F)c2)n1)C1CCN(c2ccnc(/C=C/CN(C)C)n2)CC1] | A553 |
| C₂₆H₃₄FN₉ | |
| 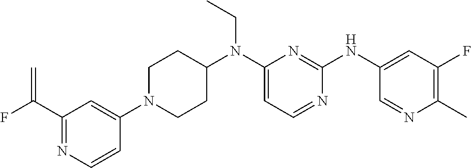 [SMILES: C=C(F)c1cc(N2CCC(N(CC)c3ccnc(Nc4cnc(C)c(F)c4)n3)CC2)ccn1] | A554 |
| C₂₄H₂₇F₂N₇ | |
| 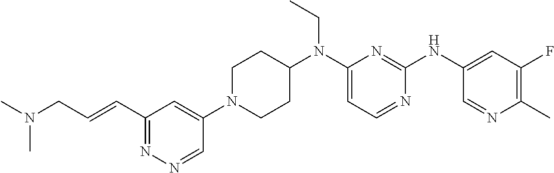 [SMILES: CCN(c1ccnc(Nc2cnc(C)c(F)c2)n1)C1CCN(c2cnnc(/C=C/CN(C)C)c2)CC1] | A555 |
| C₂₆H₃₄FN₉ | |
| 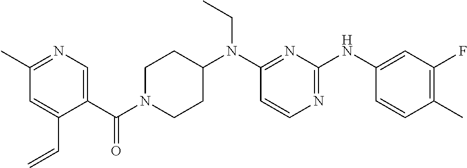 [SMILES: C=Cc1cc(C)ncc1C(=O)N1CCC(N(CC)c2ccnc(Nc3ccc(C)c(F)c3)n2)CC1] | A556 |
| C₂₇H₃₁FN₆O | |
| 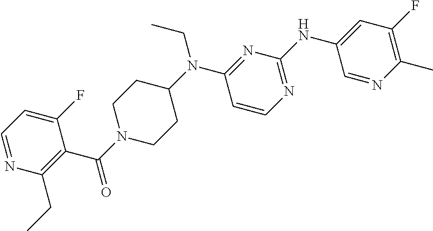 [SMILES: CCc1nccc(F)c1C(=O)N1CCC(N(CC)c2ccnc(Nc3cnc(C)c(F)c3)n2)CC1] | A557 |
| C₂₅H₂₉F₂N₇O | |
| 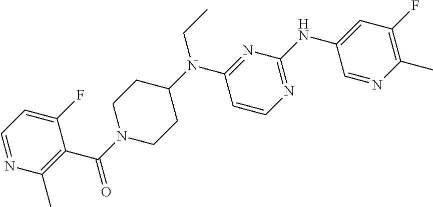 [SMILES: CCN(c1ccnc(Nc2cnc(C)c(F)c2)n1)C1CCN(C(=O)c2c(F)ccnc2C)CC1] | A558 |
| C₂₄H₂₇F₂N₇O | |
| 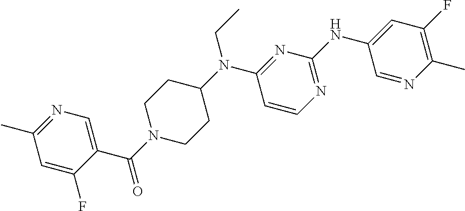 | A559 |
| C₂₄H₂₇F₂N₇O | |
| 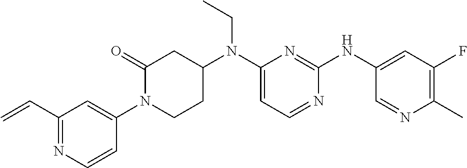 [SMILES: C=Cc1cc(N2CCC(N(CC)c3ccnc(Nc4cnc(C)c(F)c4)n3)CC2=O)ccn1] | A560 |
| C₂₄H₂₆FN₇O | |
| 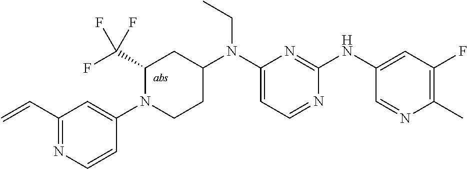 [SMILES: C=Cc1cc(N2CCC(N(CC)c3ccnc(Nc4cnc(C)c(F)c4)n3)C[C@H]2C(F)(F)F)ccn1] | A561 |
| C₂₅H₂₇F₄N₇ | |
| 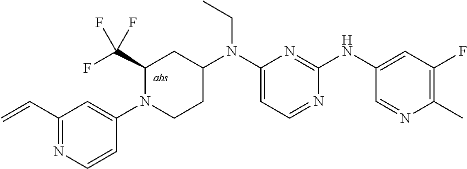 [SMILES: C=Cc1cc(N2CCC(N(CC)c3ccnc(Nc4cnc(C)c(F)c4)n3)C[C@@H]2C(F)(F)F)ccn1] | A562 |
| C₂₅H₂₇F₄N₇ | |
| 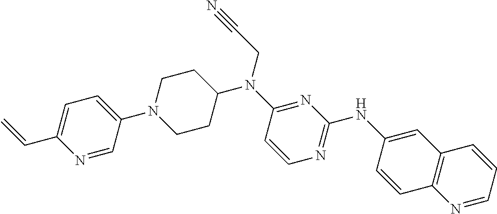 [SMILES: C=Cc1ccc(N2CCC(N(CC#N)c3ccnc(Nc4ccc5ncccc5c4)n3)CC2)cn1] | A563 |
| C₂₇H₂₆N₈ | |
| 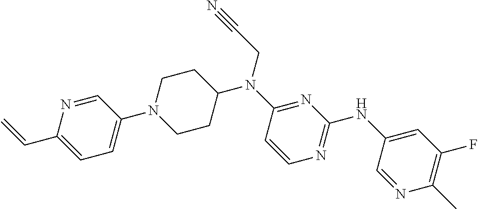 [SMILES: C=Cc1ccc(N2CCC(N(CC#N)c3ccnc(Nc4cnc(C)c(F)c4)n3)CC2)cn1] | A564 |
| C₂₄H₂₅FN₈ | |
| 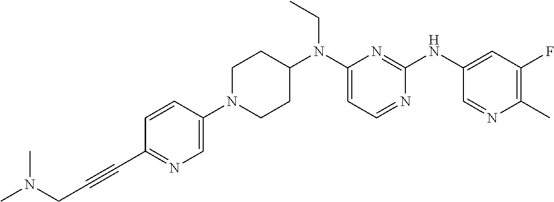 [SMILES: CCN(c1ccnc(Nc2cnc(C)c(F)c2)n1)C1CCN(c2ccc(C#CCN(C)C)nc2)CC1] | A565 |
| C₂₇H₃₃FN₈ | |
| 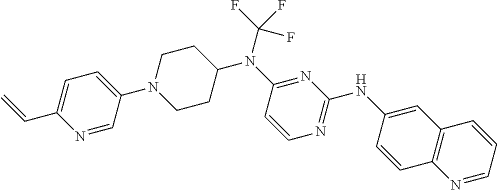 [SMILES: C=Cc1ccc(N2CCC(N(c3ccnc(Nc4ccc5ncccc5c4)n3)C(F)(F)F)CC2)cn1] | A566 |
| C₂₆H₂₄F₃N₇ | |
| 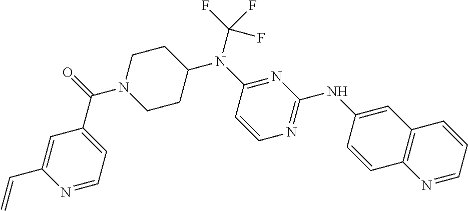 [SMILES: C=Cc1cc(C(=O)N2CCC(N(c3ccnc(Nc4ccc5ncccc5c4)n3)C(F)(F)F)CC2)ccn1] | A567 |
| C₂₇H₂₄F₃N₇O | |
| 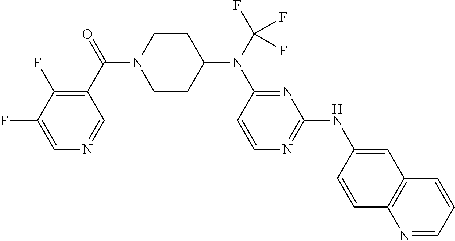 [SMILES: O=C(c1cncc(F)c1F)N1CCC(N(c2ccnc(Nc3ccc4ncccc4c3)n2)C(F)(F)F)CC1] | A568 |
| C₂₅H₂₀F₅N₇O | |
| 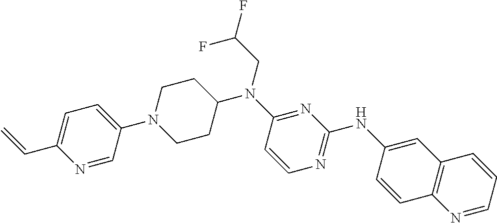 [SMILES: C=Cc1ccc(N2CCC(N(CC(F)F)c3ccnc(Nc4ccc5ncccc5c4)n3)CC2)cn1] | A569 |
| C₂₇H₂₇F₂N₇ | |
| 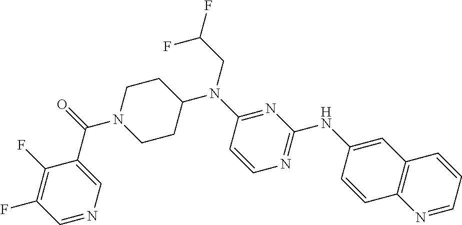 [SMILES: O=C(c1cncc(F)c1F)N1CCC(N(CC(F)F)c2ccnc(Nc3ccc4ncccc4c3)n2)CC1] | A570 |
| C₂₆H₂₃F₄N₇O | |
| 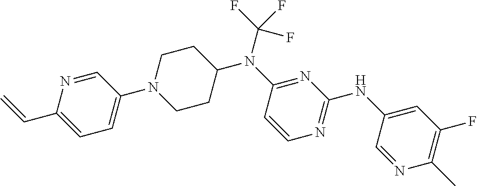 [SMILES: C=Cc1ccc(N2CCC(N(c3ccnc(Nc4cnc(C)c(F)c4)n3)C(F)(F)F)CC2)cn1] | A571 |
| C₂₃H₂₃F₄N₇ | |
| 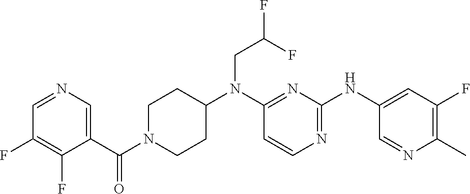 [SMILES: Cc1ncc(Nc2nccc(N(CC(F)F)C3CCN(C(=O)c4cncc(F)c4F)CC3)n2)cc1F] | A572 |
| C₂₃H₂₂F₅N₇O | |
| 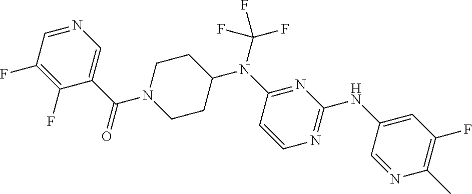 [SMILES: Cc1ncc(Nc2nccc(N(C3CCN(C(=O)c4cncc(F)c4F)CC3)C(F)(F)F)n2)cc1F] | A573 |
| C₂₂H₁₉F₆N₇O | |
| 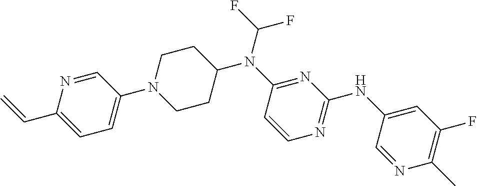 [SMILES: C=Cc1ccc(N2CCC(N(c3ccnc(Nc4cnc(C)c(F)c4)n3)C(F)F)CC2)cn1] | A574 |
| C₂₃H₂₄F₃N₇ | |
| 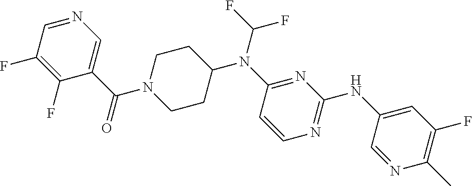 [SMILES: Cc1ncc(Nc2nccc(N(C(F)F)C3CCN(C(=O)c4cncc(F)c4F)CC3)n2)cc1F] | A575 |
| C₂₂H₂₀F₅N₇O | |
| 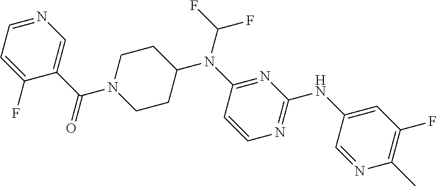 [SMILES: Cc1ncc(Nc2nccc(N(C(F)F)C3CCN(C(=O)c4cnccc4F)CC3)n2)cc1F] | A576 |
| C₂₂H₂₁F₄N₇O | |
| 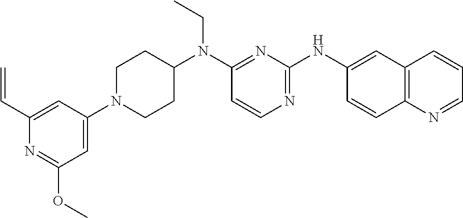 [SMILES: C=Cc1cc(N2CCC(N(CC)c3ccnc(Nc4ccc5ncccc5c4)n3)CC2)cc(OC)n1] | A577 |
| 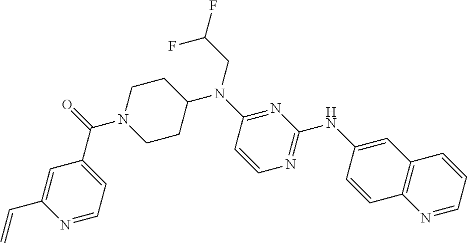 [SMILES: C=Cc1cc(C(=O)N2CCC(N(CC(F)F)c3ccnc(Nc4ccc5ncccc5c4)n3)CC2)ccn1] | A578 |
| 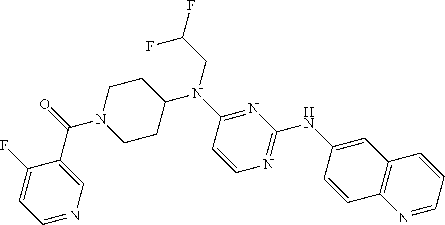 [SMILES: O=C(c1cnccc1F)N1CCC(N(CC(F)F)c2ccnc(Nc3ccc4ncccc4c3)n2)CC1] | A579 |
| 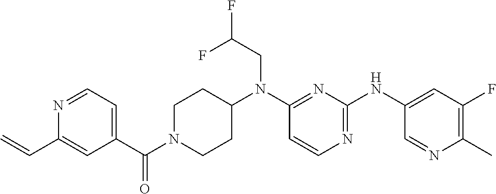 [SMILES: C=Cc1cc(C(=O)N2CCC(N(CC(F)F)c3ccnc(Nc4cnc(C)c(F)c4)n3)CC2)ccn1] | A580 |
| 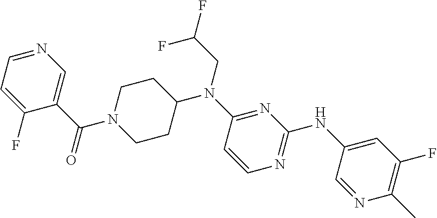 [SMILES: Cc1ncc(Nc2nccc(N(CC(F)F)C3CCN(C(=O)c4cnccc4F)CC3)n2)cc1F] | A581 |
| 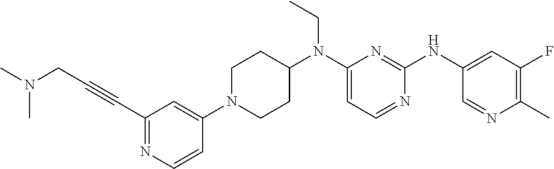 [SMILES: CCN(c1ccnc(Nc2cnc(C)c(F)c2)n1)C1CCN(c2ccnc(C#CCN(C)C)c2)CC1] | A582 |
| C₂₇H₃₃FN₈ | |
| 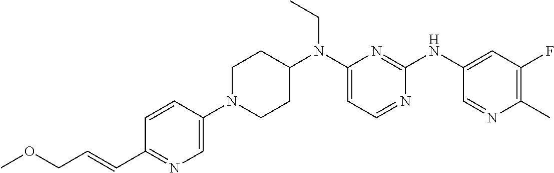 | A583 |
| C₂₆H₃₂FN₇O | |
| 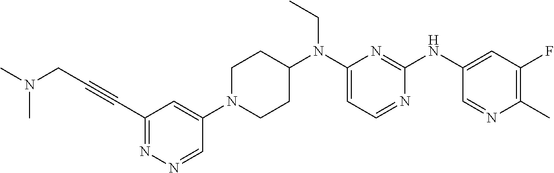 [SMILES: CCN(c1ccnc(Nc2cnc(C)c(F)c2)n1)C1CCN(c2cnnc(C#CCN(C)C)c2)CC1] | A584 |
| C₂₆H₃₂FN₉ | |
| 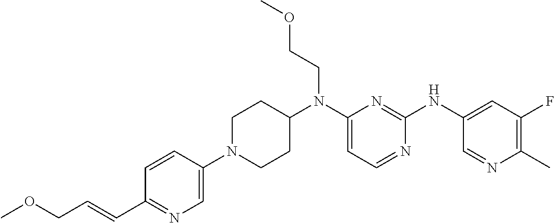 | A585 |
| C₂₇H₃₄FN₇O₂ | |
| 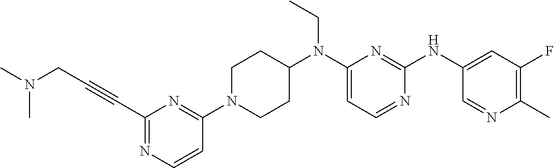 [SMILES: CCN(c1ccnc(Nc2cnc(C)c(F)c2)n1)C1CCN(c2ccnc(C#CCN(C)C)n2)CC1] | A586 |
| C₂₆H₃₂FN₉ | |
| 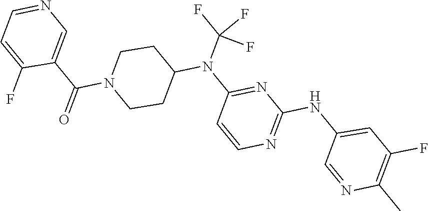 [SMILES: Cc1ncc(Nc2nccc(N(C3CCN(C(=O)c4cnccc4F)CC3)C(F)(F)F)n2)cc1F] | A587 |
| C₂₂H₂₀F₅N₇O | |
| 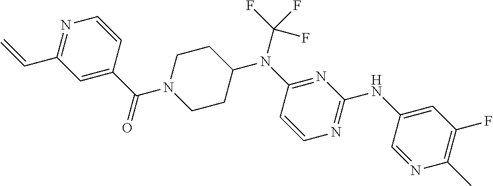 [SMILES: C=Cc1cc(C(=O)N2CCC(N(c3ccnc(Nc4cnc(C)c(F)c4)n3)C(F)(F)F)CC2)ccn1] | A588 |
| C₂₄H₂₃F₄N₇O | |
| 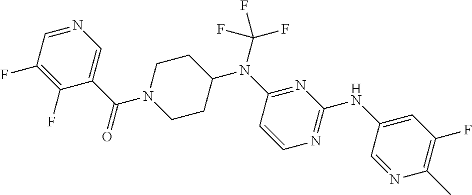 [SMILES: Cc1ncc(Nc2nccc(N(C3CCN(C(=O)c4cncc(F)c4F)CC3)C(F)(F)F)n2)cc1F] | A589 |
| C₂₂H₁₉F₆N₇O | |
| 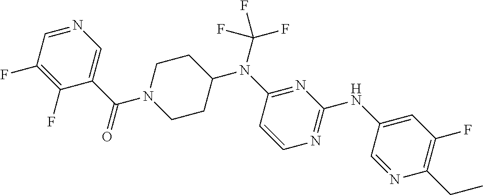 [SMILES: CCc1ncc(Nc2nccc(N(C3CCN(C(=O)c4cncc(F)c4F)CC3)C(F)(F)F)n2)cc1F] | A590 |
| C₂₃H₂₁F₆N₇O | |
| 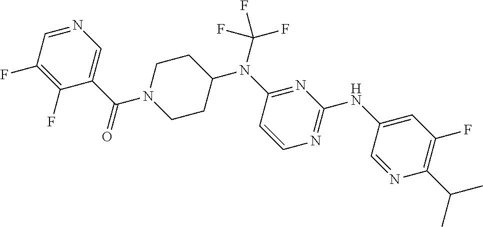 [SMILES: CC(C)c1ncc(Nc2nccc(N(C3CCN(C(=O)c4cncc(F)c4F)CC3)C(F)(F)F)n2)cc1F] | A591 |
| C₂₄H₂₃F₆N₇O | |
| 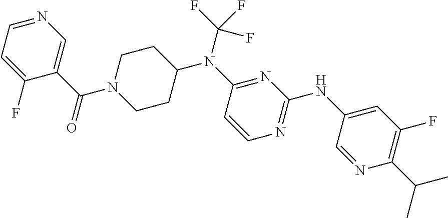 [SMILES: CC(C)c1ncc(Nc2nccc(N(C3CCN(C(=O)c4cnccc4F)CC3)C(F)(F)F)n2)cc1F] | A592 |
| C₂₄H₂₄F₅N₇O | |
| 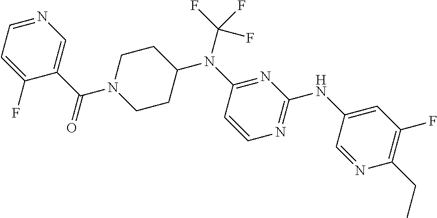 [SMILES: CCc1ncc(Nc2nccc(N(C3CCN(C(=O)c4cnccc4F)CC3)C(F)(F)F)n2)cc1F] | A593 |
| C₂₃H₂₂F₅N₇O | |
| 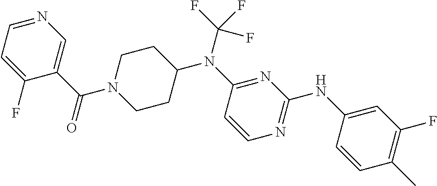 [SMILES: Cc1ccc(Nc2nccc(N(C3CCN(C(=O)c4cnccc4F)CC3)C(F)(F)F)n2)cc1F] | A594 |
| C₂₃H₂₁F₅N₆O | |
| 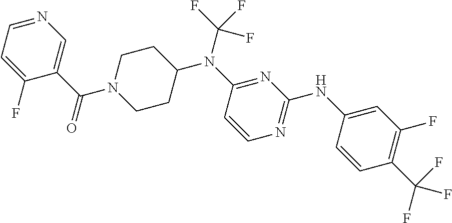 [SMILES: O=C(c1cnccc1F)N1CCC(N(c2ccnc(Nc3ccc(C(F)(F)F)c(F)c3)n2)C(F)(F)F)CC1] | A595 |
| C₂₃H₁₈F₈N₆O | |
| 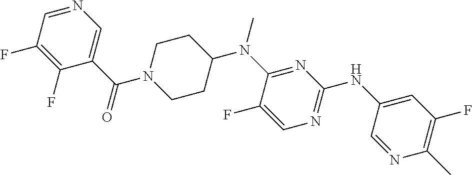 [SMILES: Cc1ncc(Nc2ncc(F)c(N(C)C3CCN(C(=O)c4cncc(F)c4F)CC3)n2)cc1F] | A596 |
| C₂₂H₂₁F₄N₇O | |
| 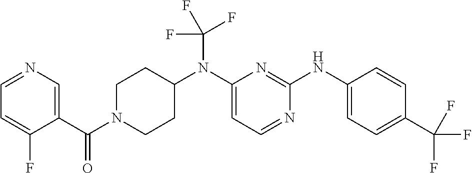 [SMILES: O=C(c1cnccc1F)N1CCC(N(c2ccnc(Nc3ccc(C(F)(F)F)cc3)n2)C(F)(F)F)CC1] | A597 |
| C₂₃H₁₉F₇N₆O | |
| 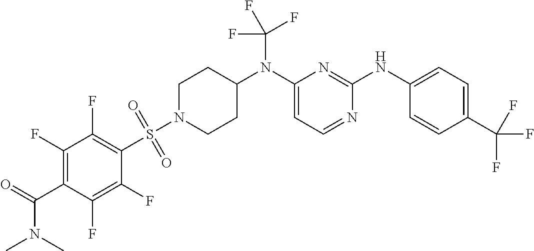 [SMILES: CN(C)C(=O)c1c(F)c(F)c(S(=O)(=O)N2CCC(N(c3ccnc(Nc4ccc(C(F)(F)F)cc4)n3)C(F)(F)F)CC2)c(F)c1F] | A598 |
| C₂₆H₂₂F₁₀N₆O₃S | |
| 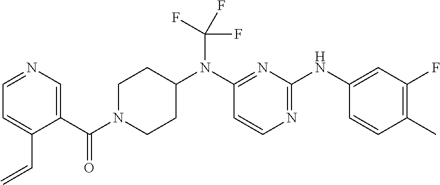 [SMILES: C=Cc1ccncc1C(=O)N1CCC(N(c2ccnc(Nc3ccc(C)c(F)c3)n2)C(F)(F)F)CC1] | A599 |
| C₂₅H₂₄F₄N₆O | |
| 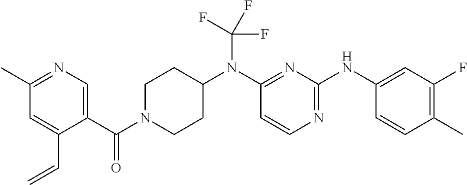 [SMILES: C=Cc1cc(C)ncc1C(=O)N1CCC(N(c2ccnc(Nc3ccc(C)c(F)c3)n2)C(F)(F)F)CC1] | A600 |
| C₂₆H₂₆F₄N₆O | |
| 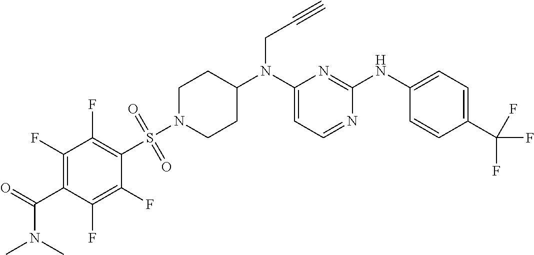 [SMILES: C#CCN(c1ccnc(Nc2ccc(C(F)(F)F)cc2)n1)C1CCN(S(=O)(=O)c2c(F)c(F)c(C(=O)N(C)C)c(F)c2F)CC1] | A601 |
| C₂₈H₂₅F₇N₆O₃S | |
| 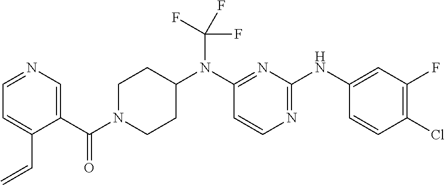 [SMILES: C=Cc1ccncc1C(=O)N1CCC(N(c2ccnc(Nc3ccc(Cl)c(F)c3)n2)C(F)(F)F)CC1] | A602 |
| C₂₄H₂₁ClF₄N₆O | |
| 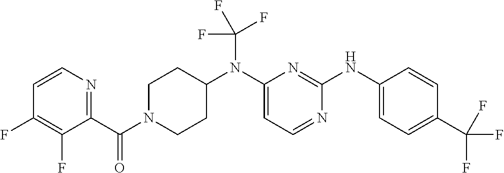 [SMILES: O=C(c1nccc(F)c1F)N1CCC(N(c2ccnc(Nc3ccc(C(F)(F)F)cc3)n2)C(F)(F)F)CC1] | A603 |
| C₂₃H₁₈F₈N₆O | |
| 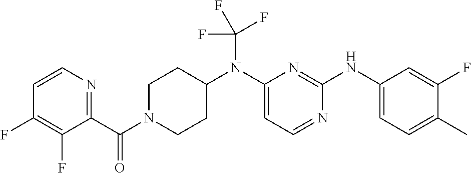 [SMILES: Cc1ccc(Nc2nccc(N(C3CCN(C(=O)c4nccc(F)c4F)CC3)C(F)(F)F)n2)cc1F] | A604 |
| C₂₃H₂₀F₆N₆O | |
| 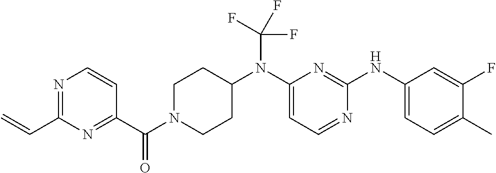 [SMILES: C=Cc1nccc(C(=O)N2CCC(N(c3ccnc(Nc4ccc(C)c(F)c4)n3)C(F)(F)F)CC2)n1] | A605 |
| C₂₄H₂₃F₄N₇O | |
| 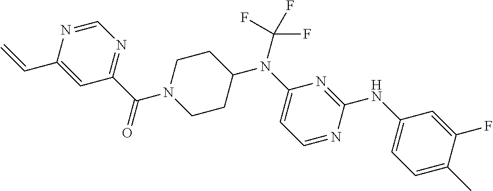 [SMILES: C=Cc1cc(C(=O)N2CCC(N(c3ccnc(Nc4ccc(C)c(F)c4)n3)C(F)(F)F)CC2)ncn1] | A606 |
| C₂₄H₂₃F₄N₇O | |
| 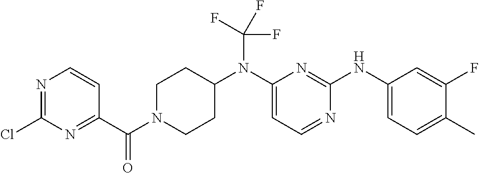 [SMILES: Cc1ccc(Nc2nccc(N(C3CCN(C(=O)c4ccnc(Cl)n4)CC3)C(F)(F)F)n2)cc1F] | A607 |
| C₂₂H₂₀ClF₄N₇O | |
| 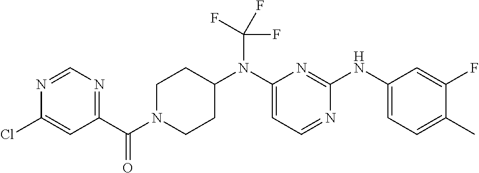 [SMILES: Cc1ccc(Nc2nccc(N(C3CCN(C(=O)c4cc(Cl)ncn4)CC3)C(F)(F)F)n2)cc1F] | A608 |
| C₂₂H₂₀ClF₄N₇O | |
| 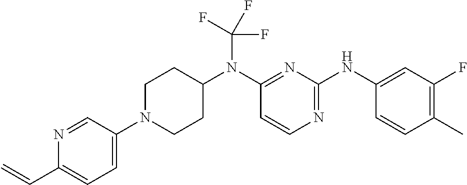 [SMILES: C=Cc1ccc(N2CCC(N(c3ccnc(Nc4ccc(C)c(F)c4)n3)C(F)(F)F)CC2)cn1] | A609 |
| C₂₄H₂₄F₄N₆ | |
| 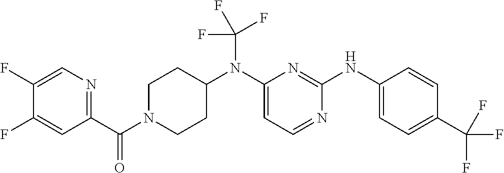 [SMILES: O=C(c1cc(F)c(F)cn1)N1CCC(N(c2ccnc(Nc3ccc(C(F)(F)F)cc3)n2)C(F)(F)F)CC1] | A610 |
| C₂₃H₁₈F₈N₆O | |
| 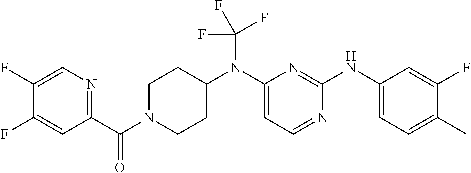 [SMILES: Cc1ccc(Nc2nccc(N(C3CCN(C(=O)c4cc(F)c(F)cn4)CC3)C(F)(F)F)n2)cc1F] | A611 |
| C₂₃H₂₀F₆N₆O | |
| 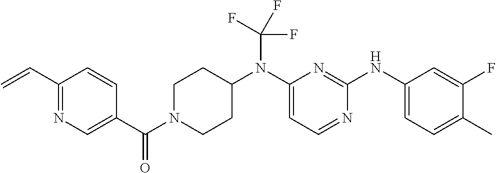 [SMILES: C=Cc1ccc(C(=O)N2CCC(N(c3ccnc(Nc4ccc(C)c(F)c4)n3)C(F)(F)F)CC2)cn1] | A612 |
| C₂₅H₂₄F₄N₆O | |
| 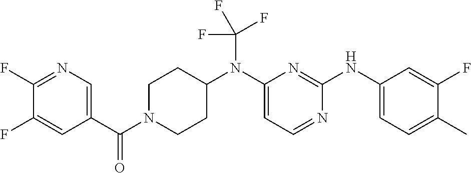 [SMILES: Cc1ccc(Nc2nccc(N(C3CCN(C(=O)c4cnc(F)c(F)c4)CC3)C(F)(F)F)n2)cc1F] | A613 |
| C₂₃H₂₀F₆N₆O | |
| 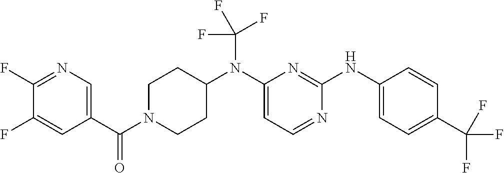 [SMILES: O=C(c1cnc(F)c(F)c1)N1CCC(N(c2ccnc(Nc3ccc(C(F)(F)F)cc3)n2)C(F)(F)F)CC1] | A614 |
| C₂₃H₁₈F₈N₆O | |
| 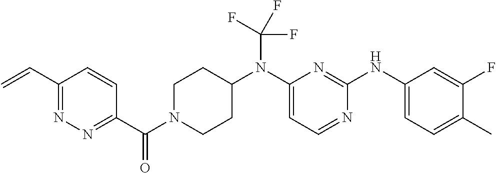 [SMILES: C=Cc1ccc(C(=O)N2CCC(N(c3ccnc(Nc4ccc(C)c(F)c4)n3)C(F)(F)F)CC2)nn1] | A615 |
| C₂₄H₂₃F₄N₇O | |
| 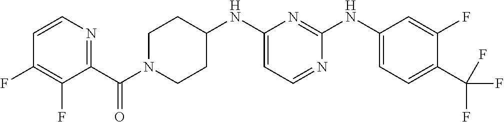 [SMILES: O=C(c1nccc(F)c1F)N1CCC(Nc2ccnc(Nc3ccc(C(F)(F)F)c(F)c3)n2)CC1] | A616 |
| C₂₂H₁₈F₆N₆O | |
| 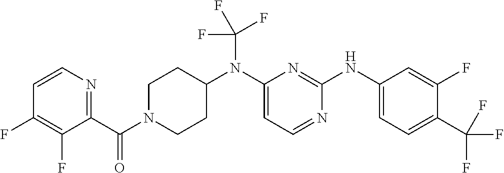 [SMILES: O=C(c1nccc(F)c1F)N1CCC(N(c2ccnc(Nc3ccc(C(F)(F)F)c(F)c3)n2)C(F)(F)F)CC1] | A617 |
| C₂₃H₁₇F₉N₆O | |
| 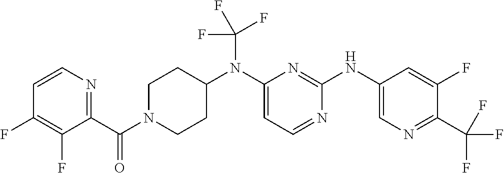 [SMILES: O=C(c1nccc(F)c1F)N1CCC(N(c2ccnc(Nc3cnc(C(F)(F)F)c(F)c3)n2)C(F)(F)F)CC1] | A618 |
| C₂₂H₁₇F₈N₇O | |
| 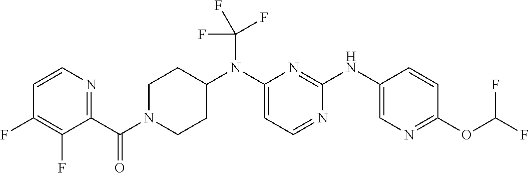 [SMILES: O=C(c1nccc(F)c1F)N1CCC(N(c2ccnc(Nc3ccc(OC(F)F)nc3)n2)C(F)(F)F)CC1] | A619 |
| C₂₂H₁₈F₇N₇O₂ | |
| 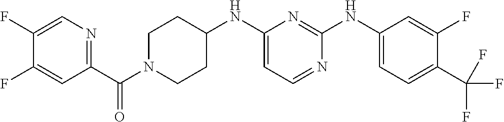 [SMILES: O=C(c1cc(F)c(F)cn1)N1CCC(Nc2ccnc(Nc3ccc(C(F)(F)F)c(F)c3)n2)CC1] | A620 |
| C₂₂H₁₈F₆N₆O | |
| 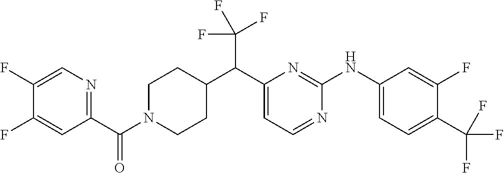 [SMILES: O=C(c1cc(F)c(F)cn1)N1CCC(C(c2ccnc(Nc3ccc(C(F)(F)F)c(F)c3)n2)C(F)(F)F)CC1] | A621 |
| C₂₃H₁₇F₉N₆O | |
| 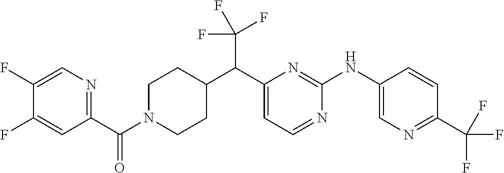 [SMILES: O=C(c1cc(F)c(F)cn1)N1CCC(C(c2ccnc(Nc3ccc(C(F)(F)F)nc3)n2)C(F)(F)F)CC1] | A622 |
| C₂₂H₁₇F₈N₇O | |
| 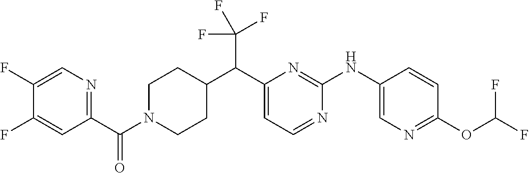 [SMILES: O=C(c1cc(F)c(F)cn1)N1CCC(C(c2ccnc(Nc3ccc(OC(F)F)nc3)n2)C(F)(F)F)CC1] | A623 |
| C₂₂H₁₈F₇N₇O₂ | |
| 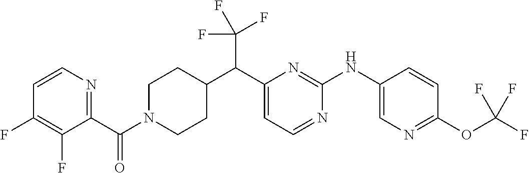 [SMILES: O=C(c1nccc(F)c1F)N1CCC(C(c2ccnc(Nc3ccc(OC(F)(F)F)nc3)n2)C(F)(F)F)CC1] | A624 |
| C₂₂H₁₇F₈N₇O₂ | |
| 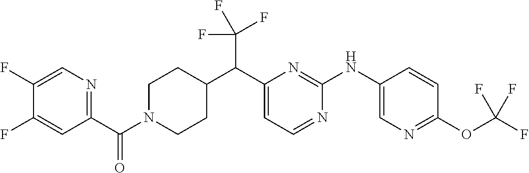 [SMILES: O=C(c1cc(F)c(F)cn1)N1CCC(C(c2ccnc(Nc3ccc(OC(F)(F)F)nc3)n2)C(F)(F)F)CC1] | A625 |
| C₂₂H₁₇F₈N₇O₂ | |
| 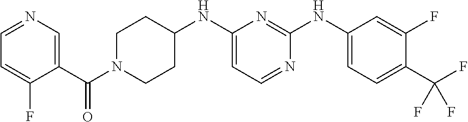 [SMILES: O=C(c1cnccc1F)N1CCC(Nc2ccnc(Nc3ccc(C(F)(F)F)c(F)c3)n2)CC1] | A626 |
| C₂₂H₁₉F₅N₆O | |
| 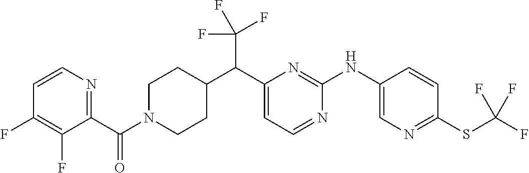 [SMILES: O=C(c1nccc(F)c1F)N1CCC(C(c2ccnc(Nc3ccc(SC(F)(F)F)nc3)n2)C(F)(F)F)CC1] | A627 |
| C₂₂H₁₇F₈N₇OS | |
| 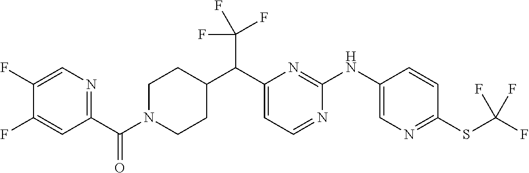 [SMILES: O=C(c1cc(F)c(F)cn1)N1CCC(C(c2ccnc(Nc3ccc(SC(F)(F)F)nc3)n2)C(F)(F)F)CC1] | A628 |
| C₂₂H₁₇F₈N₇OS | |
| 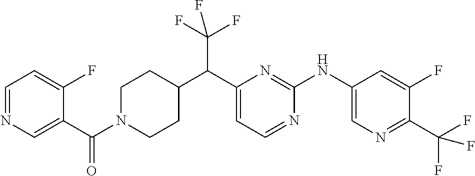 [SMILES: O=C(c1cnccc1F)N1CCC(C(c2ccnc(Nc3cnc(C(F)(F)F)c(F)c3)n2)C(F)(F)F)CC1] | A629 |
| C₂₂H₁₇F₈N₇O | |
| 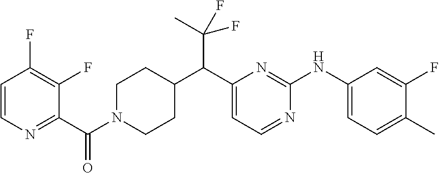 [SMILES: Cc1ccc(Nc2nccc(C(C3CCN(C(=O)c4nccc(F)c4F)CC3)C(C)(F)F)n2)cc1F] | A630 |
| C₂₄H₂₃F₅N₆O | |
| 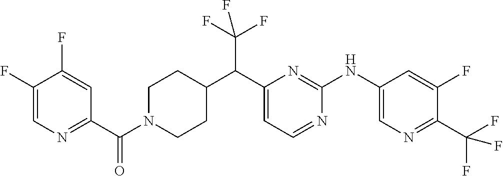 [SMILES: O=C(c1cc(F)c(F)cn1)N1CCC(C(c2ccnc(Nc3cnc(C(F)(F)F)c(F)c3)n2)C(F)(F)F)CC1] | A631 |
| C₂₂H₁₆F₉N₇O | |
| 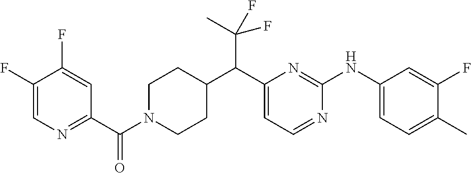 [SMILES: Cc1ccc(Nc2nccc(C(C3CCN(C(=O)c4cc(F)c(F)cn4)CC3)C(C)(F)F)n2)cc1F] | A632 |
| C₂₄H₂₃F₅N₆O | |
| 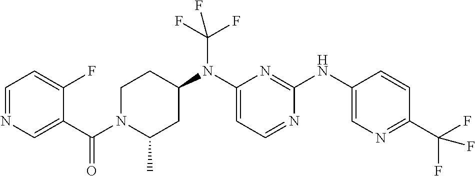 [SMILES: C[C@H]1C[C@H](N(c2ccnc(Nc3ccc(C(F)(F)F)nc3)n2)C(F)(F)F)CCN1C(=O)c1cnccc1F] | A633 |
| C₂₃H₂₀F₇N₇O | |
| 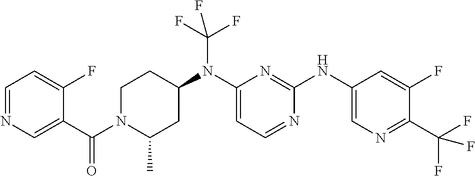 [SMILES: C[C@H]1C[C@H](N(c2ccnc(Nc3cnc(C(F)(F)F)c(F)c3)n2)C(F)(F)F)CCN1C(=O)c1cnccc1F] | A634 |
| C₂₃H₁₉F₈N₇O | |
| 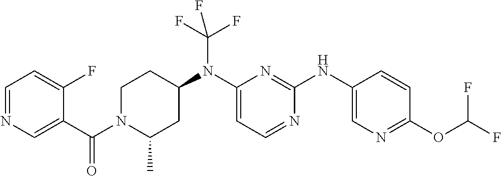 [SMILES: C[C@H]1C[C@H](N(c2ccnc(Nc3ccc(OC(F)F)nc3)n2)C(F)(F)F)CCN1C(=O)c1cnccc1F] | A635 |
| C₂₃H₂₁F₆N₇O₂ | |
| 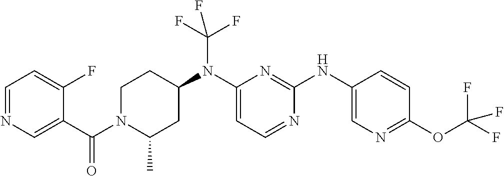 [SMILES: C[C@H]1C[C@H](N(c2ccnc(Nc3ccc(OC(F)(F)F)nc3)n2)C(F)(F)F)CCN1C(=O)c1cnccc1F] | A636 |
| C₂₃H₂₀F₇N₇O₂ | |
| 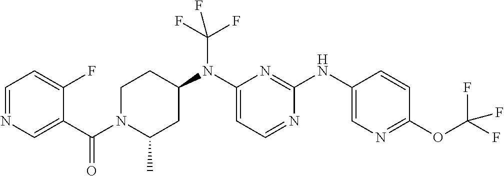 [SMILES: C[C@H]1C[C@H](N(c2ccnc(Nc3ccc(OC(F)(F)F)nc3)n2)C(F)(F)F)CCN1C(=O)c1cnccc1F] | A637 |
| C₂₃H₁₉F₈N₇O | |
| 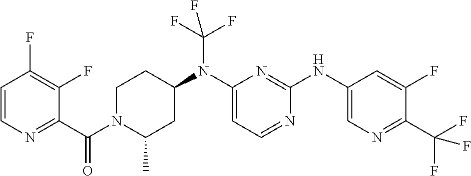 [SMILES: C[C@H]1C[C@H](N(c2ccnc(Nc3cnc(C(F)(F)F)c(F)c3)n2)C(F)(F)F)CCN1C(=O)c1nccc(F)c1F] | A638 |
| C₂₃H₁₈F₉N₇O | |
| 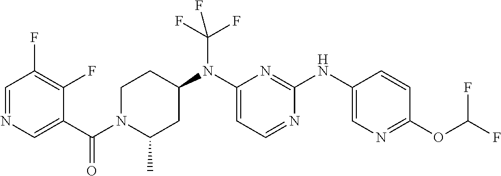 [SMILES: C[C@H]1C[C@H](N(c2ccnc(Nc3ccc(OC(F)F)nc3)n2)C(F)(F)F)CCN1C(=O)c1cncc(F)c1F] | A639 |
| C₂₃H₂₀F₇N₇O₂ | |
| 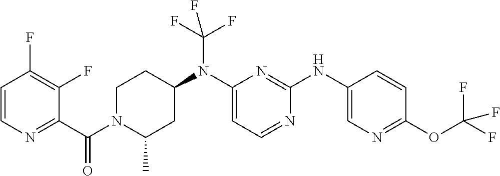 [SMILES: C[C@H]1C[C@H](N(c2ccnc(Nc3ccc(OC(F)(F)F)nc3)n2)C(F)(F)F)CCN1C(=O)c1nccc(F)c1F] | A640 |
| C₂₃H₁₉F₈N₇O₂ | |
| 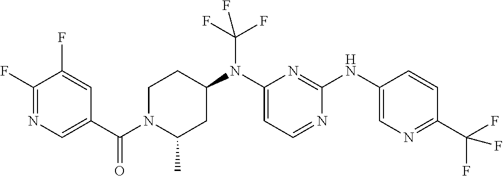 [SMILES: C[C@H]1C[C@H](N(c2ccnc(Nc3ccc(C(F)(F)F)nc3)n2)C(F)(F)F)CCN1C(=O)c1cnc(F)c(F)c1] | A641 |
| C₂₃H₁₉F₈N₇O | |
| 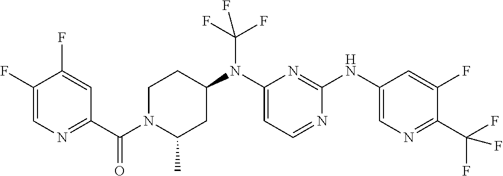 [SMILES: C[C@H]1C[C@H](N(c2ccnc(Nc3cnc(C(F)(F)F)c(F)c3)n2)C(F)(F)F)CCN1C(=O)c1cc(F)c(F)cn1] | A642 |
| C₂₃H₁₈F₉N₇O | |
| 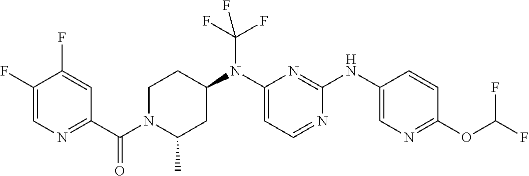 [SMILES: C[C@H]1C[C@H](N(c2ccnc(Nc3ccc(OC(F)F)nc3)n2)C(F)(F)F)CCN1C(=O)c1cc(F)c(F)cn1] | A643 |
| C₂₃H₂₀F₇N₇O₂ | |
| 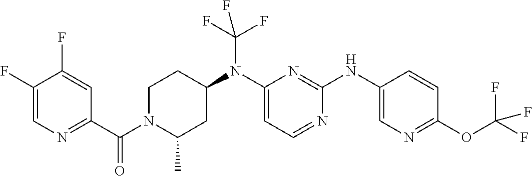 [SMILES: C[C@H]1C[C@H](N(c2ccnc(Nc3ccc(OC(F)(F)F)nc3)n2)C(F)(F)F)CCN1C(=O)c1cc(F)c(F)cn1] | A644 |
| C₂₃H₁₉F₈N₇O₂ | |

### Synthesis of Example A619

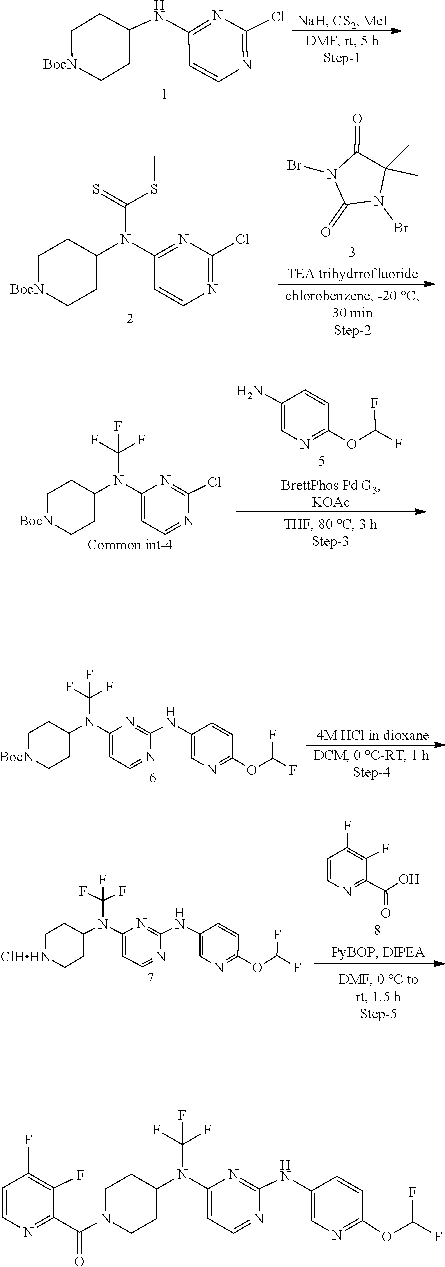

### Synthesis of tert-butyl 4-((2-chloropyrimidin-4-yl)((methylthio)carbonothioyl)amino)piperidine-1-carboxylate (2)

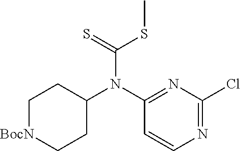 [SMILES: CSC(=S)N(c1ccnc(Cl)n1)C1CCN(C(=O)OC(C)(C)C)CC1]

To an oven dried 500 mL three-neck round-bottom flask equipped with N₂ balloon, was added tert-butyl 4-((2-chloropyrimidin-4-yl)amino)piperidine-1-carboxylate (11 g, 35.1 mmol) and DMF (150 mL). NaH (2.106 g, 52.6 mmol) was added at room temperature and stirred for 1 h. Then CS₂ (3.80 mL, 63.2 mmol) was added dropwise and stirred for 2 h at rt, then iodomethane (3.93 mL, 63.2 mmol) was added. The resulting reaction mixture was further stirred for 5 h at the same temperature. The progress of the reaction was monitored by TLC (20% Ethyl acetate/Hexane, 0.4 rf); upon completion the reaction mixture was quenched with saturated ammonium chloride solution (100 mL) and extracted with ethyl acetate (2×150 mL); the combined organic layers were washed with ice cold water (2×100 mL) and brine solution (100 mL) and dried over sodium sulfate, filtered and concentrated under reduced pressure to get crude product. Thus obtained crude was purified by flash column chromatography (silica gel, 230-400 mesh), eluted with 20-25% EtOAc in hexane to afford tert-butyl 4-((2-chloropyrimidin-4-yl)((methylthio)carbonothioyl)amino)piperidine-1-carboxylate (7.5 g) as a light yellow solid. Thus obtained solid compound was again triturated with acetonitrile (30 mL) and THF (3 mL) to afford the title compound tert-butyl 4-((2-chloropyrimidin-4-yl)((methylthio)carbonothioyl)amino)piperidine-1-carboxylate (5.8 g, 14.26 mmol, 40.6% yield) as an off white solid.

LCMS: 99.07%, RT. 2.469 min, m/z=403.0 [M+H]⁺.

1H-NMR (400 MHz, DMSO-d6): δ 9.01 (d, J=5.20 Hz, 1H), 7.79 (d, J=4.80 Hz, 1H), 5.31-5.25 (m, 1H), 3.98 (d, J=12.00 Hz, 2H), 2.78 (s, 2H), 2.57 (s, 3H), 1.99 (d, J=1.20 Hz, 2H), 1.31-1.24 (m, 11H).

### Synthesis of tert-butyl 4-((2-chloropyrimidin-4-yl)(trifluoromethyl)amino)piperidine-1-carboxylate (Common int-4)

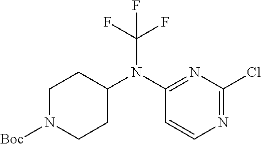 [SMILES: CC(C)(C)OC(=O)N1CCC(N(c2ccnc(Cl)n2)C(F)(F)F)CC1]

To an oven dried 100 mL two-neck round-bottom flask equipped with N₂ balloon was added tert-butyl 4-((2-chloropyrimidin-4-yl)((methylthio)carbonothioyl)amino)piperidine-1-carboxylate (1.0 g, 2.45 mmol) and Chlorobenzene (20 mL). Triethylamine trihydrofluoride (2.485 mL, 15.24 mmol) and 1,3-dibromo-5,5-dimethylhydantoin (2.179 g, 7.62 mmol) were added at −25 °C. The resulting reaction mixture was stirred at −20 °C for 30 min. The progress of the reaction was monitored by TLC (15% Ethyl acetate/Hexane, 0.4 rf). Reaction mixture was quenched with 10% sodium bicarbonate solution (10 mL) and 10% sodium hydroxide solution (10 mL) at −40 °C, to raise the pH up to 7 to 8. Then slowly the reaction mixture was warmed to room temperature and extracted with EtOAc (2×20 mL); the combined organic layers were washed with sodium bicarbonate solution (2×10 mL) followed by brine solution (10 mL) and dried over anhydrous Na₂SO₄, filtered and concentrated under reduced pressure to afford the crude compound as a colourless gummy solid. Thus obtained crude compound was purified by flash column chromatography (silica gel, 230-400 mesh) by using 12 to 15% EtOAc in hexane as an eluent to afford the title compound tert-butyl 4-((2-chloropyrimidin-4-yl)(trifluoromethyl)amino)piperidine-1-carboxylate (312 mg, 0.814 mmol, 33.1% yield) as an off-white solid.

LCMS: 99.40%, RT. 2.560 min, m/z=381.2 [M+H]⁺. 1H-NMR (400 MHz, DMSO-d6): δ 8.58 (d, J=5.60 Hz, 1H), 7.24-7.22 (m, 1H), 4.48-4.43 (m, 1H), 4.03 (d, J=12.00 Hz, 2H), 2.84 (s, 2H), 1.90-1.85 (m, 4H), 1.41 (s, 9H)

¹⁹F-NMR (376 MHz, DMSO-d6): δ −52.023 (3F)

### General procedure A: For Buchwald coupling

### Synthesis of tert-butyl 4-((2-((6-(difluoromethoxy)pyridin-3-yl)amino)pyrimidin-4-yl)(trifluoromethyl)amino)piperidine-1-carboxylate (6)

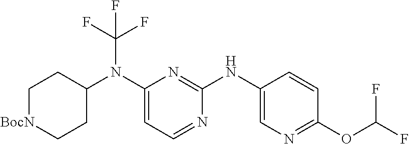 [SMILES: CC(C)(C)OC(=O)N1CCC(N(c2ccnc(Nc3ccc(OC(F)F)nc3)n2)C(F)(F)F)CC1]

To a stirred solution of tert-butyl 4-((2-chloropyrimidin-4-yl)(trifluoromethyl)amino)piperidine-1-carboxylate (600 mg, 1.576 mmol) in THF (10 ml), 6-(difluoromethoxy)pyridin-3-amine (252 mg, 1.576 mmol) and potassium acetate (464 mg, 4.73 mmol) were added. Then the reaction mixture was degassed with nitrogen for 5 min, Brettphos Pd G3 (143 mg, 0.158 mmol) was added and the reaction mixture was further degassed for 5 min and sealed. The resulting reaction mixture was stirred at 80 °C for 3 h. The progress of reaction was monitored by TLC. Upon completion, the reaction mixture was quenched with cold water, extracted with ethyl acetate (3×25 mL). The combined organic layers were washed with brine (50 mL), dried over sodium sulphate and concentrated under reduced pressure to get crude. Thus obtained crude product was purified by column chromatography using 40% to 50% Ethyl acetate in Hexane as an eluent to afford the title compound tert-butyl 4-((2-((6-(difluoromethoxy)pyridin-3-yl)amino)pyrimidin-4-yl)(trifluoromethyl)amino)piperidine-1-carboxylate (600 mg, 1.154 mmol, 73.2% yield) as a yellow solid.

LCMS: 97.73%, RT: 2.61 min, m/z=505.6 (M+H)⁺.

1H-NMR (400 MHz, CDCl₃): δ 8.34 (d, J=2.8 Hz, 1H), 8.21 (d, J=5.6 Hz, 1H), 8.05 (dd, J=2.8, 8.8 Hz, 1H), 7.42 (t, J=73.2 Hz, 1H), 7.14 (s, 1H), 6.92 (d, J=8.8 Hz, 1H), 6.50 (dd, J=2.4, 5.6 Hz, 1H), 4.66 (t, 12.4 Hz, 1H), 4.27 (s, 2H), 2.75 (t, J=12.4 Hz, 2H), 2.06-2.01 (m, 2H), 1.88-1.85 (m, 2H), 1.49 (s, 9H).

19F-NMR (376 MHz, CDCl3): δ −51.657 (3F), −88.239 to −88.433 (2F).

### General Procedure B: For Boc-Deprotection

### Synthesis of N²-(6-(difluoromethoxy)pyridin-3-yl)-N⁴-(piperidin-4-yl)-N⁴-(trifluoromethyl)pyrimidine-2,4-diamine hydrochloride (7)

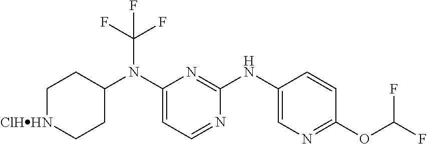

To a stirred solution of tert-butyl 4-((2-((6-(difluoromethoxy)pyridin-3-yl)amino)pyrimidin-4-yl)(trifluoromethyl)amino)piperidine-1-carboxylate (300 mg, 0.595 mmol) in DCM (15 mL) was added 4M HCl in dioxane (0.196 mL, 5.95 mmol) at 0 °C and the mixture was then stirred at 25 °C for 1 h. The progress of the reaction was monitored by TLC and LCMS. After completion of the reaction, volatiles were removed under reduced pressure to get crude. Thus obtained crude was triturated with MTBE to afford N²-(6-(difluoromethoxy)pyridin-3-yl)-N⁴-(piperidin-4-yl)-N⁴-(trifluoromethyl)pyrimidine-2,4-diamine hydrochloride (220 mg, 0.490 mmol, 82% yield) as an off white solid.

LCMS: 90.36%, RT: 0.99 min, m/z=405.1 [M+H]⁺.

### General Procedure C: For Amide Coupling

### Synthesis of (4-((2-((6-(difluoromethoxy)pyridin-3-yl)amino)pyrimidin-4-yl)(trifluoromethyl)amino)piperidin-1-yl)(3,4-difluoropyridin-2-yl)methanone

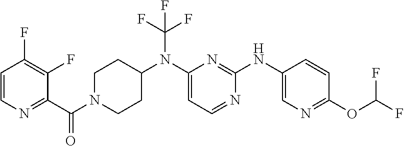 [SMILES: O=C(c1nccc(F)c1F)N1CCC(N(c2ccnc(Nc3ccc(OC(F)F)nc3)n2)C(F)(F)F)CC1]

To a stirred solution of 3,4-difluoropicolinic acid (45.1 mg, 0.284 mmol) in DMF (5 mL) was added N₂-(6-(difluoromethoxy)pyridin-3-yl)-N4-(piperidin-4-yl)-N4-(trifluoromethyl)pyrimidine-2,4-diamine hydrochloride (125 mg, 0.284 mmol), DIPEA (110 mg, 0.851 mmol) and PyBOP (221 mg, 0.425 mmol) at 0 °C. The resulting reaction mixture was stirred at room temperature for 1 h. The progress of the reaction was monitored by LCMS. Then the reaction mixture was diluted with cold water and extracted with ethyl acetate. The combined organic layers were dried over sodium sulfate, concentrated under reduced pressure to get crude. Thus obtained crude compound was purified by reverse phase chromatography by using a 100 g C18 silica column, eluted with 0.01% formic acid in water and acetonitrile. The collected fractions were lyophilized to afford the title compound (4-((2-((6-(difluoromethoxy)pyridin-3-yl)amino)pyrimidin-4-yl)(trifluoromethyl)amino)piperidin-1-yl)(3,4-difluoropyridin-2-yl)methanone (65 mg, 0.119 mmol, 41.8% yield) as off white solid.

LCMS: 99.91%, RT: 1.75 min, m/z=546.3 (M+H)⁺.

LCMS Method: Method 1

HPLC: 99.50%. RT: 6.709 min.

¹HNMR (400 MHz, DMSO-d6, VT): δ 9.53 (s, 1H), 8.53 (d, J=2.4 Hz, 1H), 8.47 (t, J=0.8 Hz, 1H), 8.36 (d, J=5.6 Hz, 1H), 8.17 (dd, J=2.4, 8.8 Hz, 1H), 7.65-7.36 (m, 2H), 7.06 (d, J=8.8 Hz, 1H), 6.59-6.57 (m, 1H), 4.64 (q, J=11.6 Hz, 2H), 3.53 (d, J=12.0 Hz, 1H), 3.19 (t, J=9.6 Hz, 1H), 2.93 (t, J=11.2 Hz, 1H), 2.07-1.89 (m, 4H).

19F-NMR (376 MHz, DMSO-d6): −50.488 (3F), −86.336 to −86.531 (2F), −126.375 to −126.476 (1F), −151.844 to −151.917 (1F).

### Synthesis of Example A625

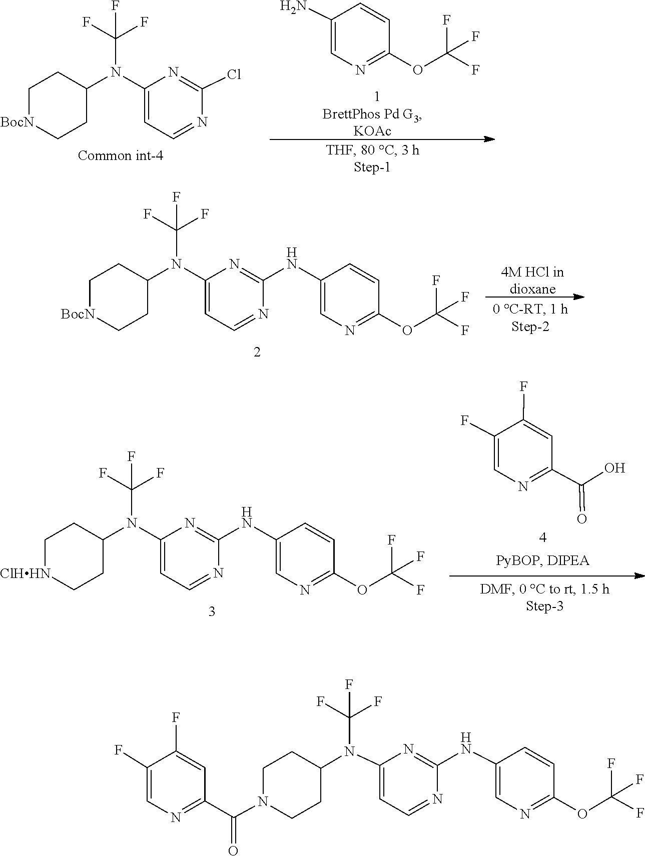

### Synthesis of tert-butyl 4-((2-((6-(trifluoromethoxy)pyridin-3-yl)amino)pyrimidin-4-yl)(trifluoromethyl)amino)piperidine-1-carboxylate (2)

Synthesized by using general procedure A for Buchwald coupling

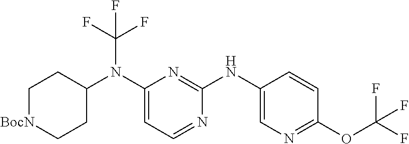 [SMILES: CC(C)(C)OC(=O)N1CCC(N(c2ccnc(Nc3ccc(OC(F)(F)F)nc3)n2)C(F)(F)F)CC1]

Isolated (600 mg, 43% yield) as off white solid.

LCMS: 99.58%, RT: 2.151 min, m/z=523.2 (M+H)⁺.

¹HNMR (400 MHz, CDCl3): δ 8.45 (d, J=3.2 Hz, 1H), 8.24-8.18 (m, 2H), 7.05 (d, J=8.8 Hz, 1H), 6.55-6.51 (m, 1H), 4.67 (t, J=12.0 Hz, 1H), 4.28 (s, 2H), 2.80-2.70 (m, 1H), 2.14-2.04 (m, 2H), 1.89-1.86 (m, 2H), 1.50 (s, 9H). (NH is missing due to NMR recorded in CDCl₃)

19F-NMR (376 MHz, CDCl3): −51.663 (3F), −56.651 (3F).

### Synthesis of N4-(piperidin-4-yl)-N2-(6-(trifluoromethoxy)pyridin-3-yl)-N4-(trifluoromethyl)pyrimidine-2,4-diamine hydrochloride (3)

Synthesized by using general procedure B for Boc-deprotection:

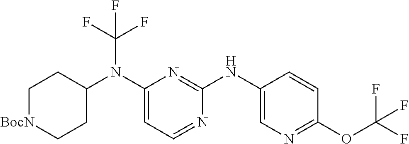 [SMILES: CC(C)(C)OC(=O)N1CCC(N(c2ccnc(Nc3ccc(OC(F)(F)F)nc3)n2)C(F)(F)F)CC1]

Crude (400 mg, 90% yield) as off white solid.

LCMS: 99.56%, RT: 1.18 min, m/z=423.25 [M+H]⁺.

### Synthesis of (4,5-difluoropyridin-2-yl)(4-((2-((6-(trifluoromethoxy)pyridin-3-yl)amino)pyrimidin-4-yl)(trifluoromethyl)amino)piperidin-1-yl)methanone

Synthesized by using general procedure C for Amide coupling:

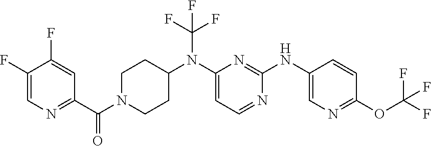 [SMILES: O=C(c1cc(F)c(F)cn1)N1CCC(N(c2ccnc(Nc3ccc(OC(F)(F)F)nc3)n2)C(F)(F)F)CC1]

Isolated (75 mg, 0.193 mmol, yield 37.5%) as Off-white solid.

LCMS: 99.92%, RT: 1.918 min, m/z=564.2 [M+H]⁺.

LCMS Method: Method 9

HPLC PURITY: 99.81%, RT: 7.341 min.

1H-NMR (400 MHz, DMSO-d6): δ 9.96 (s, 1H), 8.81 (d, J=2.40 Hz, 1H), 8.79 (d, J=2.40 Hz, 1H), 8.40 (d, J=5.60 Hz, 1H), 8.28 (dd, J=2.80, 8.80 Hz, 1H), 7.85 (dd, J=6.40, 10.80 Hz, 1H), 7.30 (d, J=8.80 Hz, 1H), 6.64-6.62 (m, 1H), 4.69-4.62 (m, 2H), 3.81 (d, J=13.60 Hz, 1H), 3.15 (t, J=11.60 Hz, 1H), 2.88 (t, J=10.40 Hz, 1H), 2.07-1.85 (m, 4H),

19-NMR (376 MHz, DMSO-d6): δ −50.503 (3F), −55.281 (3F), −126.741 to −126.686 (1F), −150.861 to −150.791 (1F).

### Synthesis of Example A621

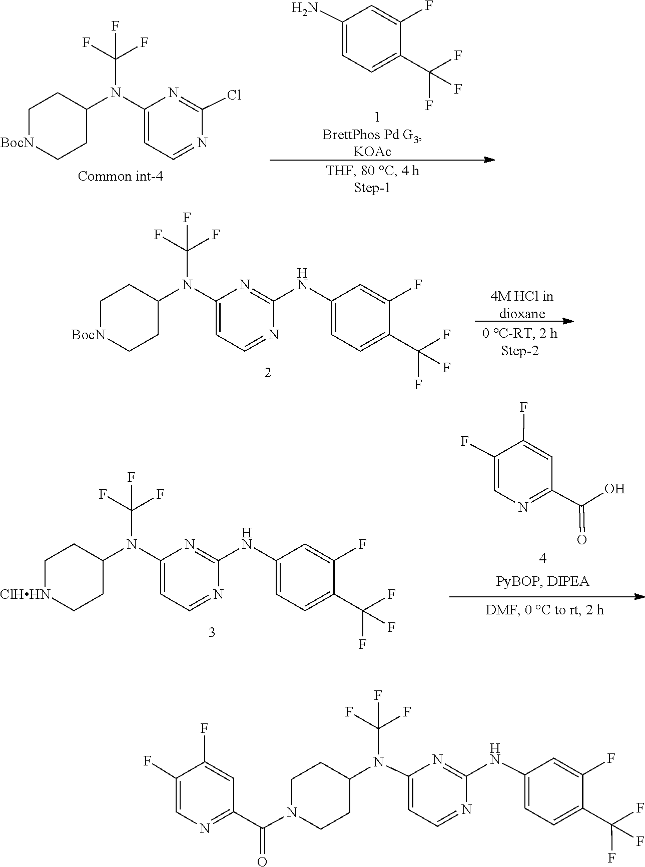

### Synthesis of tert-butyl 4-((2-((3-fluoro-4-(trifluoromethyl)phenyl)amino)pyrimidin-4-yl)(trifluoromethyl)amino)piperidine-1-carboxylate (2)

### Synthesized by Using General Procedure a for Buchwald Coupling:

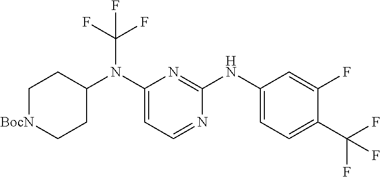 [SMILES: CC(C)(C)OC(=O)N1CCC(N(c2ccnc(Nc3ccc(C(F)(F)F)c(F)c3)n2)C(F)(F)F)CC1]

Isolated (340 mg, 0.607 mmol, 66.7% yield) as pale yellow solid.

LCMS: 93.49%, RT: 2.137 min, m/z=522.2 [M−H]⁺.

### Synthesis of N₂-(3-fluoro-4-(trifluoromethyl)phenyl)-N4-(piperidin-4-yl)-N4-(trifluoromethyl)pyrimidine-2,4-diamine hydrochloride (3)

### Synthesized by Using General Procedure B for Boc-Deprotection:

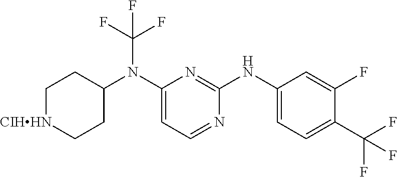

Crude (300 mg, 92% yield) is off white solid.

LCMS: 95.44%, RT: 1.00 min, m/z=424.2 [M+H]⁺.

### Synthesis of (4,5-difluoropyridin-2-yl)(4-((2-((3-fluoro-4-(trifluoromethyl)phenyl)amino)pyrimidin-4-yl)(trifluoromethyl)amino)piperidin-1-yl)methanone

### Synthesized by Using General Procedure C for Amide Coupling:

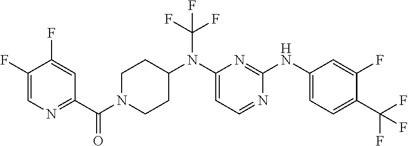 [SMILES: O=C(c1cc(F)c(F)cn1)N1CCC(N(c2ccnc(Nc3ccc(C(F)(F)F)c(F)c3)n2)C(F)(F)F)CC1]

Isolated (110 mg, 0.193 mmol, 32.3% yield) as Off-white solid.

LCMS: 99.652%, RT: 1.994 min, m/z=565.2 [M+H]⁺.

LCMS Method: Method 9

HPLC: 97.754%, RT: 7.848 min.

1H-NMR (400 MHz, DMSO-d6): δ 10.31 (s, 1H), 8.82-8.79 (m, 1H), 8.47 (d, J=5.60 Hz, 1H), 8.05 (d, J=14.40 Hz, 1H), 7.88-7.84 (m, 1H), 7.70-7.66 (m, 1H), 7.57 (d, J=9.20 Hz, 1H), 6.72 (d, J=4.40 Hz, 1H), 4.72-4.63 (m, 2H), 3.84 (d, J=13.20 Hz, 1H), 3.22-3.16 (m, 1H), 2.95-2.90 (m, 1H), 2.11 (d, J=12.40 Hz, 1H), 2.00-1.93 (m, 3H), 19-NMR (376 MHz, DMSO-d6): δ −50.571 (3F), −58.635 to −58.667 (3F), −114.718 to −114.809 (1F), −126.632 to −126.743 (1F), −150.748 to −150.822 (1F).

### Synthesis of Example A614

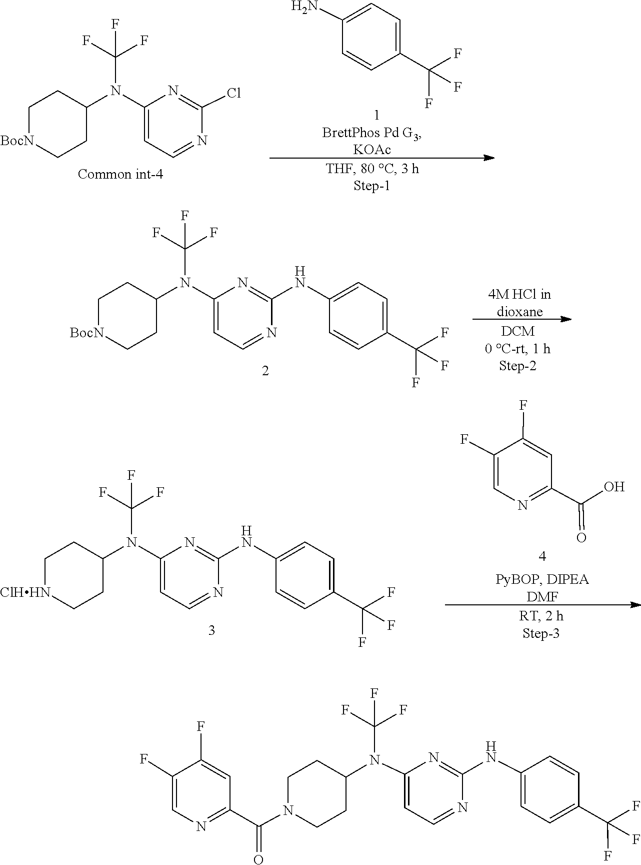

### Synthesis of tert-butyl 4-((trifluoromethyl)(2-((4-(trifluoromethyl)phenyl)amino)pyrimidin-4-yl)amino)piperidine-1-carboxylate (2)

### Synthesized by Using General Procedure a for Buchwald Coupling:

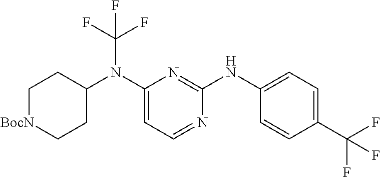 [SMILES: CC(C)(C)OC(=O)N1CCC(N(c2ccnc(Nc3ccc(C(F)(F)F)cc3)n2)C(F)(F)F)CC1]

Isolated (70 mg, 0.103 mmol, 78% yield) as brown solid.

LCMS: 74.16%, RT: 2.82 min, m/z=506.1 [M+H]⁺.

### Synthesis of N4-(piperidin-4-yl)-N4-(trifluoromethyl)-N2-(4-(trifluoromethyl)phenyl)pyrimidine-2,4-diamine hydrochloride (3)

### Synthesized by Using General Procedure B for Boc-Deprotection:

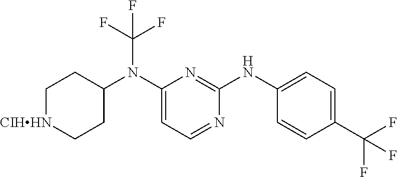

Crude (900 mg, 1.912 mmol, 97% yield) as Off white solid.

LCMS: 93.84%, RT: 1.71 min, m/z=406.2 [M+H]⁺.

### Synthesis of (5,6-difluoropyridin-3-yl)(4-((trifluoromethyl)(2-((4-(trifluoromethyl)phenyl)amino)pyrimidin-4-yl)amino)piperidin-1-yl)methanone

### Synthesized by Using General Procedure C for Amide Coupling:

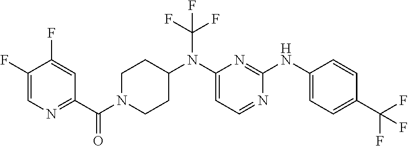 [SMILES: O=C(c1cc(F)c(F)cn1)N1CCC(N(c2ccnc(Nc3ccc(C(F)(F)F)cc3)n2)C(F)(F)F)CC1]

Isolated (145.25 mg, 0.263 mmol, 53.4% yield) as off white solid.

LCMS: 95.1% RT: 2.57 min, m/z=547.1 [M+H]⁺.

LCMS Method: Method 9

HPLC: 99.9%. RT: 12.16 min.

1H-NMR (400 MHz, DMSO-d6): δ 10.06 (s, 1H), 8.42 (d, J=5.60 Hz, 1H), 8.21-8-14 (m, 2H), 7.93 (d, J=8.40 Hz, 2H), 7.66 (d, J=8.80 Hz, 2H), 6.54 (q, J=1.6 Hz, 1H), 4.64 (m, 2H), 3.71 (m, 1H), 3.33-3.23 (m, 1H), 2.90 (m, 1H), 2.04-1.88 (m, 1H).

19F-NMR (376 MHz, DMSO-d6): δ −50.72 (s, 3F), −59.877 (s, 3F), −87.25 to 87.35 (q, 1F), −139.88 to 139.98 (q, 1F).

### Synthesis of Example A618

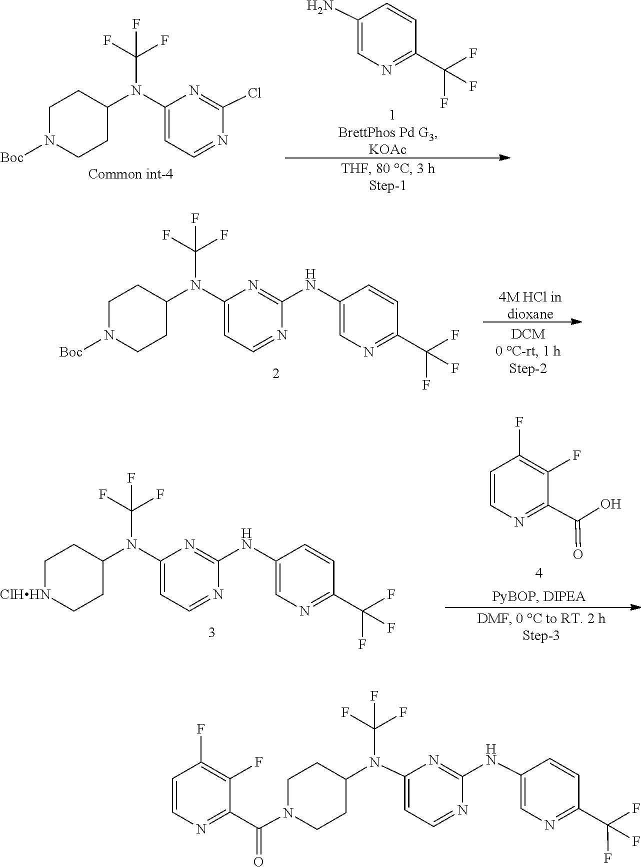

### Synthesis of tert-butyl 4-((trifluoromethyl)(2-((6-(trifluoromethyl)pyridin-3-yl)amino)pyrimidin-4-yl)amino)piperidine-1-carboxylate (2)

### Synthesized by Using General Procedure a for Buchwald Coupling:

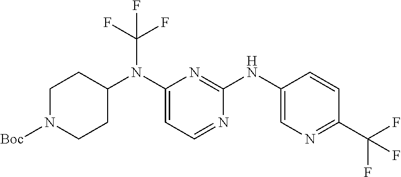 [SMILES: CC(C)(C)OC(=O)N1CCC(N(c2ccnc(Nc3ccc(C(F)(F)F)nc3)n2)C(F)(F)F)CC1]

Isolated (350 mg, 0.679 mmol, 74.6% yield) as pale yellow solid.

LCMS: 98.22%, RT: 2.603 min, m/z=505.3 [M−H]⁺.

### Synthesis of N4-(piperidin-4-yl)-N4-(trifluoromethyl)-N2-(6-(trifluoromethyl)pyridin-3-yl)pyrimidine-2,4-diamine hydrochloride (3)

### Synthesized by Using General Procedure B for Boc-Deprotection:

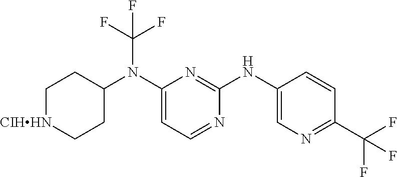

Crude (300 mg, 90% yield) as off white solid.

LCMS: 95%, RT:1.00 min, m/z=407.3 [M+H]⁺.

### Synthesis of (3,4-difluoropyridin-2-yl)(4-((trifluoromethyl)(2-((6-(trifluoromethyl)pyridin-3-yl)amino)pyrimidin-4-yl)amino)piperidin-1-yl)methanone

### Synthesized by Using General Procedure C for Amide Coupling:

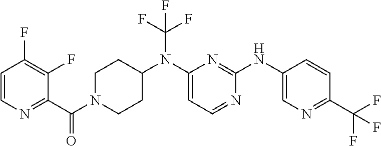 [SMILES: O=C(c1nccc(F)c1F)N1CCC(N(c2ccnc(Nc3ccc(C(F)(F)F)nc3)n2)C(F)(F)F)CC1]

Isolated (135.8 mg, 0.247 mmol, 39.3% yield) as Off-white solid.

LCMS:99.39%; RT: 2.613 min, m/z=548.1 [M+H]⁺.

LCMS Method: Method 3

HPLC: HPLC-99.60%; RT: 6.974 min

¹H-NMR (400 MHz, DMSO-d6): δ 10.26 (s, 1H), 9.01 (d, J=2.0 Hz, 1H), 8.51-8.46 (m, 2H), 8.40 (d, J=8.4 Hz, 1H), 7.86 (d, J=8.4 Hz, 1H), 7.71 (q, J=6.0 Hz, 1H), 6.72-6.71 (m, 1H), 4.68-4.65 (m, 2H), 3.54 (d, J=13.2 Hz, 1H), 3.26-3.19 (m, 1H), 2.96 (t, J=10.4 Hz, 1H), 2.13-2.08 (m, 1H), 1.97-1.91 (m, 3H).

¹⁹F-NMR (376 MHz, DMSO-d6): δ −50.553 (3F), −65.309 (3F), −126.412 (1F), −151.897 (1F).

### Synthesis of Example A613

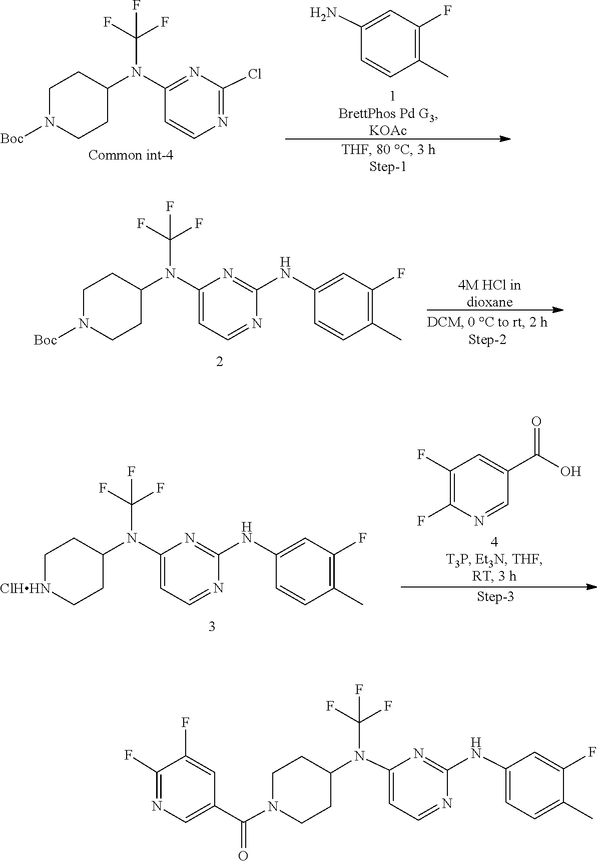

### Synthesis of tert-butyl 4-((2-((3-fluoro-4-methylphenyl)amino)pyrimidin-4-yl)(trifluoromethyl)amino)piperidine-1-carboxylate (2)

### Synthesized by Using General Procedure a for Buchwald Coupling:

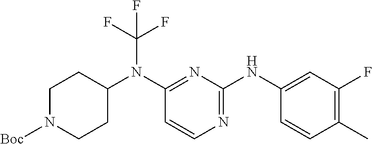 [SMILES: Cc1ccc(Nc2nccc(N(C3CCN(C(=O)OC(C)(C)C)CC3)C(F)(F)F)n2)cc1F]

Isolated (3.2 g, 6.82 mmol, 87% yield) as pale yellow solid.

LCMS: 95.91%, RT: 2.92 min, m/z=470.2 (M+H)⁺.

### Synthesis of N₂-(3-fluoro-4-methylphenyl)-N4-(piperidin-4-yl)-N4-(trifluoromethyl)pyrimidine-2,4-diamine hydrochloride (3)

### Synthesized by Using General Procedure B for Boc-Deprotection:

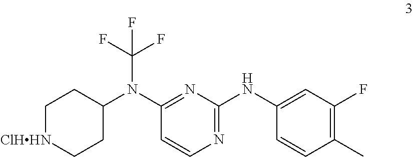

Crude (1.6 g, 4.20 mmol, 99% yield) as yellow solid.

LCMS: 96.90%, RT: 1.477 min, m/z=370.2 (M+H)⁺.

### Synthesis of (5,6-difluoropyridin-3-yl)(4-((2-((3-fluoro-4-methylphenyl)amino)pyrimidin-4-yl)(trifluoromethyl)amino)piperidin-1-yl)methanone

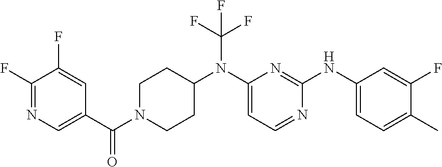 [SMILES: Cc1ccc(Nc2nccc(N(C3CCN(C(=O)c4cnc(F)c(F)c4)CC3)C(F)(F)F)n2)cc1F]

To a stirred solution of 5,6-difluoronicotinic acid (141 mg, 0.887 mmol) in THF (10 mL), was added propanephosphonic acid anhydride 50% solution in DMF (706 mg, 1.109 mmol), N₂-(3-fluoro-4-methylphenyl)-N4-(piperidin-4-yl)-N4-(trifluoromethyl)pyrimidine-2,4-diamine hydrochloride (300 mg, 0.739 mmol) and triethylamine (224 mg, 2.218 mmol) at 25 °C. The resulting mixture was stirred at 25 °C for 1 h. The progress of reaction was monitored by LCMS. Upon completion, the reaction mixture was diluted with water and extracted with ethyl acetate. The combined organic layers were washed with ice-cold water, dried over Na₂SO₄, concentrated under reduced pressure to get crude. The crude compound was purified by reverse phase column chromatography by using a C18 column (120 g) and eluted with 0.1% formic acid in water and acetonitrile to afford (5,6-difluoropyridin-3-yl)(4-((2-((3-fluoro-4-methylphenyl)amino)pyrimidin-4-yl)(trifluoromethyl)amino)piperidin-1-yl)methanone (80 mg, 0.155 mmol, 20.91% yield) as an off white solid.

LCMS: 98.62%, RT: 2.384 min, m/z=511.2 [M+H]⁺.

LCMS Method: Method 8

HPLC: 98.13%. RT: 11.67 min.

1H-NMR (400 MHz, DMSO-d6): δ 9.76 (s, 1H), 8.37 (d, J=5.60 Hz, 1H), 8.21-8.14 (m, 2H), 7.67 (d, J=12.80 Hz, 1H), 7.33 (dd, J=1.60, 8.20 Hz, 1H), 7.18 (t, J=8.80 Hz, 1H), 6.56 (t, J=1.60 Hz, 1H), 4.65 (s, 2H), 3.69 (s, 1H), 3.34 (s, 1H), 2.92-2.89 (m, 1H), 2.17 (s, 3H), 2.08-1.87 (m, 4H),

19F-NMR (376 MHz, DMSO-d6): −50.624 (3F), −87.236 to −87.336 (1F), −116.68 to −116.742 (1F), −139.887 to −139.964 (1F).

### Biological Activity

### Example: PIKfyve Protein Expression and Purification

Full length wild type PIKfyve (aa 1-2098; UniProt ID: Q9Y2I7-1) construct with N-terminal 3×FLAG and C-terminal His tag was cloned into pcDNA3.1 vector and transfected into Expi293F cells (Thermo, Cat. No. A14527). Protein was expressed for 72 hours at 30 °C after transfection. The cells were harvested by centrifugation and flash-frozen for storage. Frozen cell pellets were re-suspended in ice-cold lysis buffer (50 mM HEPES, 500 mM NaCl, 10% Glycerol, 0.1% Tween 20, 1 mM TCEP, 1/200 (v/v) Halt™ protease inhibitor cocktail (Thermo, Cat. No. 78429), 0.1 mg Benzonase/1 L culture, pH 8.0) and transferred into a Dounce homogenizer and lysed with 10 strokes. The supernatant after centrifugation was incubated with Anti-FLAG-G1 affinity resin (GenScript, Cat. No. L00432) for 1.5 hours. Then, the resin was washed 3 times with 10× bed volumes of wash buffer (50 mM HEPES, 300 mM NaCl, 10% Glycerol, 1 mM TCEP, pH 8.0) and eluted 3 times with 2× bed volume of elution buffer (50 mM HEPES, 300 mM NaCl, 10% Glycerol, 1 mM TCEP, 300 μg/mL FLAG peptide [GenScript, Cat. No. RP10586], pH 8.0). The elution fractions were pooled and further purified using a HiLoad 16/600 Superose™ 6 pg (Cytiva, Cat. No. 29323952) gel filtration column pre-equilibrated with gel filtration buffer (50 mM HEPES, 600 mM NaCl, 5% Glycerol, 0.5 mM TCEP, pH 8.0). Peak fractions of interest were collected and flash frozen in liquid nitrogen for storage at −80 °C. Quality control after one freeze-thaw cycle was performed to confirm protein was a monodisperse sample with no indication of aggregation or oligomerization using a Superose™ 6 Increase 5/150 GL (Cytiva, Cat. No. 29091597) gel filtration column pre-equilibrated with gel filtration buffer.

### Enzymatic Assay for Inhibition of PIKfyve

The inhibition of Recombinant PIKfyve protein [hPIKfyve(1-2098) or commercial hPIKfyve (Carna, cat no. 11-118)] was measured using ADP-Glo kinase-assay kit (Promega). 300 nl of compounds (dissolved in DMSO) in serial dilution were transferred to 384-well Optiplates (Perkin Elmer) using Echo 650 series liquid handler (Beckman Coulter). 5 μl of Recombinant PIkfyve in assay buffer (25 mM HEPES pH 7.5, 10 mM MgCl2, 1 μM CaCl₂), 2 mM DTT, 0.05% BSA, and 0.002% Triton-X100) is added to the compounds and preincubated for 120 minutes at room temperature. To initiate the reaction, 5 ul of ATP and PI(3)P:PS substrate are added. For hPIKfyve(1-2098) the final concentration of recombinant protein, ATP, and Substrate are 5 nM, 20 uM, and 62.5:500 uM, respectively while for hPIKfyve (Carna), the final concentration of recombinant protein, ATP, and Substrate are 5 nM, 50 uM, and 62.5:500 uM, respectively. The plate was incubated for 90 minutes at room temperature then 10 ul of the ADP-GLO™ reagent was added and incubated for 60 minutes. 20 ul of the kinase detection reagent was added and incubated for an additional 30 minutes then luminescence (LUM) was detected using the Pherastar microplate reader (BMG Labtech).

The enzyme activity was normalized using control wells containing negative control (0% inhibition) of 3% DMSO or positive control (100% inhibition) of 10 μM YM-201636 (MedChemExpress).

% Inhibition=100×[1−(LUMcmpd−LUMpos)/(LUMneg−LUMpos)]

Where LUMcmpd, LUMpos, and LUMneg are the relative luminescence units of compound, YM-201636, and DMSO treated wells respectively. The % inhibition values were plotted as a function of compound concentration and the IC50 values of compounds were determined using a four-parameter logistic fit Y=Min+[(Max−Min)/(1+(X/IC50) Hill slope)] or three-parameter fit if Max or Min of curves were fixed to 10000 or 000 respectively.

Table 6 shows the activity of exemplar compounds against the PIKfyve enzyme using the ADP-Glo assay format described above expressed as the concentration required to inhibit 500% of the enzymatic activity: IC50=A≤10 nM<B≤100 nM<C≤1000 nM

| Cmpd | IC50 | Cmp | IC50 | Cmp | IC50 | Cmp | IC50 |
|---|---|---|---|---|---|---|---|
| A1 | A | A35 | C | A70 | B | A105 | A |
| A2 | A | A36 | B | A71 | A | A106 | B |
| A3 | A | A37 | C | A72 | B | A107 | B |
| A4 | B | A38 | C | A73 | B | A108 | B |
| A5 | A | A39 | C | A74 | B | A109 | A |
| A6 | B | A40 | B | A75 | B | A110 | A |
| A7 | A | A41 | C | A76 | B | A111 | B |
| A8 | A | A42 | B | A77 | B | A112 | A |
| A9 | A | A43 | B | A78 | B | A113 | B |
| A10 | B | A44 | B | A79 | A | A114 | A |
| A11 | A | A45 | C | A80 | B | A115 | B |
| A12 | A | A46 | C | A81 | C | A116 | C |
| A13 | A | A47 | B | A82 | A | A117 | B |
| A14 | C | A48 | C | A83 | B | A118 | B |
| A15 | A | A49 | C | A84 | C | A119 | A |
| A16 | C | A50 | A | A85 | C | A121 | C |
| A17 | C | A51 | A | A86 | B | A122 | A |
| A18 | A | A52 | B | A87 | B | A123 | C |
| A19 | C | A53 | C | A88 | B | A124 | A |
| A20 | A | A54 | B | A89 | A | A125 | C |
| A21 | B | A55 | A | A91 | C | A126 | A |
| A22 | B | A56 | A | A95 | A | A127 | A |
| A23 | C | A57 | A | A96 | A | A128 | A |
| A24 | A | A58 | A | A97 | A | A129 | A |
| A25 | A | A59 | A | A98 | A | A130 | A |
| A26 | A | A60 | A | A99 | A | A131 | B |
| A27 | C | A62 | A | A100 | A | A132 | B |
| A28 | C | A63 | B | A101 | A | A133 | B |
| A29 | A | A64 | B | A102 | A | A134 | A |
| A30 | C | A65 | A | A103 | A | | |
| A31 | A | A66 | B | A104 | C | | |
| A32 | A | A67 | C | | | | |
| A33 | A | A68 | B | | | | |
| A34 | C | A69 | B | | | | |
| Cmp | IC50 | Cmp | IC50 | Cmp | IC50 | Cmp | IC50 |
| A135 | B | A235 | A | A330 | A | A425 | A |
| A137 | C | A236 | C | A331 | A | A426 | A |
| A138 | C | A237 | C | A332 | A | A427 | A |
| A139 | A | A238 | A | A333 | A | A428 | A |
| A140 | B | A239 | B | A334 | A | A429 | A |
| A141 | C | A240 | C | A335 | C | A430 | A |
| A142 | B | A241 | C | A336 | B | A431 | A |
| A143 | C | A242 | C | A337 | B | A432 | A |
| A144 | A | A243 | B | A338 | B | A433 | A |
| A145 | A | A244 | C | A339 | B | A434 | B |
| A146 | A | A245 | B | A341 | C | A435 | A |
| A147 | B | A246 | B | A342 | C | A436 | A |
| A148 | C | A247 | B | A343 | B | A437 | A |
| A149 | C | A248 | B | A344 | A | A438 | A |
| A150 | C | A249 | A | A345 | A | A439 | A |
| A151 | A | A250 | A | A346 | A | A440 | C |
| A152 | B | A251 | A | A347 | C | A441 | A |
| A153 | A | A252 | C | A348 | B | A442 | A |
| A154 | A | A253 | C | A349 | A | A443 | A |
| A155 | A | A254 | C | A351 | C | A444 | B |
| A156 | B | A255 | C | A352 | B | A445 | A |
| A157 | B | A256 | C | A353 | A | A446 | A |
| A158 | A | A257 | B | A354 | A | A447 | A |
| A159 | B | A258 | A | A355 | B | A448 | A |
| A160 | A | A259 | A | A356 | B | A449 | A |
| A161 | A | A260 | A | A357 | A | A450 | A |
| A162 | A | A261 | B | A358 | B | A451 | C |
| A163 | A | A262 | C | A359 | A | A452 | A |
| A164 | B | A263 | A | A360 | B | A453 | A |
| A165 | C | A264 | A | A361 | B | A454 | A |
| A166 | C | A265 | A | A362 | B | A455 | A |
| A167 | C | A266 | A | A363 | A | A456 | A |
| A168 | A | A267 | A | A364 | A | A457 | A |
| A169 | C | A268 | B | A365 | A | A458 | A |
| A170 | B | A269 | A | A366 | A | A459 | A |
| A171 | A | A270 | A | A367 | A | A460 | A |
| A172 | C | A271 | A | A368 | A | A461 | A |
| A173 | A | A272 | A | A369 | A | A462 | B |
| A175 | C | A273 | B | A370 | A | A463 | B |
| A176 | A | A274 | C | A371 | A | A464 | A |
| A177 | B | A275 | B | A372 | A | A465 | A |
| A178 | B | A276 | C | A373 | A | A466 | A |
| A179 | B | A277 | B | A374 | A | A467 | A |
| A180 | C | A278 | B | A375 | A | A468 | B |
| A181 | A | A279 | C | A376 | A | A469 | B |
| A182 | A | A280 | A | A377 | A | A470 | A |
| A183 | C | A281 | C | A378 | A | A471 | B |
| A184 | B | A282 | C | A379 | A | A472 | B |
| A185 | A | A283 | B | A380 | A | A473 | A |
| A186 | A | A284 | B | A381 | A | A474 | A |
| A187 | A | A285 | B | A382 | A | A475 | A |
| A188 | C | A286 | B | A383 | A | A476 | A |
| A190 | A | A287 | A | A384 | A | A477 | A |
| A191 | A | A288 | B | A385 | A | A478 | B |
| A193 | A | A289 | A | A386 | A | A479 | A |
| A194 | A | A290 | A | A387 | A | A480 | A |
| A195 | A | A291 | C | A388 | C | A481 | A |
| A196 | C | A292 | B | A390 | C | A482 | A |
| A197 | A | A293 | B | A391 | A | A483 | A |
| A198 | A | A294 | B | A392 | A | A484 | A |
| A199 | C | A295 | A | A393 | A | A485 | A |
| A200 | B | A296 | A | A394 | C | A486 | A |
| A201 | B | A297 | B | A395 | A | A487 | A |
| A202 | C | A298 | B | A396 | C | A488 | A |
| A203 | C | A300 | A | A397 | A | A489 | A |
| A205 | C | A301 | A | A398 | A | A490 | A |
| A207 | B | A302 | A | A399 | A | A491 | A |
| A208 | B | A303 | A | A400 | A | A492 | A |
| A209 | B | A304 | A | A401 | A | A493 | A |
| A211 | A | A305 | A | A402 | A | A494 | A |
| A213 | B | A306 | A | A403 | A | A495 | A |
| A214 | A | A307 | C | A404 | A | A496 | C |
| A215 | A | A308 | B | A405 | A | A497 | A |
| A216 | B | A309 | A | A406 | A | A498 | A |
| A217 | C | A310 | A | A407 | A | A499 | C |
| A218 | C | A311 | A | A408 | A | A500 | C |
| A219 | A | A312 | A | A409 | A | A501 | A |
| A220 | A | A313 | A | A410 | A | A503 | A |
| A221 | B | A314 | A | A411 | A | A504 | A |
| A222 | C | A315 | C | A412 | A | A505 | A |
| A223 | B | A316 | A | A413 | A | A506 | A |
| A224 | C | A317 | A | A414 | A | A507 | A |
| A225 | C | A318 | A | A415 | A | A508 | A |
| A226 | C | A319 | A | A416 | A | A509 | C |
| A227 | B | A321 | B | A417 | A | A510 | A |
| A228 | C | A322 | A | A418 | A | A511 | A |
| A229 | C | A323 | C | A419 | B | A512 | B |
| A230 | C | A325 | B | A420 | A | A513 | A |
| A231 | B | A326 | C | A421 | A | A514 | C |
| A232 | B | A327 | B | A422 | A | A515 | A |
| A233 | B | A328 | C | A423 | B | A516 | B |
| A234 | B | A329 | A | A424 | A | A517 | B |
| | | | | | | A518 | C |
| A519 | B | A520 | C | A521 | A | A522 | C |
| A566 | B | A567 | A | A568 | A | A569 | A |
| A570 | A | A571 | B | A572 | A | A577 | B |
| A578 | A | A579 | A | A580 | A | A581 | A |
| A582 | A | A594 | A | A606 | A | A618 | B |
| A583 | B | A595 | A | A607 | B | A619 | B |
| A584 | A | A596 | A | A608 | A | A620 | C |
| A585 | B | A597 | A | A609 | B | A621 | B |
| A586 | A | A598 | C | A610 | B | A622 | B |
| A587 | A | A599 | A | A611 | A | A623 | B |
| A588 | B | A600 | A | A612 | A | A624 | B |
| A589 | A | A601 | B | A613 | B | A625 | B |
| A590 | A | A602 | A | A614 | B | A626 | A |
| A591 | A | A603 | B | A615 | A | | |
| A592 | A | A604 | A | A616 | C | | |
| A593 | A | A605 | A | A617 | B | | |

While this invention has been particularly shown and described with references to preferred embodiments thereof, it will be understood by those skilled in the art that various changes in form and details may be made therein without departing from the scope of the invention encompassed by the appended claims.

1. A compound of formula (I): 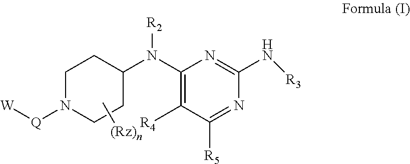
or a pharmaceutically acceptable salt thereof,
wherein:
R₂ is independently selected from hydrogen, D, halogen, a substituted or unsubstituted alkyl, a substituted or unsubstituted alkoxy, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, a substituted or unsubstituted heteroaryl, and a substituted or unsubstituted heteroalkyl;
R₃ is selected from substituted or unsubstituted aryl, or substituted or unsubstituted heteroaryl, wherein the aryl or heteroaryl can be optionally fused or bridged;
R₄ and R₅ are each independently selected from hydrogen, D, halogen, substituted or unsubstituted alkyl, substituted or unsubstituted alkoxy, substituted or unsubstituted fluoroalkyl, and —CN; or R₄ and R₅ can be taken together with the carbons to which they are attached to form a substituted or unsubstituted 4, 5, 6, 7, or 8 membered ring;
R₂ and R₄ can be taken together to form a substituted or unsubstituted hetercycle, including a lactam;
Q is selected from absent, —C(O)—, —CR_{B}R_{C}—, —S(O)₂—, —S(O)—, —NR_{A}—, —C(O)O—, —NR_{A}C(O)—, —C(O)—CR_{B}R_{C}—, and —C(O)NR_{A}—; R_{B} and R_{C} are independently H, halogen, a substituted or unsubstituted alkyl, substituted or unsubstituted alkenyl, substituted or unsubstituted alkynyl, or substituted or unsubstituted alkoxy, and R_{A} is hydrogen, substituted or unsubstituted alkyl, substituted or unsubstituted haloalkyl, substituted or unsubstituted heteroalkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocycloalkyl; or R_{B} and R_{C} can be taken together with the atom to which they are attached to form a substituted or unsubstituted 4, 5, 6, 7, or 8 membered ring;
W is a substituted or unsubstituted alkyl, a substituted or unsubstituted alkenyl, a substituted or unsubstituted aryl or a substituted or unsubstituted heteroaryl;
Each R_{Z} is independently selected from D, halogen, —CN, a substituted or unsubstituted alkyl, a substituted or unsubstituted alkoxy, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, a substituted or unsubstituted heteroaryl, and a substituted or unsubstituted heteroalkyl; alternatively, (R_{Z})ₙ, together with the heterocyclic ring to which R_{Z} is attached, forms a bridged, fused or spiro ring structure; and
n is 0, 1, 2, 3, or 4.

2. The compound of claim 1, wherein R₂ is hydrogen, a substituted or unsubstituted alkyl, a substituted or unsubstituted alkoxyl, a substituted or unsubstituted heterocycloalkyl, or a substituted or unsubstituted heteroalkyl.

3. The compound of claim 1, wherein R₂ is H, a C₁₋₆ alkyl, a substituted or unsubstituted heterocycloalkyl (e.g., oxetane, azetidine), —C₁₋₆ alkyl-OR_{A}, —C₁₋₆ alkyl-NR_{B}R_{C}, or —C₁₋₆ alkyl-C(O)NR_{B}R_{C}; wherein R_{A} is hydrogen, C₁₋₄ alkyl, C₁₋₄ fluoroalkyl, a C₃₋₈ cycloalkyl, a heterocycloalkyl (e.g., oxetane, azetidine), an aryl; R_{B} and R_{C} are independently selected from H, C₁₋₄ alkyl, C₁₋₄ fluoroalkyl; or R_{B} and R_{C} can be taken together with the nitrogen to which they are attached to form a substituted or unsubstituted 4, 5, 6, 7, or 8 membered ring.

4. The compound of claim 1, wherein R₂ is —C₁₋₄ alkyl-OR_{A}; wherein R_{A} is H, —CH₃, —CF₃, —C₂H₅, —C₃H₇, —C(CH₃)₃, —CHF₂, or a C₃₋₆ cycloalkyl.

5. The compound of claim 1, wherein R₂ is —C₁₋₆ alkyl-NR_{B}R_{C}, R_{B} and R_{C} are independently selected from H, C₁₋₄ alkyl, C₁₋₄ fluoroalkyl, or R_{B} and R_{C} forms a substituted or unsubstituted azetidine ring, wherein the substituents are selected from C₁₋₄ alkyl and halogen.

6. The compound of claim 1, wherein R₃ is a substituted or unsubstituted aryl, or a substituted or unsubstituted heteroaryl.

7. The compound of claim 6, wherein R₃ is phenyl or substituted phenyl.

8. The compound of claim 1, wherein R₃ is selected from one of the following by removal of a hydrogen atom: 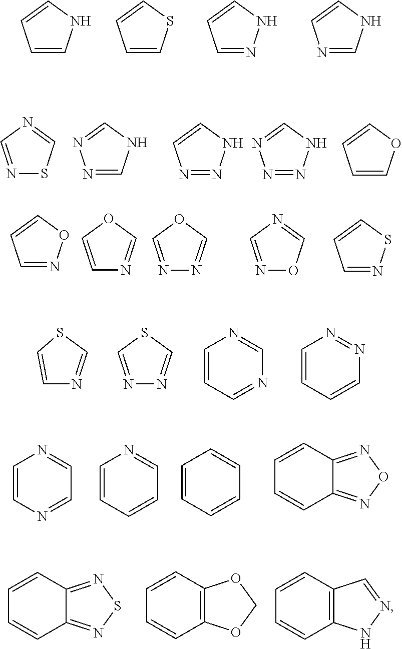
wherein each of these groups is substituted or unsubstituted.

9. The compound of claim 8, wherein R₃ is substituted with one to three groups selected from halogen (preferably F, Cl), C₁₋₄ alkyl (preferably methyl or ethyl), C₁₋₄ fluoroalkyl, —OR_{A}, —NR_{B}R_{C}, —NR_{A}C(O)R_{B}, —C(O)NR_{B}R_{C}, a C₃₋₈ cycloalkyl, a heterocycloalkyl; wherein R_{A}, R_{B}, and R_{C} are independently selected from H and a C₁₋₄ alkyl.

10. The compound of claim 1, wherein R₄ is selected from hydrogen or halogen.

11. The compound of claim 1, wherein R₅ is selected from hydrogen or halogen.

12. The compound of claim 1, wherein Q is absent, —C(O)—, —S(O)₂—, —S(O)—.

13. The compound of claim 1, wherein W is a substituted or unsubstituted aryl or a substituted or unsubstituted heteroaryl.

14. The compound of claim 13, wherein W is a substituted or unsubstituted phenyl, a substituted or unsubstituted pyridine, a substituted or unsubstituted pyridazine, a substituted or unsubstituted pyrimidine, a substituted or unsubstituted pyrazine, a substituted or unsubstituted 1,2,4-triazine, or a substituted or unsubstituted 1,3,5-triazine.

15. The compound of claim 1, wherein W is a substituted phenyl, substituted by one to five substituents selected from halogen (preferably F, Cl), C₁₋₄ alkyl (preferably methyl or ethyl), C₁₋₄ fluoroalkyl, —CF₃, —OCF₃, —CN, —CH═CH₂, —CONR_{D}R_{E}, —C₃₋₆ cycloalkyl (such as cyclopropyl), −3- to 6-membered heterocycloalkyl (such as oxetane), a fluoro-substituted —C₃₋₆ cycloalkyl (such as fluoro-substituted cyclopropyl), a fluoro-substituted 3- to 6-membered heterocycloalkyl (such as a fluoro-substituted oxetane); wherein R_{D} and R_{E} are independently H, C₁₋₄ alkyl, or C₁₋₄ fluoroalkyl.

16. The compound of claim 1, wherein W is a substituted pyridine, substituted by one to five substituents selected from halogen (preferably F, Cl), C₁₋₄ alkyl (preferably methyl or ethyl), C₁₋₄ fluoroalkyl, —CF₃, —OCF₃, —CN, —CH═CH₂, —CONR_{D}R_{E}, —C₃₋₆ cycloalkyl (such as cyclopropyl), -3- to 6-membered heterocycloalkyl (such as oxetane), a fluoro-substituted —C₃₋₆ cycloalkyl (such as fluoro-substituted cyclopropyl), a fluoro-substituted 3- to 6-membered heterocycloalkyl (such as a fluoro-substituted oxetane); wherein R_{D} and R_{E} are independently H, C₁₋₄ alkyl, or C₁₋₄ fluoroalkyl.

17. The compound of claim 1, wherein W is a substituted pyrimidine, substituted by one to five substituents selected from halogen (preferably F, Cl), C₁₋₄ alkyl (preferably methyl or ethyl), C₁₋₄ fluoroalkyl, —CF₃, —OCF₃, —CN, —CH═CH₂, —CONR_{D}R_{E}, —C₃₋₆ cycloalkyl (such as cyclopropyl), -3- to 6-membered heterocycloalkyl (such as oxetane), a fluoro-substituted —C₃₋₆ cycloalkyl (such as fluoro-substituted cyclopropyl), a fluoro-substituted 3- to 6-membered heterocycloalkyl (such as a fluoro-substituted oxetane); wherein R_{D} and R_{E} are independently H, C₁₋₄ alkyl, or C₁₋₄ fluoroalkyl.

18. The compound of claim 1, wherein each R_{Z} is independently selected from halogen, —CN, a substituted or unsubstituted alkyl, a substituted or unsubstituted alkoxy; and n is 1, 2, 3, or 4.

19. The compound of claim 1, wherein n is 0.

20. A compound selected from compound A1 through compound A644.
